(19) United States
(12) Patent Application Publication
(10) Pub. No.: US 2023/0046947 A1
(43) Pub. Date: Feb. 16, 2023

(54) REACTIVE CONJUGATES

(71) Applicant: Debiopharm Research & Manufacturing S.A., Martigny (CH)

(72) Inventors: Origéne Franz Nyanguile, Grimisuat (CH); Jean-Manuel Segura, Vevey (CH); Patrick Garrouste, Saxon (CH); Viktoriia Postupalenko, Sion (CH); Léo Marx, Martigny (CH); Frédéric Levy, Lausanne (CH)

(73) Assignee: Debiopharm Research & Manufacturing S.A., Martigny (CH)

(21) Appl. No.: 17/781,687

(22) PCT Filed: Dec. 3, 2020
(86) PCT No.: PCT/EP2020/084512

§ 371 Date: Jun. 1, 2022

(30) Foreign Application Priority Data
Dec. 3, 2019 (EP) PCT/EP2019/083542

(51) Int. Cl. A61K 51/10; A61K 47/68; C07K 1/04

(52) U.S. Cl. CPC A61K 51/1096; A61K 47/6889; A61K 47/6803; C07K 1/04; A61K 47/6811

(57) ABSTRACT

The present invention relates to compounds (reactive conjugates) for the chemical modification of therapeutic antibodies or proteins. The compounds enable the regioselective attachment of a payload to an antibody or antibody fragment in one single step, thereby producing a modified antibody or modified antibody fragment, which can be used for diagnosing, monitoring, imaging or treating disease.

### FIELD OF THE INVENTION

The present invention relates to compounds (hereinafter sometimes referred to as “reactive conjugates”) for the chemical modification of therapeutic antibodies. The compounds enable the regioselective attachment of a payload to an antibody or antibody fragment in one single step, thereby producing a modified antibody or modified antibody fragment, which can be used for diagnosing, monitoring, imaging or treating disease.

### BACKGROUND OF THE INVENTION

Traditional cancer treatments e.g. chemotherapy, can not only be extremely grueling (because of the severe side effects caused by their toxicity) but can also be extremely hit and miss, with treatments effective in one patient being completely ineffective in another. As a result, the development of new less toxic and/or more effective treatments is an ever-present need, as is the ability to monitor the effectiveness of a treatment e.g. enabling the distinction between “responder” and “non-responder” patients.

In response to these needs, a new class of therapeutic agents referred to as Antibody-drug-conjugates (ADCs) has emerged. ADCs harness the targeting power of antibodies e.g. Monoclonal antibodies (mAbs), to deliver a payload e.g. a cytotoxic agent or labelling agent, directly to a cancer cell. The specific targeting of cancer cells enables the therapeutic effects of payloads to be maximized, whilst toxic effects on healthy cells are minimized. Depending on the payload ADC's can fulfill a variety of roles e.g. diagnostic, monitoring and/or therapeutic.

ADCs can be prepared by a variety of methods. However, the majority of said methods lead to heterogeneous mixtures of chemically distinct ADCs having varying payload (drug) antibody ratios (DAR) and conjugation sites. This heterogeneity can complicate manufacturing resulting in high batch to batch variability and sometimes unpredictable safety and efficacy. In consequence, methods that can result in the preparation of homogeneous mixtures e.g. regioselective or site-specific conjugation methods, are of growing interest. Such methods can drastically increase the predictability of the DAR, and the payload (drug) conjugation site, and can serve to simplify the development and manufacturing of more defined ADCs products having more predictable safety or efficacy.

Several approaches have been developed for the regiospecific and site-specific conjugation of payloads to antibodies. However, often known approaches require the modification/engineering of the antibody e.g. through the incorporation of non-natural amino acids or through the modification of carbohydrate moieties. Such modifications may negatively affect the therapeutic efficacy/safety of a corresponding ADC e.g. because of undesirable effects with respect to activity, targeting, metabolism, and/or excretion of the antibody, as well as the immune response to the antibody. Other approaches involve multiple steps e.g. the approach set out in WO 2018/199337. Such multi-step approaches may be costly and/or laborious, making them less attractive or even unsuitable for applications where a quick and simple antibody modification process is desirable (e.g. for point-of-care diagnostic applications).

Accordingly, there is still a need to find alternative ways for the region or site-specific conjugation of payloads to antibodies or antibody fragments, in particular there is a need for ways that do not require the engineering of the antibody or antibody fragment therebefore. Further, there is a need to find ways of preparing antibody drug conjugates in as few steps as possible, and preferably in one single step.

In view of the foregoing, it is an object of the present invention to provide compounds (reactive conjugates), enabling the regioselective conjugation of a payload to an antibody or antibody fragment in one single step, without the need to engineer and/or modify the antibody or antibody fragment therebefore. It is a further object to provide kits comprising such compounds.

It is yet another object of the present invention to provide a method for producing a modified antibody or modified antibody fragment (e.g. ADCs), which can be used in a method of diagnosing, monitoring, imaging or treating disease.

### SUMMARY OF THE PRESENT INVENTION

The present invention provides a compound, which enables the regioselective attachment of a payload to an antibody (e.g. to a therapeutic antibody) or an antibody fragment that is optionally incorporated into an Fc-fusion protein. This regioselective attachment may be accomplished in one single step. The resulting modified antibody or modified antibody fragment (e.g. ADC or antibody-radionuclide conjugate) can be used in a method of diagnosing, monitoring, imaging or treating disease, in particular cancer.

The compound (reactive conjugate) of the present invention can be represented by the following formula (1):

P—Y—S—V (1)
wherein
P is a payload;
Y is a reactive moiety capable of reacting with the side chain of an amino acid e.g. lysine or cysteine, preferably a moiety capable of reacting with the side chain of lysine;
V is a vector capable of interacting with the fragment crystallizable (Fc) region of an antibody or fragment thereof, the antibody fragment being optionally incorporated into an Fc-fusion protein;
S is a spacer having a length Z, wherein Z is a length such that when the vector V interacts with the Fc region of an antibody or fragment thereof, the reactive moiety Y is able to react with the side chain of an amino acid residue on said antibody or antibody fragment.

The present invention also relates to a kit for the regioselective modification of an antibody or antibody fragment, the antibody fragment being optionally incorporated into an Fc-fusion protein, wherein said kit comprises the compound as described hereinbefore, optionally immobilized on a solid phase matrix (e.g. beads) and a buffer.

Furthermore, the present invention relates to a method for the regioselective modification of an antibody or antibody fragment, the antibody fragment being optionally incorporated into an Fc-fusion protein, wherein said method employs the compound described hereinbefore.

Additionally, the present invention relates to a modified antibody or modified antibody fragment (e.g. obtainable or obtained by the method described hereinbefore), the antibody fragment being optionally incorporated into an Fc-fusion protein, for use in a method of diagnosing, monitoring, imaging and/or treating disease, especially cancer.

The present invention, in particular, includes the following embodiments (“Items”):

1. Compound represented by the following formula (1):

P—Y—S—V (1)
wherein,
P is a payload;
Y is a reactive moiety capable of reacting with the side chain of an amino acid, preferably a moiety capable of reacting with the side chain of lysine;
V is a vector capable of interacting with the fragment crystallizable (Fc) region of an antibody or fragment thereof, said antibody fragment being optionally incorporated into an Fc-fusion protein;
S is a spacer having a length Z, wherein Z is a length such that when the vector V interacts with the Fc region of an antibody or fragment thereof, the reactive moiety Y is able to react with the side chain of an amino acid residue on said antibody or antibody fragment.

2. The compound of item 1, wherein the payload comprises a moiety selected from:
(i) a moiety selected from
   a labelling moiety which may include a radionuclide, preferably a chelating agent such as 1,4,7,10-tetraatacyclododecane-1,4,7,10-tetraacetic acid (DOTA), diethylenetriamine pentaacetic acid (DTPA), cyclohexyl diethylenetriamine pentaacetic acid (CH-X-DTPA), 3,6,9,15-Tetraazabicyclo[9.3.1]pentadeca-1(15), 11,13-triene-3,6,9-triacetic acid (PCTA) or desferrioxamine (DFO), wherein said chelating agent optionally chelates a radionuclide;
   a chromophore;
   a fluorophore such as fluorescein or rhodamine; and
   a labelling moiety containing a radionuclide such as ¹²⁵I, ¹²³I, ¹³¹I, ¹⁸F, ¹¹C, ¹⁵O, ¹⁸F, e.g. a moiety derived from 4-hydroxyphenylpropionate containing a radionuclide such as ¹²⁵I, ¹²³I or ¹³¹I;
(ii) a moiety selected from a moiety comprising a conjugation group including an optionally substituted conjugated diene; an optionally substituted tetrazine; an optionally substituted alkyne or azide; an optionally substituted dibenzocyclooctyne (DBCO); an optionally substituted trans-cyclooctene (TCO), an optionally substituted bicyclo[6.1.0]nonyne (BCN); an optionally substituted aldehyde; an optionally substituted ketone; and an optionally substituted hydrazine;
(iii) a moiety derived from a drug selected from
   an antineoplastic agent such as a DNA-alkylating agent e.g. duocarmycin;
   a topoisomerase inhibitor e.g. doxorubicin;
   an RNA-polymerase II inhibitor e.g. alpha-amanitin;
   a DNA cleaving agent e.g. calicheamicin;
   an antimitotic agent or microtubule disruptor e.g. a taxane an auristatin or a maytansinoid;
   an anti-metabolite;
   a kinase inhibitor such as ipatasertib;
   an immunomodulatory agent;
   an anti-infectious disease agent;
   and radioisotopes and/or pharmaceutically acceptable salts thereof;

3. The compound of item 1 or 2, wherein the payload is a chelating agent that optionally chelates a radionuclide, which chelating agent is preferably a moiety derived from DTPA, CH-X-DTPA, DFO, 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), DOTA, 1,4,7-triazacyclononane-1,4,7-triacetic acid (NOTA), ethylenediaminetetraacetic acid (EDTA), ethylenediaminediacetic acid, triethylenetetraminehexaacetic acid (TTNA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4, 11-diaceticacid (CB-TE2A), 2,2′,2″-(1,4,7,10-tetraazacyclododecane-1,4,7-triyl)triacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), PCTA, 2,2′,2″,2″′-(1,4,7,10-tetraazacyclotridecane-1,4,7,10-tetrayl)tetraacetic acid (TRITA), 2,2′,2″,2″′-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)triacetamide (TRITRAM), trans-N-dimethyl-cyclam, 2,2′,2″-(1,4,7-triazacyclononane-1,4,7-triyl)tracetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), or derivatives thereof; more preferably a moiety derived from DTPA, DOTA, DFO, NOTA, PCTA, CH-X-DTPA, NODAGA or DOTAGA.

4. The compound of item 2 or 3, wherein the radionuclide is selected from 124I, ¹³¹I, ⁸⁶Y, ⁹⁰Y, ¹⁷⁷Lu, ¹¹¹In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²Bi, ²¹³Bi, ⁷²As, ²¹¹At, ²²⁵Ac, ²²³Ra, ⁹⁷Ru, ¹⁴⁹Tb, ¹⁵²Tb, ¹⁶¹Tb, ^{99m}Tc, ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F, preferably from ⁸⁹Zr, ¹¹¹In, ⁶⁴Cu, ¹⁷⁷Lu, ⁶⁸Ga, ^{99m}Tc, ²⁰³Pb, ⁷²As, ⁵⁵Co, ⁹⁷Ru, ²⁰¹Ti, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, in particular ¹¹¹In.

5. The compound of item 1 or 2, wherein the payload is a moiety derived from exatecan, PNU-159682, amanitin, duocarmycin, auristatin, maytansine, tubulysin, calicheamicin, SN-38, taxol, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof.

6. The compound of any of items 1 to 5, wherein P is represented by the following formula (2):

P¹-L-*′ (₂)
wherein,
P¹ is a payload as defined in any of items 2 to 5;
L is a linker, preferably a linker comprising one or more atoms selected from carbon, nitrogen, oxygen, and sulfur, which is optionally cleavable;
*′ indicates covalent attachment to the reactive moiety (Y).

The compound of item 6, wherein the linker is selected from
(a1) an alkylene group having from 1 to 12 carbon atoms, preferably an alkylene group having from 2 to 6 carbon atoms such as a propylene group;
(b1) a polyalkylene oxide group with 2 or 3 carbon atoms having from 1 to 36 repeating units; preferably a group represented by the formula —NH—(CH₂CH₂O)ₙ₁—CH₂CH₂— wherein n1 is an integer of 0 to 35, e.g. 1 to 20;
(c1) a peptidic group having 2 to 12 amino acids.

8. The compound of any of items 1 to 7, wherein the reactive moiety is represented by the following formula (3a):

**—(F1-RC—F2)-* (3a)
wherein,
RC is a reactive center, preferably an electrophilic reactive center, and more preferably a group selected from C═O and C═S;
F1 is a single covalent bond, an atom, or a group of atoms; preferably an atom selected from O and S, or a group of atoms comprising one or more atoms selected from C, N, O, and S; more preferably an atom selected from O and S;
F2 represents an atom, or a group of atoms; preferably an atom selected from O, and S, or a group of atoms comprising one or more atoms selected from C, N, O, and S; more preferably an atom selected from O and S;
* indicates covalent attachment to the spacer (S); and
** indicates covalent attachment to the payload (P).

9. The compound of item 8, wherein the reactive moiety is represented by one of the following formulae (4a) to (4m):

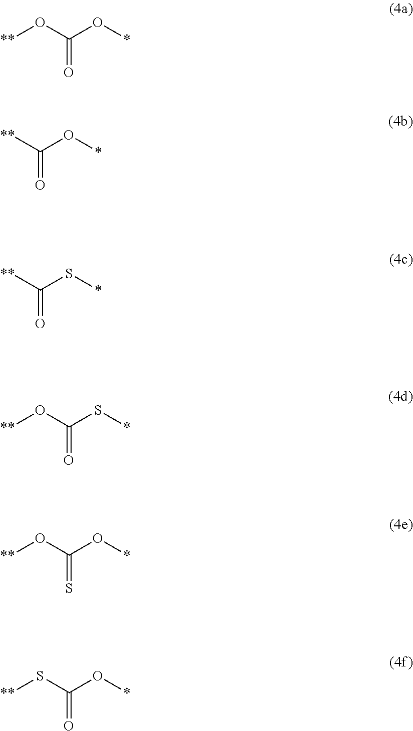

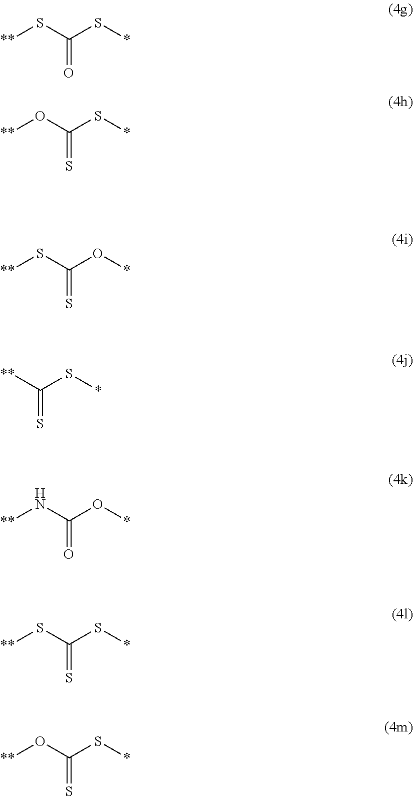

wherein * indicates covalent attachment to the spacer (S), and ** indicates covalent attachment to the payload (P).

10. The compound of any of items 1 to 7, wherein the reactive moiety is represented by the following formula (3b):

**—(F1-RC—F2)-(M)-* (3b)
wherein,
RC is a reactive center, preferably an electrophilic reactive center, and more preferably a group selected from C═O and C═S;
F1 is a single covalent bond, an atom, or a group of atoms; preferably an atom selected from O and S, or a group of atoms comprising one or more atoms selected from C, N, O, and S; more preferably an atom selected from O and S;
F2 represents an atom, or a group of atoms; preferably an atom selected from O, and S, or a group of atoms comprising one or more atoms selected from C, N, O, and S; more preferably an atom selected from O and S;
M is a group capable of modulating the electron density and stability of F2, preferably a group capable of withdrawing electrons;
* indicates covalent attachment to the spacer (S); and
** indicates covalent attachment to the payload (P).

11. The compound of item 10, wherein the group capable of modulating the electron density and stability of F2 is represented by the following formula (3c):

***′-M′-B—C—* (3c)
wherein,
M′ is an aryl group having 6, 10 or 14 ring members and 1, 2 or 3 condensed rings, respectively, or a heteroaryl group having 5 to 20 ring members, 1, 2 or 3 condensed rings and 1 to 4 heteroatoms independently selected from N, O and S, which may be substituted with one or more substituents; preferably a phenyl group, a naphthyl group, a pyridyl group, a quinolinyl group, an isoquinolinyl group or a benzotriazolyl group, which may be substituted with one or more substituents, each substituent being preferably selected from —F, —Br, —Cl, —I, —NO₂, —CN, —C₁₋₆-alkoxy, —C₁₋₆-amido such as —C(═O)NH₂, and combinations thereof such as —CCl₃, —CF₃ or —CH₂NO₂;
B is a single covalent bond, O, S, NR′ wherein R′ represents a hydrogen atom, OH, an alkyl group or a cycloalkyl group, a C₂₋₆-alkenylene, a C₂₋₆-alkynylene, a group having the general formula:

—(CH₂)ₙ₁—(H¹)ₓ₁—(CH₂)ₙ₂—(H²)ₓ₂—(CH₂)ₙ₃—(H³)ₓ₃—(CH₂)ₙ₄— (3c′)
wherein,
each of n1, n2, n3 and n4 represents an integer independently selected from 0 to 10 such that n1+n2+n3+n4 is 10 or less,
each of x1, x2 and x3 is independently selected from 0 and 1, and
each H¹, H² and H³ is an atom independently selected from N, O and S, provided that if x1+x2=2, n2≥1, if x2+x3=2, n3≥0, if x1+x3=2, n2≥1 or n3≥1, and if x1+x2+x3 is 3, n2≥1 and n3≥1;
or any combination thereof; preferably a single covalent bond, NH or a C₁₋₁₀-alkylene group; more preferably a single covalent bond;
C is C═O, C═S, C(═NR″) wherein R″ represents a hydrogen atom, OH, an alkyl group or a cycloalkyl group, S═O, or S(═O)₂; preferably C═O;
* indicates covalent attachment to the spacer (S); and
***′ indicates covalent attachment to F2.

12. The compound of item 10 or 11, wherein the moiety (F1-RC—F2) is represented by one of the following formulae (4a′) to (4m′) and/or M is independently represented by one of the following formulae (5a) to (5j′):

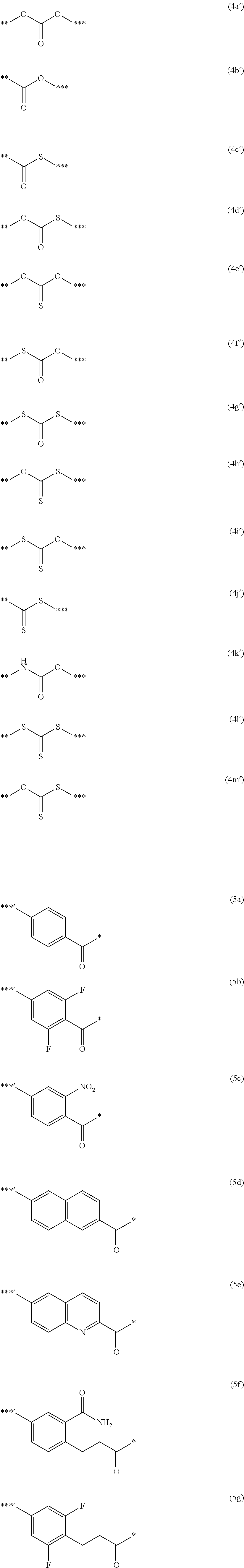

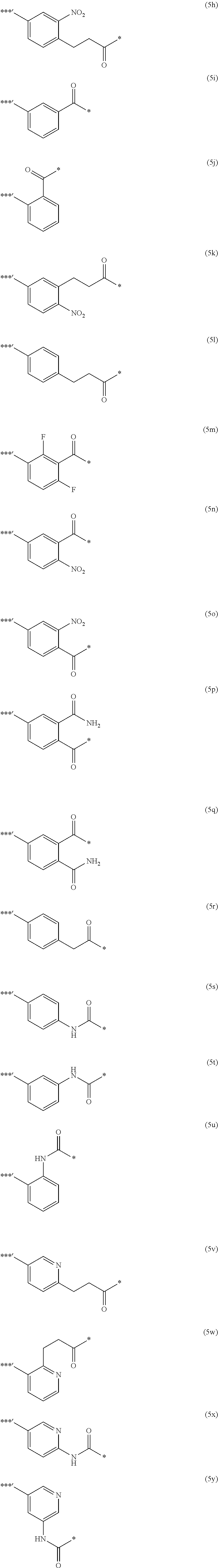

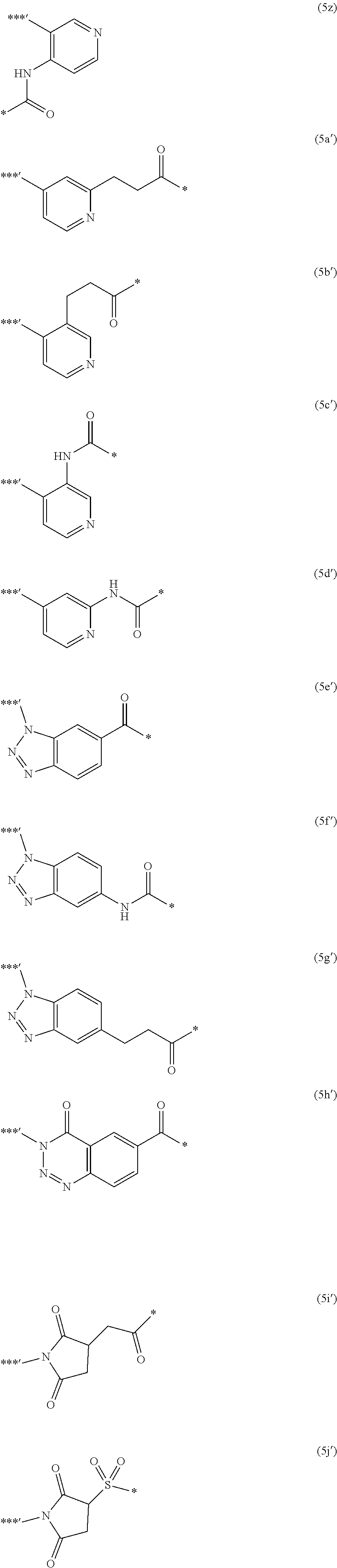

wherein * indicates covalent attachment to the spacer (S), ** indicates covalent attachment to the payload (P), *** indicates covalent attachment to M, and ***′ indicates covalent attachment to F2.

13. The compound of any of items 10 to 12, wherein the reactive moiety is represented by one of the following formulae (6a) to (61′):

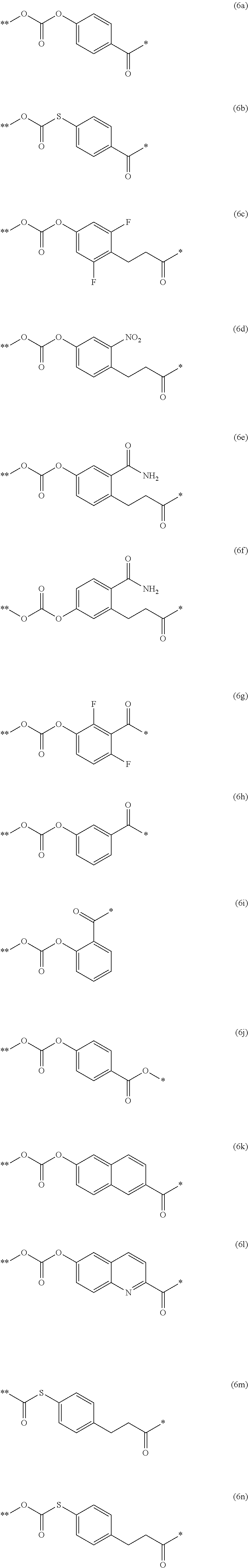

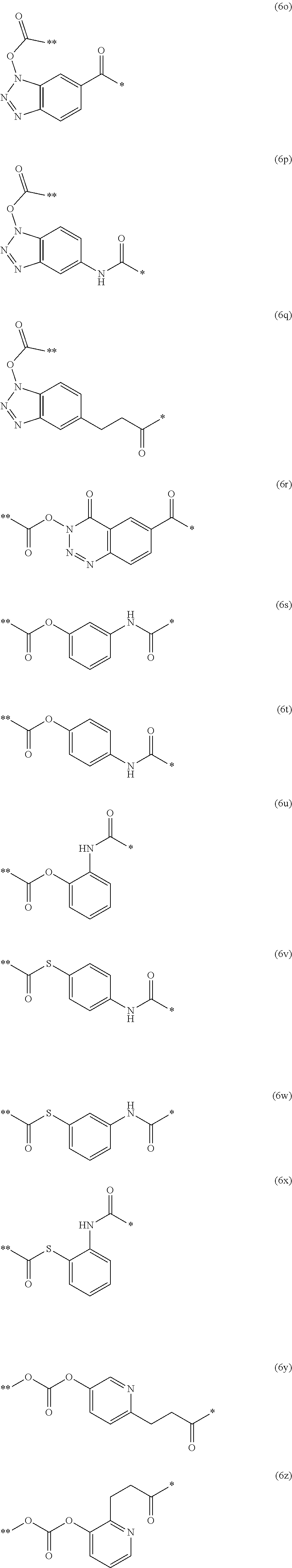

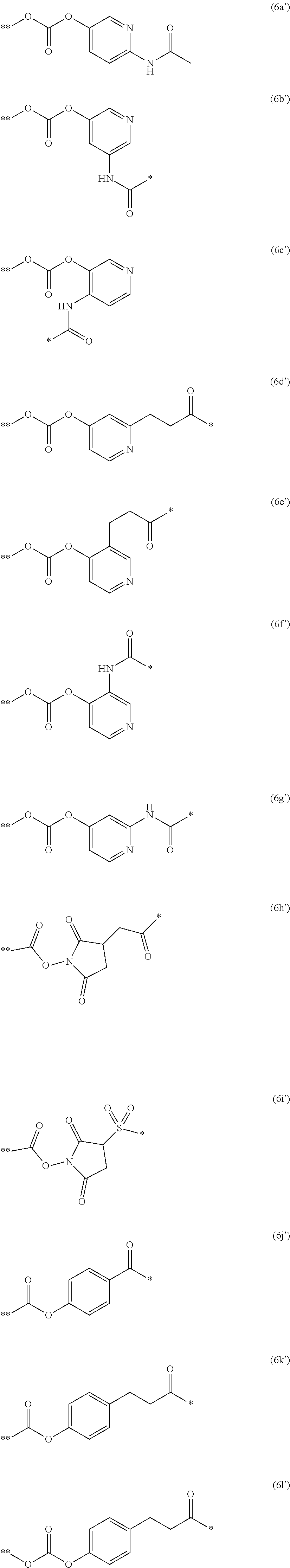

wherein * indicates covalent attachment to the spacer (S), and ** indicates covalent attachment to the payload (P).

14. The compound of any of items 1 to 13, wherein the spacer has a length of 10 to 35 Å; and preferably is a group having in a main chain from 12 to 120 atoms, e.g. 16 to 80 atoms, said atoms being selected from carbon, nitrogen, oxygen, and sulfur; more preferably a group selected from:
(a2) a polyalkylene oxide group having from 6 to 36 repeating units, for instance 8 to 24 repeating units; preferably a group represented by the following formula (7):

—X¹—(CH₂CH₂O)ₙ₂—CH₂CH₂—X²— (7)
wherein
X¹ is NH, O or S; preferably NH;
X² is NH or C═O, preferably C═O if X² is covalently bonded to the vector; and
n2 is an integer of 4 to 28, preferably 6 to 20, e.g. 10;
(b2) a peptidic group having 6 to 25 amino acids in the main chain, e.g. 9 amino acids in the main chain, each amino acid being preferably selected from Pro, Gly, Ala, Asn, Asp, Thr, Glu, Gln, and Ser; more preferably Pro, Gly or Ser.

15. The compound of any of items 1 to 13, wherein the spacer comprises a polyethylene oxide group having 4 to 36 repeating units, preferably 6 to 28 repeating units, and more preferably 7 to 24 repeating units.

16. The compound of any of items 1 to 15, wherein the vector is a peptide comprising a sequence of 11 to 17 amino acids, e.g. 13 to 17 amino acids, preferably a peptide represented by one of the following formulae (8a) and (8b):

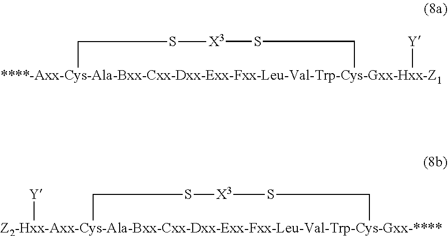

wherein,
Bxx, Cxx, Dxx, Exx, Fxx each independently represent an amino acid;
Axx represents an amino acid, a dicarboxylic acid, or a peptide moiety represented by the following formula (9a):

-Axx1-Axx2-Axx3- (9a)
wherein, in formula (9a),
Axx1 represents a single covalent bond, or an amino acid such as Arg;
Axx2 represents an amino acid such as Gly or Cys; and
Axx3 represents an amino acid such Asp or Asn;
Gxx represents an amino acid, or a peptide moiety represented by the following formula (9b):

-Gxx1-Gxx2-Gxx3- (9b)
wherein, in formula (9b),
Gxx1 represents an amino acid such as Thr;
Gxx2 represents an amino acid such as Tyr or Cys; and
Gxx3 represents a single covalent bond, or an amino acid such His; and the side chain of Axx2 may be covalently bonded to the side of Gxx2 to form a ring;
if Axx2 is Cys, and Gxx2 is Cys preferably the side chains of Axx2 and Gxx2 are linked together to form a group of formula —(S—X⁴—S)—, wherein X⁴ represents a single covalent bond or a divalent group comprising one or more atoms selected from carbon, nitrogen and oxygen such as a divalent maleimide group, a divalent acetone group or a divalent arylene group, preferably a single covalent bond;
Hxx represents a single covalent bond, or a trifunctional amino acid such as a diamino-carboxylic acid;
Z₁ represents
   a group covalently bonded to the C-terminus of Gxx if Hxx is a single covalent bond, which is selected from —N(H)(R), wherein R represents a hydrogen atom, an alkyl group or a cycloalkyl group, and a moiety derived from a compound containing a conjugation group selected from biotin, DBCO, TCO, BCN, an alkyne, an azide, a bromoacetamide, a maleimide and a thiol;
   a group covalently bonded to the C-terminus of Hxx if Hxx is a trifunctional amino acid and Y′ is bonded to the side chain of Hxx, preferably N(H)(R), wherein R represents a hydrogen atom, an alkyl group or a cycloalkyl group, if Z₁ is covalently bonded to the C-terminus of Hxx; or
   a hydrogen atom bonded to the side chain of Hxx if Hxx is trifunctional amino acid and Y′ is bonded to the C-terminus of Hxx.
Z₂ represents
   a group covalently bonded to the N-terminus of Axx if Hxx is a single covalent bond, which is selected from a hydrogen atom, a carbonyl-containing group such as an acetyl group, and a group containing a conjugation moiety such as biotin;
   a group covalently bonded to the N-terminus of Hxx if Hxx is a trifunctional amino acid and Y′ is bonded to the side chain of Hxx, which is selected from a hydrogen atom and a carbonyl-containing group such as an acetyl group; or
   a hydrogen atom bonded to the side chain of Hxx if Hxx is trifunctional amino acid and Y′ is bonded to the N-terminus of Hxx.
Y′ is present only if Hxx is a trifunctional amino acid and it represents a moiety covalently bonded to
   the side chain of Hxx if Z₁ is bonded to the C-terminus of Hxx or if Z₂ is bonded to the N-terminus of Hxx,
   the C-terminus of Hxx if Z₁ is bonded to the side chain of Hxx, or
   the N-terminus of Hxx if Z₂ is bonded to the side chain of Hxx;
Y′ is derived from a compound containing a conjugation group, which is preferably selected from biotin, DBCO, TCO, BCN, an alkyne, an azide, a bromoacetamide, a maleimide, and a thiol;
X³ represents a single covalent bond or a divalent group comprising one or more atoms selected from carbon, nitrogen and oxygen such as a divalent maleimide group, a divalent acetone group or a divalent arylene group, preferably a single covalent bond;
**** indicates covalent attachment to the spacer (S).

17. The compound of item 16, wherein at least one of Axx, Bxx, Cxx, Dxx, Exx, Fxx, Gxx and Hxx is defined as follows:

Axx represents an amino acid selected from Ala, 2,3-diamino-propionic acid (Dap), Asp, Glu, 2-amino suberic acid, α-amino butyric acid, Asn and Gln, a dicarboxylic acid selected from succinic acid, glutaric acid and adipic acid; preferably Ala, Asp or Asn; more preferably Asp; or a peptide moiety of formula (9a), wherein Axx1 is a single covalent bond, Axx2 is Cys, and Axx3 is Asp;

Bxx represents an amino acid selected from Trp, Phe, Tyr, phenyl glycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;

Cxx represents an amino acid selected from His, Ala, 3-pyridin-2-yl-L-alanine, meta-tyrosine (mTyr) and Phe; preferably His, Ala or mTyr; more preferably His;

Dxx represents an amino acid selected from Ala, Abu, Gly, Leu, Ile, Val, Met, cyclohexyl alanine (Cha), Phe, Thr, Cys, Tyr, and norleucine (Nle); preferably Ala, Nle or Leu; more preferably Leu;

Exx represents an amino acid selected from Ala, Gly, Asn, Ser, Abu, and Asp; preferably Ala or Gly; more preferably Gly;

Fxx represents an amino acid selected from Ala, Glu, Asp, Gln, His, Arg, Ser, and Asn; preferably Asp or Glu; more preferably Glu;

Gxx represents an amino acid selected from Thr, Ser, Ala, Asn, Val, 2-amino-butyric acid (Abu), Ile, Met, Leu, Pro, Gln, and Cys; preferably Thr or Ser; more preferably Thr; or a peptide moiety of formula (9b), wherein Gxx1 is Thr, Gxx2 is Cys, and Gxx3 is a single covalent bond; and

Hxx represents an amino acid selected from Dap, Dab, Lys, Orn and homo-lysine (homo-Lys), preferably an amino acid selected from Dap, Dab, Lys, Orn and homo-Lys.

18. The compound of any of items 1 to 17, wherein the vector is a peptide represented by one of the following formulae (8a′) to (8d′):

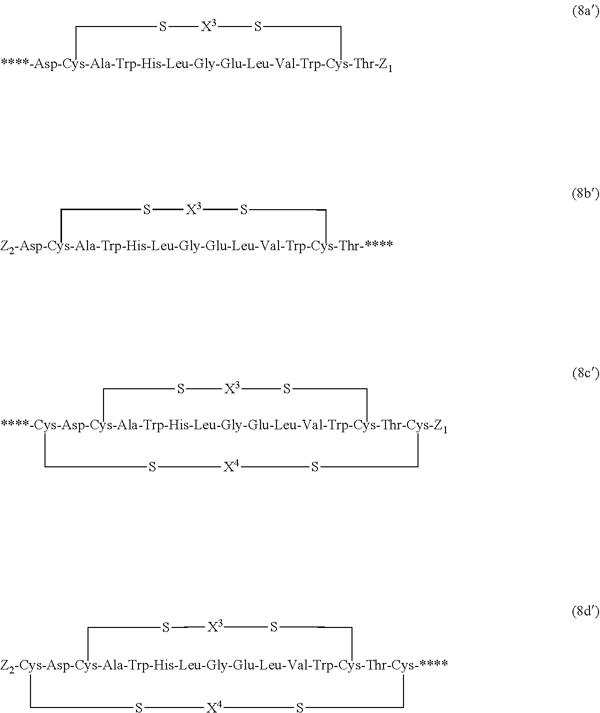

wherein,
Z₁, Z₂, X³, X⁴ and **** are as defined in item 16;

preferably a peptide represented by formula (8a′) or (8b′).

19. The compound of any of items 1 to 18, which is selected from

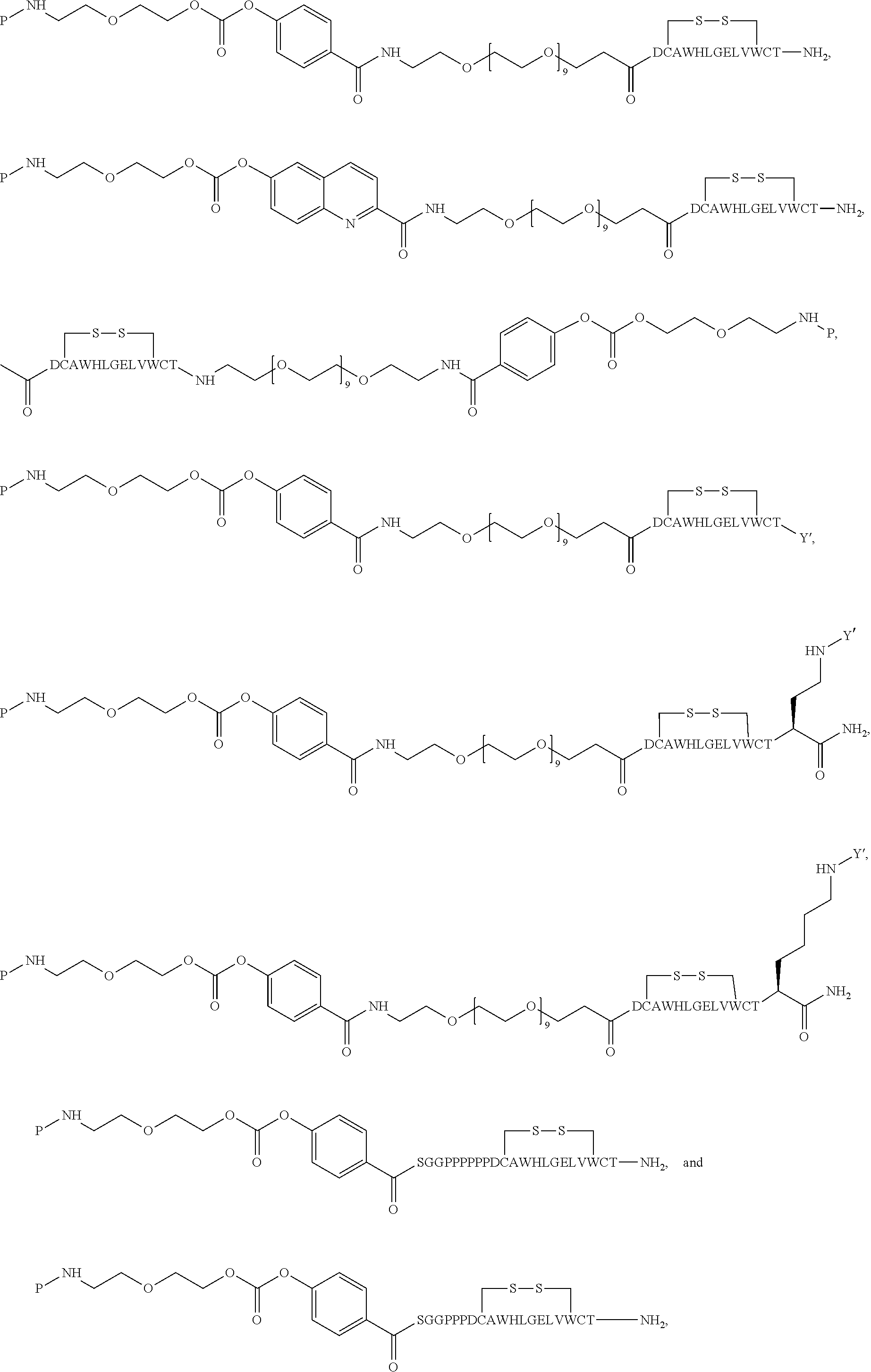

wherein P is as defined in any of items 1 to 5, and Y′ is as defined in item 16.

20. The compound of any of items 1 to 19, which is selected from

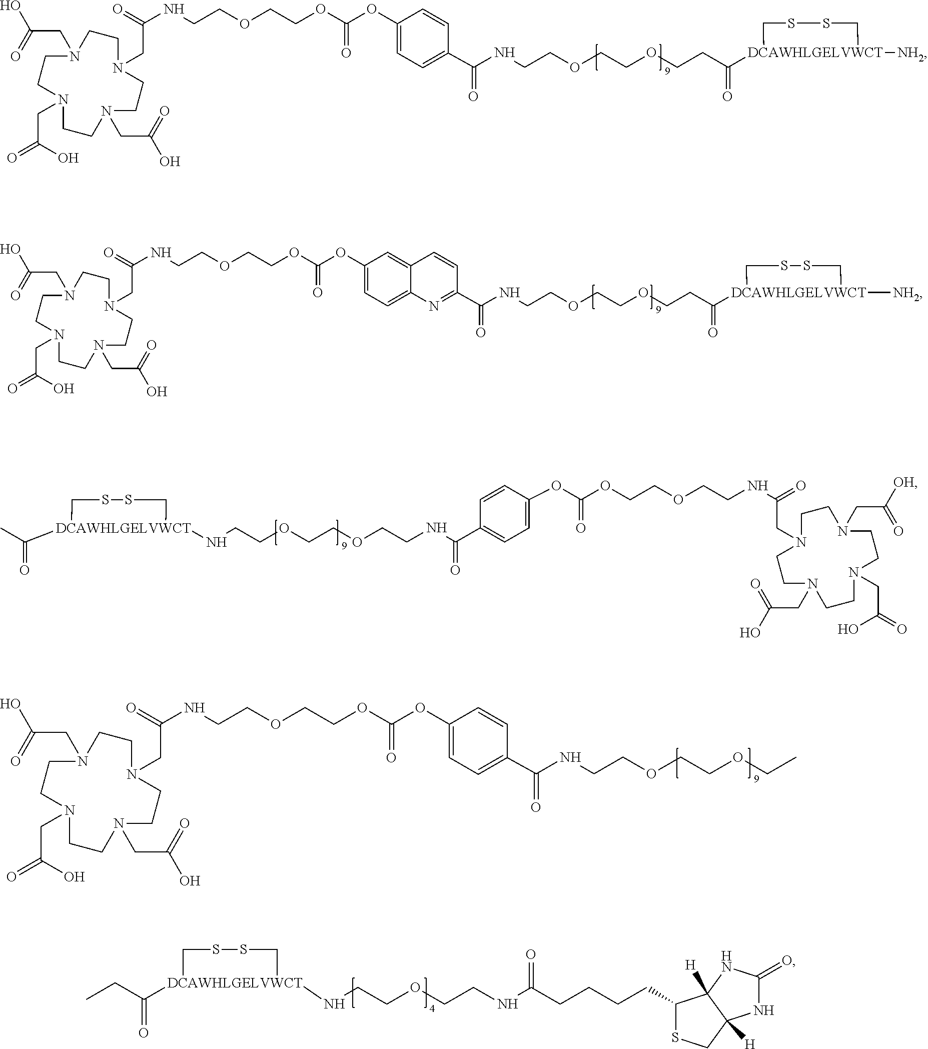

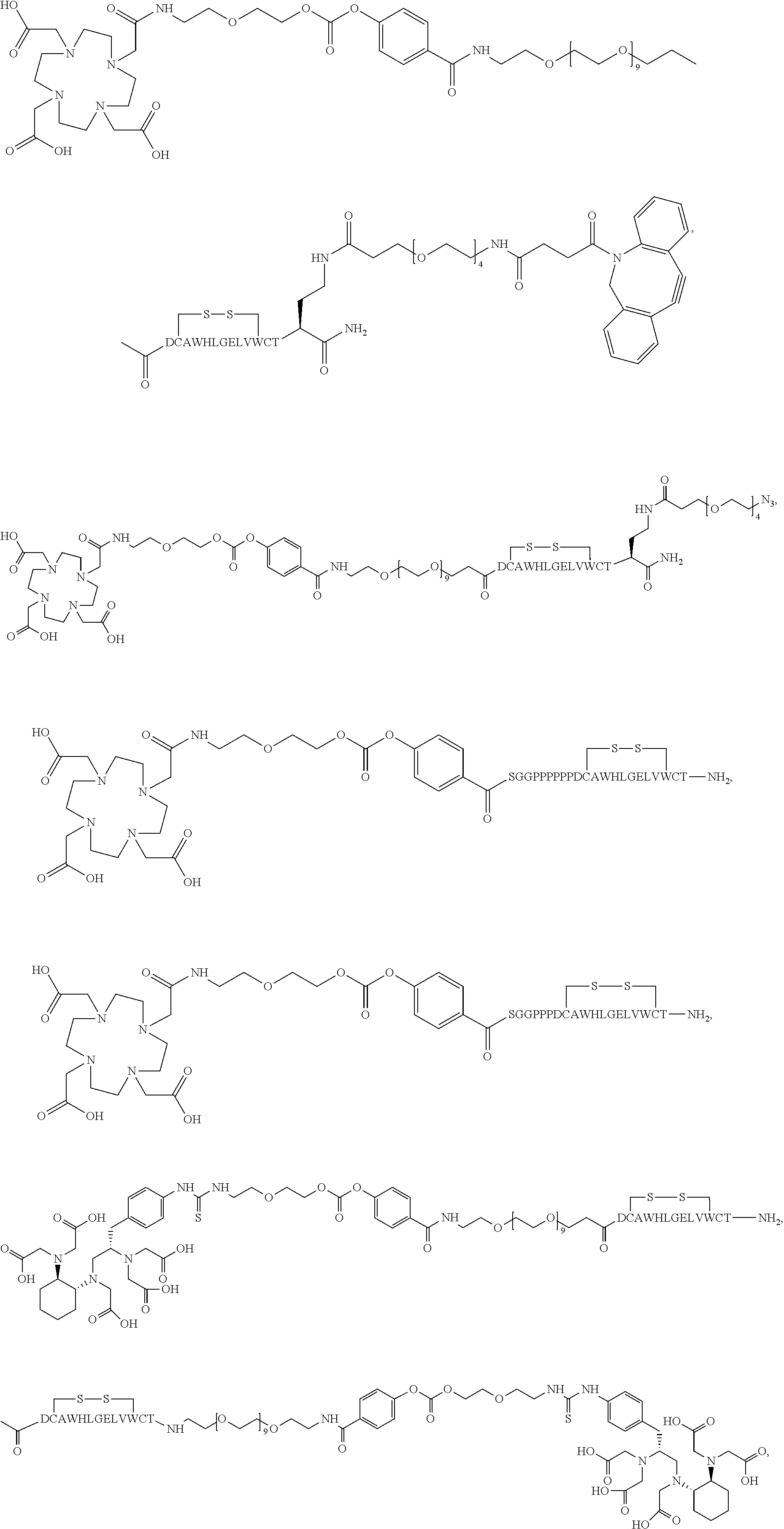

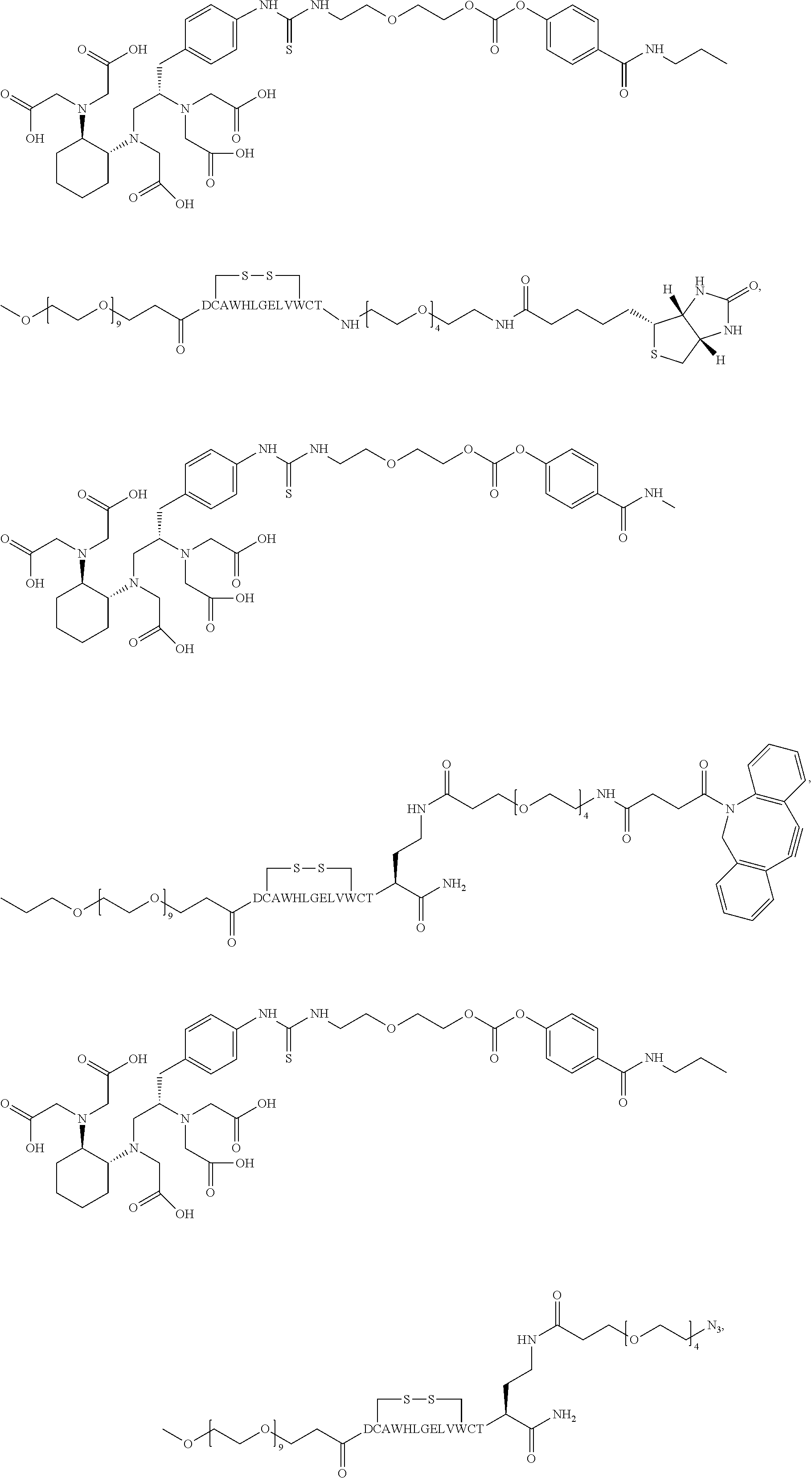

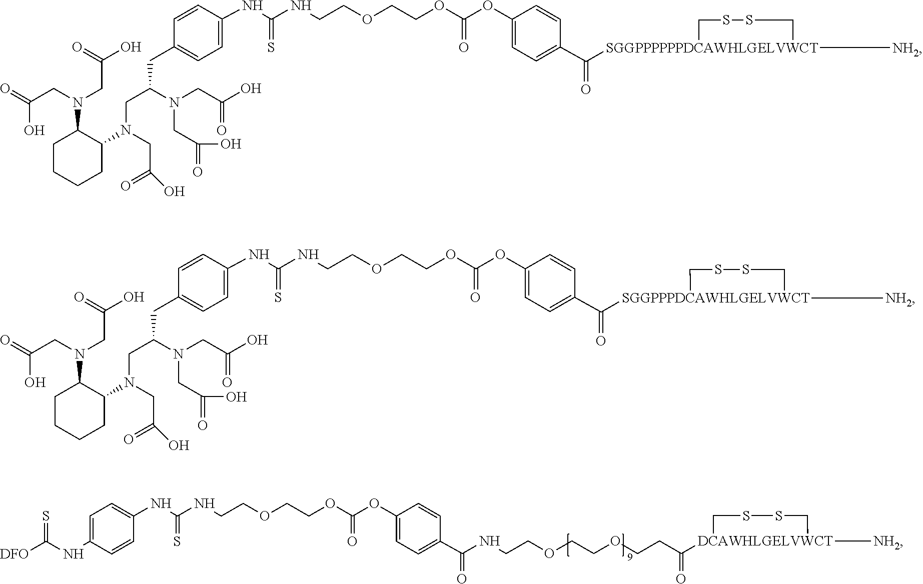

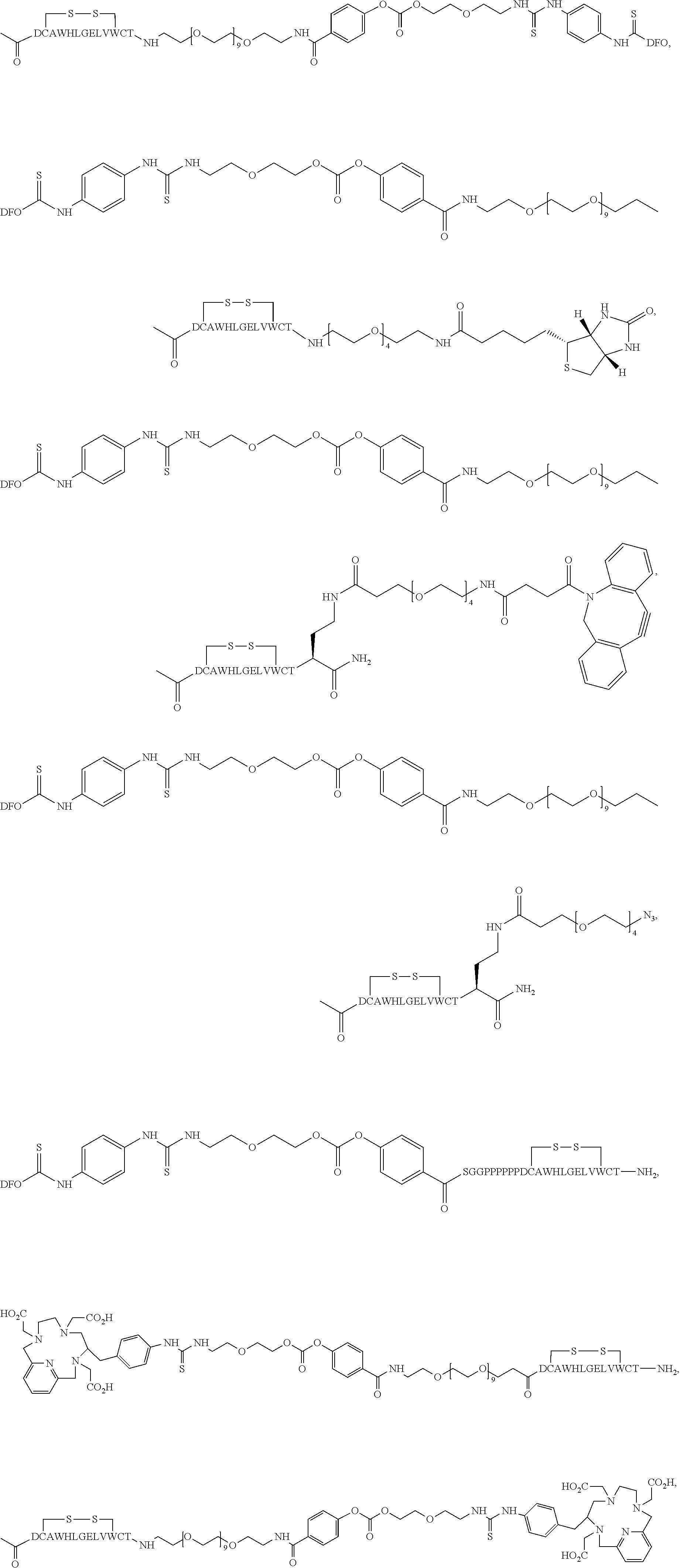

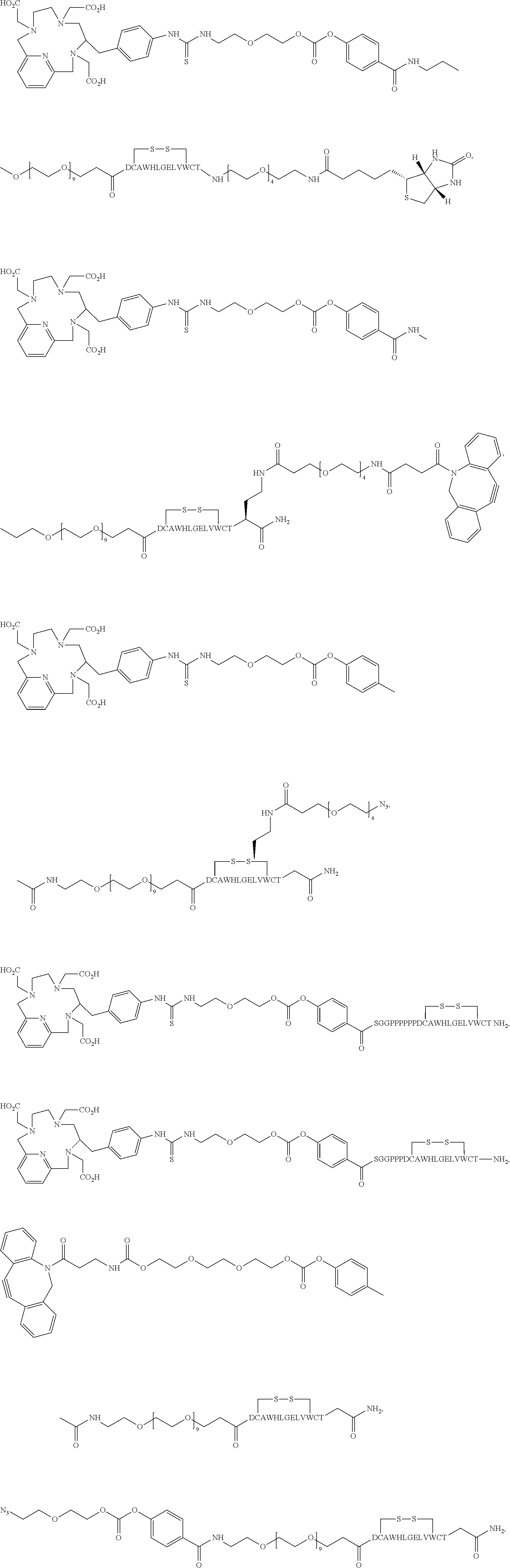

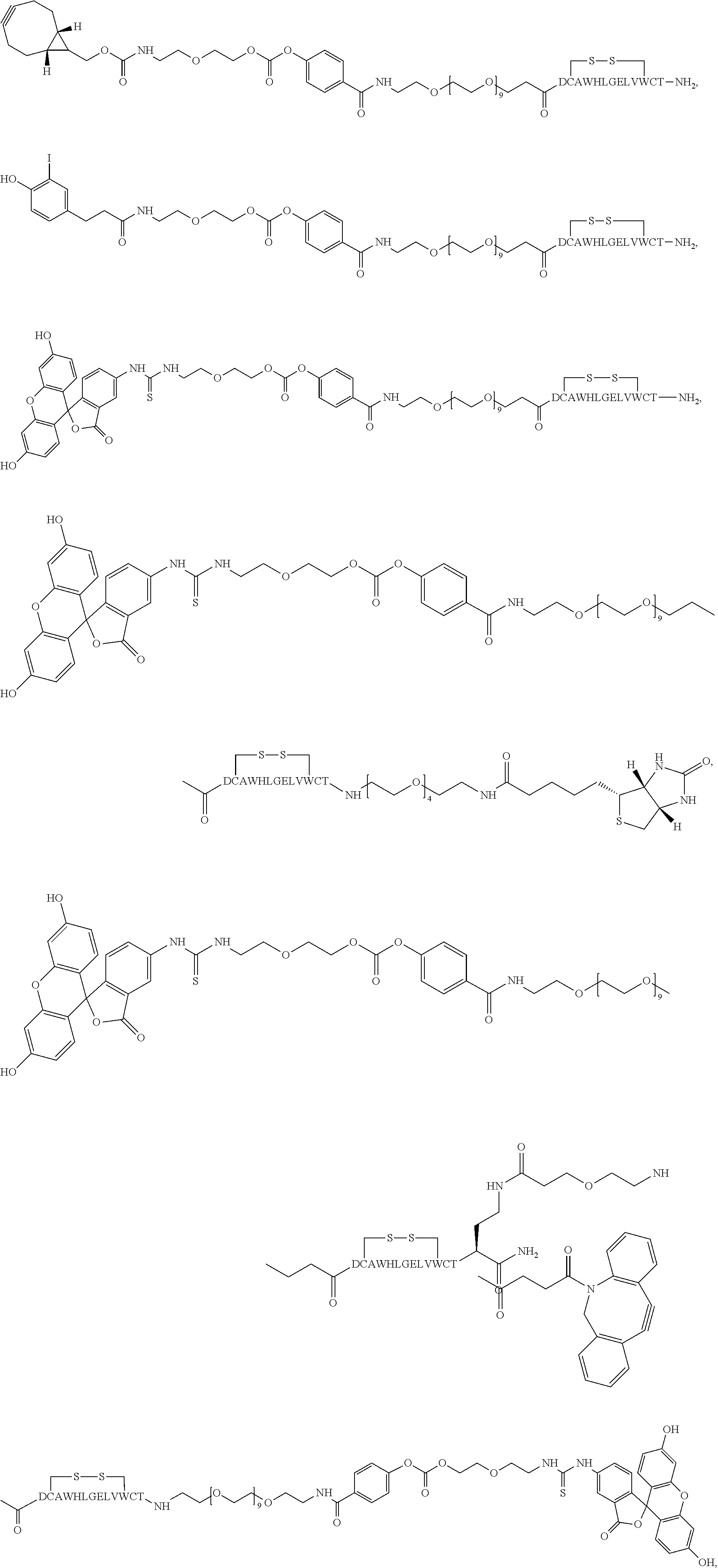

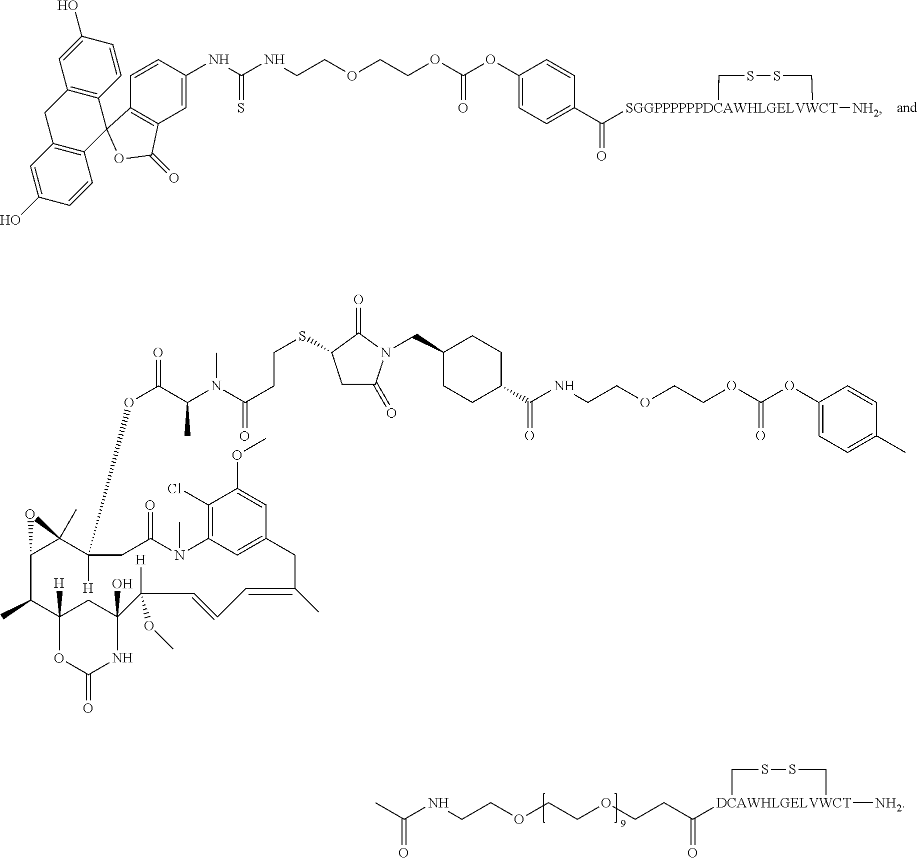

21. Kit for the site-specific modification of an antibody or fragment thereof, the antibody fragment being optionally incorporated into an Fc-fusion protein, comprising the compound of any of items 1 to 20 and a buffer; wherein the buffer has preferably a pH of 5.5 to 11, more preferably of 7.5 to 9.5.

22. The kit for the regioselective modification of an antibody or fragment thereof of item 21, wherein the compound is immobilized on a solid phase matrix, e.g. beads.

23. Method for the regioselective modification of an antibody or fragment thereof comprising reacting an antibody or fragment thereof, the antibody fragment being optionally incorporated into an Fc-fusion protein, with a compound according to any of items 1 to 20.

24. The method of item 23, wherein
the antibody is a monoclonal antibody, preferably an antibody selected from the group consisting of adalimumab, aducanumab, alemtuzumab, altumomab pentetate, atezolizumab, anetumab, avelumab, bapineuzumab, basiliximab, bectumomab, bermekimab, besilesomab, bevacizumab, bezlotoxumab, brentuximab, brentuximab vedotin, brodalumab, blinatumomab, catumaxomab, cemiplimab, cetuximab, cinpanemab, clivatuzumab, clivatuzumab tetraxetan, crenezumab tetraxetan, daclizumab, daratumumab, denosumab, dinutuximab, durvalumab, edrecolomab, elotuzumab, emapalumab, enfortunab, enfortunab vedotin, epratuzumab, epratuzumab-SN38, etaracizumab, gemtuzumab, gemtuzumab ozogamycin, girentuximab, gosuranemab, ibritumomab, inebilizumab, infliximab, inotuzumab, inotuzumab ozogamicin, ipilimumab, isatuximab, ixekizumab, J591 PSMA-antibody, labetuzumab, lecanemab, mogamulizumab, necitumumab, nimotuzumab, natalizumab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, siltuximab, sacituzumab, sacituzumab govitecan, semorinemab, siltuximab, solanezumab, tacatuzumab, tetrotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably atezolizumab, durvalumab, prembolizumab, rituximab and trastuzumab; or
the antibody fragment is incorporated into an Fc-fusion protein, which is preferably selected from belatacept, aflibercept, ziv-aflibercept, dulaglutide, rilonacept, romiplostim, abatacept, and alefacept.

25. Modified antibody or modified antibody fragment obtainable by reacting an antibody or antibody fragment, the antibody fragment being optionally incorporated into an Fc-fusion protein, with a compound according to any of items 1 to 20, wherein the antibody or antibody fragment has preferably the same definition as in item 24.

26. Modified antibody or modified antibody fragment as defined in item 25 for use in a method of diagnosing, monitoring, imaging or treating a disease, the method comprising administering the modified antibody or modified antibody fragment to a subject.

27. Method for diagnosing, monitoring, imaging or treating a disease comprising administering the modified antibody or modified antibody fragment according to item 25 to a subject in need thereof.

28. The modified antibody or modified antibody fragment for use according to item 26, or the method according to item 27, wherein the disease is a neurological disease, a cardiovascular disease, an auto-immune disease or a cancer.

29. The modified antibody or modified antibody fragment for use according to item 26 or 28, or the method according to item 27 or 28, wherein the disease or treatment thereof is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina including unstable angina, Aortic aneurysm, Atherosclerosis, Cardiac transplant, Cardiotoxicity diagnosis, Coronary artery bypass graft, Heart failure including atrial fibrillation terminated systolic heart failure, hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.

30. The modified antibody or modified antibody fragment for use according to item 26 or 28, or the method according to item 27 or 28, wherein the disease involves cells selected from lymphoma cells, myeloma cells, renal cancer cells, breast cancer cells, prostate cancer cells, ovarian cancer cells, colorectal cancer cells, gastric cancer calls, squamous cancer cells, small-cell lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, small-cell lung cancer cells, lymphoma cells, colorectal cancer cells, and head-and-neck cancer cells.

### FIGURES

FIG. 1—Schematic representation of the antibody conjugation approach using the compound of the present invention. A vector capable of interacting with the Fc region of an antibody binds to the Fc region, thereby bringing the reactive moiety in close proximity to the side chain of a lysine residue exposed at the surface of the antibody. The reaction between the side chain of the lysine residue and the reactive moiety leads to covalent attachment of the payload (via a linker) to the antibody, and to the concomitant release of the vector.

FIG. 2—Synthesis of compound 29, i.e. compound comprising a labelling moiety (DOTA) as a payload and a PEG₁₀ spacer: a) 1. HATU, DMF, DIEA (pre-activation 3-5 min), 2. compound 1, 3. 20% piperidine in DMF (yield: 42% over 2 steps), b) 1. HATU, DMF, DIEA (pre-activation 3-5 min), 2. compound 7, 3. TFA (+HPLC purification).

FIG. 3—Fluorescence polarization (FP) binding assay. Binding isotherms of a fluorescein derivative of the ligand Fc-III (Fc-III-FAM) at a concentration of 5 nM to the therapeutic monoclonal antibodies trastuzumab, alemtuzumab, bevacizumab, and rituximab. The lines are fits of the data using the Hill equation yielding the half-maximal effective concentration (EC₅₀). It was confirmed that the Fc-binding ligand Fc-III-FAM binds the respective antibodies with high affinity (trastuzumab: 14 nM, alemtuzumab: 13 nm, bevacizumab: 7 nM, rituximab: 11 nM).

FIG. 4—Competitive FP binding assay. The propensity of the Fc-binding vectors of Example 1 (compounds 1 (Fc-III), 2, 9-11, 15 and 16) to bind the Fc region of trastuzumab against Fc-III-FAM was evaluated. The lines are fits of the data using the Hill equation yielding the half-maximal inhibitory concentration (IC₅₀). The results are also given in Table 3.

FIG. 5—Synthesis of compounds 17 and 19, i.e. fluorescein- and DOTA-carbonate derivatives: a) Et₃N in CH₃CN at 40° C., b) DMAP in CH₂Cl₂ at 25° C., c) TFA/CH₂Cl₂ (1/3, v/v), d) DIPEA in CH₃CN at 25° C., e) DIPEA in CH₃CN/DMF (1/1, v/v) at 25° C.

FIG. 6—High-Resolution mass spectrometry (HRMS) of trastuzumab and trastuzumab modified with compound 31, i.e. trastuzumab-DOTA conjugate. The peaks D0 to D3 correspond to trastuzumab fragments with different degrees of conjugation. The samples were deglycosylated prior to HRMS measurement.

FIG. 7—HRMS of trastuzumab-DOTA conjugate digested with GingisKhan® enzyme into Fab and Fc regions. The peaks D0 to D2 correspond to trastuzumab with different levels of conjugation.

FIG. 8—Affinity of trastuzumab-DOTA conjugate and trastuzumab for SK-BR-3 (HER2+) and MD-MB-231 (HER2−) cells. For SK-BR-3 cells, the concentration of antibody or antibody conjugate ranged from 0.003 to 30 μg/mL ( 1/10 dilutions were made). For MD-MB-231 cells, only 3 and 30 μg/mL were used. The trastuzumab and trastuzumab-DOTA conjugate were stained with a secondary rat anti-human IgG Fc antibody conjugated with Alexa 488. Dead cells were excluded with DRAQ7. Error bars: SD (n=2).

FIG. 9—Synthesis of compound 38, i.e. reactive conjugate comprising a PEG20 spacer and a labelling moiety (fluorescein (FL)) as a payload: a) 1. HATU, DMF, DIEA (pre-activation 3-5 min), 2. compound 1, 3. 20% piperidine in DMF (yield: 50% over 2 steps), b) 1. HATU, DMF, DIEA (pre-activation 3-5 min), 2. compound 10, 3. TFA (+HPLC purification; yield: 19%).

FIG. 10—Non-reducing SDS-PAGE analysis of trastuzumab-FL conjugate prepared by reacting compounds 35-41 with trastuzumab (IgGT). Conjugates after reduction (A) or IdeS protease digestion (B) were analyzed using Coomassie blue staining and fluorescence.

FIG. 11—BT-474 cells incubated with 10 μg/ml FITC-Trastuzumab (conjugate 12, random conjugation, dashed) and FL-Trastuzumab (conjugate 11, μlain) and increasing concentrations of unlabeled Trastuzumab. Plotted data represents mean of MFI scores of two independent experiments. Maximal MFI for each antibody was normalized to 1.

FIG. 12—Non-reducing SDS-PAGE analysis of trastuzumab-FL conjugate prepared by reacting compounds 38 and 40 with trastuzumab (IgGT), commercial trastuzumab (Herceptin®) (IgGH), alemtuzumab (IgGA), bevacizumab (IgGB), and rituximab (IgGR). Conjugates after IdeS protease digestion were analyzed using fluorescence and Coomasie blue staining.

FIG. 13—Schematic representation of reactive conjugate immobilization on a solid support.

FIG. 14—Schematic representation of the antibody conjugation approach using a peptide conjugate containing a DBCO group (compound 43) and any payload containing an azide group.

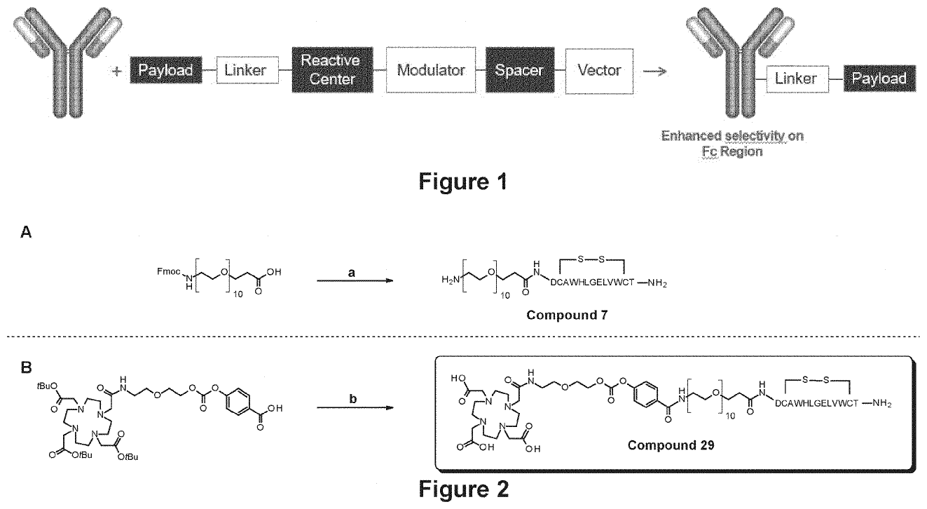

### DETAILED DESCRIPTION OF THE PRESENT INVENTION

### 1. DEFINITIONS

The term “payload” as used herein characterizes a substance (e.g. a naturally occurring or synthetic substance) which can confer a novel functionality when it is attached (conjugated) to an antibody or antibody fragment. In some embodiments, the term “payload” as used herein is to be understood as a labeling moiety (e.g. chromophore, fluorophore, radiolabeled moiety) that enables and/or facilitates the detection and/or visualization of a complementary moiety (e.g. an antibody) to which it is attached. For instance, the labeling moiety can be detected and/or visualized by functional (physiological) imaging techniques known in the art such as computed tomography (CT), positron emission tomography (PET), etc. In some embodiments, the term “payload” as used herein is to be understood as a pharmacologically active substance which can inhibit or prevent the function of cells and/or kill cells. In some embodiments, the term “payload” is to be understood as being synonymous with other terms commonly used in the art such as “cytotoxic agent”, “toxin” or “drug” used in the field of cancer therapy. Alternatively, the payload is a moiety selected from a moiety comprising a conjugation group. The payload may include a group derivable from a functional group that allows covalent attachment of the payload to the remainder of the compound (e.g. to reactive moiety Y in formula (1)) such as a carboxylic acid, a primary amine, a secondary amine, a hydroxyl group, a thiol group, or the like.

The term “peptide” as used herein may be understood as a compound comprising a continuous sequence of at least three amino acids linked to each other via peptide linkages. The term “peptide linkage” in this connection is meant to encompass (backbone) amide bonds as well as modified linkages, which can be obtained if non-natural amino acids are introduced in the peptidic sequence. In this case, the modified linkage replaces the (backbone) amide bond which is formed in the continuous peptide sequence by reacting the amino group and the carboxyl group of two amino acid residues (NH₂—CR¹—COOH+NH₂—CR²—COOH→NH₂—CR¹—(C═O)—NH—CR²—COOH). For instance, the modified linkage may be an ester (NH₂—CR¹—(C═O)—O—CR²—COOH), a thioester (NH₂—CR¹—(C═O)—S—CR²—COON or NH₂—CR¹—(C═S)—O—CR²—COOH), a carbamide (NH₂—CR¹—NH—(C═O)—NH—CR²—COOH), a thiocarbamide (NH₂—CR¹—NH—(C═S)—NH—CR²—COOH) or a triazole linkage (e.g. NH₂—CR¹—C≡CH+N₃—CR²—COOH→NH₂—CR¹—X—CR²—COOH wherein X represents a 1,4-disubstituted-1,2,3-triazole moiety). Preferably, the amino acids forming the continuous peptide sequence are linked to each other via backbone amide bonds. The peptide may be linear or branched. In one aspect, the peptide may be cyclic, for instance made of a linear chain of amino acids that has been modified to form a cycle, e.g. “head-to-tail” cyclization, or made of a linear chain of amino acids having side chains covalently attached to each other, e.g. by disulfide bond formation or any other modification. Here, the amino acids include both naturally occurring amino acids as well as non-natural (synthetic) amino acids, as described below.

The expression “labelling moiety” (or synonymously “label” or “label group”) as used herein refers to a moiety containing a group which enables and/or facilitates the detection and/or visualization by visual or instrumental means of a complementary moiety (e.g. an antibody) to which it is attached. Examples of labeling moieties include radioactive labels (e.g. radionuclides), contrast agents for magnetic resonance imaging (MRI), and chemicals that absorb or emit light, e.g. chromophores and fluorophores.

The expression “moiety derived from a drug” as used herein refers to a moiety corresponding to a native drug except for having structural modifications for bonding the native drug to the reactive group or linker comprised in the compound of the present invention. Depending on the functional groups available in the native drug, bonding may be effected using one of the functional groups already present in the native drug, or it may be effected by modifying the native drug by incorporating a new functional group. By consequence, the drug can be used for bonding in its non-modified form, or it can be chemically modified in order to incorporate one functional group allowing covalent attachment to the reactive moiety or linker comprised in the compound of the invention. The expression “moiety derived from a drug” as used herein is meant to encompass both meanings.

In an analogous manner, the term “derivative” is used in connection with other moieties to characterize the presence of covalent bonds needed for bonding to the adjacent moieties or other moieties chemically modified to incorporate one functional group allowing covalent attachment to the adjacent moieties. In other words, the term “derivative” may characterize moieties bonded to adjacent moieties, which moieties differ from the molecules from which they are derived only by the structural elements responsible for bonding to adjacent moieties. This may include covalent bonds formed by existing functional groups, for instance after removal of one hydrogen atom to provide for the required free valency for bonding, or covalent bonds and adjacent functional groups newly introduced for this purpose.

The expression “native drug” characterizes a compound, for which therapeutic efficacy has been established by in vitro and/or in vivo tests. In a preferred embodiment, the native drug is a compound for which therapeutic efficacy has been established by clinical trials. Most preferably, the native drug is a drug that is already commercially available. The type of therapeutic efficacy to be established and suitable tests to be applied depend of course on the type of medical indication to be treated.

When referring to specific classes of drug molecules, such as an antineoplastic agent, a topoisomerase inhibitor, an RNA-polymerase II inhibitor, a DNA cleaving agent, an antimitotic agent or microtubule disruptor, an anti-metabolite, a kinase inhibitor, an immunomodulatory agent, or an anti-infectious disease agent, these terms are intended to have the meaning generally accepted in the field of medicine, as reflected, for instance, in the Mosby's Medical Dictionary, Mosby, Elsevier 10^{th} ed. (2016), or in Oxford Textbook of Oncology, David J. Kerr, OUP Oxford 3^{rd} ed. (2016).

The expression “chelating agent” as used herein refers to a molecule containing two or more electron donor atoms that can form coordinate bonds to a single central metal ion, e.g. to a radionuclide. Typically, chelating agents coordinate metal ions through oxygen or nitrogen donor atoms, or both. After the first coordinate bond is formed, each successive donor atom that binds creates a ring containing the metal ion. A chelating agent may be bidentate, tridentate, tetradentate, etc., depending on whether it contains 2, 3, 4, or more donor atoms capable of binding to the metal ion. However, the chelating mechanism is not fully understood and depends on the chelating agent and/or radionuclide. For example, it is believed that DOTA can coordinate a radionuclide via carboxylate and amino groups (donor groups) thus forming complexes having high stability (Dai et al. Nature Corn. 2018, 9, 857). The expression “chelating agent” is to be understood as including the chelating agent as well as salts thereof. Chelating agents having carboxylic acid groups, e.g. DOTA, TRITA, HETA, HEXA, EDTA, DTPA etc., may, for example, be derivatized to convert one or more carboxylic acid groups to amide groups for attachment to the compound, i.e. to the reactive moiety or the linker, alternatively, for example, said compounds may be derivatized to enable attachment to the compound via one of the CH₂ groups in the chelate ring.

The term “radionuclide” as used herein refers to an atom with an unstable nucleus, which is a nucleus characterized by excess energy available to be imparted either to a newly created radiation particle within the nucleus or to an atomic electron. Radionuclides occur naturally or can be artificially produced. In some embodiments, the radionuclide to be used in the present invention is a medically useful radionuclide including, for example, positively charged ions of radiometals such as Y, In, Cu, Lu, Tc, Re, Co, and Fe. Preferably, the radionuclide is selected from ⁸⁹Zr, ¹¹¹In, ⁶⁴Cu, ¹⁷⁷Lu, ⁶⁸Ga, ^{99m}Tc, ²⁰³Pb, ⁷²As, ⁵⁵Co, ⁹⁷_{Ru,} ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷¹Lu, ⁶⁸Ga, and ^{99m}Tc, in particular ¹¹¹In.

The term “chromophore” as used herein refers to an organic or metal-organic compound which is able to absorb electromagnetic radiation in the range of from 350 nm to 1100 nm, or a subrange thereof, e.g. 350-500 nm or 500-850 nm, or 350-850 nm.

The term “fluorophore” as used herein refers to a compound which, when excited by exposure to a particular wavelength of light, emits light at a different (higher) wavelength. Fluorophores are usually described in terms of their emission profile or “color”. For example, green fluorophores such as Cy3 or FITC generally emit at wavelengths in the range of 515-540 nm, while red fluorophores such as Cy5 or tetramethylrhodamine generally emit at wavelengths in the range of 590-690 nm. The term “fluorophore” as used herein is to be understood as encompassing, in particular, organic fluorescent dyes such as fluorescein, rhodamine, or AMCA, and biological fluorophores.

The expression “pharmaceutically acceptable salts” as used herein refers to derivatives of disclosed compounds (including the reactive conjugates) wherein the parent compound is modified by making acid or base salts thereof. The pharmaceutically acceptable salts include the non-toxic salts or the quaternary ammonium salts of the parent compound formed, for example, from non-toxic inorganic or organic acids or bases. Lists of suitable salts can be found in Remington's Pharmaceutical Sciences, 17^{th} ed., Mack Publishing Company, Easton, Pa., 1985, page 1418, S. M. Berge, L. M. Bighley, and D. C. Monkhouse, “Pharmaceutical Salts,” J. Pharm. Sci. 66 (1), 1-19 (1977); P. H. Stahl and C. G. Wermuth, editors, Handbook of Pharmaceutical Salts: Properties, Selection and Use, Weinheim/Zürich, Wiley-VCH, 2008 and in A.K. Bansal et al., Pharmaceutical Technology, 3(32), 2008. The pharmaceutical salts can be synthesized from the parent compound which contains a basic or acidic moiety by conventional chemical methods. For the reactive conjugates, this can be done before or after incorporating the drug moiety into the compound of the present invention. Unless the context dictates otherwise, all references to compounds (conjugates, modified antibodies, etc.) of the invention are to be understood also as references to pharmaceutically acceptable salts of the respective compounds.

The expression “reactive moiety” as used herein refers to a moiety that can readily react with a binding partner on another molecule, e.g. a nucleophile. This is in contrast to moieties that require the addition of catalysts or highly impractical reaction conditions to react (i.e. “non-reactive” or “inert” moieties). Particularly, the expression “reactive moiety” refers to a moiety of a reactive conjugate, which reacts with the side chain of Lys of an antibody, preferably trastuzumab (Herceptin® available from Roche) at a molar ratio conjugate to trastuzumab of 2 to 1 when stirred at 1000 rpm in 50 mM NaHCO₃ pH 9.0 at room temperature for 2 hours, leading to the reaction (e.g. attachment of a payload to trastuzumab) of at least 25% of the conjugate, preferably at least 50% of the conjugate, more preferably at least 70% of the conjugate. The attachment of a payload to trastuzumab can be determined by high-resolution mass spectrometry according to the method described in section 9.3.5 below.

The expression “side chain of an amino acid” as used herein may refer to a moiety attached to the α-carbon of an amino acid. For example, the side chain of Ala is methyl, the side chain of Phe is phenylmethyl, the side chain of Cys is thiomethyl, the side chain of Tyr is 4-hydroxyphenylmethyl, etc. Both naturally occurring side chains and non-naturally occurring side chains are included by this definition. In case of non-natural amino acids, the side chain may also be present in a different position, e.g. attached to the backbone nitrogen in peptoid structures or attached to the β-carbon in some forms of β-amino acids.

The term “amino acid” as used herein refers to a compound that contains or is derived from at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. This term encompasses both naturally occurring amino acids as well as synthetic amino acids that are not found in nature. In the following, a reference to amino acids may be made by means of the 3-letter amino acid code (Arg, Phe, Ala, Cys, Gly, Gln, etc.) or by means of the 1-letter amino acid code (R, F, A, C, G, Q, etc.). Hereinafter, amino acid sequences are written from the N-terminus to the C-terminus (left to right).

The term “trifunctional” as used herein refers to a compound or moiety having three functional groups that can form or have formed three covalent bonds to adjacent moieties. Thus, the term “trifunctional amino acid” refers to a compound that contains or is derived from a compound containing at least an amino group, an acid group (e.g. a carboxyl group) and another functional group such as an amino group or a carboxyl group.

The term “C-terminal” as used herein refers to the C-terminal end of the amino acid (peptide) chain. Binding to the “C-terminus” means that a covalent bond is formed between the acid group in the main chain (backbone) of the amino acid residue and the binding partner. For instance, binding of group “X” to the C-terminus of amino acid residue Axx yields an ester or amide-type structural element —C(O)—X, wherein the carbonyl group is derived from the acid group of Axx.

The term “N-terminal” as used herein refers to the N-terminal end of the amino acid (peptide) chain. Binding to the “N-terminus” means that a covalent bond is formed between the amino group in the main chain (backbone) of the amino acid residue and the binding partner (which replaces one hydrogen atom). For instance, binding of group “X” to the N-terminus of amino acid residue Axx yields a structural element X—NH—, wherein the amino group is derived from Axx.

The expression “capable of interacting with the fragment crystallizable (Fc) region an antibody or fragment thereof” as used herein indicates that the vector can bind to the Fc region of an antibody or antibody fragment as defined hereinbefore. Said interaction/binding may give rise to a targeting effect i.e. to a local increase of the concentration of reactive moiety in proximity to the side chain of an amino acid (e.g. a lysine residue) of the antibody or antibody fragment. The interaction (binding) of a vector with the Fc region of an antibody or antibody fragment can be assessed by using fluorescence polarization techniques known in the art and described further below. In some aspects, the expression “compound capable of interacting with the Fc region of an antibody or fragment thereof” refers to a compound that retains at least 20%, preferably at least 50%, more preferably at least 80% of the binding affinity of ligand “Fc-III” for the Fc-region of IgG as described by DeLano et al. (Science 2000, 287, 1279-1283) and measured by fluorescence polarization. The compound capable of interacting with the Fc region of an antibody or fragment thereof may have superior binding affinity for the Fc region as compared with Fc-III.

The term “antibody” (also synonymously called “immunoglobulin” (Ig)) as used herein covers monoclonal antibodies, polyclonal antibodies, dimers, multimers, multi-specific antibodies (e.g. bispecific antibodies), veneered antibodies, and small immune proteins, provided that it comprises at least one fragment crystallizable (Fc) region. An antibody is a protein generated by the immune system that is capable of recognizing and binding to a specific antigen. A target antigen generally has numerous binding sites, also called epitopes, recognized by complementary-determining regions on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e. a molecule that contains an antigen-binding site that immuno-specifically binds an antigen of a target of interest or part thereof. The antibodies may be IgG e.g. IgG1, IgG2, IgG3, IgG4. Preferably, the antibody is an IgG protein and more preferably an IgG1, IgG2 or IgG4 protein. Most preferably the antibody is an IgG1 protein. The antibody can be human or derived from other species. Preferably the antibody is a human antibody.

The expression “monoclonal antibodies” as used herein characterizes antibodies that are identical because they are produced by one type of immune cell and are all clones of a single parent cell.

The expression “antibody fragment” as used herein refers to a molecule comprising at least one polypeptide chain derived from an antibody that is not full length and has at least a fragment crystallizable region enabling interaction with a ligand.

The expression “commercially formulated antibody” as used herein refers to a marketed formulation comprising a therapeutic antibody and one or more excipients. Preferably, the commercially formulated antibody is a formulation marketed in the European Union. Examples of commercially formulated antibodies include Humira®, Lemtrada®, Campath®, Tecentriq®, Bavencio®, Simulect®, LymphoScan®, Xilonix®, Scintimun®, Avastin®, Zinplava®, Blincyto®, Libtayo®, Erbitux®, hPAM4-Cide®, Zenapax®, Darzalex®, Prolia®, Unituxin®, Imfinzi®, Panorex®, Empliciti®, Gamifant®, Rencarex®, Remicade®, Besponsa®, Yervoy®, CEA-Cide®, Poteligeo®, Tysabri®, Portrazza®, Theracim®, Opdivo®, Arzerra®, Lartruvo®, Omnitarg®, Vaxira®, Cyramza®, MabThera®, Rituxan®, Sylvant®, Bexxar®, Herceptin®, Kadcyla®, Stelam®, HuMax-EGFr®, HuMax-CD4®, and biosimilars thereof. Information on commercially formulated antibodies can be found, for instance, in Allgemeine and Spezielle Pharmakologie and Toxicologie, Thomas Karow and Ruth Lang-Roth, Karow, 27^{th} ed. (2018).

Preferably, the commercially formulated antibody is Herceptin® (trastuzumab-containing formulation) as approved for marketing in the European Union by the European Medicines Agency (EMA) under authorization numbers EU/1/00/145/001 and EU/1/00/145/002 (available from Roche), or MabThera® (rituximab-containing formulation) as approved for marketing in the European Union by the EMA under authorization numbers EU/1/98/067/001, EU/1/98/067/002, EU/1/98/067/003 and EU/1/98/067/004.

The expression “Fc-fusion protein” as used herein refers to a protein comprising at least an Fc-containing antibody fragment—i.e. an immunoglobulin-derived moiety comprising at least one Fc region—and a moiety derived from a second, non-immunoglobulin protein. The Fc-containing antibody fragment forms part of the Fc-fusion protein and therefore is incorporated into the Fc-fusion protein. The Fc-containing antibody fragment can be derived from an antibody as described hereinabove, in particular from IgG e.g. IgG1, IgG2, IgG3, IgG4. Preferably, the Fc-containing moiety is derived from an IgG1 protein, more preferably from a human IgG1 protein. The non-Ig protein can be a therapeutic protein, for instance a therapeutic protein derived from erythropoietin (EPO), thrombopoietin (THPO) such as THPO-binding peptide, growth hormone, interferon (IFN) such as IFNα, IFNβ or IFNγ, platelet-derived growth factor (PDGF), interleukin (IL) such as IL1α or IL1β, transforming growth factor (TGF) such as TGFα or TGFβ, or tumor necrosis factor (TNF) such as TNFα or TNFβ, or a therapeutic protein derived from a receptor, in particular from a ligand-binding fragment of the extracellular domain of a receptor, for instance derived from cluster of differentiation 2 (CD2), CD4, CD8, CD11, CD14, CD18, CD20, CD22, CD23, CD25, CD33, CD40, CD44, CD52, CD58 (LFA3), CD80, CD86, CD147, CD164, IL2 receptor, IL4 receptor, IL6 receptor, IL12 receptor, epidermal growth factor (EGF) receptor, vascular endothelial growth factor (VEGF) receptor, epithelial cell adhesion molecule (EpCAM), or cytotoxic T-lymphocyte-associated protein 4 (CTLA4). Examples of Fc-fusion proteins include belatacept (Nulojix®), aflibercept (Eyla®), rilonacept (Arcalyst®), romiplostim (NPlate®), abtacept (Orencia®), alefacept (Amevine®), and etanercept (Enbrel®).

The term “cancer” as used herein means the physiological condition in mammals that is characterized by unregulated cell growth. A tumor comprises one or more cancer cells. Examples of cancer include carcinoma, lymphoma, blastoma, sarcoma, and leukemia or lymphoid malignancies. Further examples of cancer include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, gastrointestinal stromal tumor, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, thyroid cancer and hepatic cancer.

The expression “solid phase matrix” (or synonymously “solid support”, “solid phase” or “solid phase material”) as used herein characterizes a material that is insoluble or can be made insoluble by a subsequent reaction. Representative examples of solid phase material include polymeric or glass beads, microparticles, tubes, sheets, plates, slides, wells, and tapes.

The term “alkyl group” as used herein refers to a linear or branched hydrocarbon group having from 1 to 20 carbon atoms, preferably a methyl or an ethyl group, a cycloalkyl group having from 3 to 20 carbon atoms, preferably 5 to 8 carbon atoms. The cycloalkyl group may consist of a single ring, but it may also be formed by two or more condensed rings.

The term “aryl” as used herein refers to a radical of a monocyclic or polycyclic (e.g. bicyclic or tricyclic) 4n+2 aromatic ring system (e.g. having 6, 10, or 14 π electrons shared in a cyclic array) having 6-14 ring carbon atoms and zero heteroatoms provided in the aromatic ring system. In some embodiments, an aryl group has 6 ring carbon atoms (e.g. phenyl). In some embodiments, an aryl group has 10 ring carbon atoms (e.g. naphthyl such as 1-naphthyl and 2-naphthyl). In some embodiments, an aryl group has 14 ring carbon atoms (e.g. anthracyl). The term “aryl” as used herein is meant to encompass ring systems, wherein the aryl ring is fused with one or more carbocyclyl or heterocyclyl groups wherein the radical or point of attachment is on the aryl ring (in such instances, the number of carbon atoms designates the number of carbon atoms in the aryl ring system). Unless otherwise specified, the aryl group can be unsubstituted (an “unsubstituted aryl”) or substituted (a “substituted aryl”) with one or more (e.g. 1 to 5) substituents. Non-limiting examples of aryl groups include radicals derived from benzene, naphthalene, anthracene, biphenyl, etc.

The term “heteroaryl” as used herein refers to a radical of a 5-14 membered monocyclic or polycyclic (e.g. bicyclic, tricyclic) 4n+2 aromatic ring system (e.g. having 6, 10, or 14 π electrons shared in a cyclic array) having ring carbon atoms and 1 to 4 heteroatoms provided in the aromatic ring system, wherein each heteroatom is independently selected from nitrogen, oxygen, and sulfur. In heteroaryl groups that contain one or more nitrogen atoms, the point of attachment can be a carbon atom or a nitrogen atom, as valency permits. Heteroaryl polycyclic ring systems can include one or more heteroatoms in one or both rings. The term “heteroaryl” as used herein is meant to encompass ring systems wherein the heteroaryl ring is fused with one or more carbocyclyl or heterocyclyl groups wherein the point of attachment is on the heteroaryl ring (in such instances, the number of ring members designates the number of ring members in the heteroaryl ring system). The term “heteroaryl” is also meant to include ring systems, wherein the heteroaryl ring is fused with one or more aryl groups wherein the point of attachment is either on the aryl or heteroaryl ring (in such instances, the number of ring members designates the number of ring members in the fused polycyclic (aryl/heteroaryl) ring system).

The expression “substituted aryl group” as used herein means an aryl group in which one or more hydrogen atoms are each independently replaced with a substituent. Non-limiting examples of substituents include —Z, —R, —OR, —SR, —NR₂, —NR₃, —CZ₃, —CN, —OCN, —SCN, —NO₂, —C(O)R, —C(O)NR₂, —SO₃, —S(O)₂R, —C(S)R, —C(O)OR, —C(O)SR, where each Z is independently a halogen (i.e. —F, —Cl, —Br, or —I), and each R is independently —H, —C₁₋₂₀ alkyl or alkoxyl, —C₆₋₂₀ aryl, or —C₅₋₁₄ heteroaryl. The heteroaryl group described above may be similarly substituted.

The expression “divalent arylene group” refers to a divalent moiety derived from an optionally substituted aryl or heteroaryl group, as defined above, wherein two hydrogen atoms are replaced by covalent bonds allowing attachment to adjacent moieties. A divalent arylene-type disulfide bridge (e.g. a divalent group of formula —S—X³—S—/—S—X⁴—S— wherein X³/X⁴ represents a divalent arylene group) can be obtained by side-chain-to-side-chain cyclization according to techniques known in the art (see Stefanucci et al. in ACS Med.Chem. Left. 2017, 8, 449-454, and Beard et al. Bioorg. & Med. Chem. 2018, 26, 3039-3045).

The expression “divalent xylene group” as used herein refers to a divalent moiety derived from one of the three isomers of dimethylbenzene (i.e. ortho-xylene, meta-xylene, para-xylene), in which one hydrogen atom of each methyl group is replaced by a covalent bond allowing attachment to an adjacent moiety. Preferably, the divalent xylene group is a divalent meta-xylene group. A divalent xylene-type disulfide bridge (e.g. a divalent group of formula —S—X³—S—/—S—X⁴—S— wherein X³/X⁴ represents a divalent xylene group) can be obtained by side-chain-to-side-chain cyclization in the presence of e.g. dibromo-xylene as described by Stefanucci et al. in ACS Med.Chem. Lett. 2017, 8, 449-454.

The expression “divalent maleimide group” as used herein refers to a divalent moiety derived from maleimide, in which the hydrogen atoms at positions 2 and 3 are each replaced by a covalent bond allowing attachment to an adjacent moiety. A divalent maleimide-type disulfide bridge (e.g. a divalent group of formula —S—X³—S—/—S—X⁴—S— wherein X³/X⁴ represents a divalent maleimide group) can be obtained by side-chain-to-side-chain cyclization in the presence of e.g. 2,3-dibromomaleimide or another suitable reagent as described by Kuan et al. in Chem. Eur. J. 2016, 22, 17112-17129.

The expression “divalent acetone group” as used herein refers to a divalent moiety derived from acetone (ACE), in which one hydrogen atom of each methyl group is replaced by a covalent bond allowing attachment to an adjacent moiety. A divalent ACE-type disulfide bridge (e.g. a divalent group of formula —S—X³—S—/—S—X⁴—S— wherein X³/X⁴ represents a divalent ACE group) can be obtained by side-chain-to-side-chain cyclization in the presence of e.g. dibromoacetone or dichlororoacetone (see e.g. Assem et al. Angew. Chem. Int. Ed. Engl. 2015, 54(30), 8665-8668).

The expression “group capable of modulating the electron density and stability of X” as used herein refers to a group which can modulate (increase or decrease) the properties (electron density/stability) of the neighboring group (X), e.g. moiety (F2) in formula (3b). The modulating group (M) may withdraw or donate electrons to the neighboring group, for instance by an inductive effect and/or a mesomeric effect (see International Union of Pure and Applied Chemistry, Compendium of Chemical Technology, Gold Book 2012, 477-480). Preferably, inductive and mesomeric effects may lead to a displacement of the electronic density distribution towards the modulating group, thereby modulating the electron density and stability of the neighboring group (e.g. F2). The modulation of the electron density can be determined by ¹³C NMR spectroscopy, for instance by measuring the shifts of the carbon atom of the carbonate group and comparing the same with the shift of a reference compound e.g. compound 31. A change of the NMR shift of the carbonate signal to higher ppm values (compared to the shift of the reference compound) is indicative of a reduction of the electron density and thus a reduction of stability. A change of the NMR shift of the carbonate signal to lower ppm values (compared to the shift of the reference compound) is indicative of an increase of the electron density and increase of stability. Said modulation of electron density can be used to optimize reactivity and stability of the conjugate of the invention.

According to an embodiment of the present invention, the group capable of modulating the electron density and stability of X is selected such that, in the absence of further reagents, the conjugate is stable to degradation (e.g. hydrolysis) which means that the conjugate exhibits less than 50% degradation, preferably less than 25% degradation, more preferably less than 10% degradation, in particular less than 5% degradation, when being mixed with water/DMSO (95/5, v/v) at pH 9 at a concentration of 1 mg/mL and stirred at 500 rpm for 1 hour at 25° C., as determined by HPLC.

The expression “electron-withdrawing group” as used herein refers to a group or substituent that can withdraw electrons from the moiety to which it is bonded, i.e. reduce the electron density of this moiety in comparison with the same moiety carrying a hydrogen atom instead of the electron-withdrawing group. Typical electron withdrawing groups include, but are not limited to cyano, nitro, haloalkyl, carboxyl, aryl, sulfonyl, etc. The electron-withdrawing group can exert its electron-withdrawing effect by inductive effect and/or mesomeric effect (as indicated above). The expression “electron-withdrawing” as used herein is meant to encompass both meanings. Electron-withdrawing groups/substituents are known in the art and described e.g. by Carey & Sundberg in Advanced Organic Chemistry, Part A: Structure and Mechanisms, 4^{th} Edition.

The expression “leaving group” as used herein refers to an atom or group (which may be charged or uncharged) that becomes detached from an atom or a molecule in what is considered to be the residual or main part of the molecule taking part in a specific reaction, for instance a nucleophilic substitution reaction (Pure Appl. Chem. 1994, 66, 1134). Examples of leaving groups include thiophenolates, phenolates, carboxylates, sulfonates.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless specified otherwise or the context dictates otherwise, references to groups being “substituted” or “optionally substituted” are to be understood as references to the presence (or optional presence, as the case may be) of at least one substituent selected from F, Cl. Br, I, CN, NO₂, NH₂, NH—C₁₋₆-alkyl, N(C₁₋₆-alkyl)₂, —X—C₁₋₆-alkyl, —X—C₂₋₆-alkenyl, —X—C₂₋₆-alkynyl, —X-(5-14-membered heteroalkyl with 1-3 heteroatoms selected from N, O, S), wherein X represents a single bond, —(CH₂)—, —O—, —S—, —S(O)—, —S(O)₂—, —NH—, —CO—, or any combination thereof including, for instance, —C(O)—NH—, —NH—C(O)—. The number of substituents is not particularly limited and may range from 1 to the maximum number of valences that can be saturated with substituents. It is typically 1, 2 or 3 and usually 1 or 2, most typically 1.

Unless specified otherwise, all valencies of the individual atoms of the compounds or moieties described herein are saturated. In particular, they are saturated by the indicated binding partners. If no binding partner or a too small number of binding partners is indicated, the remaining valencies of the respective atom are saturated by a corresponding number of hydrogen atoms.

Unless specified otherwise, chiral compounds and moieties may be present in the form of a pure stereoisomer or in the form of a mixture of stereoisomers, including the 50:50 racemate. In the context of the present invention, references to specific stereoisomers are to be understood as references to compounds or moieties, wherein the designated stereoisomer is present in at least 90% enantiomeric excess (ee), more preferably at least 95% ee and most preferably 100% ee, wherein % ee is defined as (|R−S51 )/(R+S)*100% with R and S representing the amount of moles of the respective enantiomers.

Unless specified otherwise or dictated otherwise by the context, all connections between adjacent amino acid groups are formed by peptide (amide) bonds.

Unless the context dictates otherwise, and/or alternative meanings are explicitly provided herein, all terms are intended to have meanings generally accepted in the art, as reflected by IUPAC Gold Book (status of 1 Nov. 2019), or the Dictionary of Chemistry, Oxford, 6^{th} Ed.

### 2. OVERVIEW

The present invention is based on the surprising finding that the regioselective attachment of a payload to an antibody or antibody fragment can be accomplished using a compound of the invention, and more particularly that the said regioselective attachment can be accomplished in one single step, e.g. without need for further chemical reaction to cleave a covalent bond between the vector and the antibody or antibody fragment.

### 3. COMPOUND OF FORMULA (1)

The present invention relates to a compound represented by the general formula (1):

P—Y—S—V (1)

The compound of formula (1) contains a vector V capable of interacting with (having binding affinity for) the Fc region of an antibody or fragment thereof, said antibody fragment being optionally incorporated into an Fc-fusion protein, a spacer S having a length Z, a reactive moiety Y, and a payload P.

3.1 Payload (P)

The payload to be used is not particularly limited and any e.g. labelling and/or pharmaceutically active molecule can be employed as long as it can be attached to the reactive moiety.

According to one embodiment, the payload comprises a moiety selected from the following:
(i) a moiety selected from
   a labelling moiety which may include a radionuclide, preferably a chelating agent such as 1,4,7,10-tetraatacyclododecane-1,4,7,10-tetraacetic acid (DOTA), diethylenetriamine pentaacetic acid (DTPA), cyclohexyl diethylenetriamine pentaacetic acid (CH-X-DTPA), 3,6,9,15-Tetraazabicyclo[9.3.1]pentadeca-1(15), 11,13-triene-3,6,9-triacetic acid (PCTA) or desferrioxamine (DFO), wherein said chelating agent optionally chelates a radionuclide;
   a chromophore;
   a fluorophore such as fluorescein or rhodamine; and
   a labelling moiety containing a radionuclide such as ¹²⁵I, ¹²³I, ¹³¹I, ¹⁸F, ¹¹C, ¹⁵O, ¹⁸F, for instance, a moiety derived from 4-hydroxyphenylpropionate (aka Bolton-Hunter reagent) containing a radionuclide such as ¹²⁵I, ¹²³I or ¹³¹I;
(ii) a moiety selected from a moiety comprising a conjugation group to allow later attachment of a payload as specified under items (i) and (iii) herein. This may be a moiety selected from the group consisting of an optionally substituted conjugated diene; an optionally substituted tetrazine; an optionally substituted alkyne or azide; an optionally substituted dibenzocyclooctyne (DBCO); an optionally substituted trans-cyclooctene (TCO), an optionally substituted bicyclo[6.1.0]nonyne (BCN); an optionally substituted aldehyde; an optionally substituted ketone; and an optionally substituted hydrazine;
(iii) a moiety derived from a drug selected from
   an antineoplastic agent such as a DNA-alkylating agent e.g. duocarmycin;
   a topoisomerase inhibitor e.g. doxorubicin;
   an RNA-polymerase II inhibitor e.g. alpha-amanitin;
   a DNA cleaving agent e.g. calicheamicin;
   an antimitotic agent or microtubule disruptor e.g. a taxane an auristatin or a maytansinoid;
   an anti-metabolite;
   a kinase inhibitor such as ipatasertib;
   an immunomodulatory agent;
   an anti-infectious disease agent;
   and radioisotopes and/or pharmaceutically acceptable salts thereof;

According to one embodiment, the payload (P) is a chelating agent that optionally chelates a radionuclide, wherein the chelating agent is preferably a moiety derived from diethylenetriamine pentaacetic acid (DTPA), 3,6,9,15-Tetraazabicyclo[9.3.1]pentadeca-1(15), 11,13-triene-3,6,9-triacetic acid (PCTA), cyclohexyl diethylenetriamine pentaacetic acid (CHX-DTPA), desferrioxamine (DFO), 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), 1,4,7,10-tetraatacyclododecane-1,4,7,10-tetraacetic acid (DOTA), 1,4,7-triazacyclononane-1,4,7-triacetic acid (NOTA), ethylenediaminetetraacetic acid (EDTA), ethylenediaminediacetic acid, triethylenetetraminehexaacetic acid (TTNA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)tracetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a, 10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-tetraazabicyclo[9.3.1]pentadeca-1(15), 11,13-triene-3,6,9-triacetic acid (PCTA), 2,2′,2″,2″′-(1,4,7,10-tetraazacyclotridecane-1,4,7,10-tetrayl)tetraacetic acid (TRITA), 2,2′,2″,2″′-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)tracetamide (TRITRAM), trans-N-dimethyl-cyclam, 2,2′,2″-(1,4,7-triazacyclononane-1,4,7-triyl)tracetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), or derivatives thereof.

According to one preferred embodiment, the payload is a chelating agent that optionally chelates a radionuclide, which is a moiety derived from DTPA, DOTA, DFO, NOTA, PCTA, CH-X-DTPA, NODAGA, or DOTAGA, preferably a moiety derived from DTPA, DOTA, DFO, NOTA, PCTA, CH-X-DTPA, or NODAGA, more preferably a moiety derived from DTPA, DOTA, DFO, or PCTA. Most preferably, the chelating agent is DTPA.

According to one embodiment, the chelating agent chelates a radionuclide selected from ¹²⁴I, ¹³¹I, ⁸⁶Y, ⁹⁰Y, ¹⁷⁷Lu, ¹¹¹In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²Bi, ²¹³Bi, ⁷²As, ²¹¹At, ²²⁵Ac, ²²³Ra, ⁹⁷Ru, ¹⁴⁹Tb, ¹⁵²Tb, ¹⁶¹Tb, ^{99m}Tc, ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F, preferably from ⁸⁹Zr, ¹¹¹In, ⁶⁴Cu, ¹⁷⁷Lu, ⁶⁸Ga, ^{99m}Tc, ²⁰³Pb, ⁷²As, ⁵⁵Co, ⁹⁷Ru, ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, in particular ¹¹¹In.

In one preferred embodiment, the payload is DTPA that chelates ¹¹¹In.

In another preferred embodiment, the payload is selected from:
a moiety (i) derived from DOTA, PCTA, DTPA or CH-X-DTPA that chelates ¹¹¹In, most preferably CH-X-DTPA that chelates ¹¹¹In;
a moiety (i) derived from NOTA, NODAGA or PCTA that chelates ⁶⁴Cu, most preferably NOTA that chelates ⁶⁴Cu;
a moiety (i) derived from DOTA, DFO, DFO′ or DFO-cyclo′ that chelates ⁸⁹Zr, most preferably DFO that chelates ⁸⁹Zr.

According to one embodiment, the payload is a moiety selected from a moiety comprising a conjugation group to allow later attachment of a payload as specified under items (i) and (iii) herein. This may be a moiety comprising a conjugation group suitable for “click chemistry” that generates covalent bonds quickly and reliably by reacting with another moiety comprising a “click chemistry” partner group (i.e. a payload comprising a conjugation partner group), for instance, via strain-promoted cycloaddition, [2+3] dipolar cycloaddition, or Diels-Alder cycloaddition.

In one embodiment, the moiety is a moiety comprising a conjugation group selected from the group consisting of an optionally substituted conjugated diene, an optionally substituted tetrazine, an optionally substituted alkyne or azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, and an optionally substituted hydrazine.

In one embodiment, the moiety is a moiety comprising a conjugation group that can react to form covalent bonds in the absence of a metal catalyst (“metal-free”) as described e.g. by Becer et al. in “Click Chemistry beyond Metal-Catalysed Cycloaddition” Angewandte Chemie Int. Ed. 2009, 48(27), 4900-4908. Examples of conjugation groups which can react in the absence of a metal catalyst include electron-deficient alkynes, strained alkynes such as cyclooctynes, tetrazines, and azides. Preferably, the moiety is a moiety comprises a conjugation group selected from azide (N₃), TZ, TCO, BCN and DBCO, more preferably BCN or DBCO, most preferably DBCO.

According to one embodiment, the payload is a moiety derived from a drug. Hereinafter are exemplary drugs that can be used as a payload in the compound of the present invention:

(A) Antineoplastic agents such as DNA-alkylating agents e.g. duocarmycin (including synthetic analogues: adozelesin, carzelesin, bizelesin, KW-2189 and CBI-TMI), nitrogen mustard analogues (e.g. cyclophosphamide chlorambucil, melphalan, chlormethine, ifosfamide, trofosfamide, prednimustine, bendamustine, chlornaphazine, estramustine, mechlorethamine, mechlorethamine oxide hydrochloride, mannomustine, mitolactol, novembichin, phenesterine, uracil mustard), alkyl sulphonates (e.g. busulfan, treosulfan, mannosulfan, improsulfan and piposulfan), ethylene imines (e.g. thiotepa, triaziquone, carboquone); nitrosoureas (e.g. carmustine, lomustine, semustine, streptozocin, chlorozotocin, fotemustine, nimustine, ranimustine), epoxides (e.g. etoglucid), other alkylating agents (e.g. mitobronitol, pipobroman, temozolomide, dacarbazine);

(B) Topoisomerase inhibitors e.g. doxorubicin, morpholino-doxorubicin, cyanomorpholino-doxorubicin, 2-pyrrolino-doxorubicin, deoxydoxorubicin, etoposide, etoposide phosphate, irinotecan and metabolites thereof such as SN-38, teniposide, topotecan, resveratrol, epipodophyllins (e.g. 9-aminocamptothecin, camptothecin, crisnatol, daunomycin, mitoxantrone, novantrone, retinoic acids (retinols), 9-nitrocamptothecin (RFS 2000));

(C) RNA-polymerase II inhibitors e.g. alpha-amanitin, other amatoxins;

(D) DNA-cleaving agents e.g. calicheamicin;

(E) Antimitotic agents or microtubule disruptors e.g. vinca alkaloids (e.g. vincristine, vinblastine, vindesine, vinorelbine, navelbin, vinflunide, vintafolide); taxanes (e.g. paclitaxel, docetaxel, paclitaxel polyglumex, cabazitaxel) and their analogs, maytansinoids (e.g. DM1, DM2, DM3, DM4, maytansine and ansamitocins) and their analogs, cryptophycins (e.g. cryptophycin 1 and cryptophycin 8); epothilones, eleutherobin, discodermolide, bryostatins, dolostatins, auristatins (e.g. monomethyl auristatin E, monomethyl auristatin F), tubulysins, cephalostatins; pancratistatin, sarcodictyin, spongistatin, demecolcine, mitomycins;

(F) Anti-metabolites e.g. DHFR inhibitors (e.g. methotrexate, trimetrexate, denopterin, pteropterin, aminopterin (4-aminopteroic acid) or other folic acid analogues such as raltitrexed, pemetrexed, pralatrexate); IMP dehydrogenase inhibitors (e.g. mycophenolic acid, tiazofurin, ribavirin, EICAR); ribonucleotide reductase inhibitors (e.g. hydroxyurea, deferoxamine); pyrimidine analogs (e.g. cytarabine, fluorouracil, 5-fluorouracil and metabolites thereof, tegafur, carmofur, gemcitabine, capecitabine, azacitidine, decitabine, fluorouracil combinations, tegafur combinations, trifluridine combinations, cytosine arabinoside, ancitabine, floxuridine, doxifluridine), uracil analogs (e.g. 6-azauridine, deoxyuridine); cytosine analogs (e.g.

enocitabine); purine analogs (e.g. azathioprine, fludarabine, mercaptopurine, thiamiprine, thioguanine, cladribine, clofarabine, nelarabine); folic acid replenisher such as folinic acid;

(G) Kinase inhibitors e.g. ipatasertib, BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, afatinib, vemurafenib, crizotinib, regorafenib, masitinib, dabrafenib, trametinib, ibrutinib, ceritinib, lenvatinib, nintedanib, cediranib, palbocidib, osimertinib, alectinib, alectinib, rociletinib, cobimetinib, midostaurin, olmutinib, E7080 (anti-VEGFR2), mubritinib, ponatinib (AP24534), bafetinib (INNO-406), bosutinib (SKI-606), cabozantinib, vismodegib, iniparib, ruxolitinib, CYT387, tivozanib, ispinesib, temsirolimus, everolimus, Ridaforolimus;

(H) Immunomodulatory agents include immunostimulants, immunosuppressants, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, prednisone, triamcinolone acetonide, beclometasone dipropionate), DHEA, hydroxychloroquine, meloxicam, methotrexate, mofetil, mycophenylate, sirolimus, tacrolimus, everolimus, fingolimod, ibrutinib;

(I) Anti-infectious disease agents include antibacterial drugs, antimycobacterial drugs and antiviral drugs. A non-limiting example of antibiotic used in an antibiotic-antibody drug conjugate is rifalogue, a rafamycin derivative.

According to one embodiment, the payload is a moiety derived from exatecan, PNU-159682, (alpha-)amanitin, duocarmycin, auristatin, maytansine, tubulysin, calicheamicin, SN-38, taxol, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof.

In some aspects of the present invention, it can be preferable to use a payload having a certain level of hydrophilicity, for instance, in the case of a chelating agent that chelates a radionuclide, in order to avoid and/or prevent possible phenomena of aggregation. High aggregation phenomenon can be overcome, for example, by addition/increasing of/the number of PEG units of a linker present between the antibody and the payload.

This attachment of the payload to the reactive group may be made via a linking group (or “linker”). In the context of this disclosure, this linking group may be considered as being part of the payload. Accordingly, in an embodiment, the payload is represented by the following formula (2):

P¹-L-*′ (2)
wherein,
P¹ represents a payload as described hereinbefore—e.g. a chelating agent that optionally chelates a radionuclide such as ¹⁷⁷Lu-DOTA, or a moiety derived from a drug,
L represents a linker,
*′ indicates covalent attachment to the reactive moiety.

The linker is a divalent group, preferably comprising one or more atoms selected from carbon, nitrogen, oxygen, and sulfur.

In an embodiment, the linker can be selected from
(a1) an alkylene group having from 1 to 12 carbon atoms, preferably an alkylene group having from 2 to 6 carbon atoms such as an ethylene group or propylene group;
(b1) a polyalkylene oxide group with 2 or 3 carbon atoms having from 1 to 36 repeating units; preferably a group represented by the formula —NH—(CH₂CH₂₀)ₙ₁—CH₂CH₂— wherein n1 is an integer of 0 to 35, e.g. 1 to 20; and
(c1) a peptidic group having 2 to 12 amino acids.

In more specific embodiments, the linker is selected from
(a1) an alkylene group having 2 to 6 carbons (—(CH₂)₂₋₆—);
(b1) a polyalkylene group of formula —NH—(CH₂CH₂O)ₙ₁—CH₂CH₂—, n1 being an integer of 0-35; and
(c1) a peptidic linker comprising 2 to 12 amino acids, which is optionally cleavable, preferably a cleavable peptidic linker comprising a Val-Cit unit, a Val-Ala unit, a Val-Cit-PABC or a Val-Cit-PABC-DMEA unit.

The linker may be a cleavable or non-cleavable linker. In an embodiment the linker is a non-cleavable linker. In another embodiment the linker is a cleavable linker.

The cleavable linker may be a linker capable of specifically releasing the payload upon internalization in a target cell. It may utilize an inherent property of the target cell, e.g. a tumor cell, for selectively releasing the payload from the modified antibody or modified antibody fragment, namely (1) protease-sensitivity (enzyme-triggered release linker system), (2) pH-sensitivity, (3) glutathione-sensitivity, or (4) glucoronidase sensitivity. In a specific embodiment, the linker is a cleavable linker comprising a valine-citrulline (Val-Cit) or valine-alanine (Val-Ala) dipeptide that can serve as a substrate for intracellular cleavage by Cathepsin B (Cat B).

In another specific embodiment the linker is a cleavable linker comprising a self-immolative moiety capable of releasing the payload by elimination- or cyclization-based mechanism. An example of a cleavable linker comprising a self-immolative moiety is the para-amino benzyloxycarbonyl (PABC) linker as used e.g. in the bremtuximab-vedotin conjugate Adcetris® (Younes et al. N. Engl. J. Med. 2010, 363, 1812-1821; Jain et al. Pharm. Res. 2015, 32(11), 3526-3540). The PABC-containing linker comprises a protease-sensitive Val-Cit-PABC dipeptide linker unit, which can be recognized and cleaved by Cat B. The linker unit can be attached to the reactive moiety (and after antibody modification to the antibody) by means of a maleimidocaproyl moiety. Such a linker can help avoid steric conflicts in substrate recognition by Cat B. After enzymatic cleavage of the citrulline-PABC amide bond, the resulting PABC-substituted payload spontaneously undergoes a 1,6-elimination that releases the free payload as the product into the target cell. Accordingly, the group according to formula (2) may represent vedotin, i.e. a group consisting of a payload moiety derived from monomethyl auristatin E attached to the reactive moiety via a linker comprising a Val-Cit-PABC unit.

In another specific embodiment the linker is a cleavable linker comprising a C-terminal dipeptide unit capable of acting as a highly specific substrate for the exopeptidase activity of Cat B (exo-Cat B). Examples of exo-Cat B-cleavable linkers systems are described in WO 2019/096867 A1. In particular, the linker L can comprise a C-terminal dipeptide unit (“Axx-Ayy” or “Ayy-Axx”) as defined in claim 1, 2 or 3 of WO 2019/096867 A1.

3.2 Reactive Moiety (Y)

The compound of the present invention comprises a reactive moiety (Y) which can react (e.g. via a nucleophilic substitution reaction) with the side chain of an amino acid exposed at the surface of an antibody or antibody fragment. Preferably, the reactive moiety is capable of reacting with the side chain lysine. This reaction leads to the covalent attachment of the payload (P) to said antibody or antibody fragment, with the concomitant release of the spacer (S) and vector (V). When the reactive moiety (Y) reacts with the side chain of an amino acid exposed at the surface of an antibody or antibody fragment to form a covalent bond, a covalent bond within Y or between Y and S is spontaneously cleaved to release the peptide (without need for further chemical reaction such as hydrolysis or reduction).

The reactive moiety comprises a reactive center (RC) that is capable of reacting with the side chain of an amino acid, preferably with the side chain of a lysine residue, for instance via a nucleophilic substitution reaction. Preferably, the reactive center is electrophilic. Non-limiting examples of electrophilic reactive centers capable of reacting with the side chain of an amino acid include C═O and C═S. A preferred reactive center is carbonyl (C═O) or thiocarbonyl (C═S), and particularly preferred is carbonyl (C═O).

Covalently attached to one side of the reactive center is a moiety (F1) through which the reactive center (RC) is attached to the payload (P), covalently attached to the other side of said reactive center is a moiety (F2) through which the reactive center (RC) is attached via the spacer (S) to the vector (V). Accordingly, the reactive moiety (Y) may be represented by the following formula (3a):

**—F1-RC—F2-* (3a)
wherein,
RC is a reactive center, preferably an electrophilic reactive center, and more preferably a group selected from C═O and C═S, most preferably C═O;
F1 represents a single covalent bond, an atom, or a group of atoms; preferably an atom selected from O and S, or a group of atoms comprising one or more atoms selected from C, N, O and S; more preferably an atom selected from O and S;
F2 represents an atom, or a group of atoms; preferably an atom selected from O and S, or a group of atoms comprising one or more atoms selected from C, N, O, and S; more preferably an atom selected from O and S;
** indicates attachment to the payload (P), and
* indicates attachment to the spacer (S).

F1 and F2 may be identical atoms or groups of atoms. However, preferably the atom or group of atoms that constitute F2 make it a better/preferred leaving group than/to F1 in a nucleophilic substitution reaction. This ensures that when the reactive center reacts with the side chain of an amino acid residue, for instance with the side chain of a lysine residue, on the antibody or antibody fragment via a nucleophilic substitution reaction, F2 is the preferred leaving group; resulting in the payload being attached to the antibody or antibody fragment and not the vector/spacer construct.

According to one embodiment, the reactive moiety of formula (3a) is represented by one of the following formulae (4a) to (4m)

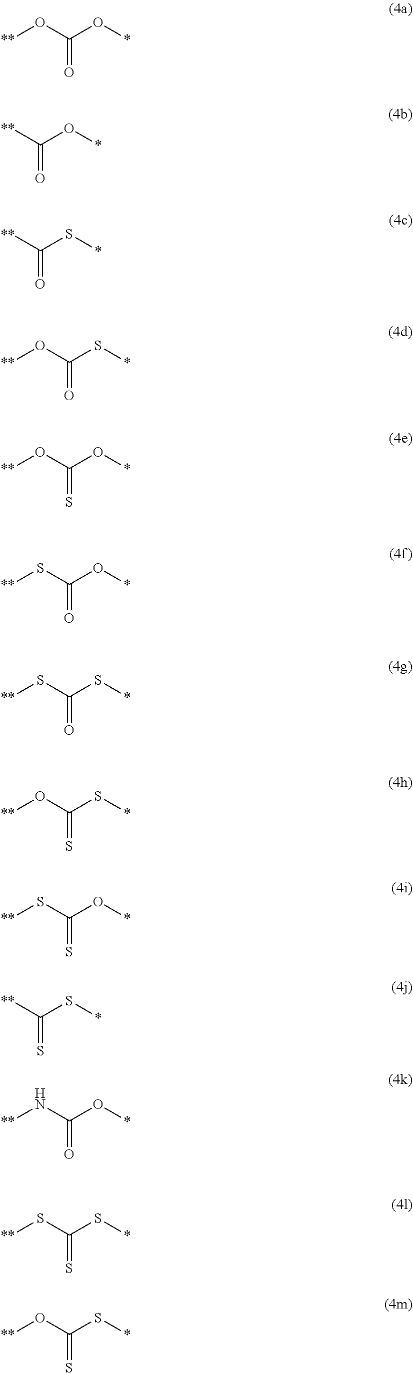

Wherein ** indicates attachment to the payload (P), and * indicates attachment to the spacer (S).

To ensure that F2 is a better or more preferred leaving group than F1 in a nucleophilic substitution reaction, especially if F1 and F2 are the same atom or group of atoms, F2 may be linked to a modifying group (M) wherein, M is a group capable of modulating the electronegativity and/or stability of the neighbouring moiety F2 e.g. by withdrawing or donating electrons.

Accordingly, in an embodiment, the reactive moiety (Y) is represented by the following formula (3b):

**—(F1-RC—F2)-(M)-* (3b)

Wherein, RC, F1, F2, **, and * are as defined in formula (3a) above, and M represents a group capable of modifying the electron density and stability of F2, preferably a group capable of withdrawing electrons.

In an embodiment, M is represented by the following formula (3c):

***′-M′-B—C—* (3c)
wherein,
M′ is an aryl group having 6, 10 or 14 ring members and 1, 2 or 3 condensed rings, respectively, or a heteroaryl group having 5 to 20 ring members, 1, 2 or 3 condensed rings and 1 to 4 heteroatoms independently selected from N, O and S, which may be substituted with one or more substituents; preferably a phenyl group, a naphthyl group, a pyridyl group, a quinolinyl group, an isoquinolinyl group or a benzotriazolyl group, which may be substituted with one or more substituents, each substituent being preferably selected from —F, —Br, —Cl, —I, —NO₂, —CN, —C₁₋₆-alkoxy, —C₁₋₆-amido such as —C(O)NH₂, and combinations thereof such as —CCl₃, —CF₃ or —CH₂NO₂;
B is a single covalent bond, O, S, NR′ wherein R′ represents a hydrogen atom, OH, an alkyl group or a cycloalkyl group, a C₂₋₆-alkenylene, a C₂₋₆-alkynylene, a group having the general formula:

—(CH₂)ₙ₁—(H¹)ₓ₁—(CH₂)ₙ₂—(H²)ₓ₂—(CH₂)ₙ₃—(H³)ₓ₃—(CH₂)ₙ₄— (3c′)
wherein,
each of n1, n2, n3 and n4 represents an integer independently selected from 0 to 10 such that n1+n2+n3+n4 is 10 or less,
each of x1, x2 and x3 is independently selected from 0 and 1, and
each H¹, H² and H³ is an atom independently selected from N, O and S, provided that if x1+x2=2, n2≥1, if x2+x3=2, n3≥0, if x1+x3=2, n2≥1 or n3≥1, and if x1+x2+x3 is 3, n2≥1 and n3≥1;
or any combination thereof; preferably a single covalent bond, NH or a C₁₋₁₀-alkylene group; more preferably a single covalent bond;
C is C═O, C═S, C(NR″) wherein R″ represents a hydrogen atom, OH, an alkyl group or a cycloalkyl group, S(═O), or S(═O)₂; preferably C═O;
* indicates covalent attachment to the spacer (S); and
***′ indicates covalent attachment to F2.

According to one embodiment, in formula (3b), the moiety (F1-RC—F2) is represented by one of the formulae (4a′) to (4m′) and/or M is independently represented by one of the following formulae (5a) to (5j′):

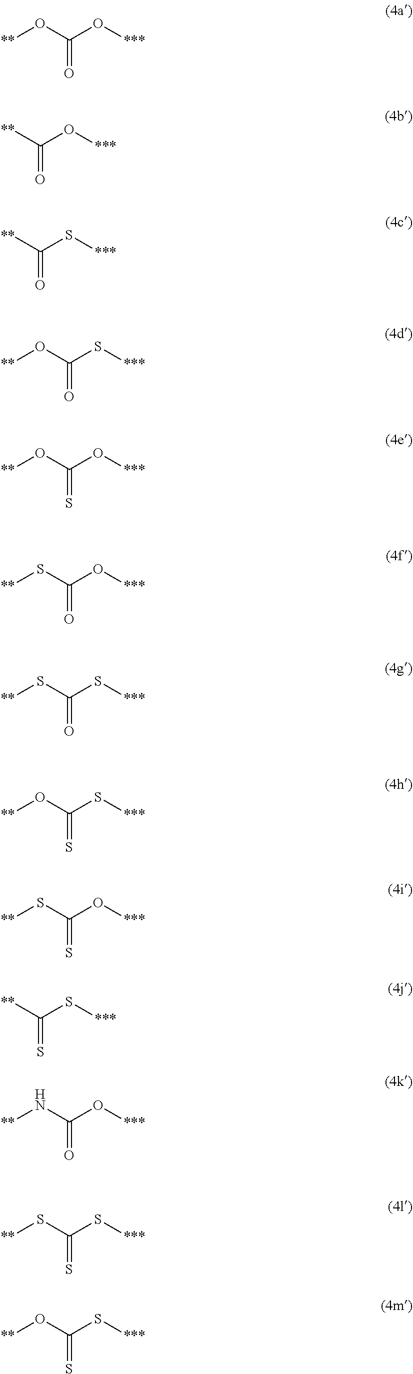

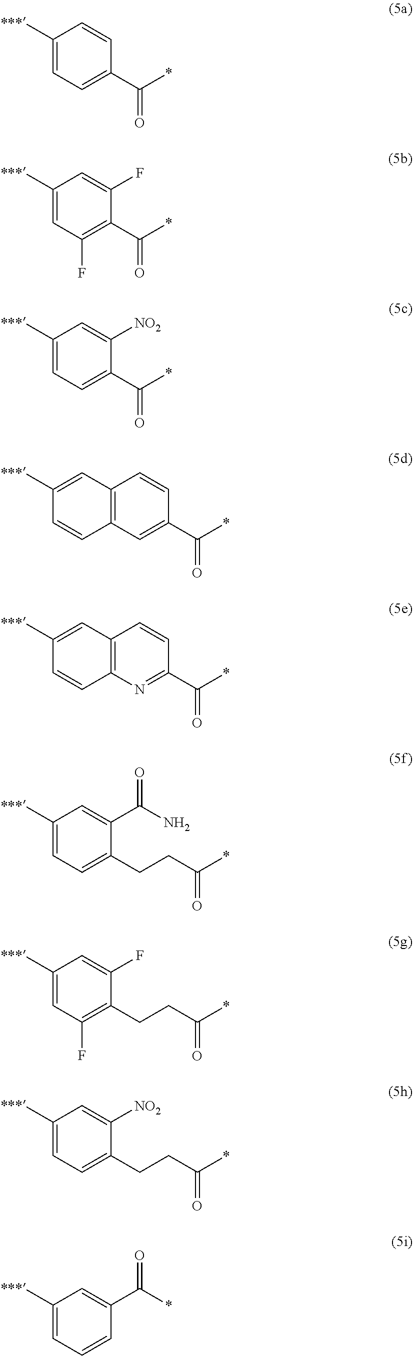

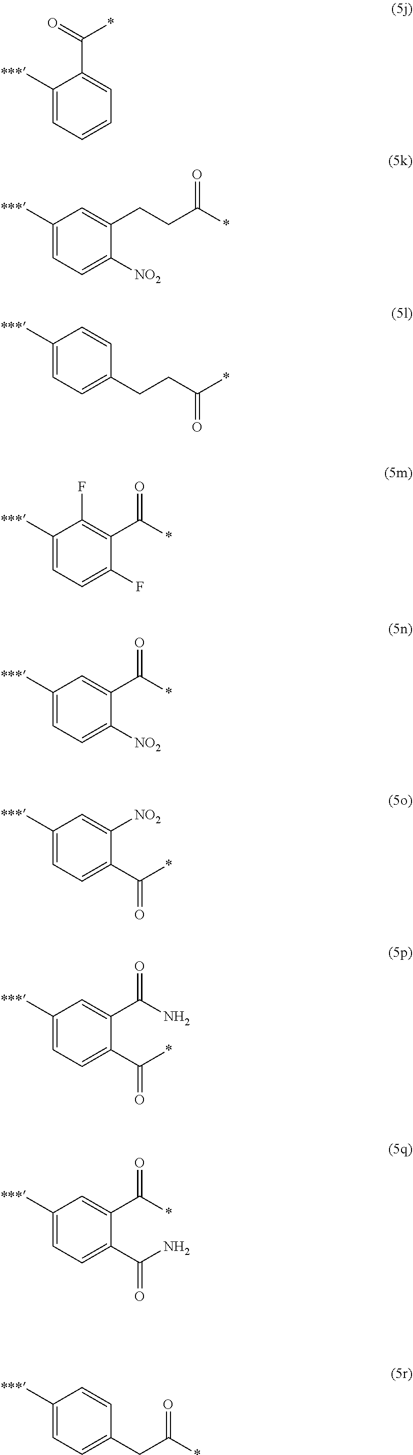

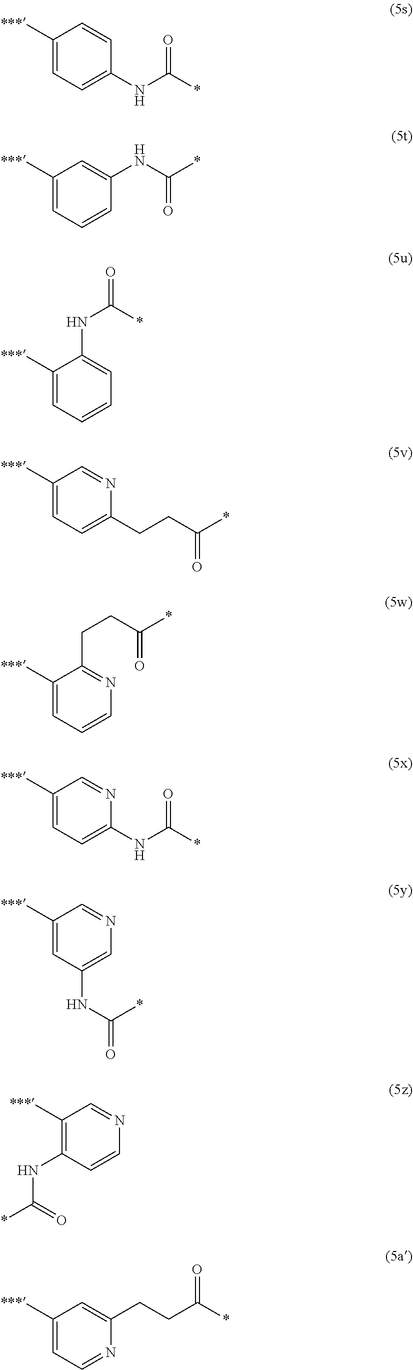

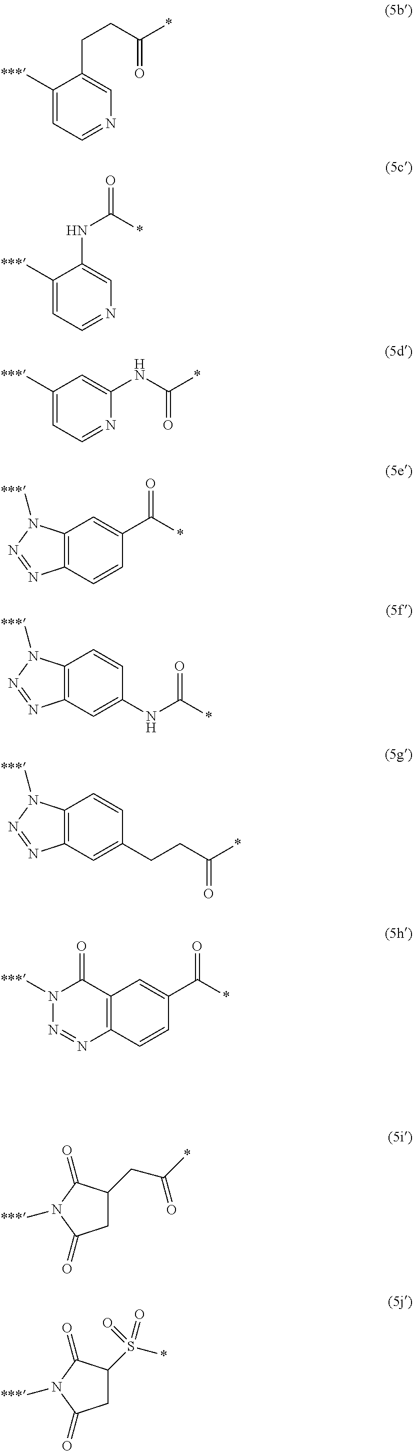

Wherein * indicates covalent attachment to the spacer (S), ** indicates covalent attachment to the payload (P), *** indicates covalent attachment to the modifying group (M), and ***′ indicates covalent attachment to F2.

In preferred embodiments, the reactive moiety is represented by one of the following formulae (6a) to (6l′):

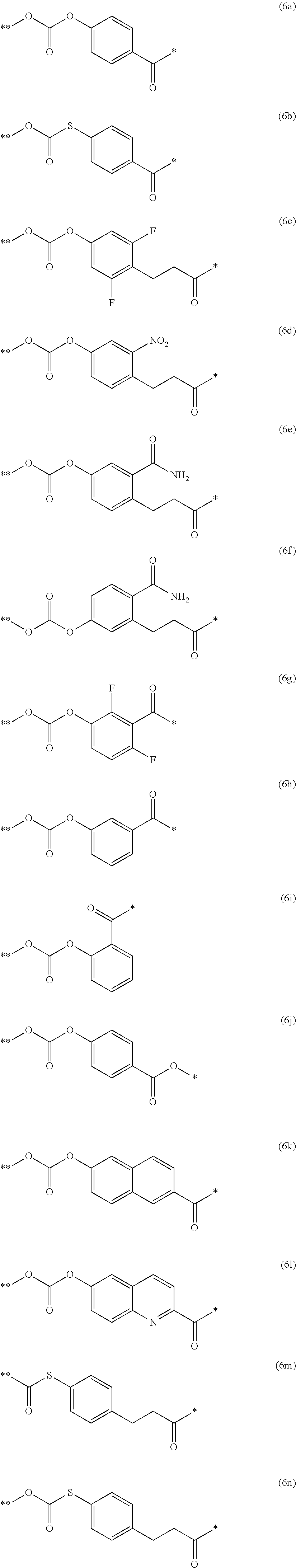

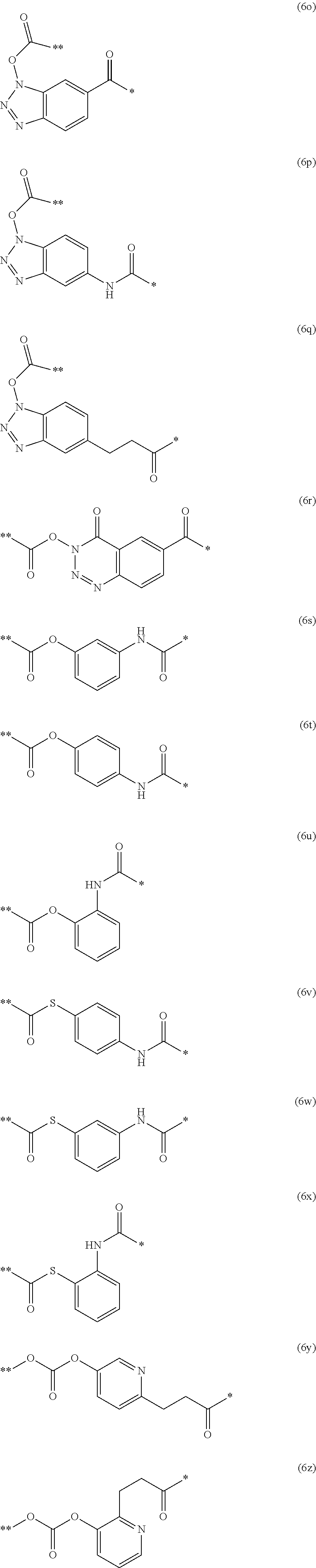

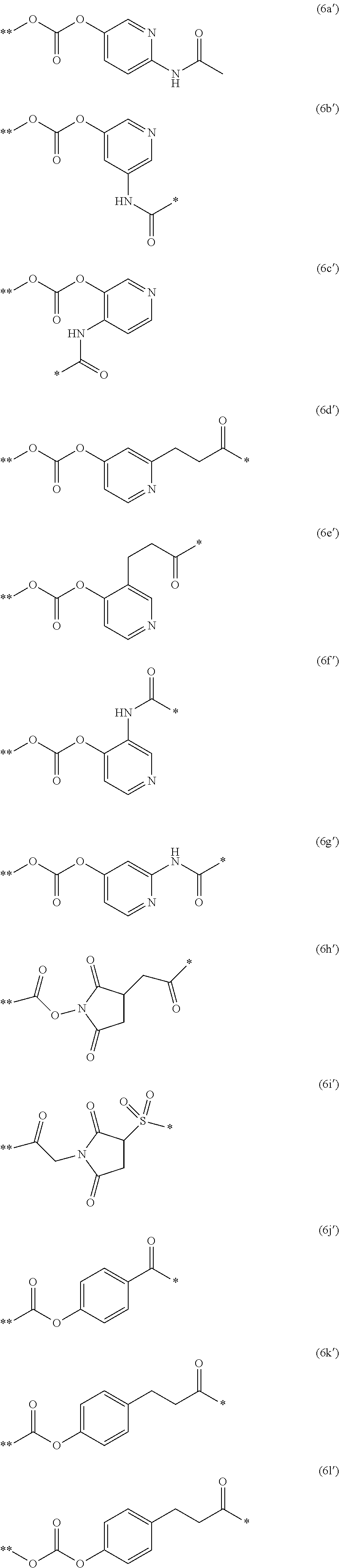

Wherein * indicates covalent attachment to the spacer (S), and ** indicates covalent attachment to the payload (P).

Most preferably, the reactive moiety is represented by one of the formulae (6a), (6b) and (6m), in particular by formula (6a).

3.3 Spacer (S)

The compound of the present invention comprises a spacer (S) having a length Z, wherein the length Z is a length such that when the vector interacts with the Fc region of an antibody or fragment thereof, the reactive moiety is able to react with the side chain of an amino acid residue exposed at the surface of the antibody or antibody fragment, leading to the regioselective attachment of the payload (and optionally the linker) to the antibody or antibody fragment. The spacer is attached to the vector (V) via a functional group (e.g. an amino group, a carboxyl group) of the vector's chemical structure. If the vector is a peptide, the spacer is attached to the N-terminus or to the C-terminus of the peptide (as described further below). For example, the spacer can be attached to an amino or carboxyl function at the N-terminal or C-terminal of the polypeptide backbone, or to the N-terminal or C-terminal amino acid side chain. Especially in case of non-peptidic vector molecules, it is preferable to identify a point of attachment of the spacer (S) such that there is no significant (<20%) reduction of binding affinity (expressed as K_{d}) in comparison with the vector not having the spacer attached.

The length Z refers to the length of the spacer in its natural conformation (not its maximal stretched length). The natural conformation may be taken when the spacer is linked to the vector and the reactive moiety as part of the construct of the reactive conjugate of the invention.

Suitable lengths for length Z can be determined by using computer modeling (Molecular Operating Environment (MOE) available from Chemical Computing Group) or X-ray crystallography to calculate an approximate distance in Angstroms (Å) between the binding site of the vector on the Fc domain of the antibody or fragment thereof and the targeted amino acid e.g. a lysine or cysteine residue, most preferably a lysine. In the case of polymers, the length Z can be determined by applying the worm-like-chain (WLC) model as described further below. A three-dimensional structure for the Fc-III/Fc-region complex at a resolution of 2.7 Å is available under the PDB identifier 1DN2 (DeLano et al. Science 2000, vol. 287, no. 5456, 1279-1283).

In an embodiment, the length Z is 13 to 30 Å, preferably 14 to 25 Å, more preferably 16 to 18 Å.

The inventors believe that a length Z in a range of 13 to 30 Å as detailed above may result in the targeting of amino acids on the Fc region of an antibody or antibody fragment (a highly conserved region across antibodies, in particular, IgG antibodies) e.g. one or more of the lysine residues found at positions 317, 326, 338, 340 and 439, and in particular, at positions 317 and/or 326, and may lead to reaction of the reactive moiety and attachment of the payload with a high degree of regioselectivity. In an embodiment, a high degree of regioselectivity is achieved if the payload loading ratio Fc/F(ab)₂ is more than 1.0, more than 1.5, more than 2.0, in particular more than 2.5. The degree of regioselectivity can be determined by measuring the payload loading ratio between the Fc and F(ab)₂ regions (selectivity Fc/F(ab)₂) as described further below.

The spacer may be any group of having the aforementioned length Z capable of linking the vector and the reactive moiety. Preferably it will be chemically inert.

In one embodiment, the spacer is preferably selected from
(a2) a polyalkylene oxide group having from 6 to 36 repeating units, for instance, 8 to 24 repeating units; preferably a group represented by the following formula (7):

—X¹—(CH₂CH₂O)ₙ₂—CH₂CH₂—X²— (7)
wherein
X¹ is NH, O or S; preferably NH;
X² is NH or C═O, preferably C═O if X² is covalently bonded to the vector; and
n2 is an integer of 4 to 28, preferably 6 to 20, more preferably 8 to 12, in particular 10;
(b2) a peptidic group having 6 to 25 amino acids in the main chain, e.g. 9 amino acids in the main chain, each amino acid being preferably selected from Pro, Gly, Ala, Asn, Asp, Thr, Glu, Gln, and Ser; more preferably Pro, Gly or Ser.

In the formula (7), in some embodiments X² is covalently bonded to the vector and X¹ is covalently bonded to the reactive moiety; and in some other embodiments X¹ is covalently bonded to the vector and X² is covalently bonded to the reactive moiety. In some specific embodiments, the point of attachment of the spacer to the vector and X¹ and X² are each independently selected such that attachment to the vector forms an amide bond. For instance, if the vector is attached to the spacer via the N-terminus (i.e. via the amino group of an N-terminal amino acid), X² may be selected to be C═O, and if the vector is attached to the spacer via the C-terminus (i.e. via the carboxyl group of a C-terminal amino acid), X¹ (or X²) may be selected to be NH.

According to one embodiment, the spacer comprises a polyethylene oxide group having 4 to 36 repeating units, preferably 6 to 28 repeating units, more preferably 7 to 24 repeating units, e.g. 10 or 20 repeating units. Most preferably the spacer comprises a polyethylene oxide group having 10 repeating units.

3.4 Vector (V)

The compound of the present invention comprises a vector (V) (or “ligand”) capable of interacting with (binding to) the fragment crystallizable (Fc) region of an antibody or fragment thereof, the antibody fragment being optionally incorporated into an Fc-fusion protein. The interaction of the vector with the Fc region leads to an increase in the concentration of the reactive moiety in proximity to the side chain of an amino acid exposed at the surface of the antibody or antibody fragment, leading to covalent attachment of the payload to the side chain. In some aspects, the interaction of the vector with the Fc region leads to a targeting effect insofar that the reactive moiety will react with the side chain of a specific amino acid exposed at the surface of the antibody or antibody fragment (e.g. the lysine residue at position 317), leading to the regioselective attachment of the payload to the antibody or antibody fragment.

Vectors capable of interacting with the Fc region of antibodies or fragments thereof are known in the art and are described e.g. in Choe et al. Materials 2016, 9, 994. Suitable Vectors are also disclosed in WO 2018/199337 A1. Non-limiting examples of vectors capable of interacting with the FC region of antibodies or fragments thereof include protein Z and Fc-III. In particular, the cyclic peptide Fc-III has been described as a peptidic vector/ligand having high affinity for the Fc region of IgG proteins with a reported dissociation constant K_{d} of about 16 nm (DeLano et al. Science 2000, 287, 1279-1283).

In one embodiment, the vector to be used in the compound of the present invention is a peptide comprising a sequence of 11 to 17 amino acids, preferably 13 to 17 amino acids. In some specific embodiments, the spacer is attached to the vector (i.e. to the aforementioned peptide sequence) via its N- or C-terminus. In some further specific embodiments, the vector is not further modified apart for the attachment of the spacer to the N- or C-terminus.

According to one preferred embodiment, the vector is a peptide represented by one of the following formulae (8a) and (8b):

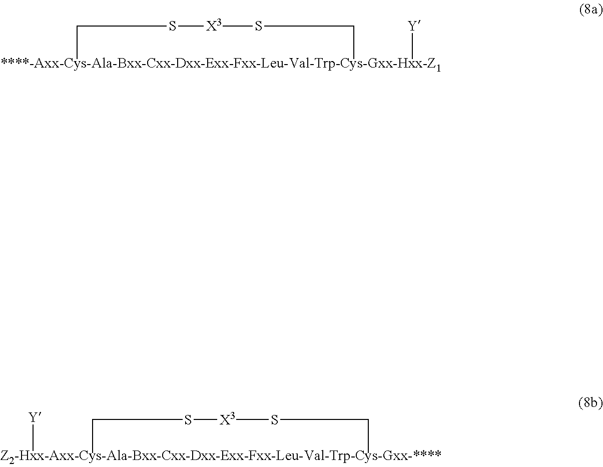

Wherein,

Bxx, Cxx, Dxx, Exx, Fxx each independently represent an amino acid;

Axx represents an amino acid, a dicarboxylic acid, or a peptide moiety represented by the following formula (9a):

-Axx1-Axx2-Axx3- (9a)
wherein,
Axx1 represents a single covalent bond or an amino acid such as Arg;
Axx2 represents an amino acid such as Gly or Cys; and
Axx3 represents an amino acid such as Asp or Asn;

Gxx represents an amino acid or a peptide moiety represented by the following formula (9b):

-Gxx1-Gxx2-Gxx3- (9b)
wherein,
Gxx1 represents an amino acid such as Thr;
Gxx2 represents an amino acid such as Tyr or Cys; and
Gxx3 represents a single covalent bond, or an amino acid such His;
and the side chain of Axx2 in formula (9a) may be covalently bonded to the side of Gxx2 in formula (9b) to form a ring; if Axx2 is Cys, and Gxx2 is Cys preferably the side chains of Axx2 and Gxx2 are linked together to form a group of formula —(S—X⁴—S)—, wherein X⁴ represents a single covalent bond or a divalent group comprising one or more atoms selected from carbon, nitrogen and oxygen such as a divalent maleimide group, a divalent acetone group or a divalent arylene group(e.g. a divalent xylene group). Preferably, X⁴ represents a single covalent bond.

Hxx represents a single covalent bond or a trifunctional amino acid such as a diamino-carboxylic acid.

Z₁ represents:
a group covalently bonded to the C-terminus of Gxx if Hxx is a single covalent bond, which is selected from —N(H)(R), wherein R represents a hydrogen atom, an alkyl group or a cycloalkyl group, and a moiety derived from a compound containing a conjugation group selected from biotin, DBCO, TCO, BCN, an alkyne, an azide, a bromoacetamide, a maleimide and a thiol;
a group covalently bonded to the C-terminus of Hxx if Hxx is a trifunctional amino acid and Y′ is bonded to the side chain of Hxx, preferably N(H)(R), wherein R represents a hydrogen atom, an alkyl group or a cycloalkyl group, if Z₁ is covalently bonded to the C-terminus of Hxx; or
a hydrogen atom bonded to the side chain of Hxx if Hxx is trifunctional amino acid and Y′ is bonded to the C-terminus of Hxx.

Z₂ represents:
a group covalently bonded to the N-terminus of Axx if Hxx is a single covalent bond, which is selected from a hydrogen atom, a carbonyl-containing group such as an acetyl group, and a group containing a conjugation moiety such as biotin;
a group covalently bonded to the N-terminus of Hxx if Hxx is a trifunctional amino acid and Y′ is bonded to the side chain of Hxx, which is selected from a hydrogen atom and a carbonyl-containing group such as an acetyl group; or
a hydrogen atom bonded to the side chain of Hxx if Hxx is trifunctional amino acid and Y′ is bonded to the N-terminus of Hxx.

Y′ is present only if Hxx is a trifunctional amino acid and it represents a moiety covalently bonded to
the side chain of Hxx if Z1 is bonded to the C-terminus of Hxx in formula (8a), or if Z₂ is bonded to the N-terminus of Hxx in formula (8b),
the C-terminus of Hxx if Z₁ is bonded to the side chain of Hxx in formula (8a), or
the N-terminus of Hxx if Z₂ is bonded to the side chain of Hxx in formula (8b);

Y′ is derived from a compound containing a conjugation group, which is preferably selected from biotin, DBCO, TCO, BCN, an alkyne, an azide, a bromoacetamide, a maleimide, and a thiol.

X³ represents a single covalent bond or a divalent group comprising one or more atoms selected from carbon, nitrogen and oxygen such as a divalent maleimide group, a divalent acetone group or a divalent arylene group (e.g. a divalent xylene group). Preferably, X³ represents a single covalent bond.

**** indicates covalent attachment to the spacer (S).

In an embodiment, the moiety Y′ is represented by the following formula (9c):

Y¹-L¹-****′ (9c)
wherein,
Y¹ is a moiety derived from a conjugation group selected from biotin, DBCO, TCO, BCN, an alkyne, an azide, a bromoacetamide, a maleimide, and a thiol;
L1 is a divalent group, preferably comprising one or more atoms selected from C, N, O and S, more preferably comprising a polyethylene oxide group having 1 to 12 repeating units e.g. 4 repeating units; and
****′ indicates covalent attachment to Hxx.

The linker is a divalent group, preferably comprising one or more atoms selected from carbon, nitrogen, oxygen, and sulfur.

In an embodiment, the linker L¹ can be selected from
(a1) an alkylene group having from 1 to 12 carbon atoms, preferably an alkylene group having from 2 to 6 carbon atoms such as an ethylene group or propylene group;
(b1) a polyalkylene oxide group with 2 or 3 carbon atoms having from 1 to 36 repeating units; preferably a group represented by the formula —NH—(CH₂CH₂₀)ₙ₁—CH₂CH₂— wherein n1 is an integer of 0 to 35, e.g. 1 to 20; and
(c1) a peptidic group having 2 to 12 amino acids.

According to one preferred embodiment, at least one of Axx, Bxx, Cxx, Dxx, Exx, Fxx, Gxx and Hxx in formulae (8a) and (8b) is defined as follows:

Axx represents an amino acid selected from Ala, 2,3-diamino-propionic acid (Dap), Asp, Glu, 2 amino suberic acid, α-amino butyric acid, Asn and Gln, a dicarboxylic acid selected from succinic acid, glutaric acid, and adipic acid; preferably Ala, Asp or Asn; more preferably Asp; or a peptide moiety of formula (9a), wherein Axx1 is a single covalent bond, Axx2 is Cys, and Axx3 is Asp;

Bxx represents an amino acid selected from Trp, Phe, Tyr, phenyl glycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;

Cxx represents an amino acid selected from His, Ala, 3-pyridin-2-yl-L-alanine, meta-tyrosine (mTyr) and Phe; preferably His, Ala or mTyr; more preferably His;

Dxx represents an amino acid selected from Ala, Abu, Gly, Leu, Ile, Val, Met, cyclohexyl alanine (Cha), Phe, Thr, Cys, Tyr, and norleucine (Nle); preferably Ala, Nle or Leu; more preferably Leu;

Exx represents an amino acid selected from Ala, Gly, Asn, Ser, Abu, and Asp; preferably Ala or Gly; more preferably Gly;

Fxx represents an amino acid selected from Ala, Glu, Asp, Gln, His, Arg, Ser, and Asn; preferably Asp or Glu; more preferably Glu;

Gxx represents an amino acid selected from Thr, Ser, Ala, Asn, Val, 2-amino-butyric acid (Abu), Ile, Met, Leu, Pro, Gln, and Cys; preferably Thr or Ser; more preferably Thr; or a peptide moiety of formula (9b), wherein Gxx1 is Thr, Gxx2 is Cys, and Gxx3 is a single covalent bond;

Hxx represents an amino acid selected from Dap, Dab, Lys, Orn, and homo-lysine (homo-Lys), preferably an amino acid selected from Dap, Dab, Lys, Orn, and homo-Lys;

According to one embodiment, the ligand V capable of interacting with the Fc region of an antibody or fragment thereof is a peptide represented by one of the following formulae (8a′) to (8d′):

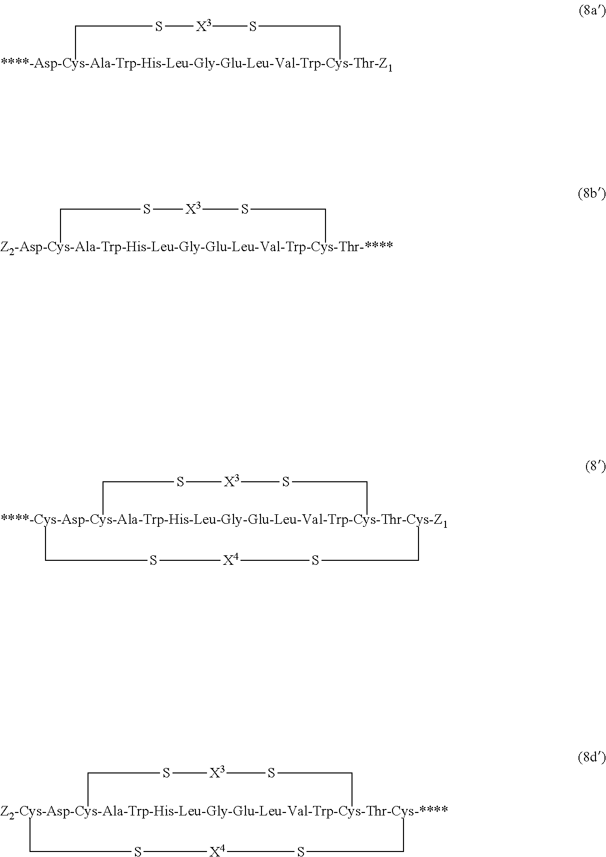

In the formulae (8a′), (8b′), (8c′) and (8d′) above, Z₁, Z₂, X³, X⁴ and **** are as described above with respect to formulae (8a) and (8b).

In an embodiment, the disulfide bridge(s) between cysteine residues in the above formulae (i.e. the disulfide bridges of formula —(S—X³—S)— or —(S—X⁴—S)—) can each independently be replaced by a divalent group suitable for side-chain-to-side-chain cyclization (sometimes called “cysteine re-bridging”; see e.g. Stefanucci et al. Scientific Reports 2019, 9:5771). Examples of suitable divalent groups include divalent xylene groups, divalent maleimide groups, divalent triazole-containing groups, divalent carbonyl-containing groups (e.g. a divalent acetone group), divalent succinimide groups (which can be obtained by reacting the cysteine side chains with e.g. an aryloxymaleimide reagent; see Marculescu et al. Chem. Commun. 2014, 50, 7139), divalent thioether groups (which can be obtained by reacting the cysteine side chains with e.g. a bis-sulfone or an allyl sulfone reagent; see Brocchini et al. Nat. Protoc. 2006, 1, 2241-2252), and divalent pyridazinedione groups (which can be obtained by reacting the cysteine side chains with e.g. a dibromopyridazinedione reagent; see Chudamasa et al. Chem. Commun. 2011, 47, 8781-8783). In particular, the disulfide bridge(s) can each independently be replaced by a divalent triazole-containing group, which may be obtained by “click” chemistry. In this case, the cysteine residues (forming a bridge in the above formulae) can be replaced by amino acids having a side chain containing a functional group suitable for click chemistry, i.e. an alkyne group or an azido group, which can be reacted to from a divalent triazole moiety (e.g. a 1,4-disubstituted-1,2,3-triazole moiety).

Preferably, the vector is a peptide represented by formula (8a′) or (8b′).

According to one embodiment, the compound of the present invention is a compound represented by a formula selected from V—S¹—(O—(C═O)—O)—P, V—S¹—(O—(C═O))—P, V—S¹—(S—(C═O))—P, V—S¹—(S—(C═O)—O)—P, V—S¹—(O—(C═S)—O)—P, V—S¹—(O—(C═O)—S)—P, V—S¹—(S—(C═O)—S)—P, V—S¹—(S—(C═S)—O)—P, V—S¹—(O—(C═S)—S)—P, V—S¹—(S—(C═S))—P, V—S¹—(O—(C═O)—NH)—P, V—S¹—(S—(C═S)—S)—P, V—S¹-(M-O—(C═O)—O)—P, V—S¹-(M-O—(C═O))—P, V—S¹-(M-S—(C═O))—P, V—S¹-(M-S—(C═O)—O)—P, V—S¹-(M-O—(C═S)—O)—P, V—S¹-(M-O—(C═O)—S)—P, V—S¹-(M-S—(C═O)—S)—P, V—S¹-(M-S—(C═S)—O)—P, V—S¹-(M-O—(C═S)—S)—P, V—S¹-(M-S—(C═S))—P, V—S¹-(M-O—(C═O)—NH)—P, V—S¹-(M-S—(C═S)—S)—P, V—S¹—(O—(C═O)—O)-L-P¹, V—S¹—(O—(C═O))-L-P¹, V—S¹—(S—(C═O))-L-P¹, V—S¹—(S—(C═O)—O)-L-P¹, V—S¹—(O—(C═S)—O)-L-P¹, V—S¹—(O—(C═O)—S)-L-P¹, V—S¹—(S—(C═O)—S)-L-P¹, V—S¹—(S—(C═S)—O)-L-P¹, V—S¹—(O—(C═S)—S)-L-P¹, V—S¹—(S—(C═S))-L-P¹, V—S¹—(O—(C═O)—NH)-L-P¹, V—S¹—(S—(C═S)—S)-L-P¹, V—S¹-(M-O—(C═O)—O)-L-P¹, V—S¹-(M-O—(C═O))-L-P¹, V—S¹-(M-S—(C═O))-L-P¹, V—S¹-(M-S—(C═O)—O)-L-P¹, V—S¹-(M-O—(C═S)—O)-L-P¹, V—S¹-(M-O—(C═O)—S)-L-P¹, V—S¹-(M-S—(C═O)—S)-L-P¹, V—S¹-(M-S—(C═S)—O)-L-P¹, V—S¹-(M-O—(C═S)—S)-L-P¹, V—S¹-(M-S—(C═S))-L-P¹, V—S¹-(M-O—(C═O)—NH)-L-P¹, and V—S¹-(M-S—(C═S)—S)-L-P¹, wherein V, P, P¹ and L are as defined above,

S¹ is a spacer S as defined above, and wherein preferably at least one—e.g. two, three, four, or more than four—of V, S¹, P/P¹, L, and M is/are defined as follows:
(α) V is a peptide of formula (8a) or (8b), preferably a peptide of formula (8a′) or (8b′);
(β) S¹ is a group selected from:
   (a2) a polyalkylene oxide group having from 6 to 36 repeating units; preferably a group represented by the following formula (7):

—X¹—(CH₂CH₂O)ₙ₂—CH₂CH₂—X²— (7)
wherein
X¹ is NH, O or S; preferably NH;
X² is NH or C═O, preferably C═O if X² is covalently bonded to the vector; and
n2 is an integer of 4 to 28, preferably 6 to 20, more preferably 10; and
(b2) a peptidic group having 6 to 25 amino acids in the main chain, each amino acid being preferably selected from Pro, Gly, Ala, Asn, Asp, Thr, Glu, Gln, and Ser; more preferably Pro, Gly or Ser;
(γ) P or P¹ is a moiety derived from:
   (γ1) NOTA, DOTA, NODAGA, DTPA, each of which may optionally chelate a radionuclide selected from ⁸⁹Zr, ¹¹¹In, ⁶⁴Cu, ¹⁷⁷Lu, ⁶⁸Ga, and ^{99m}Tc, preferably from ⁸⁹Zr, ¹¹¹In, ⁶⁴Cu,
   (γ2) N3, TZ, TCO, DBCO, BCN,
   (γ3) auristatin (e.g. MMAE) or PNU-159582;
(δ) L is a linker selected from
   (a1) an alkylene group having 2 to 6 carbons (—(CH₂)₂₋₆—),
   (b1) a polyalkylene group of formula —NH—(CH₂CH₂O)ₙ₁—CH₂CH₂—, n1 being an integer of 0-35, and
   (c1) a peptidic linker comprising 2 to 12 amino acids, which is optionally cleavable, preferably a cleavable peptidic linker comprising a Val-Cit unit, a Val-Ala unit, a Val-Cit-PABC or a Val-Cit-PABC-DMEA unit; and
(ε) M is a group of formula (5a) or (5e), preferably a group of formula (5a).

According to a preferred embodiment, in the above formulae, V, S¹ and M are defined as follows:
(α) V is a peptide of formula (8a′) or (8b′);
(β) S¹ is a group represented by the following formula (7):

—X¹—(CH₂CH₂O)ₙ₂—CH₂CH₂—X²— (7)
wherein
X¹ is NH, O or S; preferably NH;
X² is NH or C═O, preferably C═O if X2 is covalently bonded to the vector; and
n2 is an integer of 6 to 20, preferably 10; and
(ε) M is a group of formula (5a).

If (γ) P¹ is a moiety derived from auristatin, e.g. MMAE, (δ) L represents preferably a cleavable linker comprising a Val-Cit unit, a Val-Ala unit or a Val-Cit-PABC unit, more preferably a Val-Cit-PABC unit. If (γ) P¹ is a moiety derived from PNU-159582, (δ) L represents preferably a cleavable linker comprising a Val-Cit-PABC-DMEA unit.

According to one embodiment, the compound of the present invention is a compound represented by a formula selected from V¹—NH—(CH₂CH₂O)ₙ₂—CH₂CH₂—NH-(M-O—(C═O)—O)—P, V¹—NH—(CH₂CH₂O)ₙ₂—CH₂CH₂—NH-(M-O—(C═O))—P, V¹—NH—(CH₂CH₂O)ₙ₂—CH₂CH₂—NH-(M-S—(C═O))—P, V¹—NH—(CH₂CH₂O)ₙ₂—CH₂CH₂—NH-(M-S—(C═O)—O)—P, V¹—NH—(CH₂CH₂O)ₙ₂—CH₂CH₂—NH-(M-O—(C═S)—O)—P, V¹—NH—(CH₂CH₂O)ₙ₂—CH₂CH₂—NH-(M-O—(C═O)—S)—P, V¹—NH—(CH₂CH₂O)ₙ₂—CH₂CH₂—NH-(M-S—(C═O)—S)—P, V¹—NH—(CH₂CH₂O)ₙ₂—CH₂CH₂—NH-(M-S—(C═S)—O)—P, V¹—NH—(CH₂CH₂O)ₙ₂—CH₂CH₂—NH-(M-O—(C═S)—S)—P, V¹—NH—(CH₂CH₂O)ₙ₂—CH₂CH₂—NH-(M-S—(C═S))—P, V¹—NH—(CH₂CH₂O)ₙ₂—CH₂CH₂—NH-(M-O—(C═O)—NH)—P, V¹—NH—(CH₂CH₂O)ₙ₂—CH₂CH₂—NH-(M-S—(C═S)—S)—P, V¹—NH—(CH₂CH₂O)ₙ₂—CH₂CH₂—NH—(O—(C═O)—O)-L-P¹, V¹—NH—(CH₂CH₂O)ₙ₂—CH₂CH₂—NH—(O—(C═O))-L-P¹, V¹—NH—(CH₂CH₂O)ₙ₂—CH₂CH₂—NH—(S—(C═O))-L-P¹, V¹—NH—(CH₂CH₂O)ₙ₂—CH₂CH₂—NH—(S—(C═O)—O)-L-P¹, V¹—NH—(CH₂CH₂O)ₙ₂—CH₂CH₂—NH—(O—(C═S)—O)-L-P¹, V¹—NH—(CH₂CH₂O)ₙ₂—CH₂CH₂—NH—(O—(C═O)—S)-L-P¹, V¹—NH—(CH₂CH₂O)ₙ₂—CH₂CH₂—NH—(S—(C═O)—S)-L-P¹, V¹—NH—(CH₂CH₂O)ₙ₂—CH₂CH₂—NH—(S—(C═S)—O)-L-P¹, V¹—NH—(CH₂CH₂O)ₙ₂—CH₂CH₂—NH—(O—(C═S)—S)-L-P¹, V¹—NH—(CH₂CH₂O)ₙ₂—CH₂CH₂—NH—(S—(C═S))-L-P¹, V¹—NH—(CH₂CH₂O)ₙ₂—CH₂CH₂—NH—(O—(C═O)—NH)-L-P¹, V¹—NH—(CH₂CH₂O)ₙ₂—CH₂CH₂—NH—(S—(C═S)—S)-L-P¹, V¹—NH—(CH₂CH₂O)ₙ₂—CH₂CH₂—NH-(M-O—(C═O)—O)-L-P¹, V¹—NH—(CH₂CH₂O)ₙ₂—CH₂CH₂—NH-(M-O—(C═O))-L-P¹, V¹—NH—(CH₂CH₂O)ₙ₂—CH₂CH₂—NH-(M-S—(C═O))-L-P¹, V¹—NH—(CH₂CH₂O)ₙ₂—CH₂CH₂—NH-(M-S—(C═O)—O)-L-P¹, V¹—NH—(CH₂CH₂O)ₙ₂—CH₂CH₂—NH-(M-O—(C═S)—O)-L-P¹, V—NH—(CH₂CH₂O)ₙ₂—CH₂CH₂—NH-(M-O—(C═O)—S)-L-P¹, V¹—NH—(CH₂CH₂O)ₙ₂—CH₂CH₂—NH-(M-S—(C═O)—S)-L-P¹, V¹—NH—(CH₂CH₂O)ₙ₂—CH₂CH₂—NH-(M-S—(C═S)—O)-L-P¹, V¹—NH—(CH₂CH₂O)ₙ₂—CH₂CH₂—NH-(M-O—(C═S)—S)-L-P¹, V¹—NH—(CH₂CH₂O)ₙ₂—CH₂CH₂—NH-(M-S—(C═S))-L-P¹, V¹NH⁻(CH₂CH₂O)ₙ₂—CH₂CH₂—NH-(M-O—(C═O)—NH)-L-P¹, V¹—NH—(CH₂CH₂O)ₙ₂—CH₂CH₂—NH-(M-S—(C═S)—S)-L-P¹, P—(O—(C═O)—O-M)-NH—(CH₂CH₂O)ₙ₂—CH₂CH₂—(C═O)—V², P—((C═O)—O-M)-NH—(CH₂CH₂O)ₙ₂—CH₂CH₂—(C═O)—V², P—((C═O)—S-M)-NH—(CH₂CH₂O)ₙ₂—CH₂CH₂—(C═O)—V², P—(O—(C═O)—S-M)-NH—(CH₂CH₂O)ₙ₂—CH₂CH₂—(C═O)—V², P—(O—(C═S)—O-M)-NH—(CH₂CH₂O)ₙ₂—CH₂CH₂—(C═O)—V², P—(S—(C═O)—O-M)-NH—(CH₂CH₂O)ₙ₂—CH₂CH₂—(C═O)—V², P—(S—(C═O)—S-M)-NH—(CH₂CH₂O)ₙ₂—CH₂CH₂—(C═O)—V², P—(O—(C═S)—S-M)-NH—(CH₂CH₂O)ₙ₂—CH₂CH₂—(C═O)—V², P—(S—(C═S)—O-M-NH—(CH₂CH₂O)ₙ₂—CH₂CH₂—(C═O)—V², P—((C═S)—S-M)-NH—(CH₂CH₂O)ₙ₂—CH₂CH₂—(C═O)—V², P—(NH—(C═O)—O-M)-NH—(CH₂CH₂O)ₙ₂—CH₂CH₂—(C═O)—V², P—(S—(C═S)—S-M)-NH—(CH₂CH₂O)ₙ₂—CH₂CH₂—(C═O)—V², P¹-L-(O—(C═O)—O-M)-NH—(CH₂CH₂O)ₙ₂—CH₂CH₂—(C═O)—V², P¹-L-((C═O)—O-M)-NH—(CH₂CH₂O)ₙ₂—CH₂CH₂—(C═O)—V², P¹-L-((C═O)—S-M)-NH—(CH₂CH₂O)ₙ₂—CH₂CH₂—(C═O)—V², P¹-L-(O—(C═O)—S-M)-NH—(CH₂CH₂O)ₙ₂—CH₂CH₂—(C═O)—V², P¹-L-(O—(C═S)—O-M)-NH—(CH₂CH₂O)ₙ₂—CH₂CH₂—(C═O)—V², P¹-L-(S—(C═O)—O-M)-NH—(CH₂CH₂O)ₙ₂—CH₂CH₂—(C═O)—V², P¹-L-(S—(C═O)—S-M)-NH—(CH₂CH₂O)ₙ₂—CH₂CH₂—(C═O)—V², P¹-L-(O—(C═S)—S-M)-NH—(CH₂CH₂O)ₙ₂—CH₂CH₂—(C═O)—V², P¹-L-(S—(C═S)—O-M)-NH—(CH₂CH₂O)ₙ₂—CH₂CH₂—(C═O)—V², P¹-L-((C═S)—S-M)-NH—(CH₂CH₂O)ₙ₂—CH₂CH₂—(C═O)—V², P¹-L-(NH—(C═O)—O-M)-NH—(CH₂CH₂O)ₙ₂—CH₂CH₂—(C═O)—V², P¹-L-(S—(C═S)—S-M)-NH—(CH₂CH₂O)ₙ₂—CH₂CH₂—(C═O)—V², V-AA₆₋₂₅-(M-O—(C═O)—O)—P, V-AA₆₋₂₅-(M-O—(C═O))—P, V-AA₆₋₂₅-(M-S—(C═O))—P, V-AA₆₋₂₅-(M-S—(C═O)—O)—P, V-AA₆₋₂₅-(M-O—(C═S)—O)—P, V-AA₆₋₂₅-(M-O—(C═O)—S)—P, V-AA₆₋₂₅-(M-S—(C═O)—S)—P, V-AA₆₋₂₅-(M-S—(C═S)—O)—P, V-AA₆₋₂₅-(M-O—(C═S)—S)—P, V-AA₆₋₂₅-(M-S—(C═S))—P, V-AA₆₋₂₅-(M-O—(C═O)—NH)—P, V-AA₆₋₂₅-(M-S—(C═S)—S)—P, V-AA₆₋₂₅-(O—(C═O)—O)-L-P¹, V-AA₆₋₂₅-(O—(C═O))-L-P¹, V-AA₆₋₂₅-(S—(C═O))-L-P¹, V-AA₆₋₂₅-(S—(C═O)—O)-L-P¹, V-AA₆₋₂₅-(O—(C═S)—O)-L-P¹, V-AA₆₋₂₅-(O—(C═O)—S)-L-P¹, V-AA₆₋₂₅-(S—(C═O)—S)-L-P¹, V-AA₆₋₂₅-(S—(C═S)—O)-L-P¹, V-AA₆₋₂₅-(O—(C═S)—S)-L-P¹, V-AA₆₋₂₅-(S—(C═S))-L-P¹, V-AA₆₋₂₅-(O—(C═O)—NH)-L-P¹, V-AA₆₋₂₅-(S—(C═S)—S)-L-P¹, V-AA₆₋₂₅-(M-O—(C═O)—O)-L-P¹, V-AA₆₋₂₅-(M-O—(C═O))-L-P¹, V-AA₆₋₂₅-(M-S—(C═O))-L-P¹, V-AA₆₋₂₅-(M-S—(C═O)—O)-L-P¹, V-AA₆₋₂₅-(M-O—(C═S)—O)-L-P¹, V-AA₆₋₂₅-(M-O—(C═O)—S)-L-P¹, V-AA₆₋₂₅-(M-S—(C═O)—S)-L-P¹, V-AA₆₋₂₅-(M-S—(C═S)—O)-L-P¹, V-AA₆₋₂₅-(M-O—(C═S)—S)-L-P¹, V-AA₆₋₂₅-(M-S—(C═S))-L-P¹, V-AA₆₋₂₅-(M-O—(C═O)—NH)-L-P¹, and V-AA₆₋₂₅-(M-S—(C═S)S)-L-P¹, wherein V, P, P¹ and L are as defined above, V¹ is peptide of formula (8b), V² is a peptide of formula (8a), and wherein preferably at least one—e.g. two, three, four, or more than four—of V/V¹/V², n2/AA, P/P¹, L, and M is/are defined as follows:
(α) V is a peptide of formula (8a) or (8b), preferably of formula (8a′) or (8b′); V¹ is a peptide of formula (8b′), V² is a peptide of formula (8a′);
(β) n2 is an integer of 6 to 20, preferably 10; or
   each amino acid (AA) is independently selected from Pro, Gly, Ala, Asn, Asp, Thr, Glu, Gln and Ser, preferably from Pro, Gly and Ser;
(γ) P or P¹ is a moiety derived from:
   (γ1) NOTA, DOTA, NODAGA, DTPA, each of which may optionally chelate a radionuclide selected from ⁸⁹Zr, ¹¹¹In, ⁶⁴Cu, ¹⁷⁷Lu, ⁶⁸Ga, and ^{99m}Tc, preferably from ⁸⁹Zr, ¹¹¹In, ⁶⁴Cu,
   (γ2) N3, TZ, TCO, DBCO, BCN,
   (γ3) auristatin (e.g. MMAE) or PNU-159582;
(δ) L is a linker selected from
   (a1) an alkylene group having 2 to 6 carbons (—(CH₂)₂₋₆—),
   (b1) a polyalkylene group of formula —NH—(CH₂CH₂O)ₙ₁—CH₂CH₂—, n1 being an integer of 0-35, and
   (c1) a peptidic linker comprising 2 to 12 amino acids, which is optionally cleavable, preferably a cleavable peptidic linker comprising a Val-Cit unit, a Val-Ala unit, a Val-Cit-PABC or a Val-Cit-PABC-DMEA unit; and
(ε) M is a group of formula (5a) or (5e), preferably a group of formula (5a).

According to a preferred embodiment, in the above formulae, V/V¹/V², n2/AA and M are defined as follows:
(α) V is a peptide of formula (8a′) or (8b′); V¹ is a peptide of formula (8b′), V² is a peptide of formula (8a′);
(β) n2 is an integer of 6 to 20, preferably 10; or each amino acid (AA) is independently selected from Pro, Gly, Ala, Asn, Asp, Thr, Glu, Gln and Ser, preferably from Pro, Gly and Ser; and
(ε) M is a group of formula (5a).

If (γ) P¹ is a moiety derived from auristatin, e.g. MMAE, (δ) L represents preferably a cleavable linker comprising a Val-Cit unit, a Val-Ala unit or a Val-Cit-PABC unit, more preferably a Val-Cit-PABC unit. If (γ) P¹ is a moiety derived from PNU-159582, (δ) L represents preferably a cleavable linker comprising a Val-Cit-PABC-DMEA unit.

According to one embodiment, the compound of the present invention is a compound represented by a formula selected from V¹—NH—(CH₂CH₂O)₆₋₂₀—CH₂CH₂—NH-(M-O—(C═O)—O)—P, V¹—NH—(CH₂CH₂O)₆₋₂₀—CH₂CH₂—NH-(M-O—(C═O))—P, V¹—NH—(CH₂CH₂O)₆₋₂₀—CH₂CH₂—NH-(M-S—(C═O))—P, V¹—NH—(CH₂CH₂O)₆₋₂₀—CH₂CH₂—NH-(M-S—(C═O)—O)—P, V¹—NH—(CH₂CH₂O)₆₋₂₀—CH₂CH₂—NH-(M-O—(C═S)—O)—P, V¹—NH—(CH₂CH₂O)₆₋₂₀—CH₂CH₂—NH-(M-O—(C═O)—S)—P, V¹—NH—(CH₂CH₂O)₆₋₂₀—CH₂CH₂—NH-(M-S—(C═O)—S)—P, V¹—NH—(CH₂CH₂O)₆₋₂₀—CH₂CH₂—NH-(M-S—(C═S)—O)—P, V¹—NH—(CH₂CH₂O)₆₋₂₀—CH₂CH₂—NH-(M-O—(C═S)—S)—P, V¹—NH⁻(CH₂CH₂O)₆₋₂₀—CH₂CH₂—NH-(M-S—(C═S))—P, V¹—NH—(CH₂CH₂O)₆₋₂₀—CH₂CH₂—NH-(M-O—(C═O)—NH)—P, V¹—NH—(CH₂CH₂O)₆₋₂₀—CH₂CH₂—NH-(M-S—(C═S)—S)—P, V¹—NH—(CH₂CH₂O)₆₋₂₀—CH₂CH₂—NH—(O—(C═O)—O)-L-P¹, V¹—NH—(CH₂CH₂O)₆₋₂₀—CH₂CH₂—NH—(O—(C═O))-L-P¹, V¹—NH—(CH₂CH₂O)₆₋₂₀—CH₂CH₂—NH—(S—(C═O))-L-P¹, V¹—NH—(CH₂CH₂O)₆₋₂₀—CH₂CH₂—NH—(S—(C═O)—O)-L-P¹, V¹—NH⁻(CH₂CH₂O)₆₋₂₀—CH₂CH₂—NH—(O—(C═S)—O)-L-P¹, V¹—NH—(CH₂CH₂O)₆₋₂₀—CH₂CH₂—NH—(O—(C═O)—S)-L-P¹, V¹—NH—(CH₂CH₂O)₆₋₂₀—CH₂CH₂—NH—(S—(C═O)—S)-L-P¹, V¹—NH—(CH₂CH₂O)₆₋₂₀—CH₂CH₂—NH—(S—(C═S)—O)-L-P¹, V¹—NH—(CH₂CH₂O)₆₋₂₀—CH₂CH₂—NH—(O—(C═S)—S)-L-P¹, V¹—NH—(CH₂CH₂O)₆₋₂₀—CH₂CH₂—NH—(S—(C═S))-L-P¹, V¹—NH—(CH₂CH₂O)₆₋₂₀—CH₂CH₂—NH—(O—(C═O)—NH)-L-P¹, V¹—NH—(CH₂CH₂O)₆₋₂₀—CH₂CH₂—NH—(S—(C═S)—S)-L-P¹, V¹—NH—(CH₂CH₂O)₆₋₂₀—CH₂CH₂—NH-(M-O—(C═O)—O)-L-P¹, V¹—NH—(CH₂CH₂O)₆₋₂₀—CH₂CH₂—NH-(M-O—(C═O))-L-P¹, V¹—NH—(CH₂CH₂O)₆₋₂₀—CH₂CH₂—NH-(M-S—(C═O))-L-P¹, V¹—NH—(CH₂CH₂O)₆₋₂₀—CH₂CH₂—NH-(M-S—(C═O)—O)-L-P¹, V¹—NH—(CH₂CH₂O)₆₋₂₀—CH₂CH₂—NH-(M-O—(C═S)—O)-L-P¹, V—NH—(CH₂CH₂O)₆₋₂₀—CH₂CH₂—NH-(M-O—(C═O)—S)-L-P¹, V¹—NH—(CH₂CH₂O)₆₋₂₀—CH₂CH₂—NH-(M-S—(C═O)—S)-L-P¹, V¹—NH—(CH₂CH₂O)₆₋₂₀—CH₂CH₂—NH-(M-S—(C═S)—O)-L-P¹, V¹—NH—(CH₂CH₂O)₆₋₂₀—CH₂CH₂—NH-(M-O—(C═S)—S)-L-P¹, V¹—NH—(CH₂CH₂O)₆₋₂₀—CH₂CH₂—NH-(M-S—(C═S))-L-P¹, V¹—NH—(CH₂CH₂O)₆₋₂₀—CH₂CH₂—NH-(M-O—(C═O)—NH)-L-P¹, V¹—NH—(CH₂CH₂O)₆₋₂₀—CH₂CH₂—NH-(M-S—(C═S)—S)-L-P¹, P—(O—(C═O)—O-M)-NH—(CH₂CH₂O)₆₋₂₀—CH₂CH₂—(C═O)—V², P—((C═O)—O-M)-NH—(CH₂CH₂O)₆₋₂₀—CH₂CH₂—(C═O)—V², P—((C═O)—S-M)-NH—(CH₂CH₂O)₆₋₂₀—CH₂CH₂—(C═O)—V², P—(O—(C═O)—S-M)-NH—(CH₂CH₂O)₆₋₂₀—CH₂CH₂—(C═O)—V², P—(O—(C═S)—O-M)-NH—(CH₂CH₂O)₆₋₂₀—CH₂CH₂—(C═O)—V², P—(S—(C═O)—O-M)-NH—(CH₂CH₂O)₆₋₂₀—CH₂CH₂—(C═O)—V², P—(S—(C═O)—S-M)-NH—(CH₂CH₂O)₆₋₂₀—CH₂CH₂—(C═O)—V², P—(O—(C═S)—S-M)-NH—(CH₂CH₂O)₆₋₂₀—CH₂CH₂—(C═O)—V², P—(S—(C═S)—O-M)-NH—(CH₂CH₂O)₆₋₂₀—CH₂CH₂—(C═O)—V², P—((C═S)—S-M)-NH—(CH₂CH₂O)₆₋₂₀—CH₂CH₂—(C═O)—V², P—(NH—(C═O)—O-M)-NH—(CH₂CH₂O)₆₋₂₀—CH₂CH₂—(C═O)—V², P—(S—(C═S)—S-M)-NH—(CH₂CH₂O)₆₋₂₀—CH₂CH₂—(C═O)—V², P¹-L-(O—(C═O)—O-M)-NH—(CH₂CH₂O)₆₋₂₀—CH₂CH₂—(C═O)—V², P¹-L-((C═O)—O-M)-NH—(CH₂CH₂O)₆₋₂₀—CH₂CH₂—(C═O)—V², P¹-L-((C═O)—S-M)-NH—(CH₂CH₂O)₆₋₂₀—CH₂CH₂—(C═O)—V², P¹-L-(O—(C═O)—S-M)-NH—(CH₂CH₂O)₆₋₂₀—CH₂CH₂—(C═O)—V², P¹-L-(O—(C═S)—O-M)-NH—(CH₂CH₂O)₆₋₂₀—CH₂CH₂—(C═O)—V², P¹-L-(S—(C═O)—O-M)-NH—(CH₂CH₂O)₆₋₂₀—CH₂CH₂—(C═O)—V², P¹-L-(S—(C═O)—S-M)-NH—(CH₂CH₂O)₆₋₂₀—CH₂CH₂—(C═O)—V², P¹-L-(O—(C═S)—S-M)-NH—(CH₂CH₂O)₆₋₂₀—CH₂CH₂—(C═O)—V², P¹-L-(S—(C═S)—O-M)-NH—(CH₂CH₂O)₆₋₂₀—CH₂CH₂—(C═O)—V², P¹-L-((C═S)—S-M)-NH—(CH₂CH₂O)₆₋₂₀—CH₂CH₂—(C═O)—V², P¹-L-(NH—(C═O)—O-M)-NH—(CH₂CH₂O)₆₋₂₀—CH₂CH₂—(C═O)—V², and P¹-L-(S—(C═S)—S-M)-NH—(CH₂CH₂O)₆₋₂₀—CH₂CH₂—(C═O)—V², wherein V, P, P¹ and L are as defined above, V¹ is peptide of formula (8b), V² is a peptide of formula (8a), and wherein preferably at least one—e.g. two, three, four, or more than four—of V¹/V², P/P¹, L, and M is/are defined as follows:
(α) V¹ is a peptide of formula (8b′), V² is a peptide of formula (8a′);
(γ) P or P¹ is a moiety derived from:
   (γ1) NOTA, DOTA, NODAGA, DTPA, each of which may optionally chelate a radionuclide selected from ⁸⁹Zr, ¹¹¹In, ⁶⁴Cu, ¹⁷⁷Lu, ⁶⁸Ga, and ^{99m}Tc, preferably from ⁸⁹Zr, ¹¹¹In, ⁶⁴Cu,
   (γ2) N3, TZ, TCO, DBCO, BCN,
   (γ3) auristatin (e.g. MMAE) or PNU-159582;
(δ) L is a linker selected from
   (a1) an alkylene group having 2 to 6 carbons (—(CH₂)₂₋₆—),
   (b1) a polyalkylene group of formula —NH—(CH₂CH₂O)ₙ₁—CH₂CH₂—, n1 being an integer of 0-35, and
   (c1) a peptidic linker comprising 2 to 12 amino acids, which is optionally cleavable, preferably a cleavable peptidic linker comprising a Val-Cit unit, a Val-Ala unit, a Val-Cit-PABC or a Val-Cit-PABC-DMEA unit; and
(ε) M is a group of formula (5a) or (5e), preferably a group of formula (5a).

According to a preferred embodiment, in the above formulae, V¹/V² and M are defined as follows:
(α) V¹ is a peptide of formula (8b′), V² is a peptide of formula (8a′); and
(ε) M is a group of formula (5a).

If (γ) P¹ is a moiety derived from auristatin, e.g. MMAE, (δ) L represents preferably a cleavable linker comprising a Val-Cit unit, a Val-Ala unit or a Val-Cit-PABC unit, more preferably a Val-Cit-PABC unit. If (γ) P¹ is a moiety derived from PNU-159582, (δ) L represents preferably a cleavable linker comprising a Val-Cit-PABC-DMEA unit.

According to one embodiment, the compound of the present invention is a compound represented by a formula selected from V¹—NH—(CH₂CH₂O)₁₀—CH₂CH₂—NH-(M-O—(C═O)—O)—P, V¹—NH—(CH₂CH₂O)₁₀—CH₂CH₂—NH-(M-O—(C═O))—P, V¹—NH—(CH₂CH₂O)₁₀—CH₂CH₂—NH-(M-S—(C═O))—P, V¹—NH—(CH₂CH₂O)₁₀—CH₂CH₂—NH-(M-S—(C═O)—O)—P, V¹—NH—(CH₂CH₂O)₁₀—CH₂CH₂—NH-(M-O—(C═S)—O)—P, V¹—NH—(CH₂CH₂O)₁₀—CH₂CH₂—NH-(M-O—(C═O)—S)—P, V¹—NH—(CH₂CH₂O)₁₀—CH₂CH₂—NH-(M-S—(C═O)—S)—P, V¹—NH—(CH₂CH₂O)₁₀—CH₂CH₂—NH-(M-S—(C═S)—O)—P, V¹—NH—(CH₂CH₂O)₁₀—CH₂CH₂—NH-(M-O—(C═S)—S)—P, V¹—NH—(CH₂CH₂O)₁₀—CH₂CH₂—NH-(M-S—(C═S))—P, V¹—NH—(CH₂CH₂O)₁₀—CH₂CH₂—NH-(M-O—(C═O)—NH)—P, V¹—NH—(CH₂CH₂O)₁₀—CH₂CH₂—NH-(M-S—(C═S)—S)—P, V¹—NH—(CH₂CH₂O)₁₀—CH₂CH₂—NH—(O—(C═O)—O)-L-P¹, V¹—NH—(CH₂CH₂O)₁₀—CH₂CH₂—NH—(O—(C═O))-L-P¹, V¹—NH—(CH₂CH₂O)₁₀—CH₂CH₂—NH—(S—(C═O))-L-P¹, V¹—NH—(CH₂CH₂O)₁₀—CH₂CH₂—NH—(S—(C═O)—O)-L-P¹, V¹—NH—(CH₂CH₂O)₁₀—CH₂CH₂—NH—(O—(C═S)—O)-L-P¹, V¹—NH—(CH₂CH₂O)₁₀—CH₂CH₂—NH—(O—(C═O)—S)-L-P¹, V¹—NH—(CH₂CH₂O)₁₀—CH₂CH₂—NH—(S—(C═O)—S)-L-P¹, V¹—NH—(CH₂CH₂O)₁₀—CH₂CH₂—NH—(S—(C═S)—O)-L-P¹, V¹—NH—(CH₂CH₂O)₁₀—CH₂CH₂—NH—(O—(C═S)—S)-L-P¹, V¹—NH—(CH₂CH₂O)₁₀—CH₂CH₂—NH—(S—(C═S))-L-P¹, V¹—NH—(CH₂CH₂O)₁₀—CH₂CH₂—NH—(O—(C═O)—NH)-L-P¹, V¹—NH—(CH₂CH₂O)₁₀—CH₂CH₂—NH—(S—(C═S)—S)-L-P¹, V¹—NH—(CH₂CH₂O)₁₀—CH₂CH₂—NH-(M-O—(C═O)—O)-L-P¹, V¹—NH—(CH₂CH₂O)₁₀—CH₂CH₂—NH-(M-O—(C═O))-L-P¹, V¹—NH—(CH₂CH₂O)₁₀—CH₂CH₂—NH-(M-S—(C═O))-L-P¹, V¹—NH—(CH₂CH₂O)₁₀—CH₂CH₂—NH-(M-S—(C═O)—O)-L-P¹, V¹—NH—(CH₂CH₂O)₁₀—CH₂CH₂—NH-(M-O—(C═S)—O)-L-P¹, V—NH—(CH₂CH₂O)₁₀—CH₂CH₂—NH-(M-O—(C═O)—S)-L-P¹, V¹—NH—(CH₂CH₂O)₁₀—CH₂CH₂—NH-(M-S—(C═O)—S)-L-P¹, V¹—NH—(CH₂CH₂O)₁₀—CH₂CH₂—NH-(M-S—(C═S)—O)-L-P¹, V¹—NH—(CH₂CH₂O)₁₀—CH₂CH₂—NH-(M-O—(C═S)—S)-L-P¹, V¹—NH—(CH₂CH₂O)₁₀—CH₂CH₂—NH-(M-S—(C═S))-L-P¹, V¹—NH—(CH₂CH₂O)₁₀—CH₂CH₂—NH-(M-O—(C═O)—NH)-L-P¹, V¹—NH—(CH₂CH₂O)₁₀—CH₂CH₂—NH-(M-S—(C═S)—S)-L-P¹, P—(O—(C═O)—O-M)-NH—(CH₂CH₂O)₁₀—CH₂CH₂—(C═O)—V², P—((C═O)—O-M)-NH—(CH₂CH₂O)₁₀—CH₂CH₂—(C═O)—V², P—((C═O)—S-M)-NH—(CH₂CH₂O)₁₀—CH₂CH₂—(C═O)—V², P—(O—(C═O)—S-M)-NH—(CH₂CH₂O)₁₀—CH₂CH₂—(C═O)—V², P—(O—(C═S)—O-M)-NH—(CH₂CH₂O)₁₀—CH₂CH₂—(C═O)—V², P—(S—(C═O)—O-M)-NH—(CH₂CH₂O)₁₀—CH₂CH₂—(C═O)—V², P—(S—(C═O)—S-M)-NH—(CH₂CH₂O)₁₀—CH₂CH₂—(C═O)—V², P—(O—(C═S)—S-M)-NH—(CH₂CH₂O)₁₀—CH₂CH₂—(C═O)—V², P—(S—(C═S)—O-M)-NH—(CH₂CH₂O)₁₀—CH₂CH₂—(C═O)—V², P—(S—(C═S)—S-M)-NH—(CH₂CH₂O)₁₀—CH₂CH₂—(C═O)—V², P—(NH—(C═O)—O-M)-NH—(CH₂CH₂O)₁₀—CH₂CH₂—(C═O)—V², P—(S—(C═S)—S-M)-NH—(CH₂CH₂O)₁₀—CH₂CH₂—(C═O)—V², P¹-L-(O—(C═O)—O-M)-NH—(CH₂CH₂O)₁₀—CH₂CH₂—(C═O)—V², P¹-L-((C═O)—O-M)-NH—(CH₂CH₂O)₁₀—CH₂CH₂—(C═O)—V², P¹-L-((C═O)—S-M)-NH—(CH₂CH₂O)₁₀—CH₂CH₂—(C═O)—V², P¹-L-(O—(C═O)—S-M)-NH—(CH₂CH₂O)₁₀—CH₂CH₂—(C═O)—V², P¹-L-(O—(C═S)—O-M)-NH—(CH₂CH₂O)₁₀—CH₂CH₂—(C═O)—V², P¹-L-(S—(C═O)—O-M)-NH—(CH₂CH₂O)₁₀—CH₂CH₂—(C═O)—V², P¹-L-(S—(C═O)—S-M)-NH—(CH₂CH₂O)₁₀—CH₂CH₂—(C═O)—V², P¹-L-(O—(C═S)—S-M)-NH—(CH₂CH₂O)₁₀—CH₂CH₂—(C═O)—V², P¹-L-(S—(C═S)—O-M)-NH—(CH₂CH₂O)₁₀—CH₂CH₂—(C═O)—V², P¹-L-((C═S)—S-M)-NH—(CH₂CH₂O)₁₀—CH₂CH₂—(C═O)—V², P¹-L-(NH—(C═O)—O-M)-NH—(CH₂CH₂O)₁₀—CH₂CH₂—(C═O)—V², and P¹-L-(S—(C═S)—S-M)-NH—(CH₂CH₂O)₁₀—CH₂CH₂—(C═O)—V², wherein V, P, P¹ and L are as defined above, V¹ is peptide of formula (8b), V² is a peptide of formula (8a), and wherein preferably at least one—e.g. two, three, four, or more than four—of V¹/V², P/P¹, L, and M is/are defined as follows:
(α) V¹ is a peptide of formula (8b′), V² is a peptide of formula (8a′);
(γ) P or P¹ is a moiety derived from:
   (γ1) NOTA, DOTA, NODAGA, DTPA, each of which may optionally chelate a radionuclide selected from ⁸⁹Zr, ¹¹¹In, ⁶⁴Cu, ¹⁷⁷Lu, ⁶⁸Ga, and ^{99m}Tc, preferably from ⁸⁹Zr, ¹¹¹In, ⁶⁴Cu,
   (γ2) N3, TZ, TCO, DBCO, BCN,
   (γ3) auristatin (e.g. MMAE) or PNU-159582;
(δ) L is a linker selected from
   (a1) an alkylene group having 2 to 6 carbons (—(CH₂)₂₋₆—),
   (b1) a polyalkylene group of formula —NH—(CH₂CH₂O)ₙ₁—CH₂CH₂—, n1 being an integer of 0-35, and
   (c1) a peptidic linker comprising 2 to 12 amino acids, which is optionally cleavable, preferably a cleavable peptidic linker comprising a Val-Cit unit, a Val-Ala unit, a Val-Cit-PABC or a Val-Cit-PABC-DMEA unit; and
(ε) M is a group of formula (5a) or (5e), preferably a group of formula (5a).

According to a preferred embodiment, in the above formulae, V¹/V² and M are defined as follows:
(α) V¹ is a peptide of formula (8b′), V² is a peptide of formula (8a′); and
(ε) M is a group of formula (5a).

If (γ) P¹ is a moiety derived from auristatin, e.g. MMAE, (δ) L represents preferably a cleavable linker comprising a Val-Cit unit, a Val-Ala unit or a Val-Cit-PABC unit, more preferably a Val-Cit-PABC unit. If (γ) P¹ is a moiety derived from PNU-159582, (δ) L represents preferably a cleavable linker comprising a Val-Cit-PABC-DMEA unit.

In one embodiment, the compound of formula (1) is selected from:

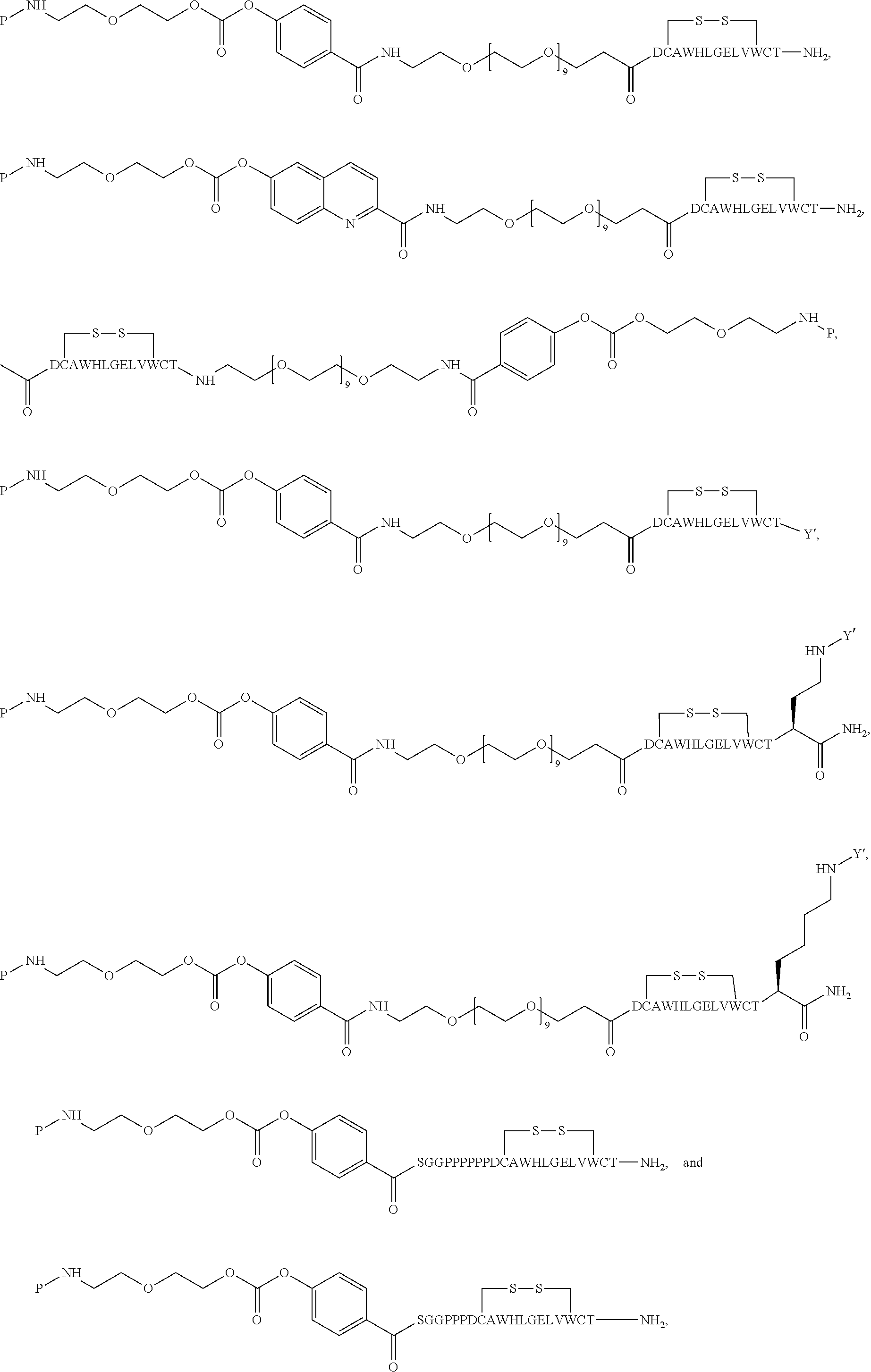

Wherein P represents a payload as defined hereinabove, preferably a chelating agent that optionally chelates a radionuclide, more preferably a moiety derived from DTPA, DOTA, DFO, NOTA, PCTA, CH-X-DTPA, NODAGA or DOTAGA; and Y′ represents a moiety derived from a compound containing a conjugation group, which is preferably selected from biotin, DBCO, TCO, BCN, an alkyne, an azide, a bromoacetamide, a maleimide, and a thiol, more preferably selected from biotin, DBCO, BCN and an azide. The number of repetitions of the spacer polyethylene oxide moiety in the above compounds (i.e. 9) can be replaced by any of 5 to 35, preferably by 7 to 19, a spacer having 9 polyethylene oxide repeating units being the most preferred option.

In one embodiment, the compound of formula (1) is selected from:

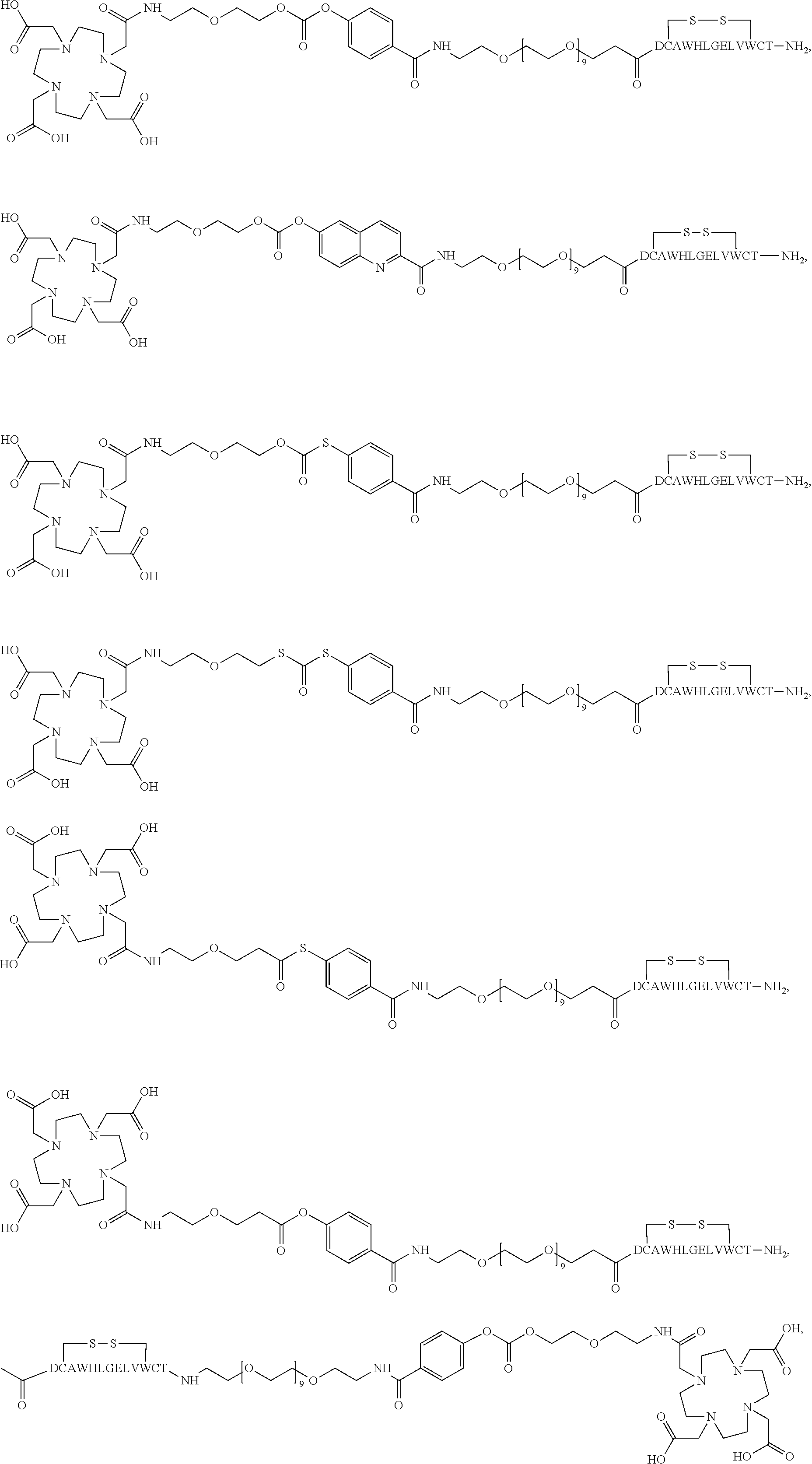

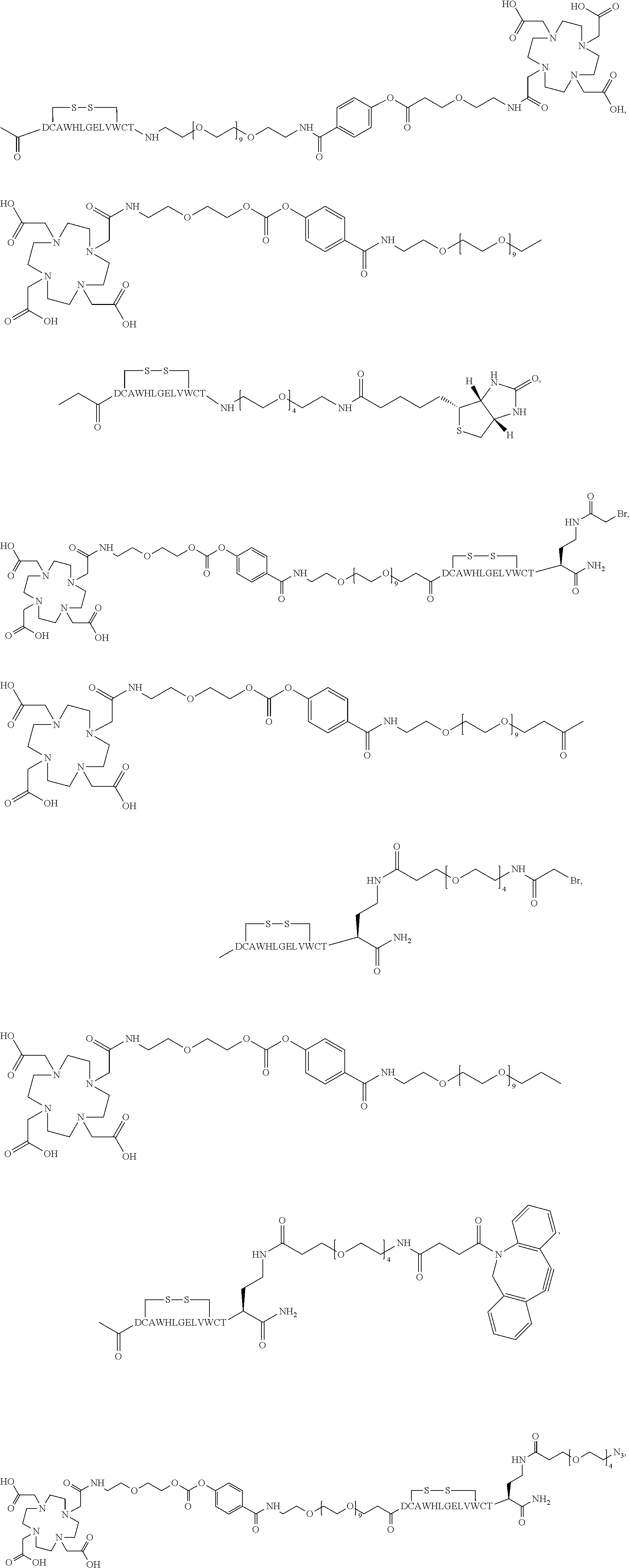

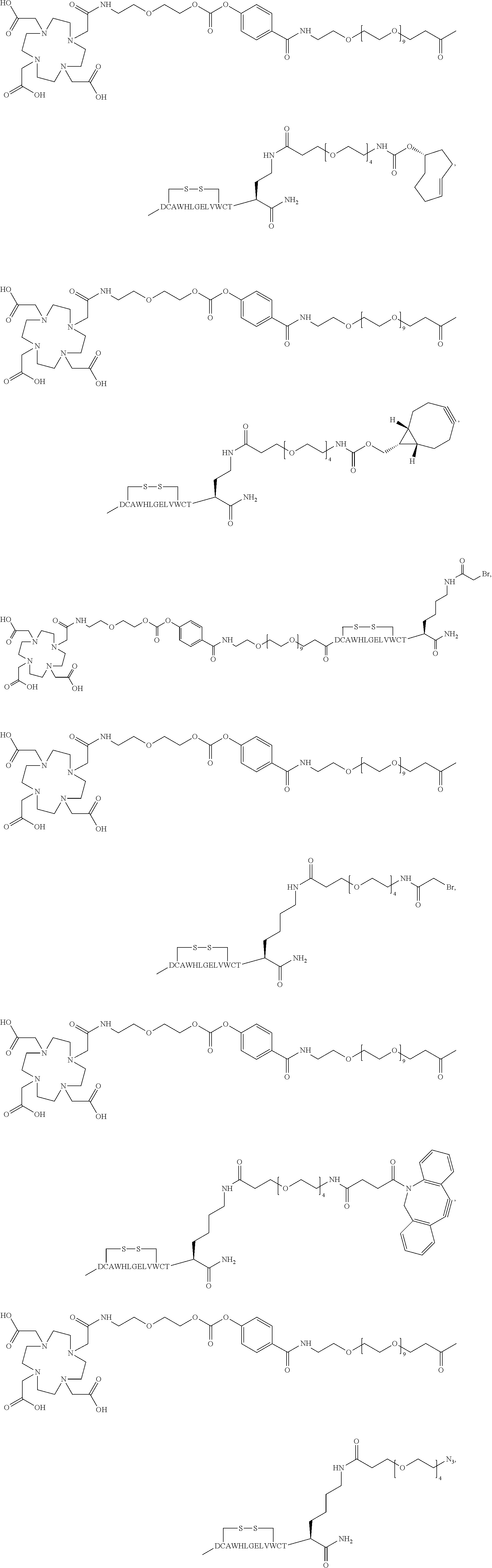

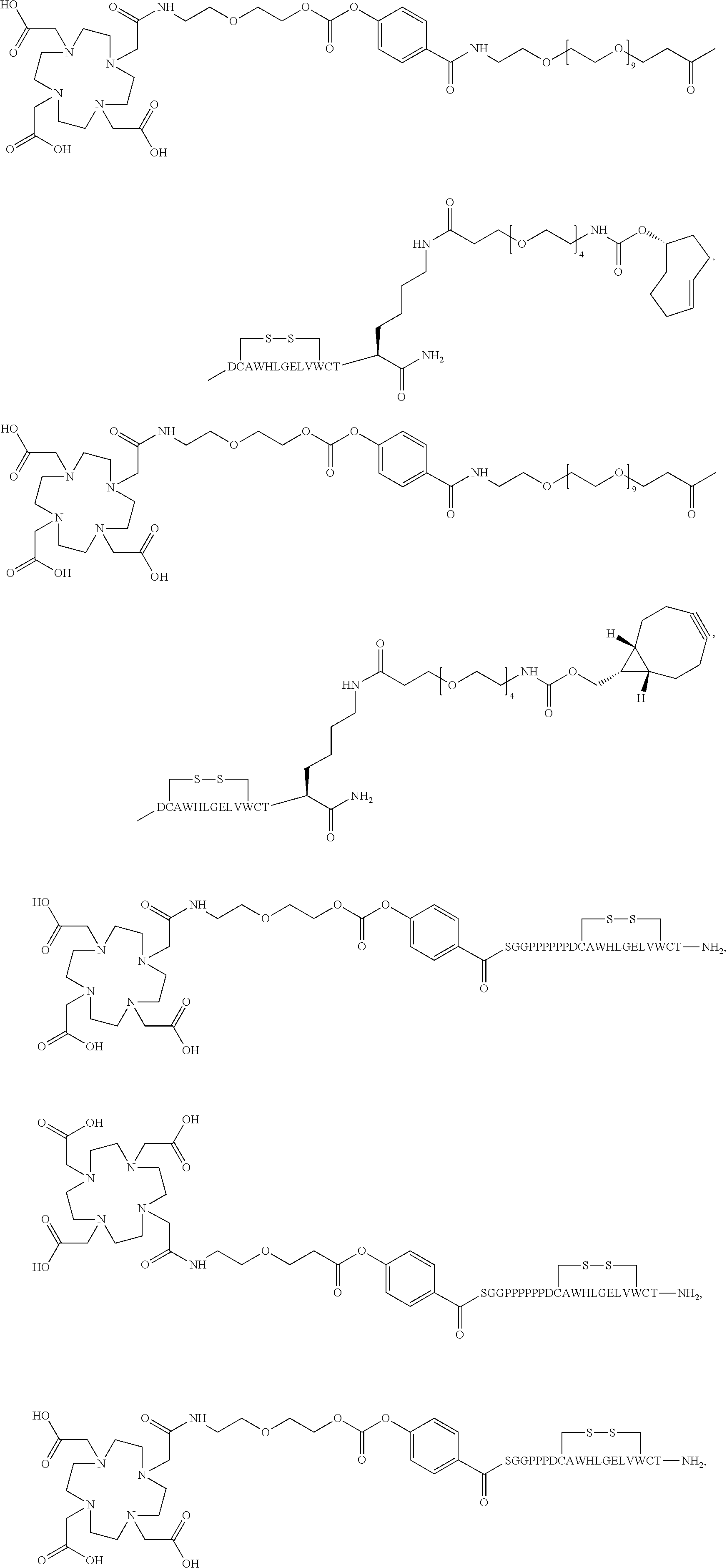

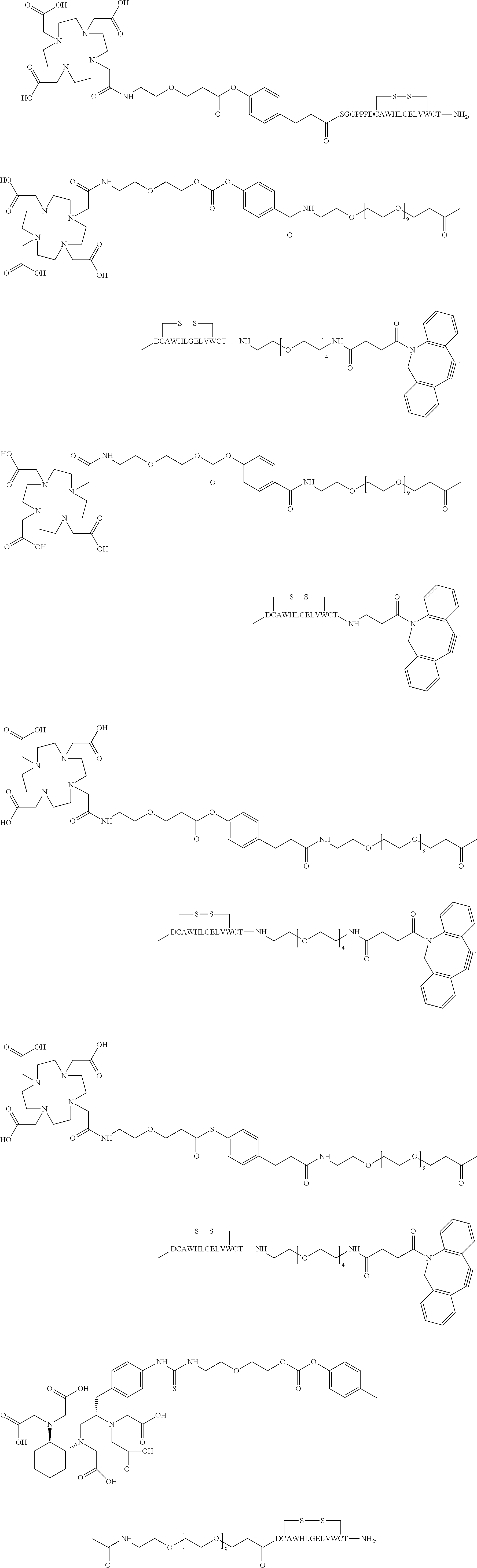

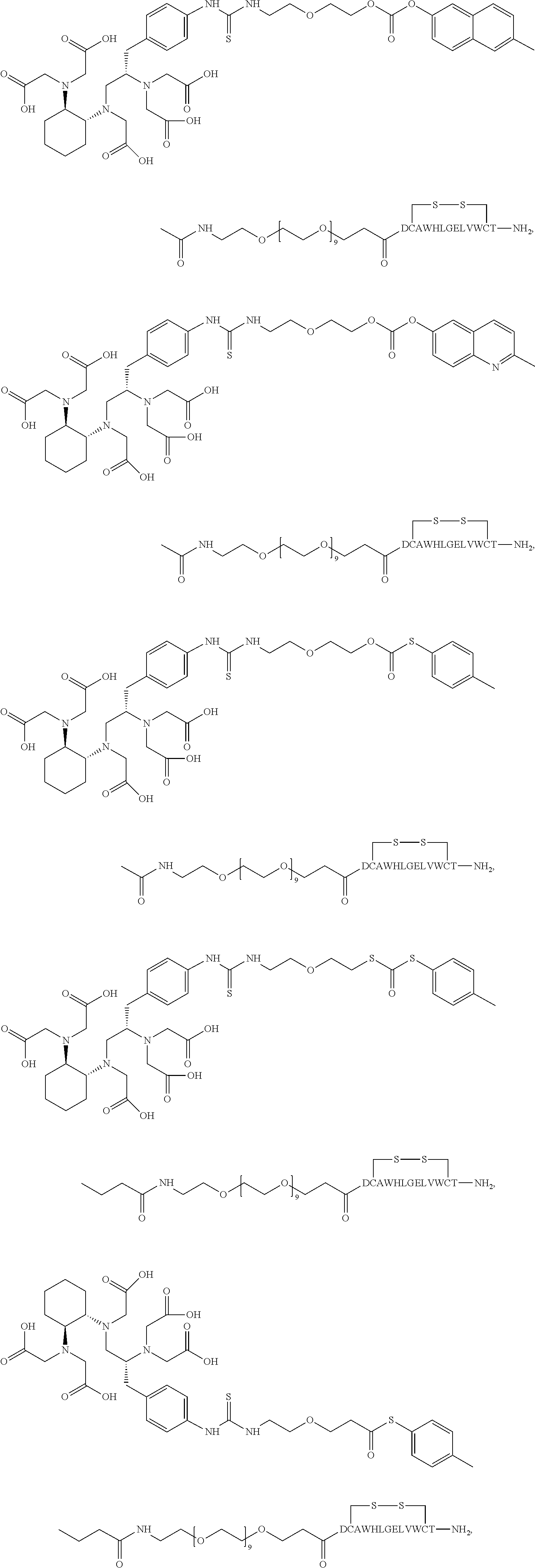

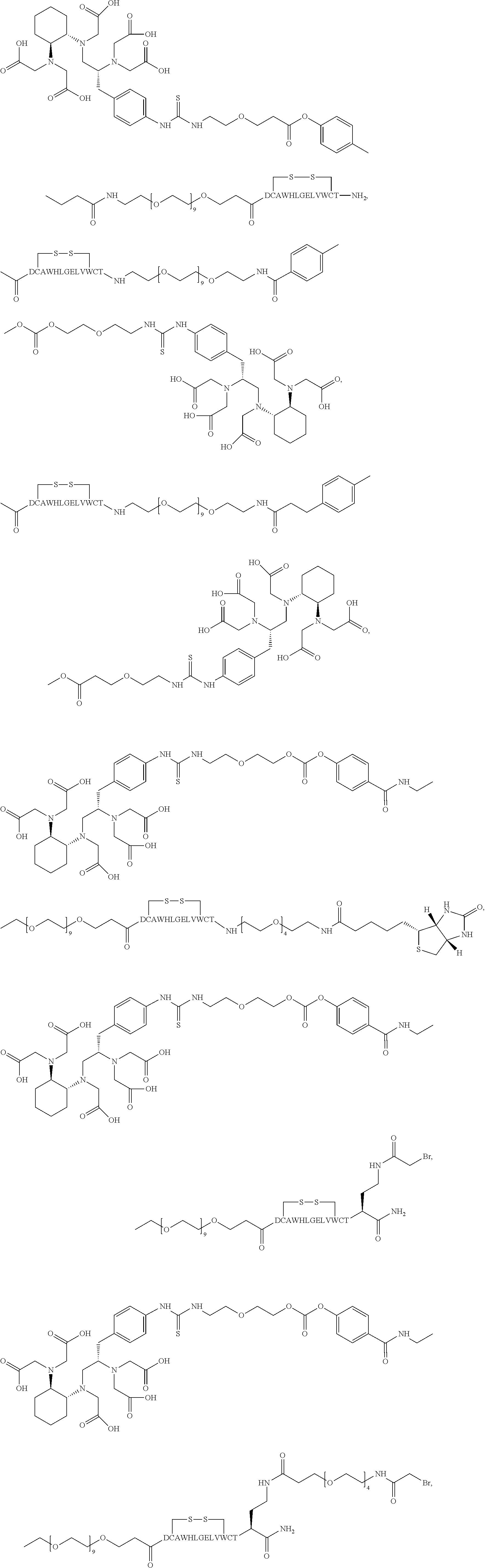

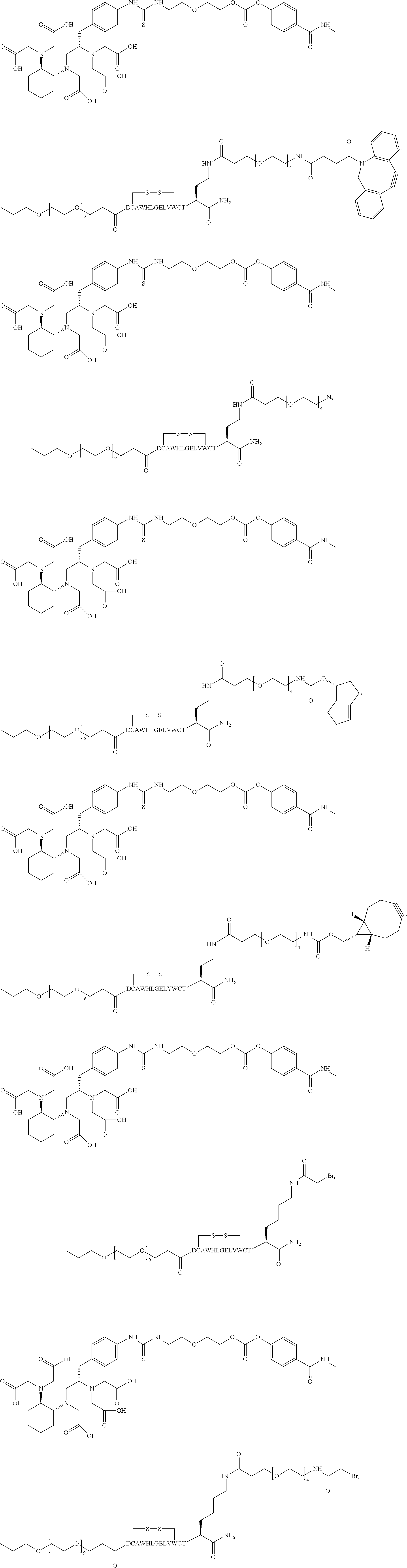

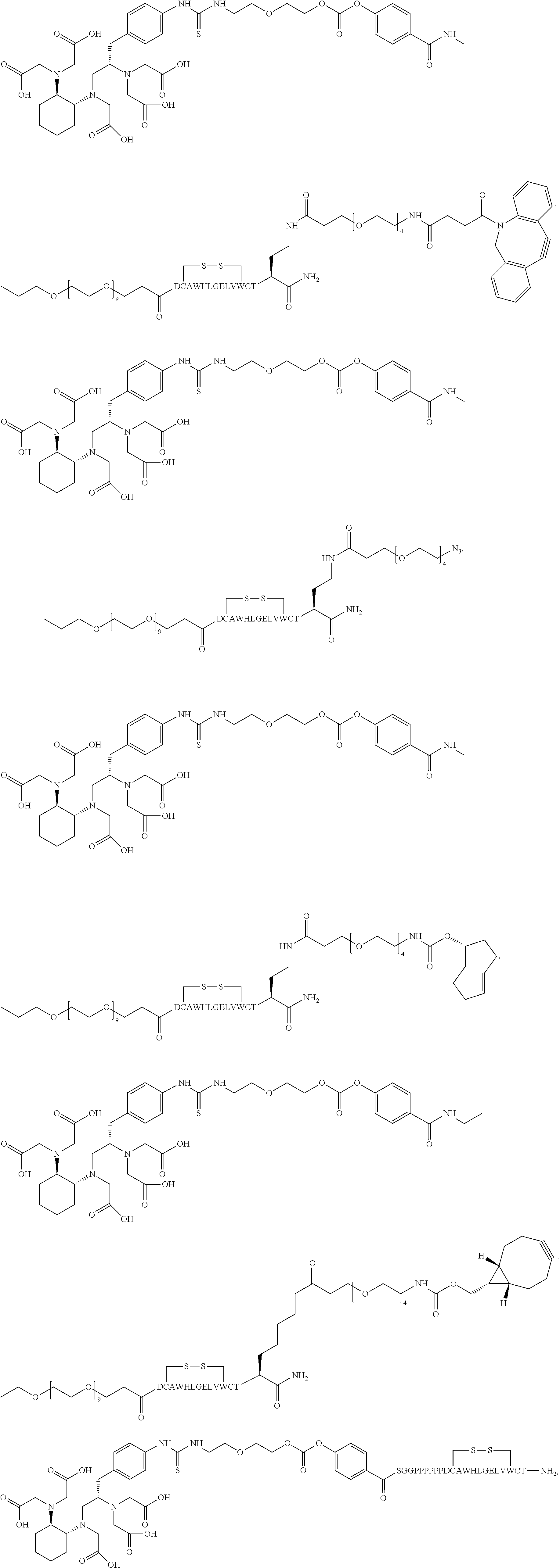

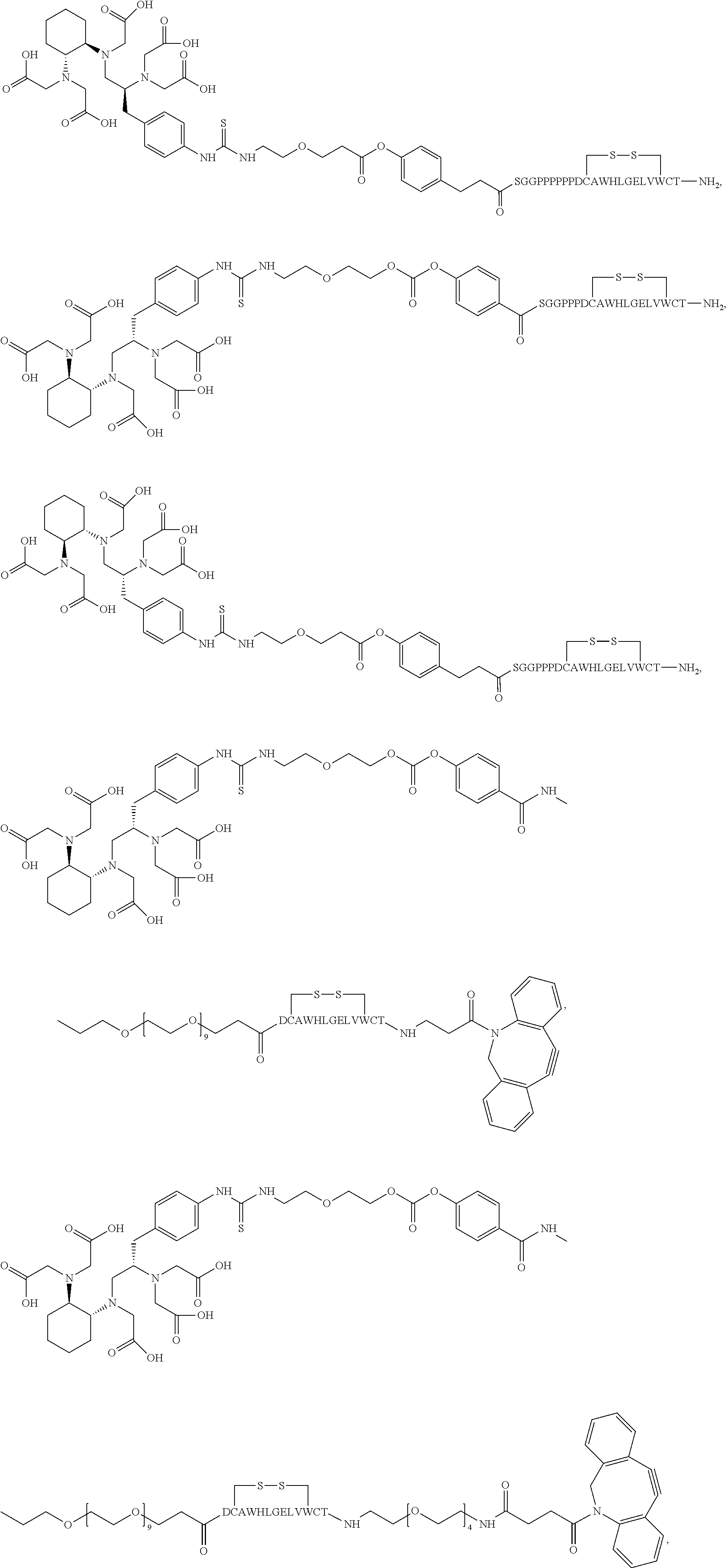

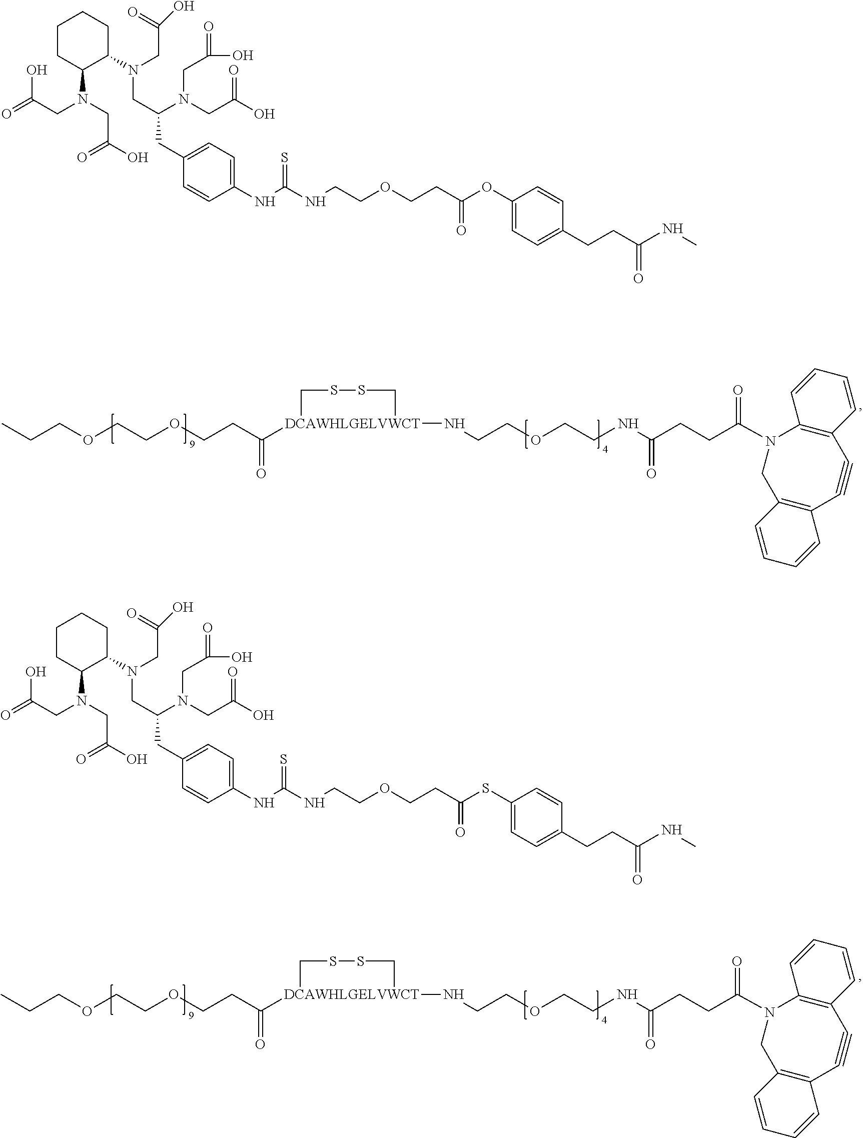

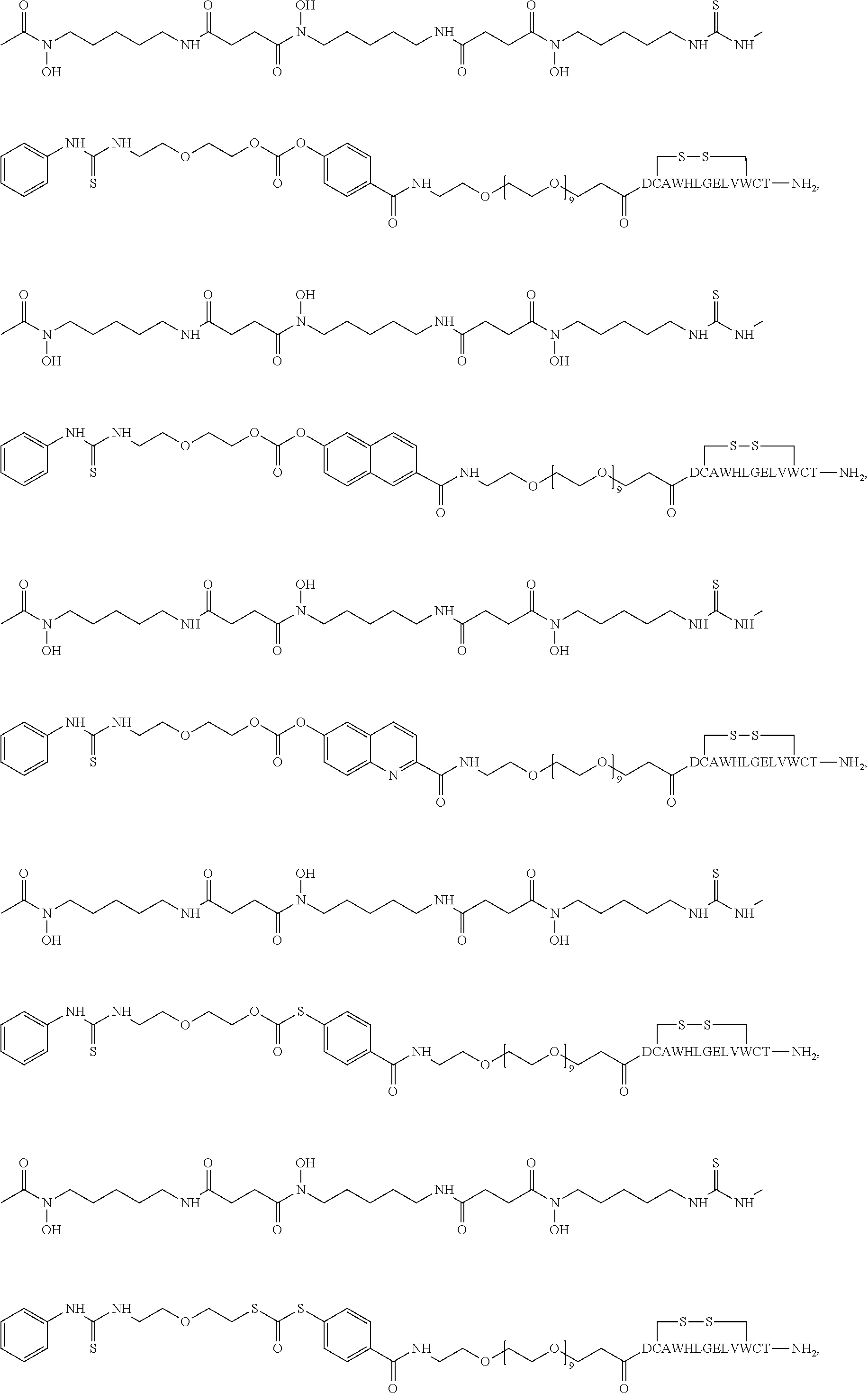

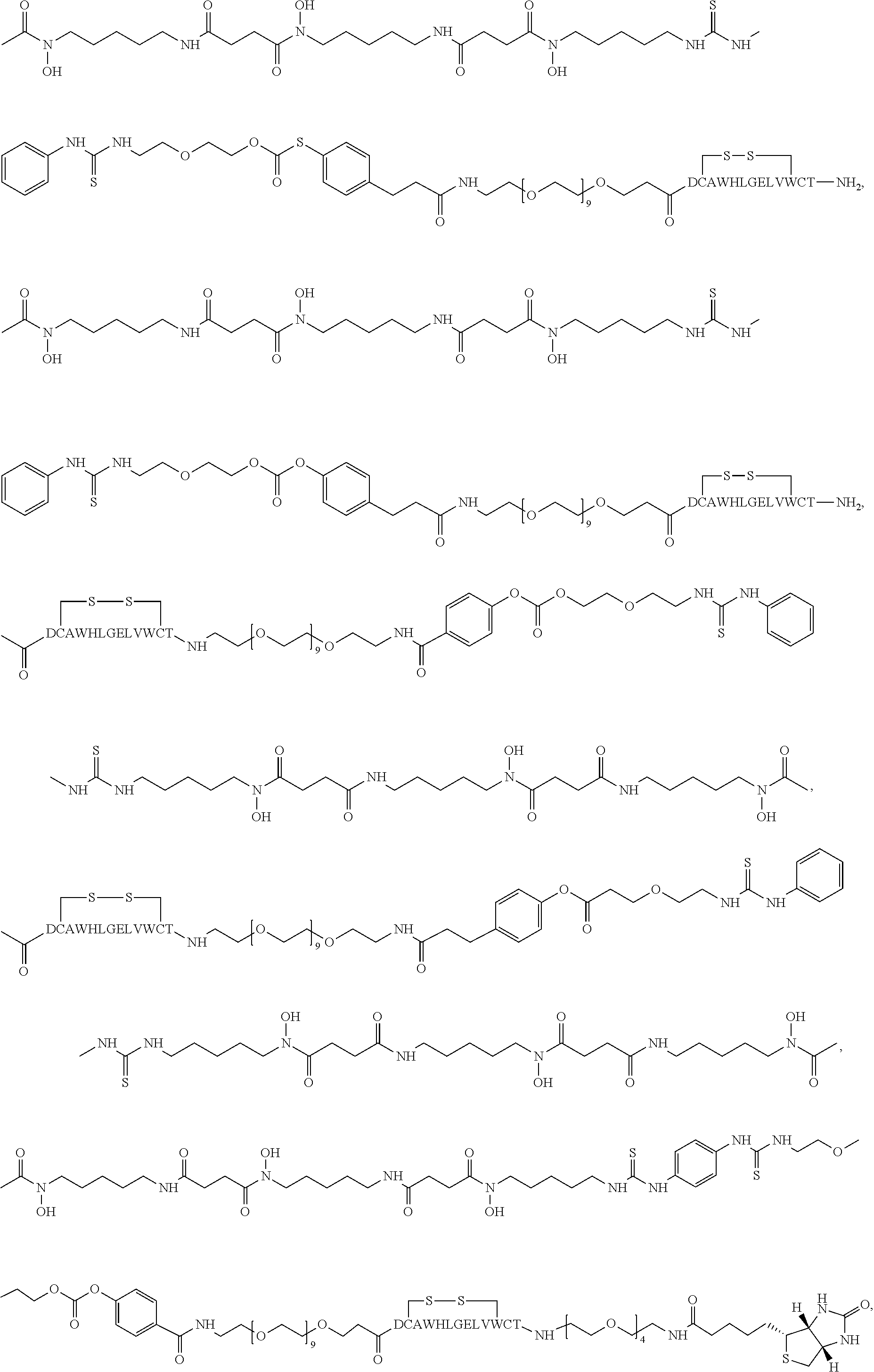

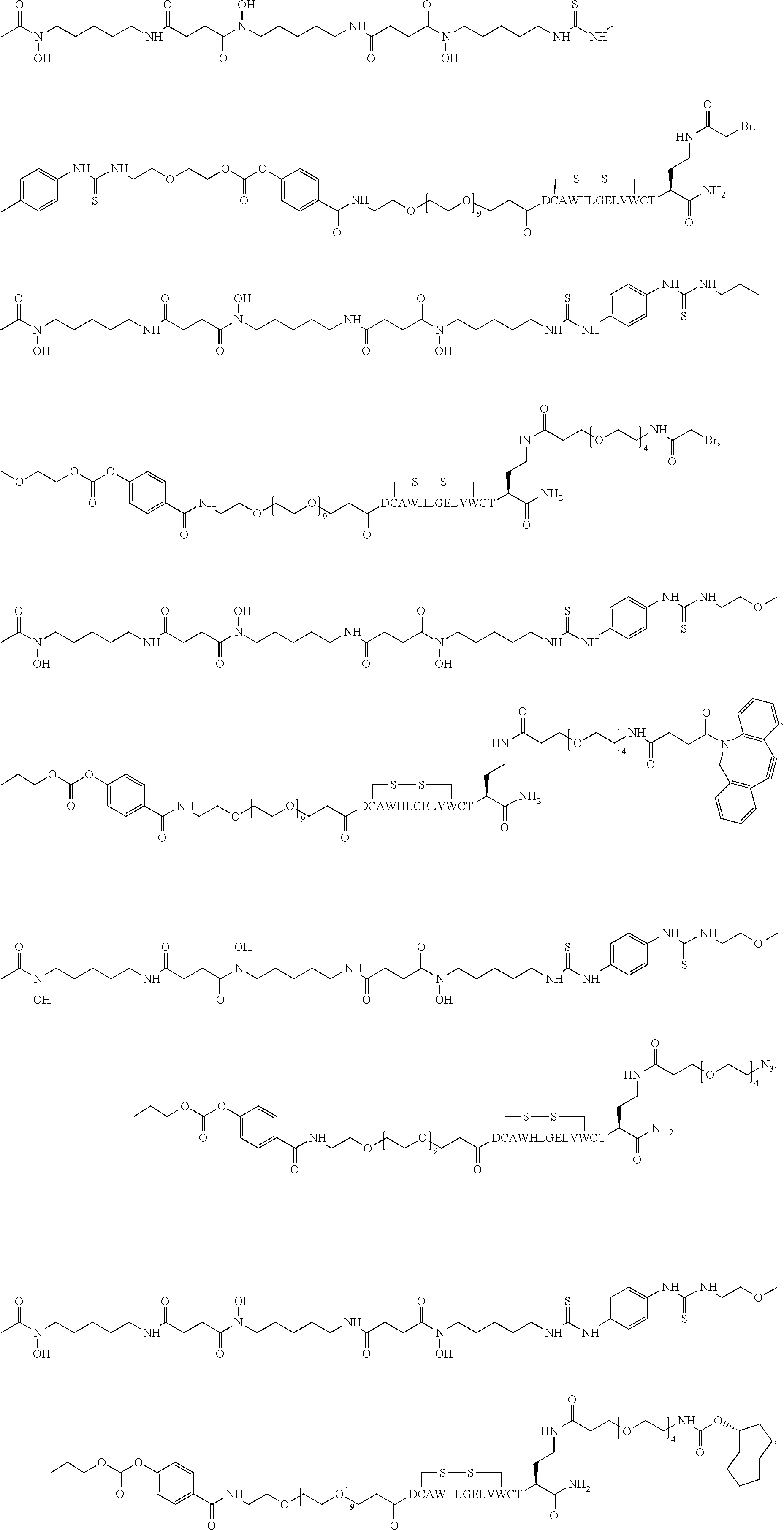

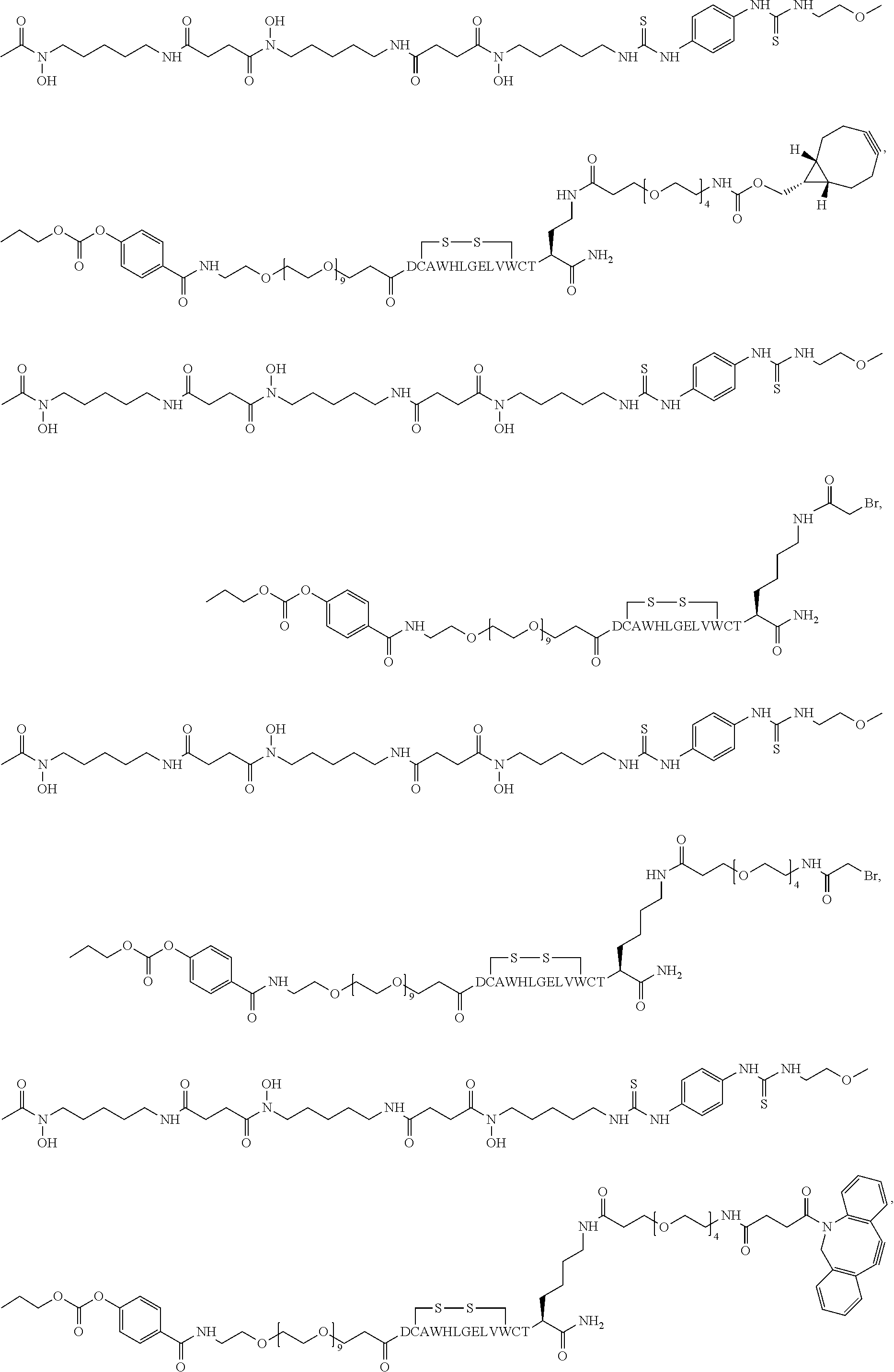

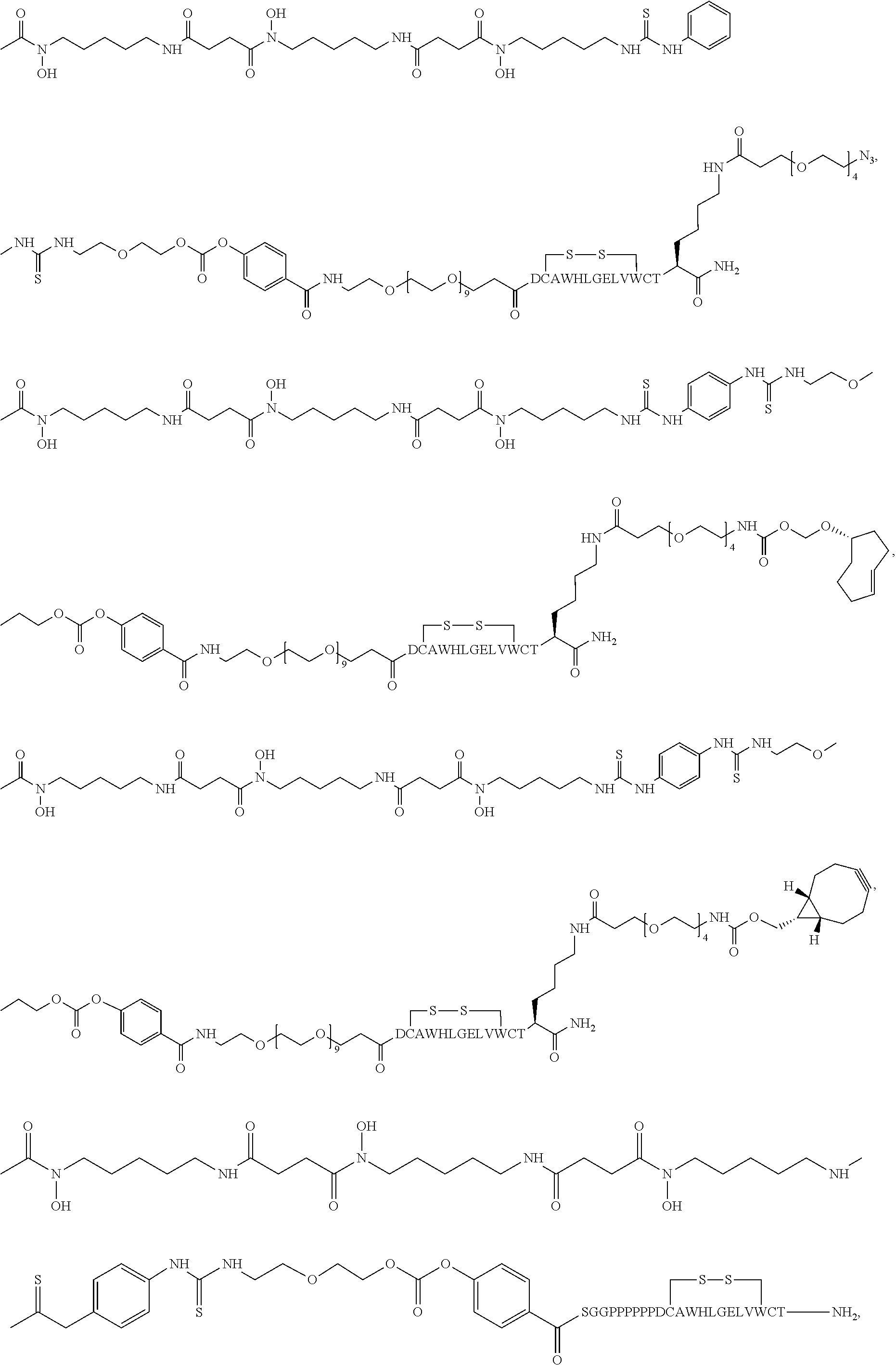

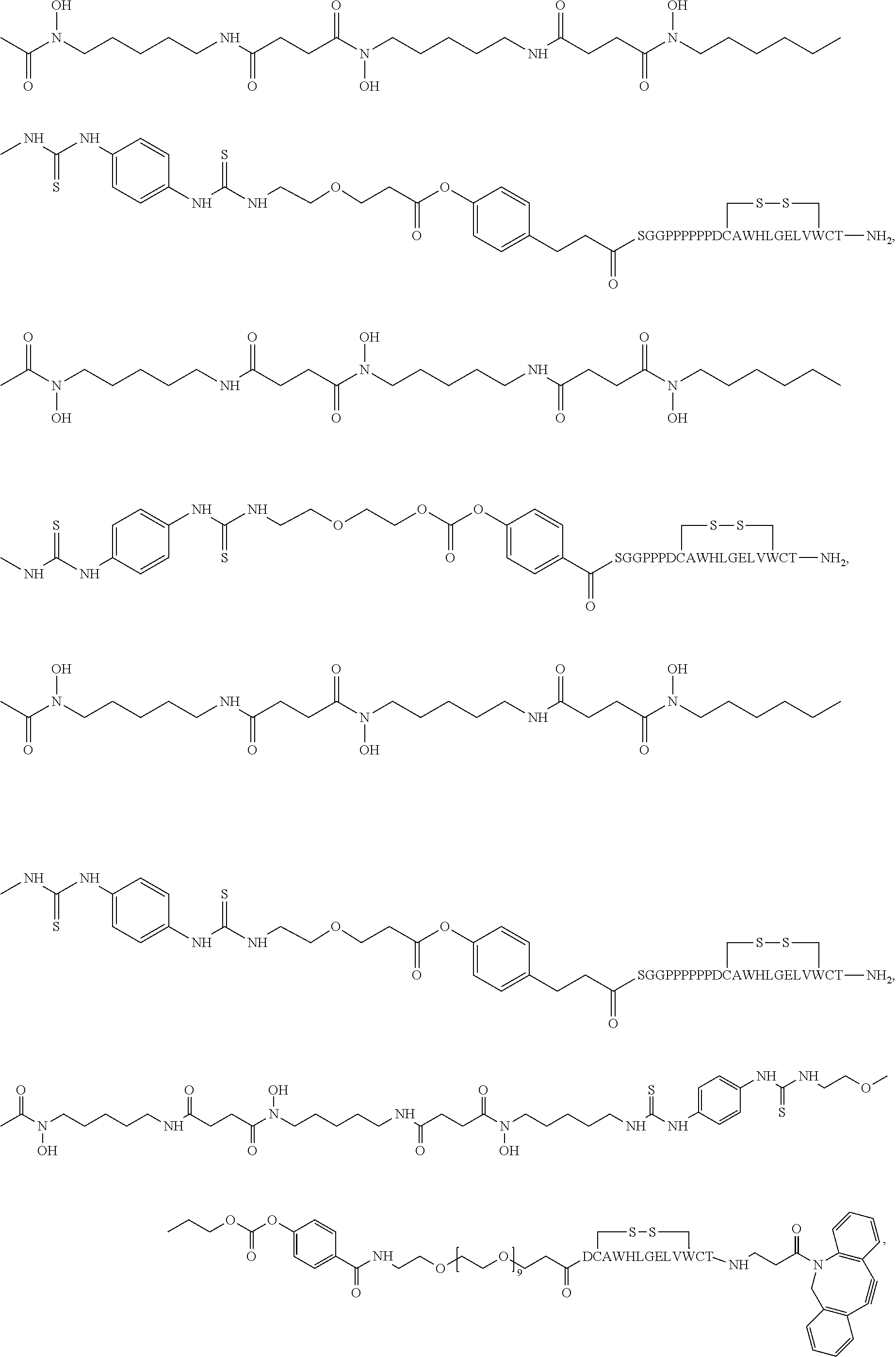

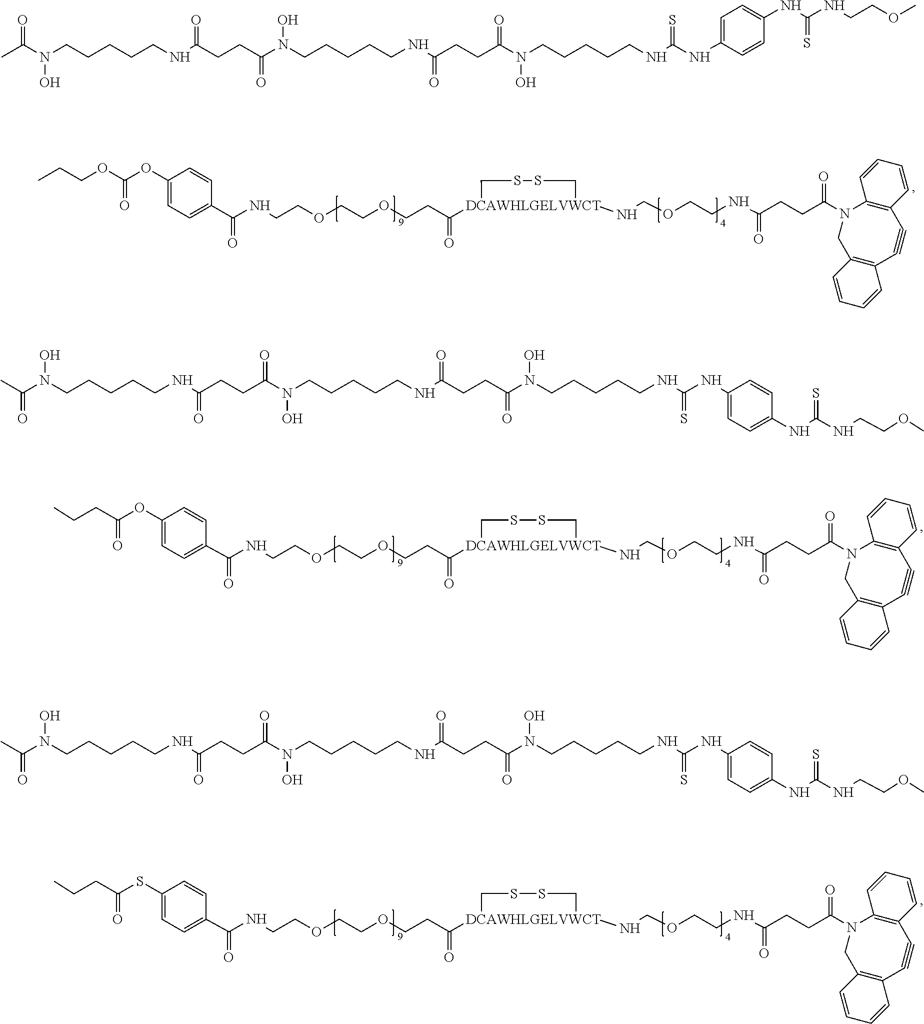

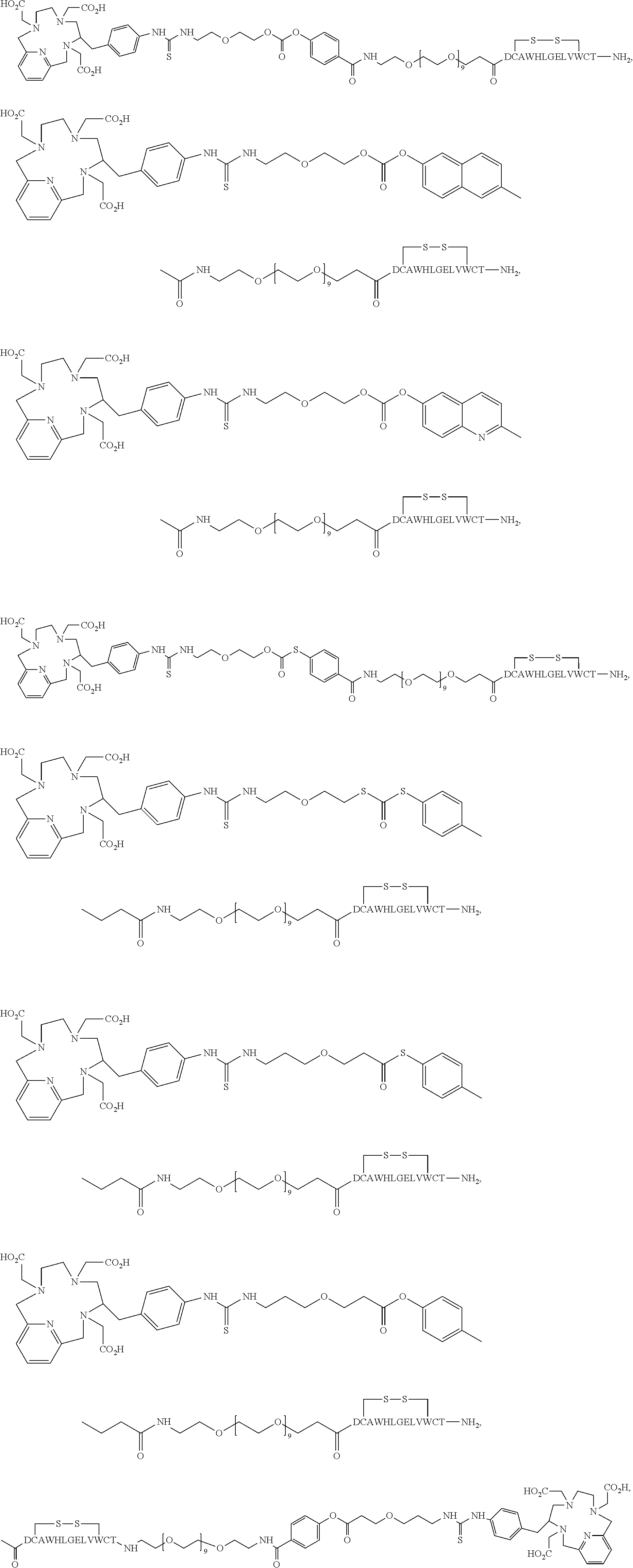

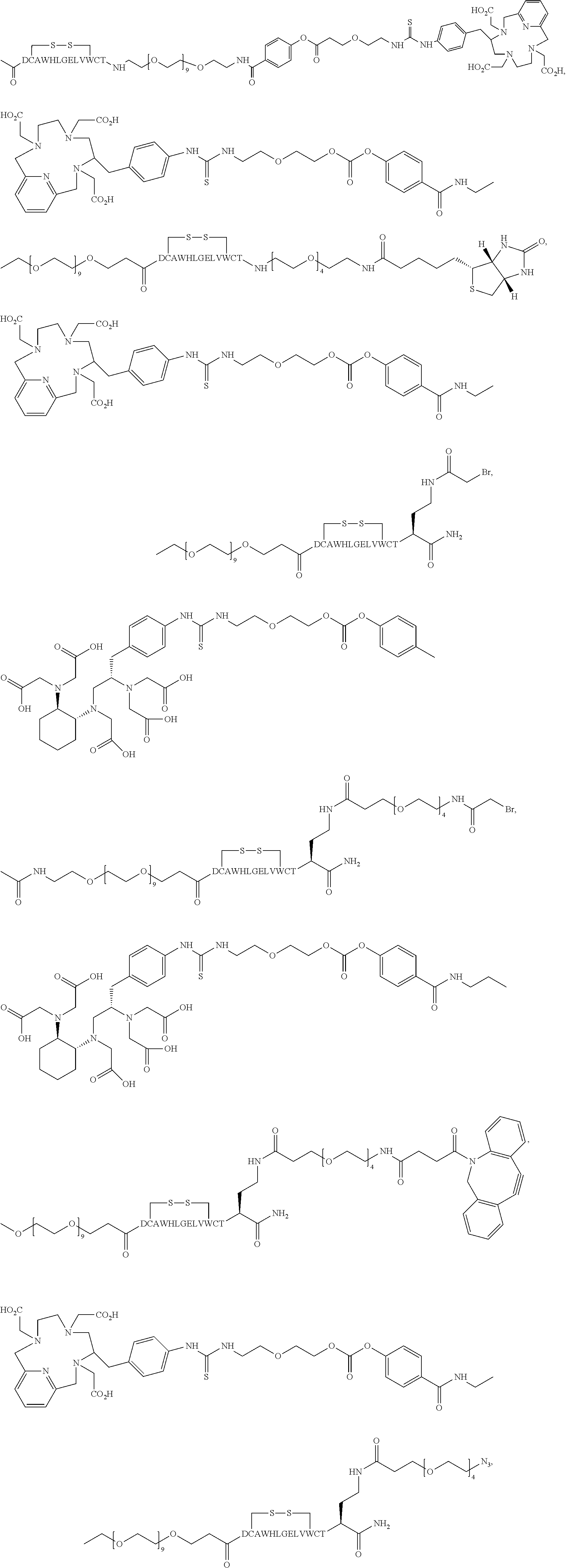

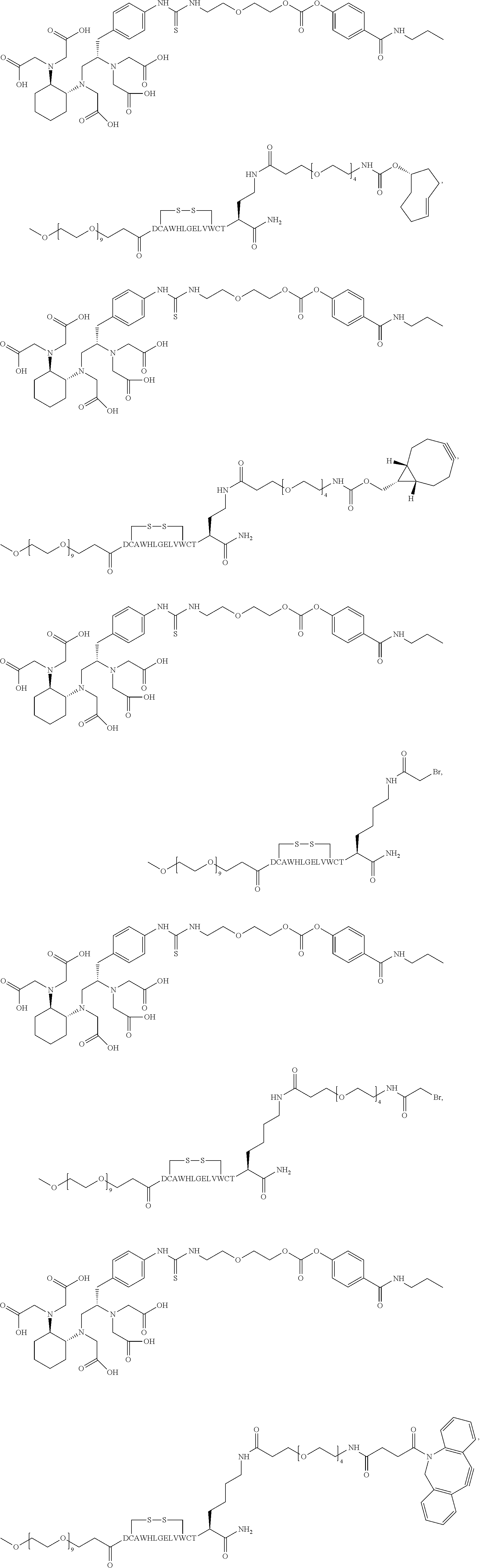

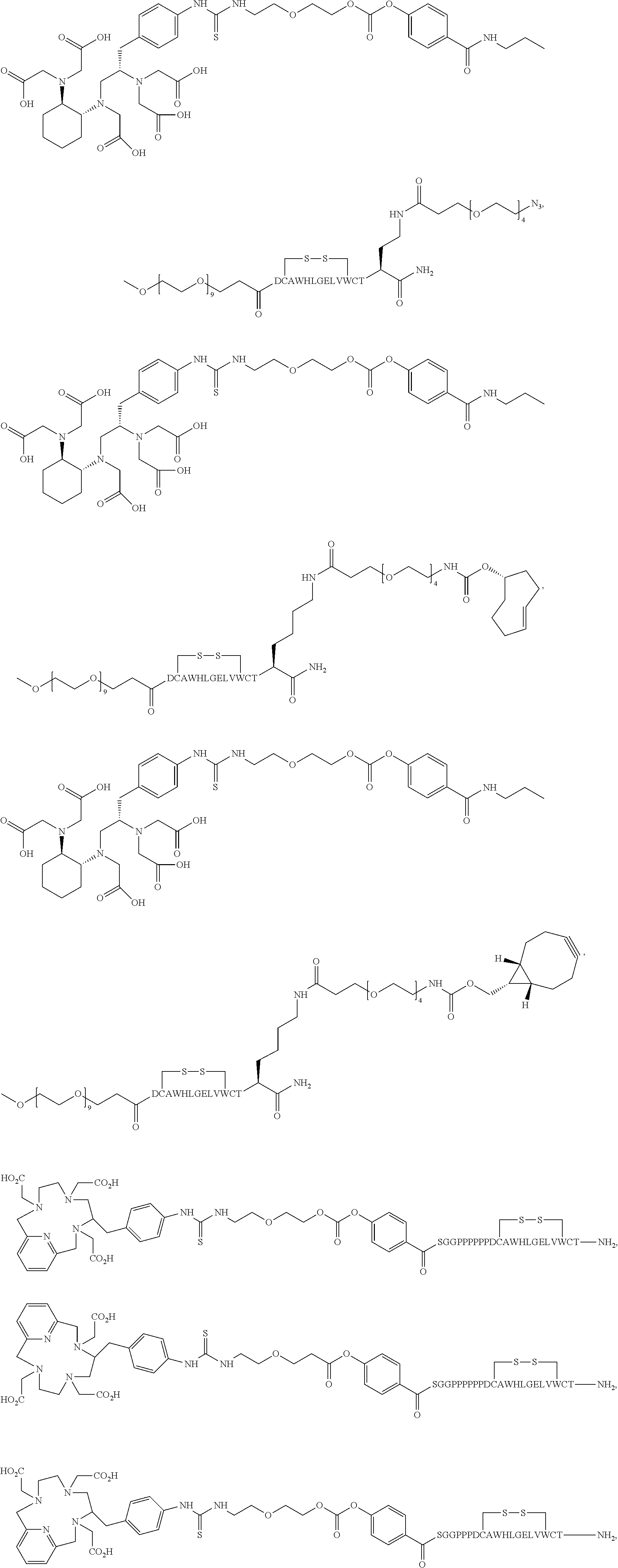

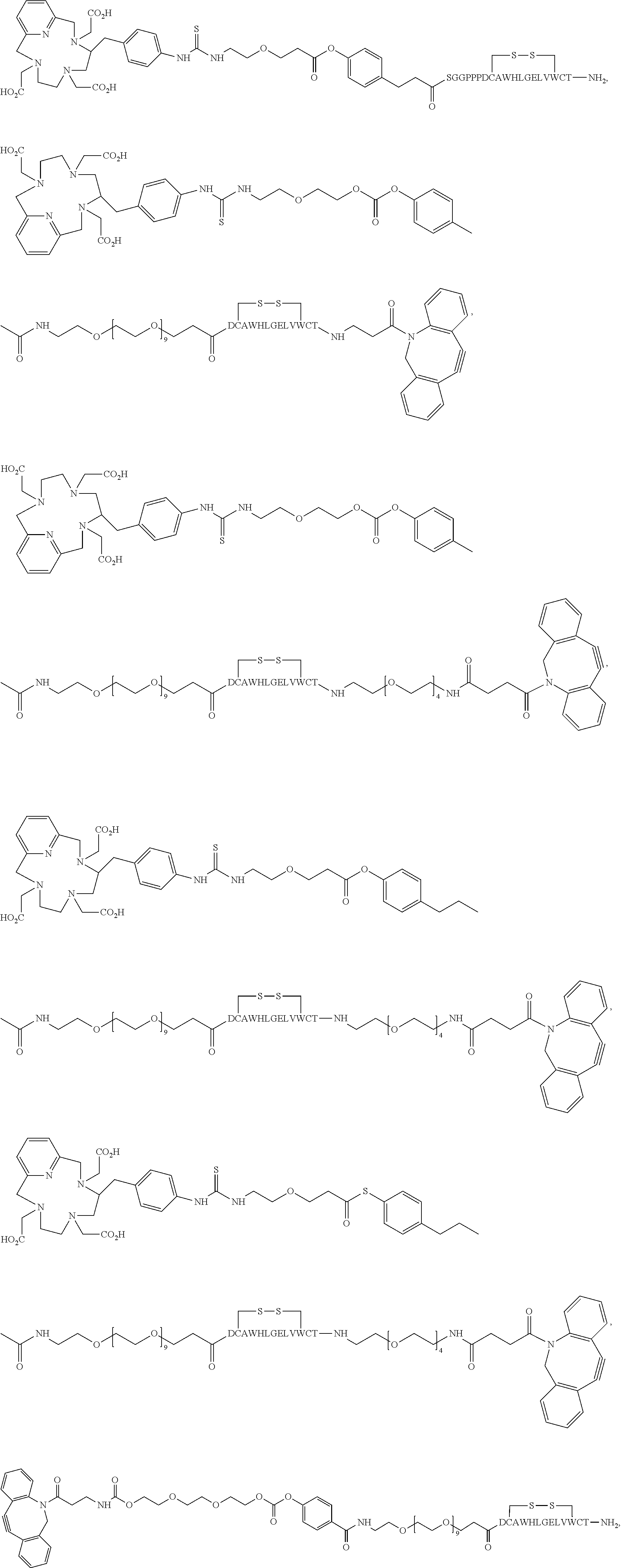

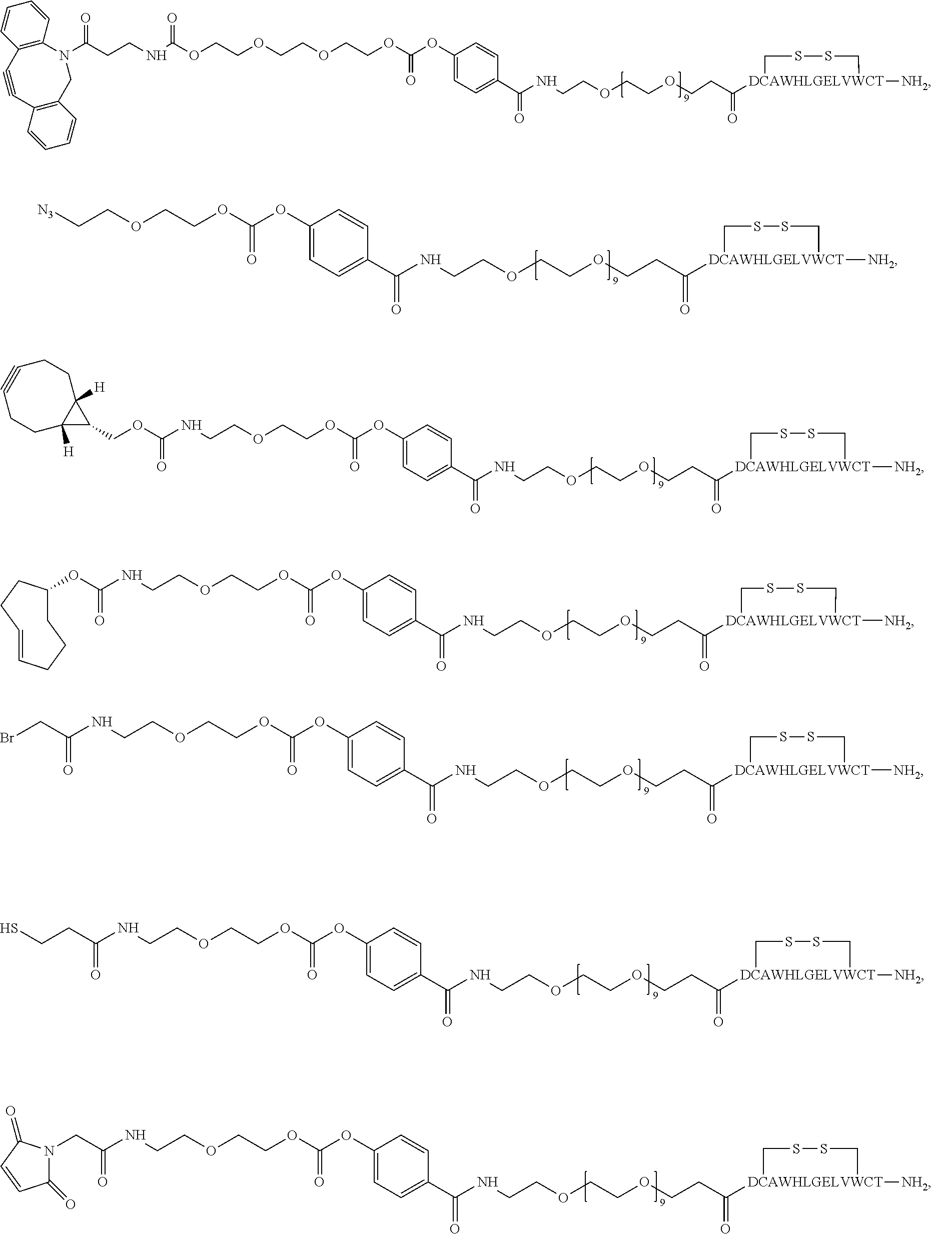

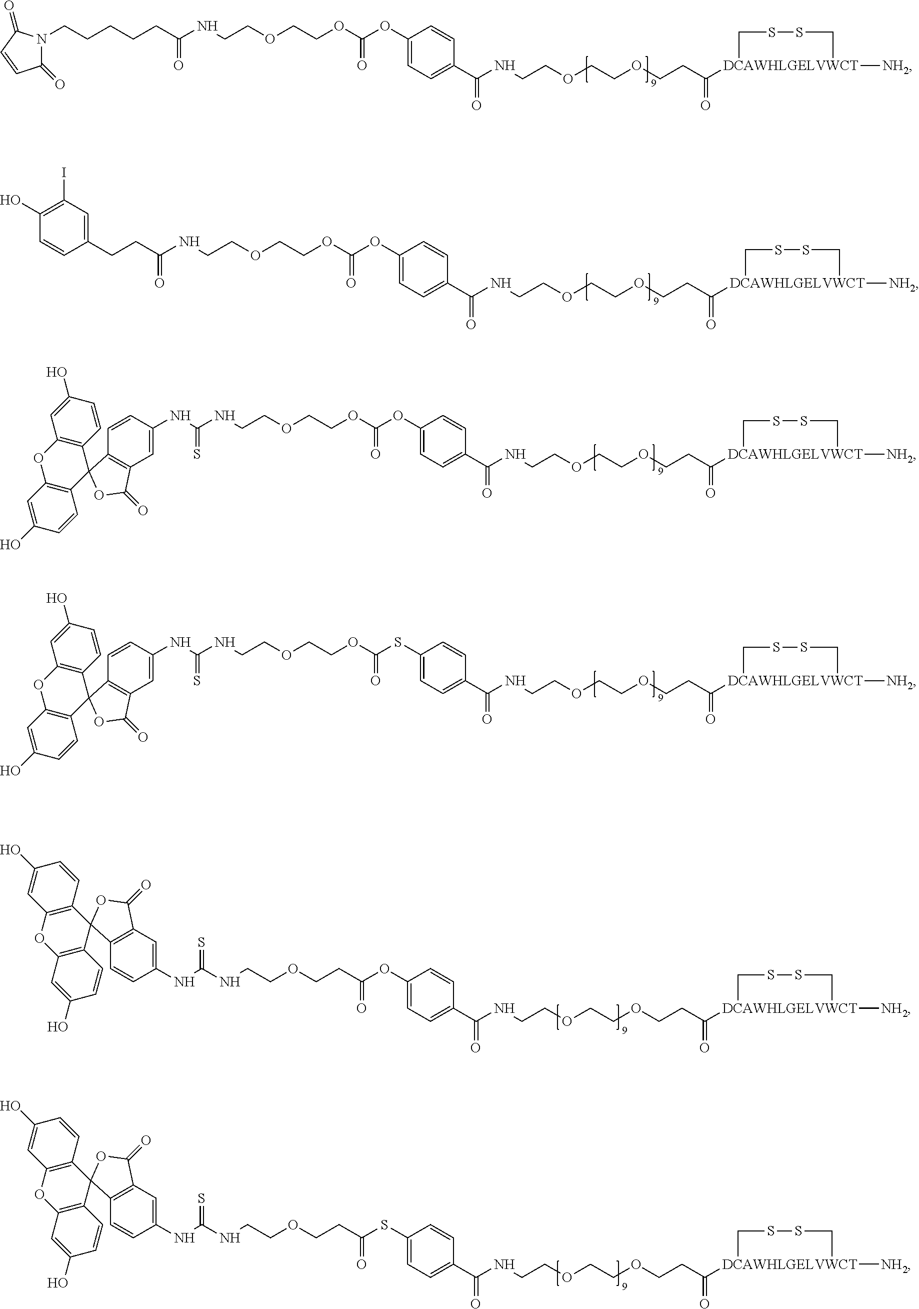

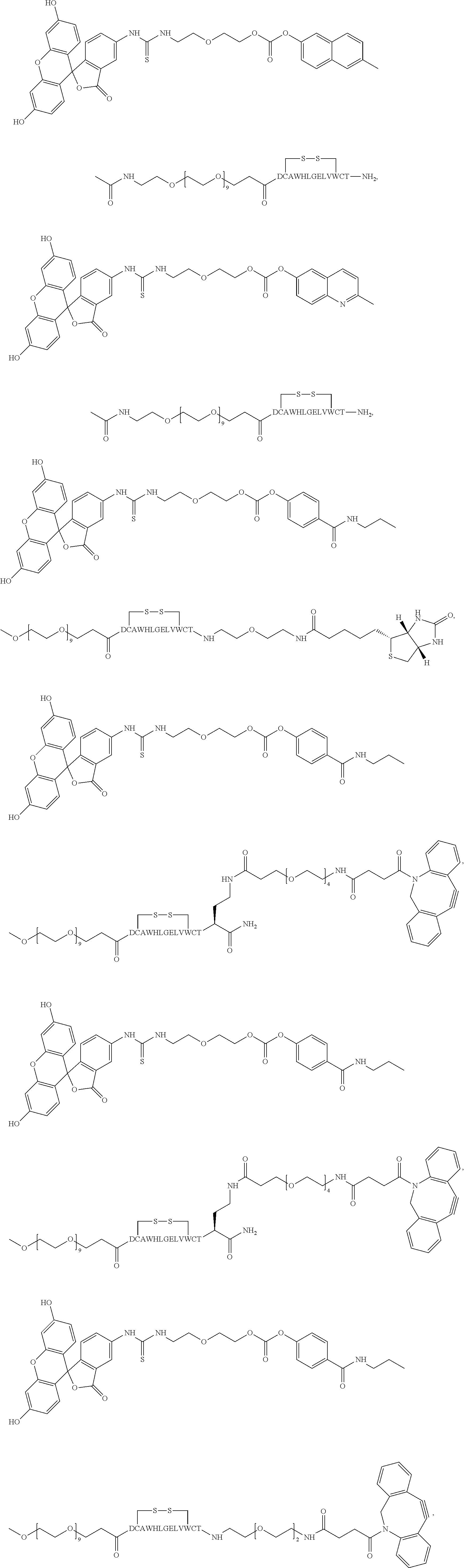

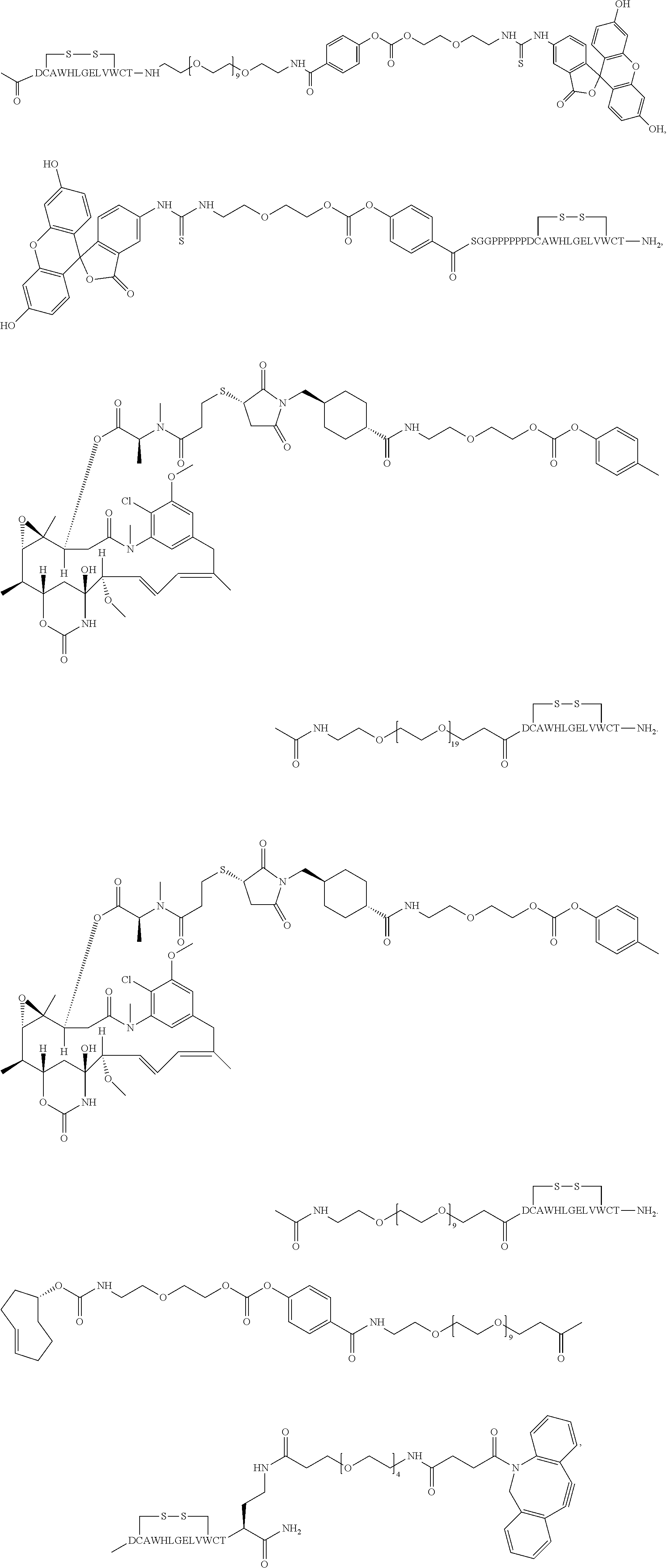

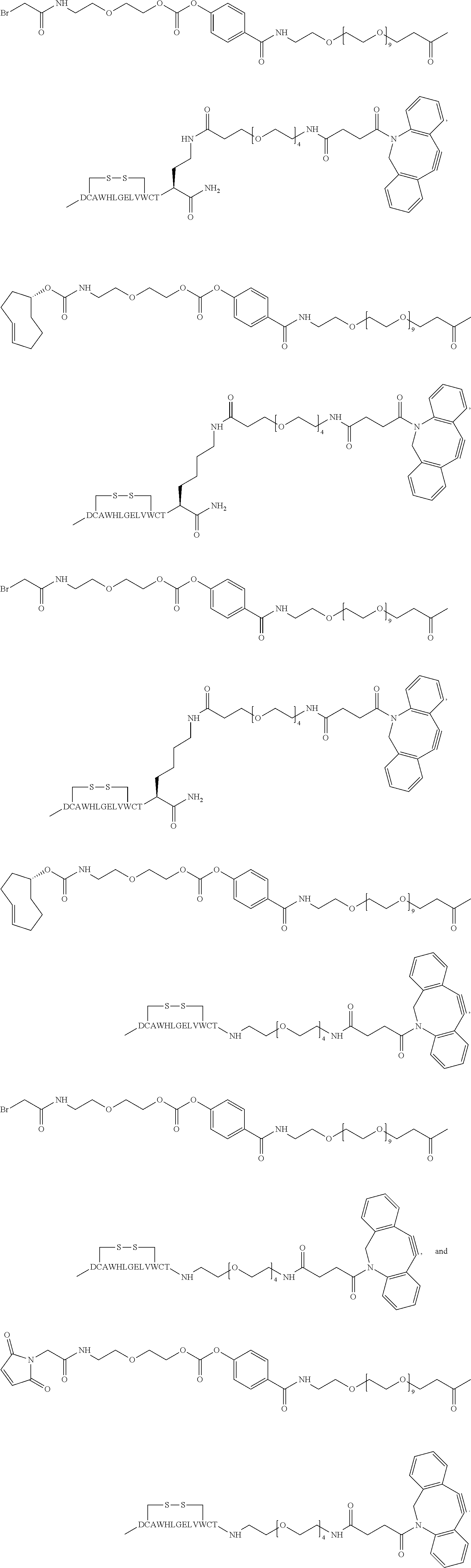

In one embodiment, the number of repetitions of the spacer polyethylene oxide moiety in the above compounds (i.e. 9) can be replaced by any of 5 to 35, preferably by 7 to 19, a spacer having 9 polyethylene oxide repeating units being the most preferred option.

In a preferred embodiment, the compound of formula (1) is selected from:

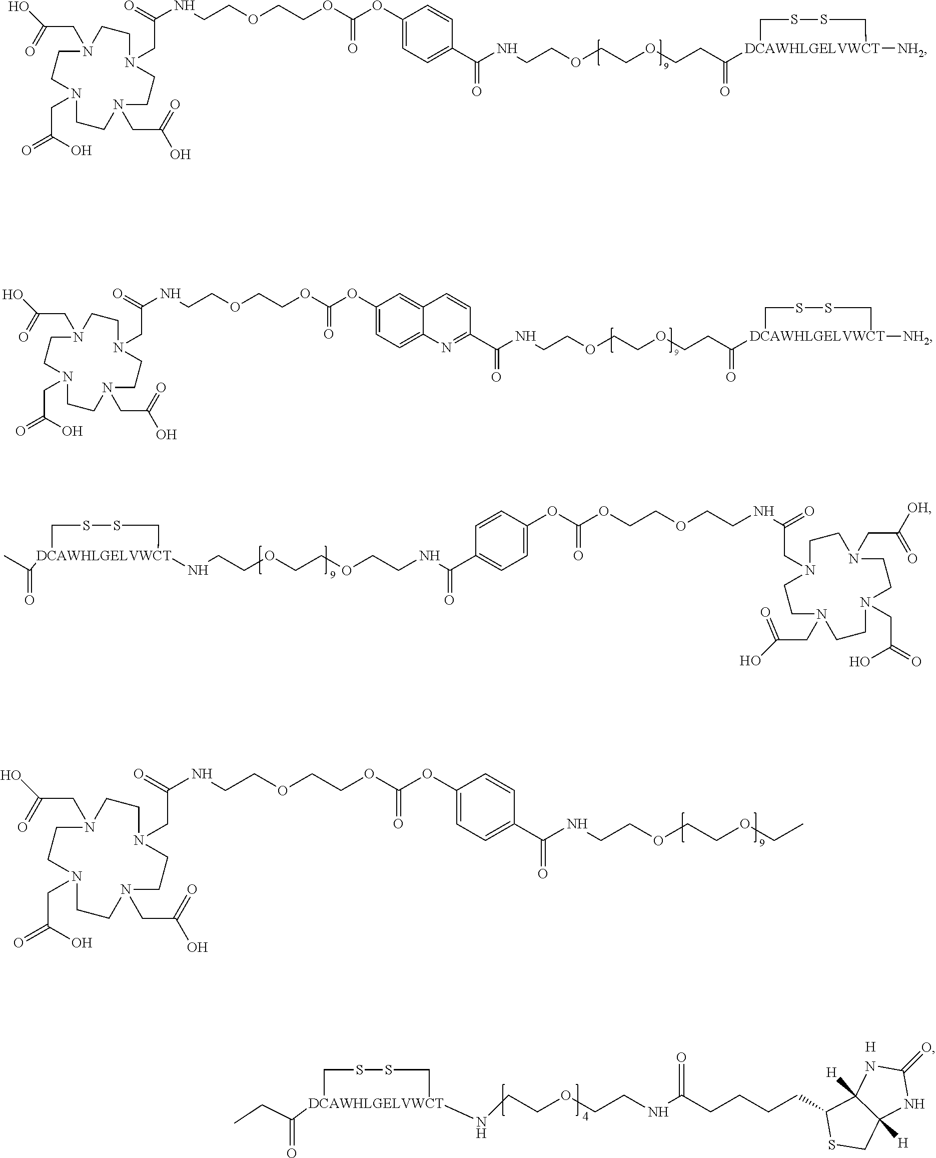

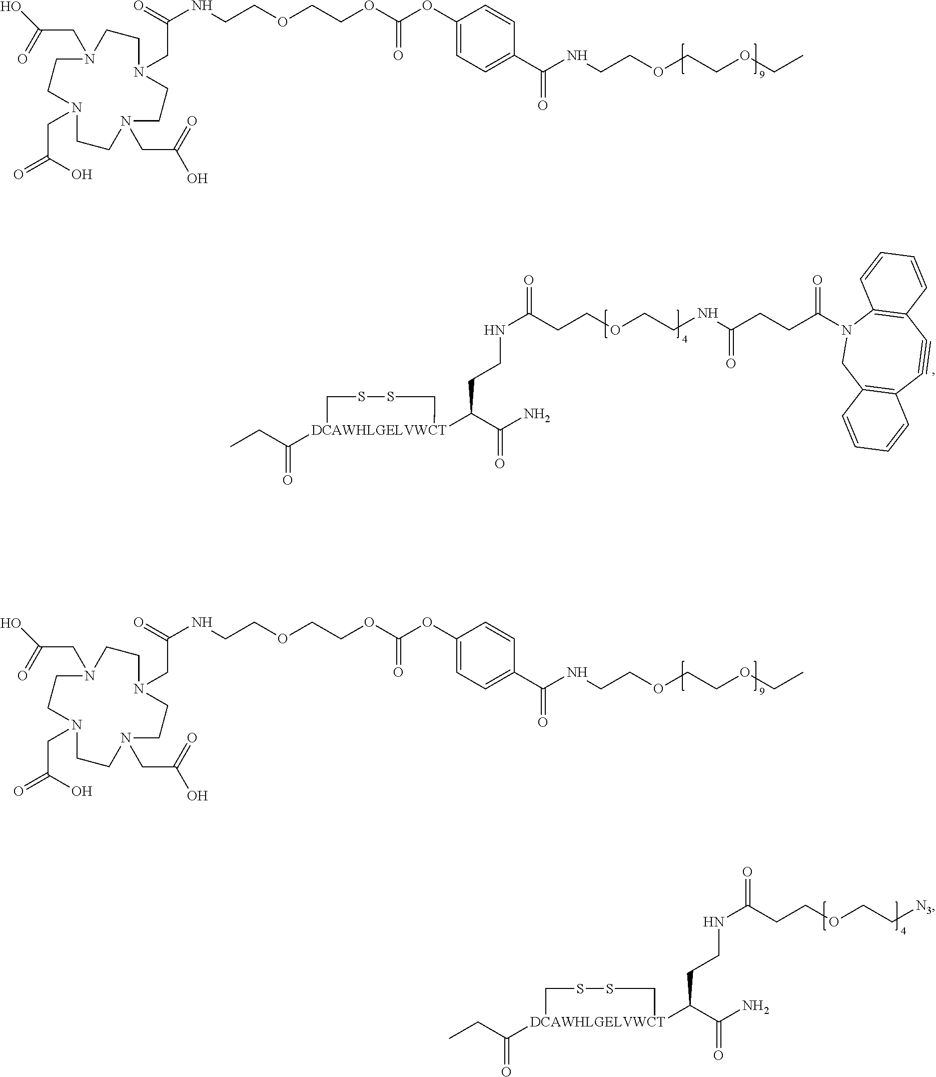

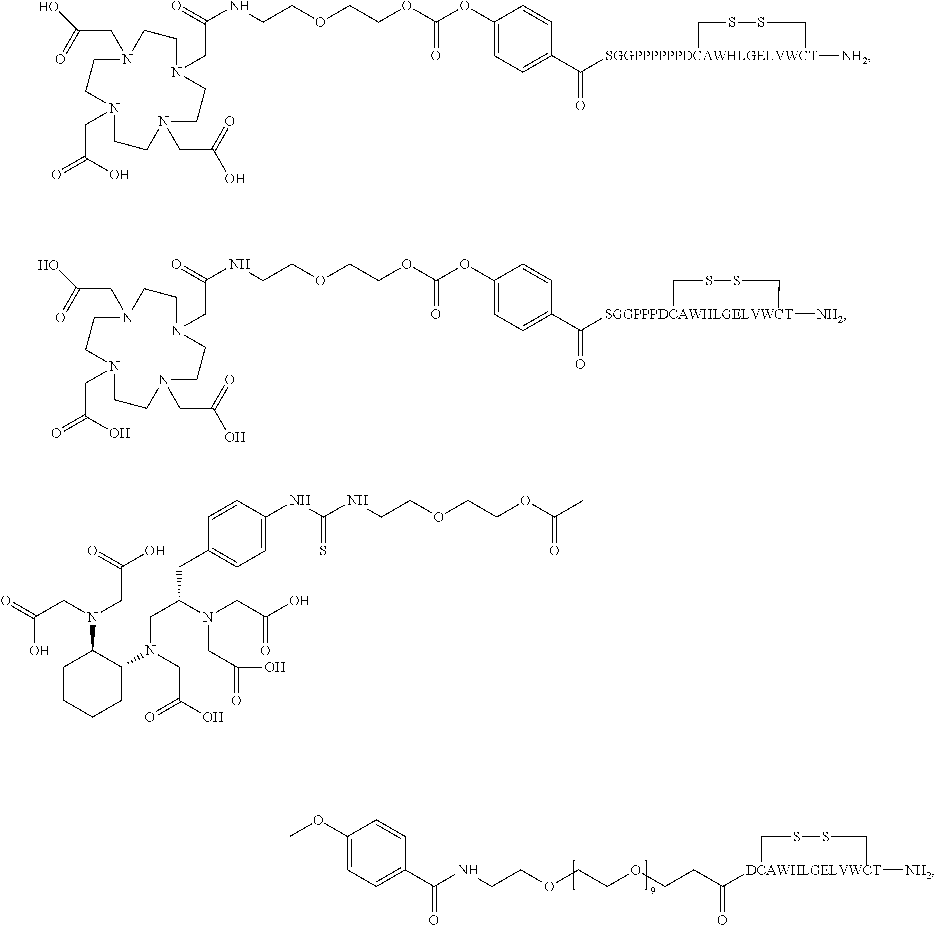

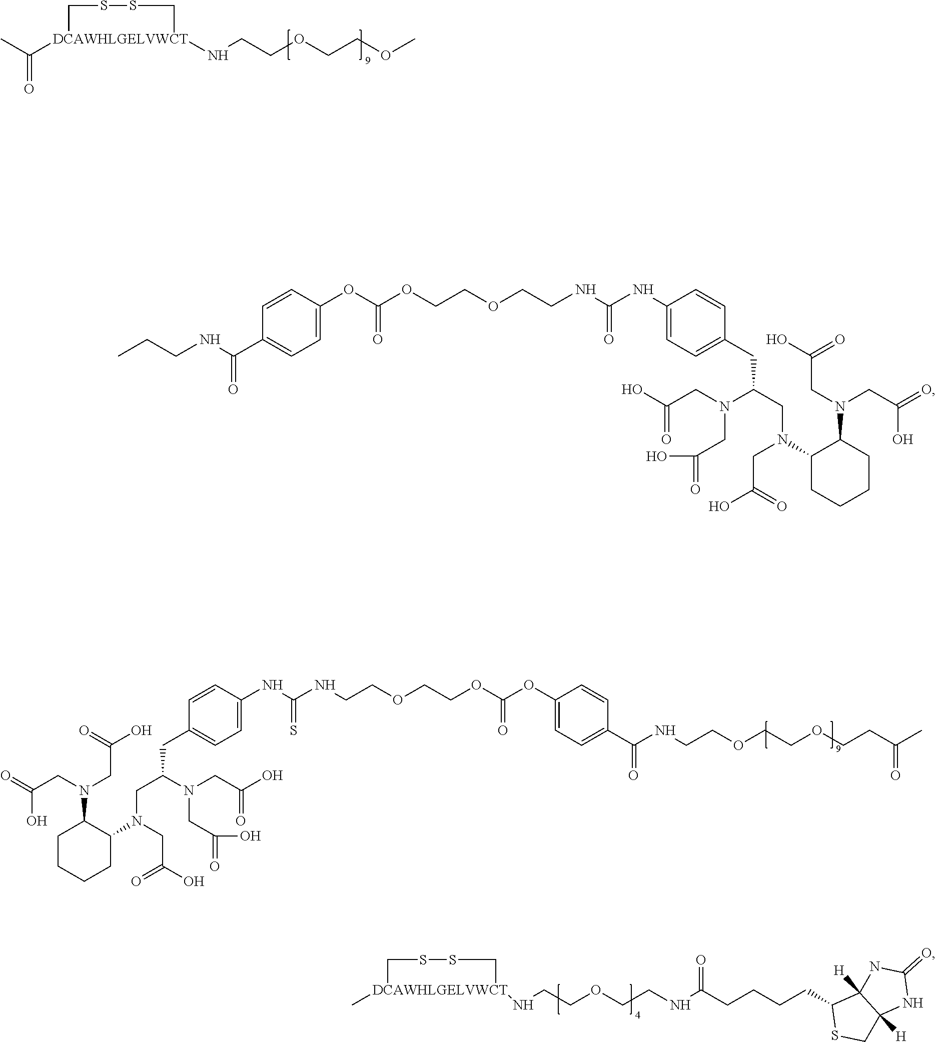

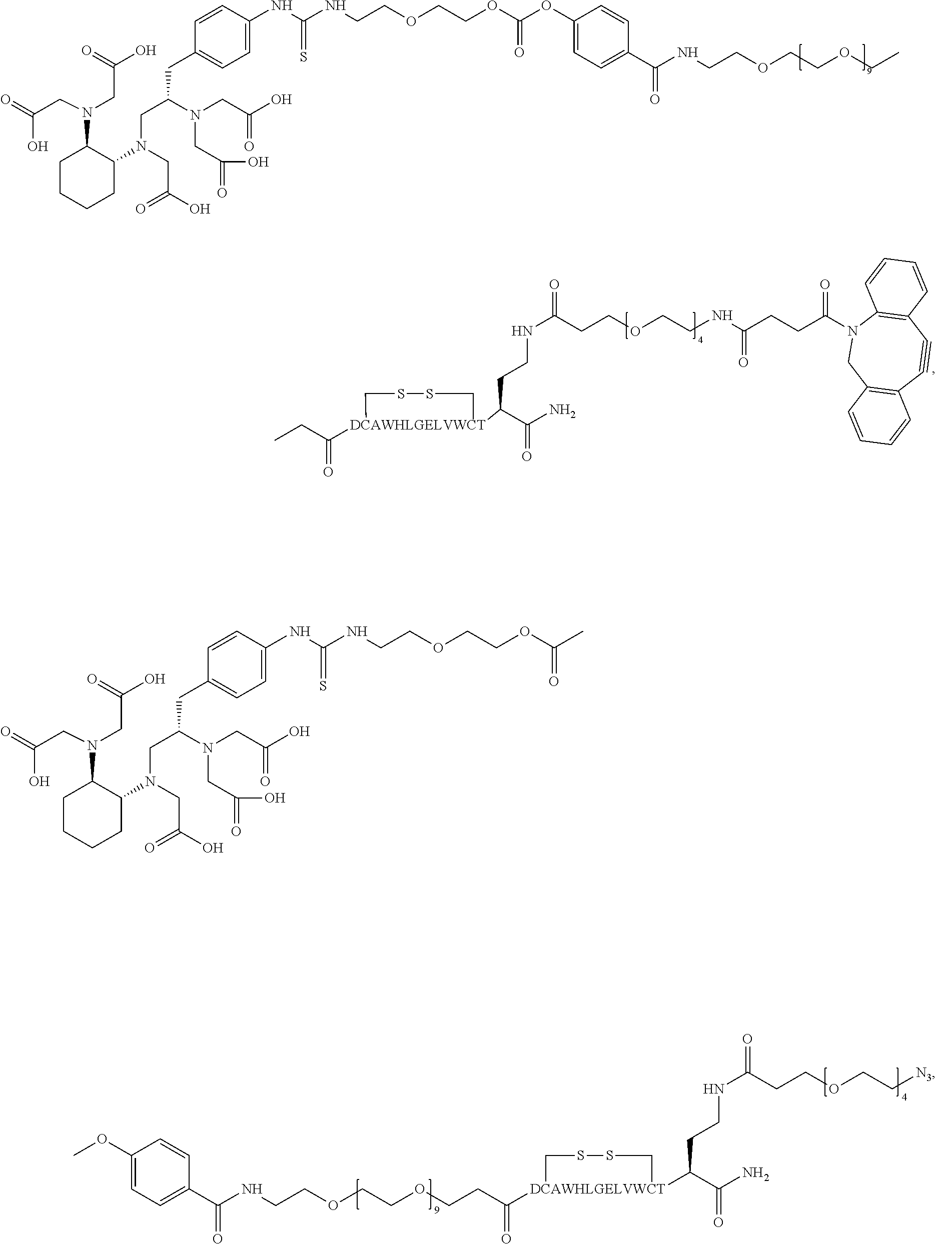

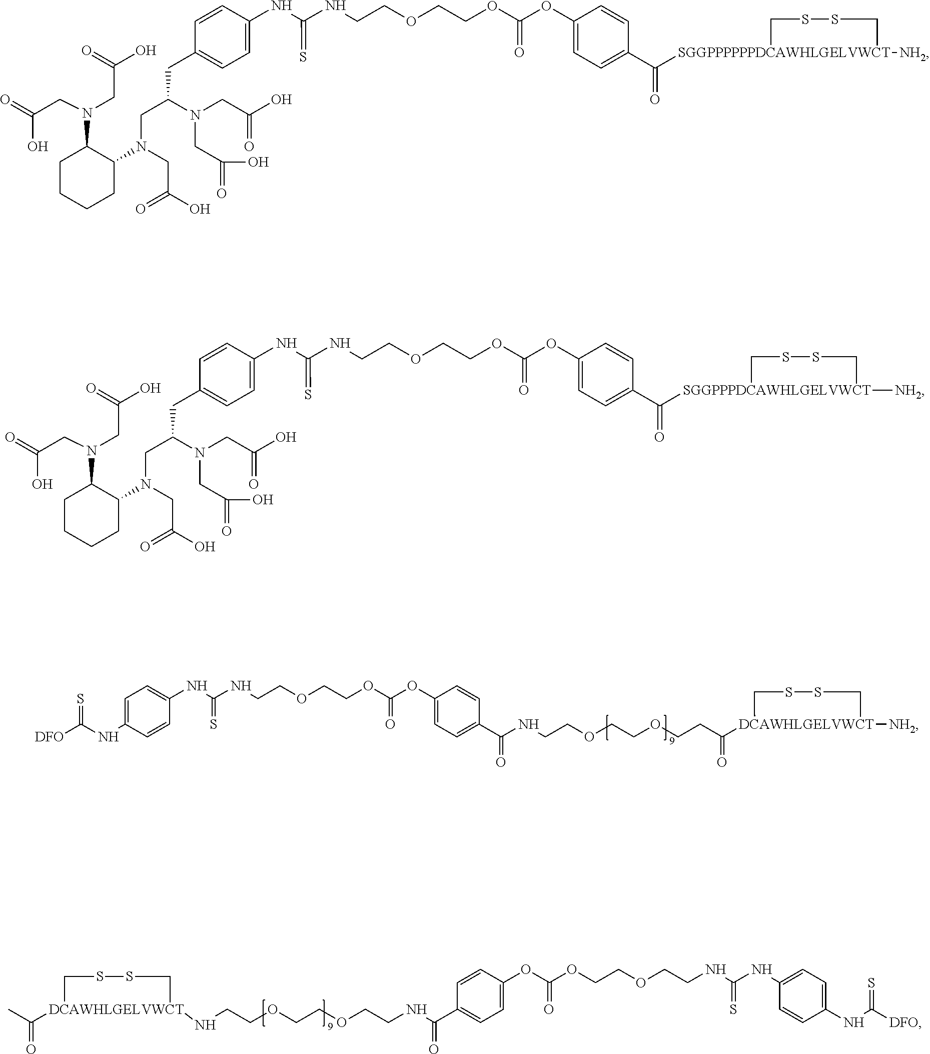

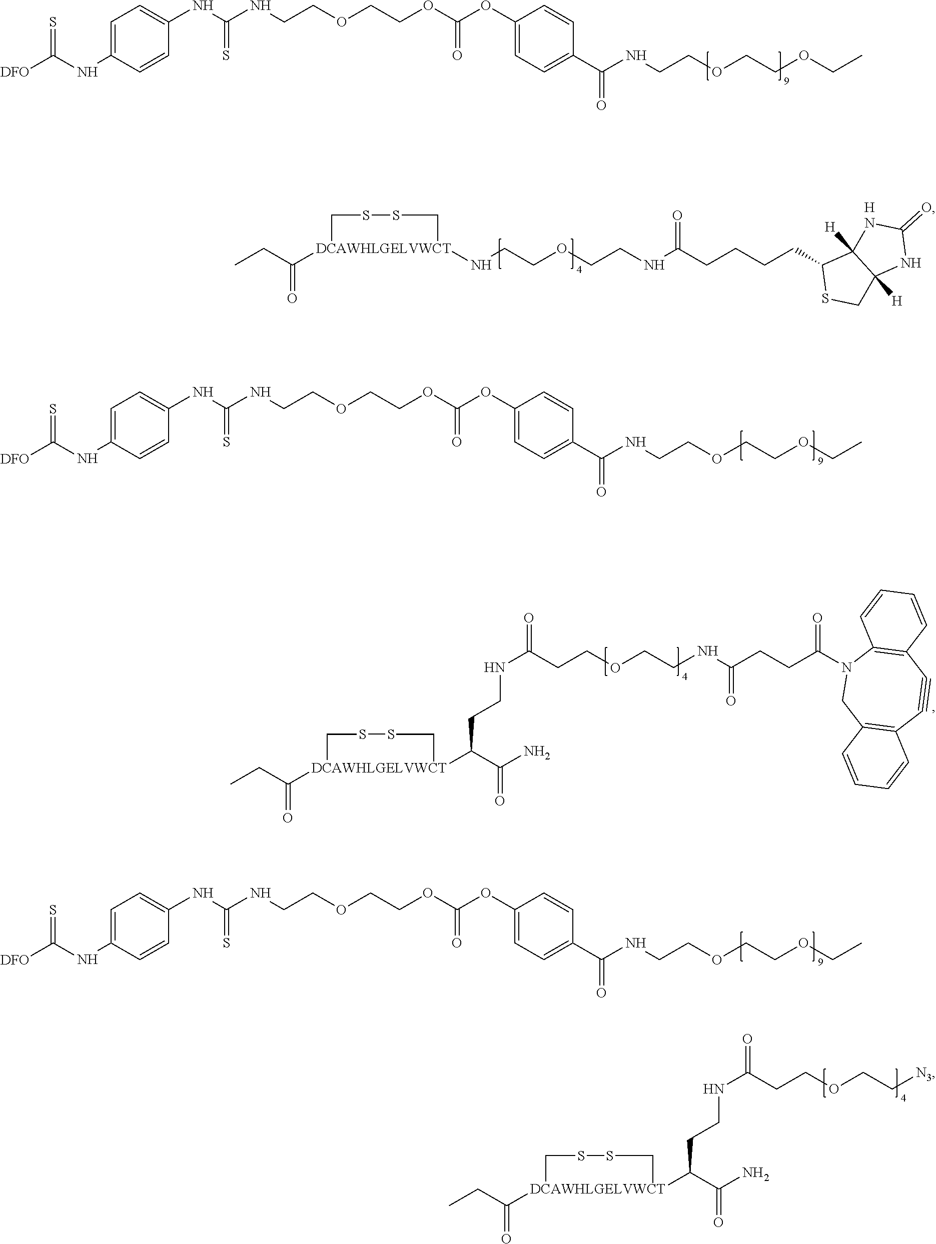

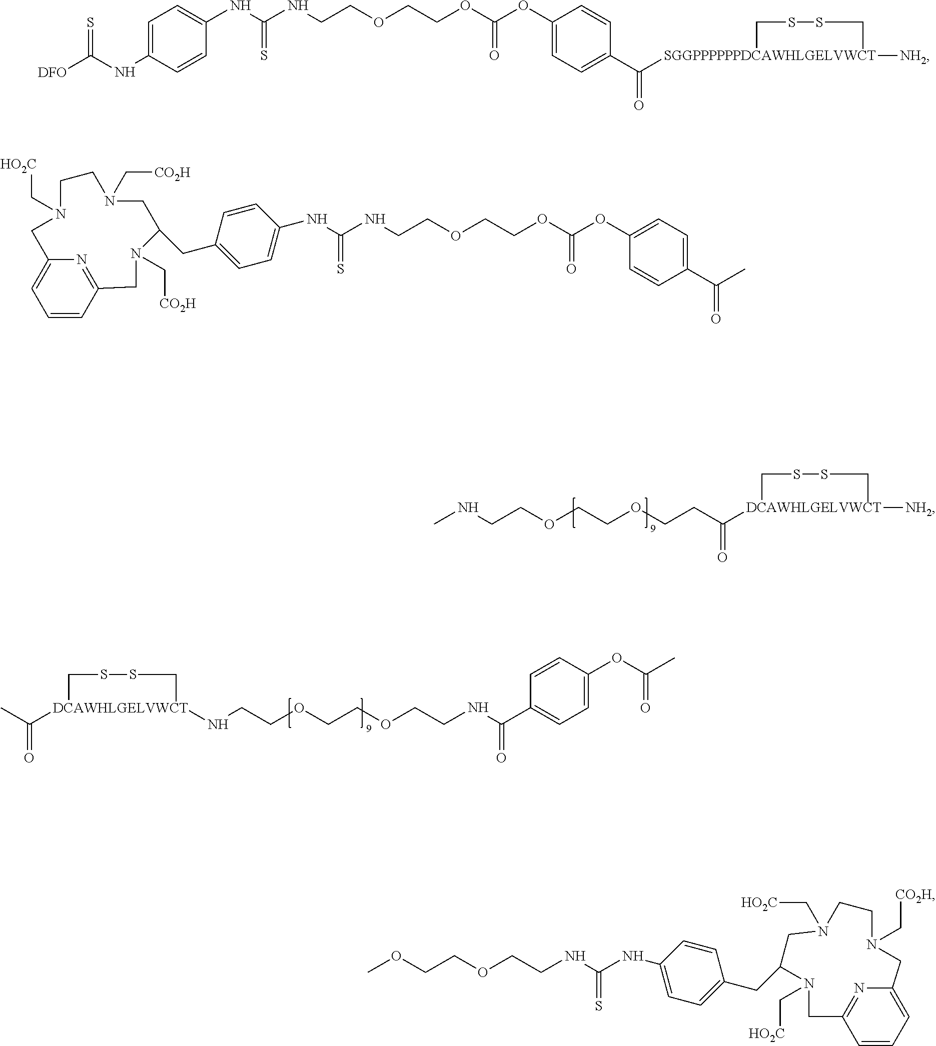

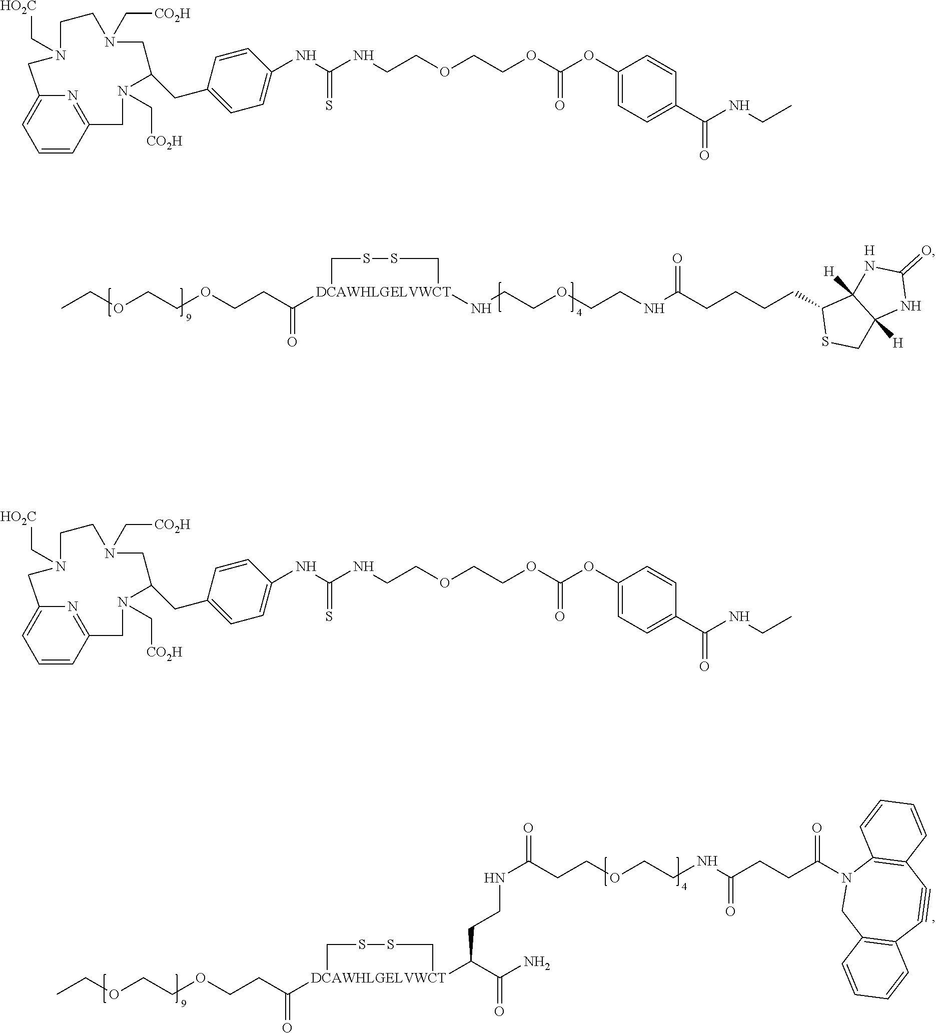

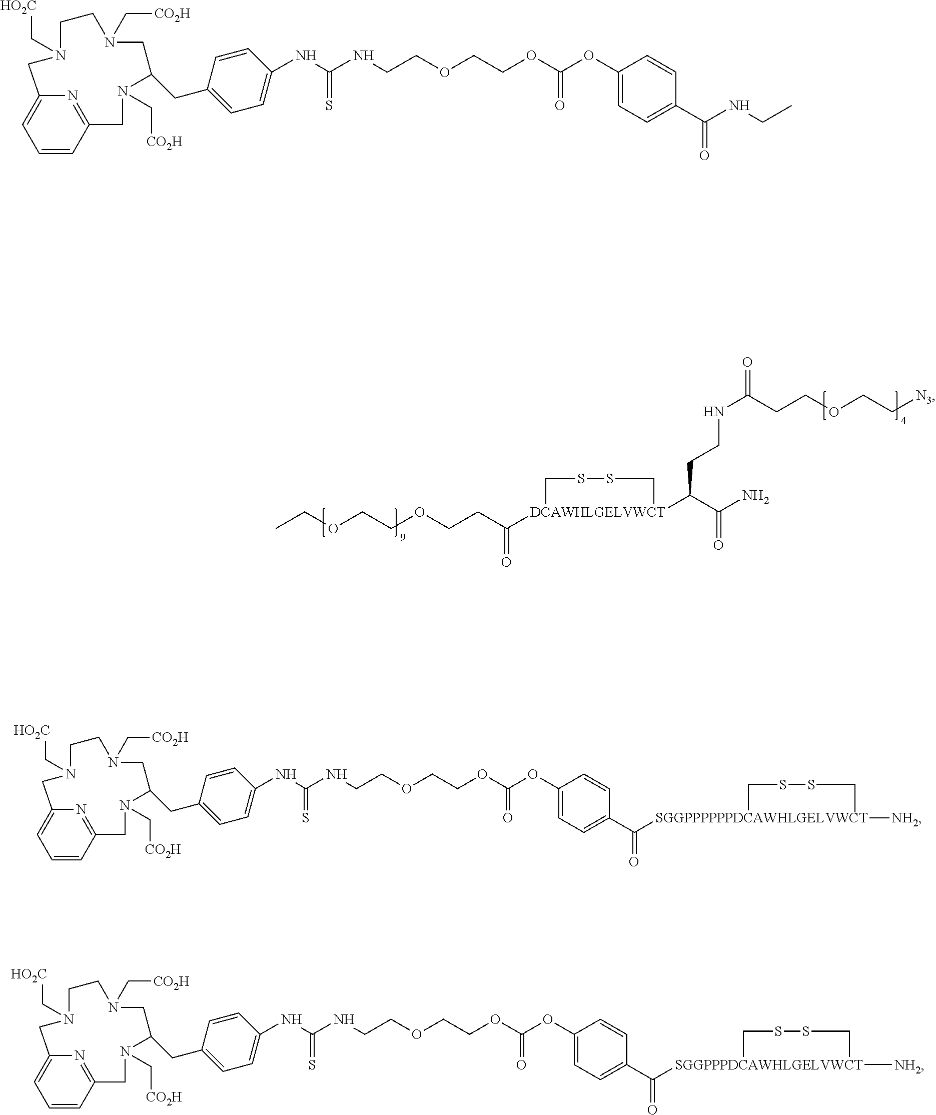

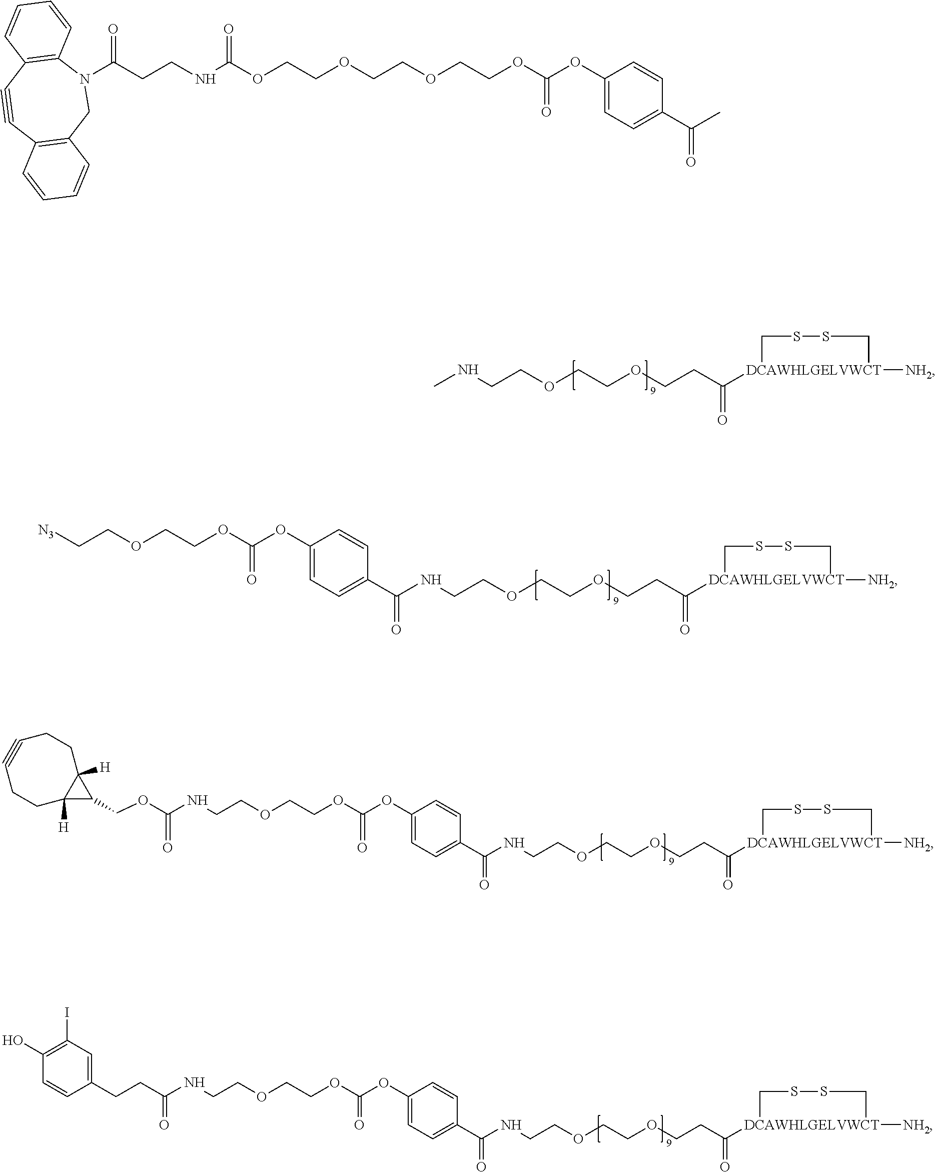

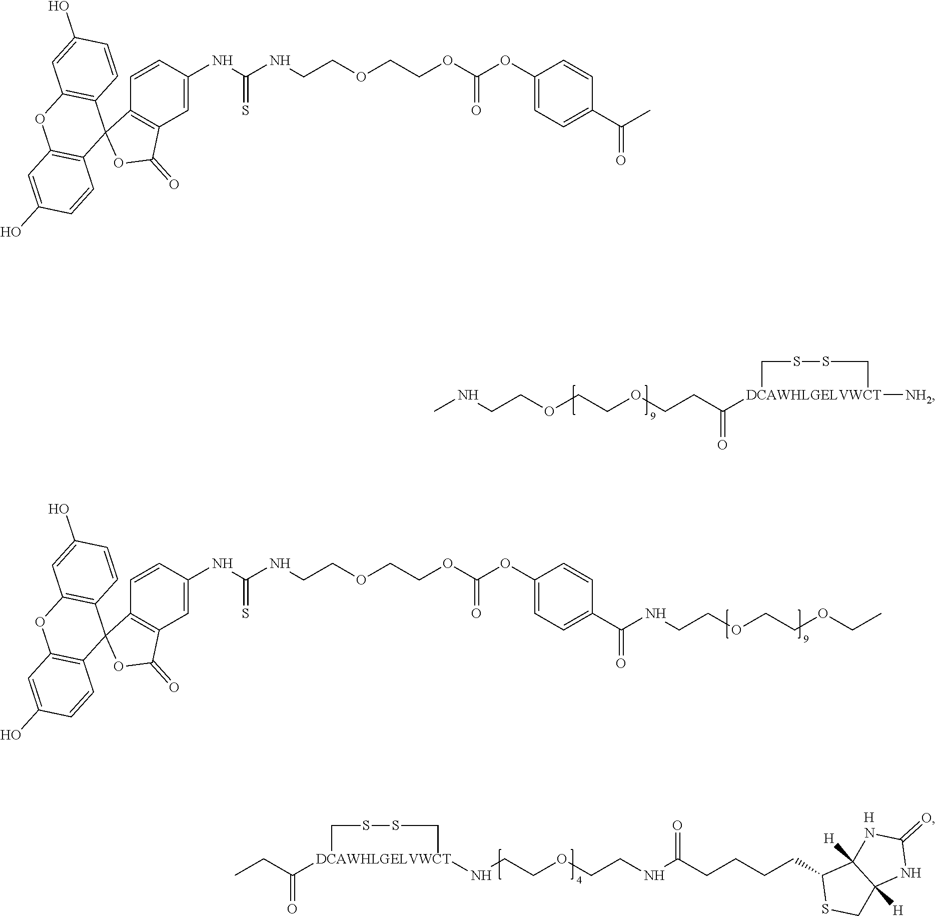

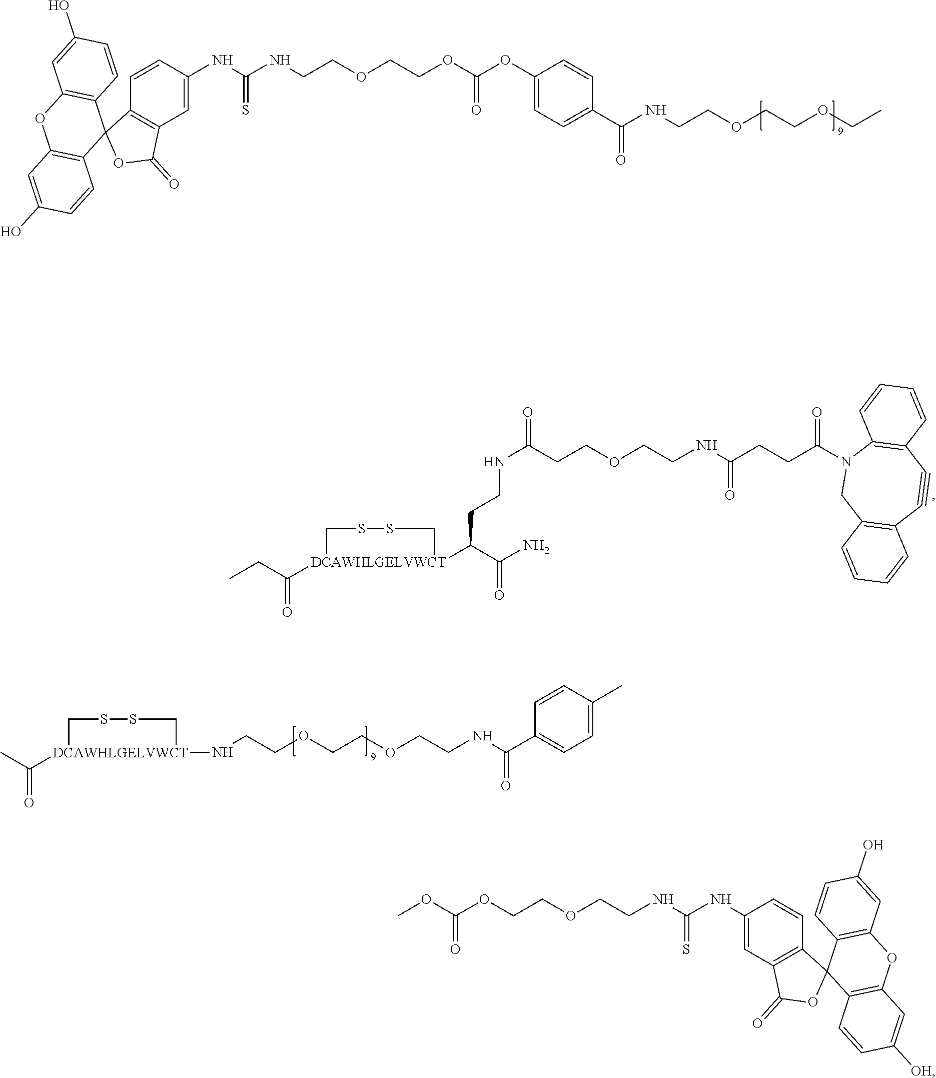

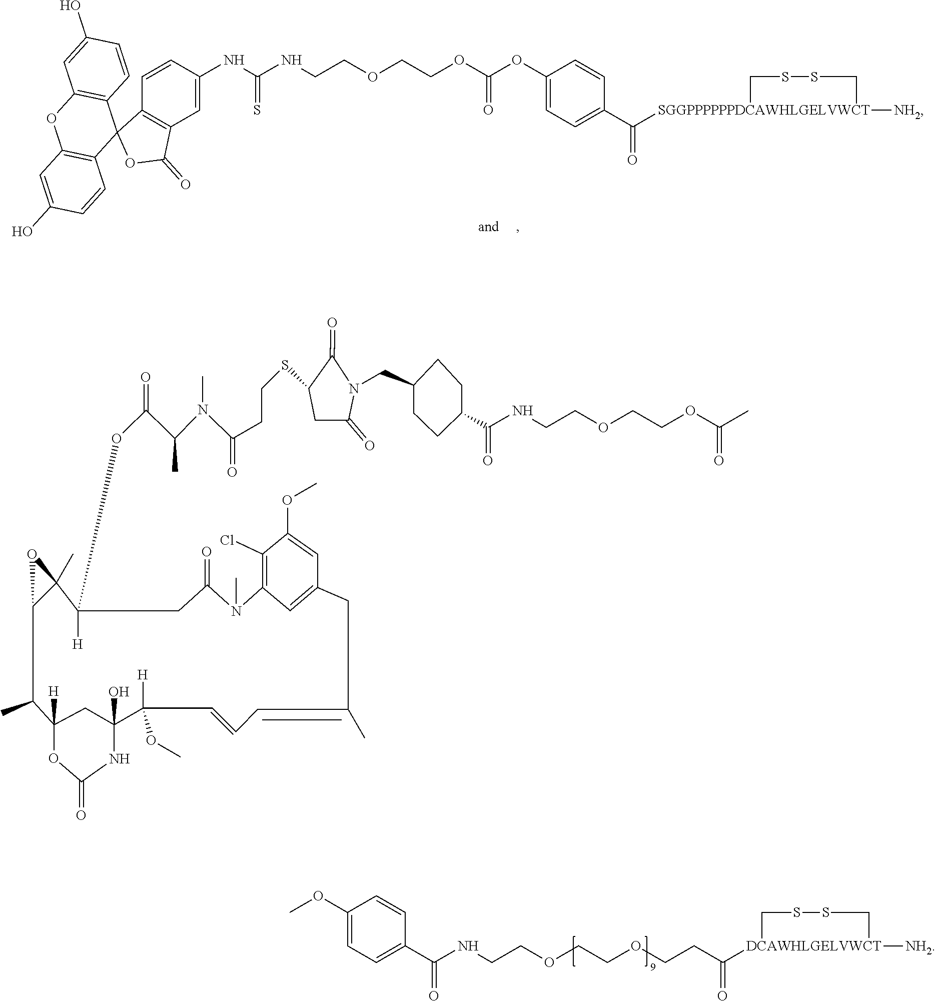

The number of repetitions of the spacer polyethylene oxide moiety in the above compounds (i.e. 9) can be replaced by any of 5 to 35, preferably by 7 to 19, a spacer having 9 polyethylene oxide repeating units being the most preferred option.

In a more preferred embodiment, the compound of formula (1) is selected from:

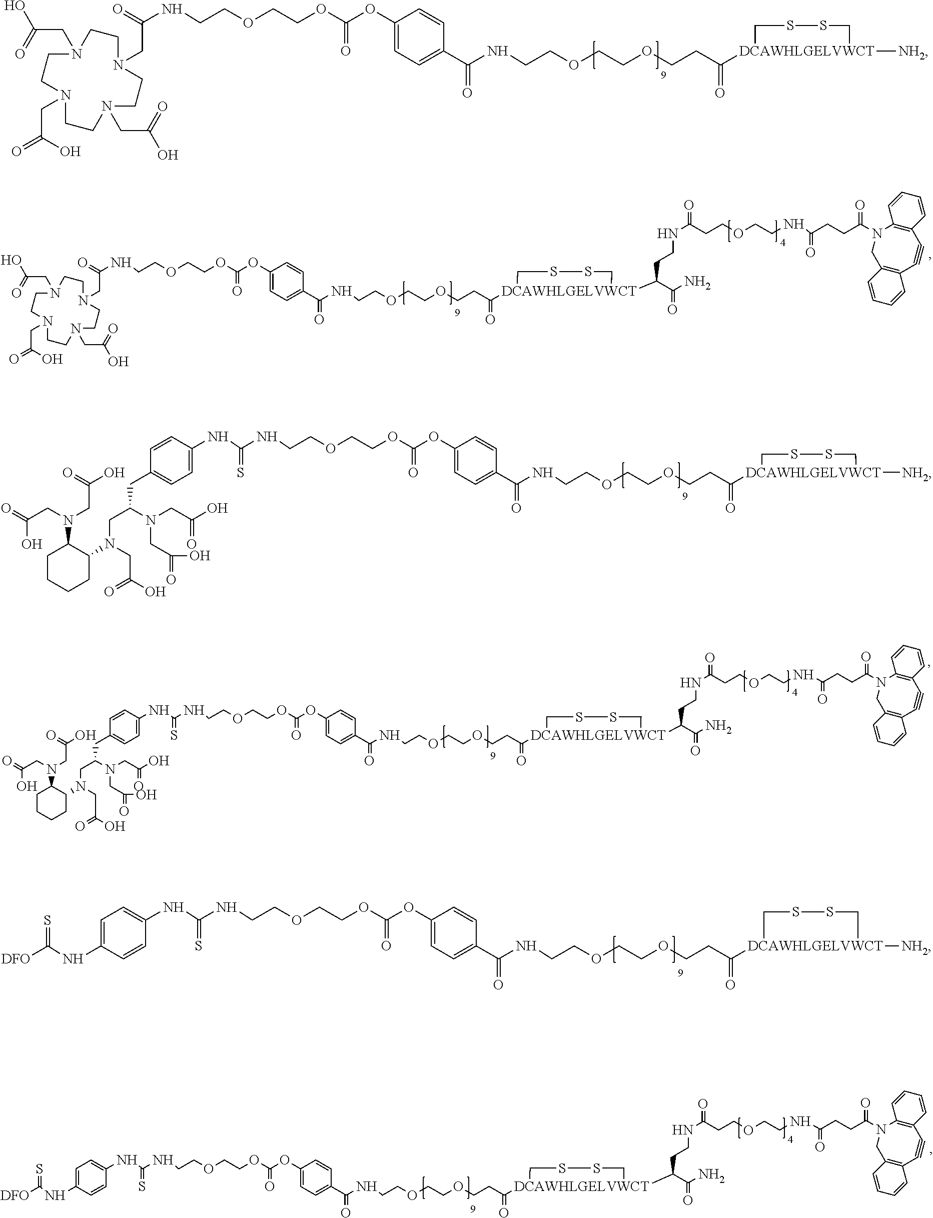

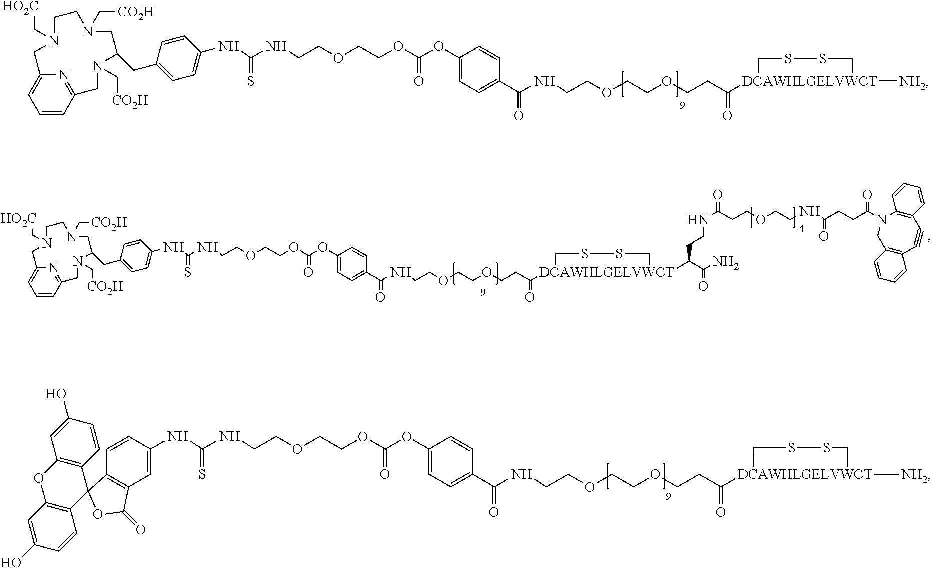

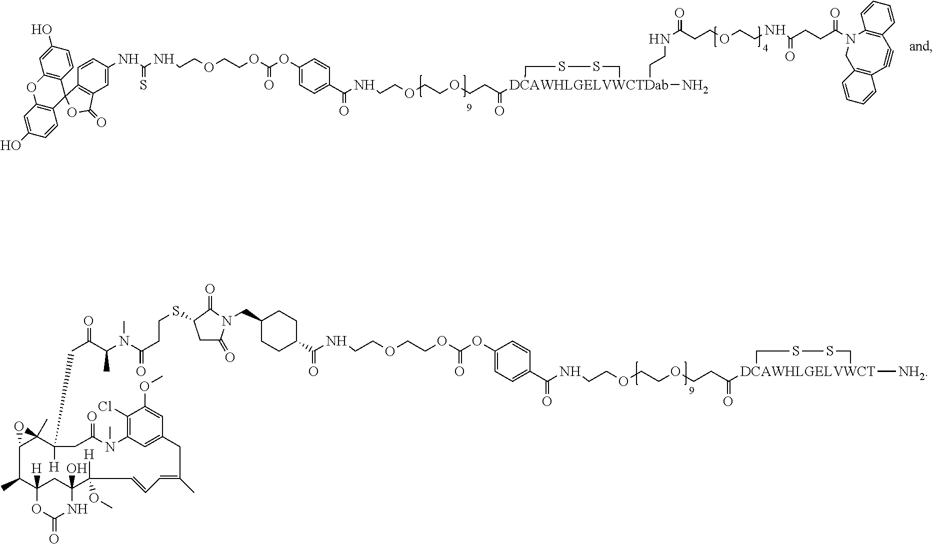

In the above compounds, DFO represents a desferrioxamine group that is attached to the remainder of the molecule via its amino group to form a thiourea group together with the thiocarbonyl-containing group to which it is attached. The number of repetitions of the spacer polyethylene oxide moiety in the above compounds (i.e. 9) can be replaced by any of 5 to 35, preferably by 7 to 19, a spacer having 9 polyethylene oxide repeating units being the most preferred option.

### 4. KIT FOR THE SITE-SPECIFIC MODIFICATION OF ANTIBODIES OR ANTIBODY FRAGMENTS

In some aspects, the present invention relates to a kit comprising the compound described hereinbefore and a buffer, which can be used for the regioselective modification (e.g. for the labelling) of antibodies or fragments thereof, the antibody fragments being optionally incorporated into Fc-fusion proteins, in particular for the regioselective modification of therapeutic antibodies.

The compound of the present invention and the buffer (together forming the kit) can be presented individually, e.g. in separate primary containers (which may be shipped to the customer in a single box), which can be stored for a prolonged period, without degradation. The compound and buffer can be formulated and proportioned for a given amount of antibody or fragment thereof to be modified. In some aspects, the compound of the present invention is presented as a solid (e.g. as a lyophilized powder, or non-covalently adsorbed or covalently bound to a solid phase matrix as described further below), or as a solution in a suitable solvent, such as a water-miscible, polar aprotic solvent (e.g. DMF, DMSO), which can be mixed with the buffer shortly prior to antibody or antibody fragment modification.

The buffer to be used in the kit of the present invention is not particularly limited. Preferably, the buffer has a pH of 5.5 to 11, more preferably of 7.5 to 9.5. The buffer can be selected from e.g. 2-bis(2-hydroxyethyl)amino acetic acid (Bicine), carbonate-bicarbonate, tris(hydroxymethyl)methylamino propane sulfonic acid (TAPS), 4-(2-hydroxyethyl)-1-piperazineethane sulfonic acid (HEPES). Preferably, the buffer is a carbonate-bicarbonate or bicine buffer with a pH of 7.5 to 9.5 e.g. about 9.0.

According to one embodiment, the compound of the present invention is immobilized on a solid phase matrix (solid support), e.g. immobilized on beads. The compound can be immobilized using methods known in the art such as high-affinity (e.g. biotin-streptavidin, biotin-neutravidin) binding, “click” chemistry (as defined by Kolb et al. in “Click Chemistry: Diverse Chemical Function from a Few Good Reactions” Angewandte Chemie Int. Ed. 2001, 40(11), 2004-2021), hydrazone ligation etc. Preferably, the solid phase matrix is an inert matrix, such as a polymeric gel, comprising a three-dimensional structure, lattice or network of material. More preferably, the solid phase matrix is a material used for affinity chromatography such as a xerogel. Such gels shrink on drying to a compact solid comprising only the gel matrix. When the dried xerogel is resuspended in a liquid, the gel matrix imbibes the liquid, swells and returns to the gel state. Examples of xerogels which can be suitably used in the present invention include polymeric gels, such as cellulose, crosslinked-dextran gels (e.g. Sephadex®), agarose, cross-linked agarose, polyacrylamide gels, polyacrylamide-agarose gels.

In one embodiment, the compound is immobilized on the solid phase matrix by means of conjugation group Y′ in formula (8a), e.g. by high-affinity binding such as biotin-streptavidin or biotin-neutravidin binding (in this case, Y′ in formula (8a) represents e.g. a biotin-containing group), by click chemistry (in this case, Y′ represents e.g. a DBCO-, azide- or alkyne-containing group), by tetrazine ligation (in this case, Y′ in formula (8a) represents a TCO- or TZ-containing group), by reaction between a thiol and maleimide or between a thiol and an acetamide (in this case, Y′ in formula (8a) represents e.g. a maleimide- or (chloro)acetamide-containing group).

### 5. USE OF REACTIVE CONJUGATES IN METHODS FOR THE REGIOSELECTIVE MODIFICATION OF ANTIBODIES OR ANTIBODY FRAGMENTS

The compound of the present invention can be used in a method for the regioselective modification of an antibody or fragment thereof, the antibody fragment being optionally incorporated into an Fc-fusion protein. The method produces a modified antibody or modified antibody fragment (e.g. an ADC), which can be used in a method of diagnosing, monitoring e.g. monitoring the effectiveness of a treatment e.g. over time, imaging or treating disease as described further below.

In one embodiment, the method comprises the step of reacting (contacting) an antibody or fragment thereof with the compound, which may be comprised in the kit described hereinbefore. The reaction mixture can be purified by techniques known in the art such as gel permeation chromatography using a suitable solvent.

When the compound of the present invention is immobilized on a solid phase matrix, the immobilized compound is contacted with a sample containing the antibody or antibody fragment to be modified, and thereafter the solid phase matrix is washed with a suitable solvent that will remove substantially all the material in the sample except the antibody, which is bound to the solid phase matrix. Finally, the solid phase matrix is washed with another suitable solvent, such as a glycine buffer pH 2.5 that will release the modified antibody/antibody fragment (e.g. the ADC) from the solid phase matrix.

The method of the present invention can be applied to any antibody (e.g. IgG protein), antibody fragment, or Fc-fusion protein provided that it comprises an Fc region for interaction with ligand V. In one embodiment, the antibody to be modified is a monoclonal antibody (mAb), preferably an antibody selected from the group consisting of adalimumab, aducanumab, alemtuzumab, altumomab pentetate, atezolizumab, anetumab, avelumab, bapineuzumab, basiliximab, bectumomab, bermekimab, besilesomab, bevacizumab, bezlotoxumab, brentuximab, brentuximab vedotin, brodalumab, blinatumomab, catumaxomab, cemiplimab, cetuximab, cinpanemab, clivatuzumab, clivatuzumab tetraxetan, crenezumab tetraxetan, daclizumab, daratumumab, denosumab, dinutuximab, durvalumab, edrecolomab, elotuzumab, emapalumab, enfortumab, enfortumab vedotin, epratuzumab, epratuzumab-SN-38, etaracizumab, gemtuzumab, gemtuzumab ozogamycin, girentuximab, gosuranemab, ibritumomab, inebilizumab, infliximab, inotuzumab, inotuzumab ozogamicin, ipilimumab, isatuximab, ixekizumab, J591 PSMA-antibody, labetuzumab, lecanemab, mogamulizumab, necitumumab, nimotuzumab, natalizumab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, siltuximab, sacituzumab, sacituzumab govitecan, semorinemab, siltuximab, solanezumab, tacatuzumab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably atezolizumab, durvalumab, pembrolizumab, rituximab or trastuzumab.

In one embodiment, the antibody or fragment thereof to be modified is a commercially formulated antibody, preferably a commercially formulated antibody having a marketing authorization delivered by the EMA or the Food and Drug Administration (FDA) of the United States of America. According to one embodiment, the commercially formulated antibody is selected from Humira®, Lemtrada®, Campath®, Tecentriq®, Bavencio®, Simulect®, LymphoScan®, Xilonix®, Scintimun®, Avastin®, Zinplava®, Blincyto®, Libtayo®, Erbitux®, hPAM4-Cide®, Zenapax®, Darzalex®, Prolia®, Unituxin®, Imfinzi®, Panorex®, Empliciti®, Gamifant®, Rencarex®, Remicade®, Besponsa®, Yervoy®, CEA-Cide®, Poteligeo®, Tysabri®, Portrazza®, Theracim®, Opdivo®, Arzerra®, Lartruvo®, Omnitarg®, Vaxira®, Cyramza®, MabThera®, Rituxan®, Sylvant®, Bexxar®, Herceptin®, Kadcyla®, Stelara®, HuMax-EGfr®, HuMax-CD4®, and biosimilars thereof; preferably from MabThera® and Herceptin®.

Commercially available antibodies are often formulated with Histidine for stability. When a commercially available antibody is mixed with a reactive conjugate, histidine would be expected to react in competitive manner onto the reactive center and therefore degrade the reactive conjugate, which would lead to decreased yields with respect to the ADCs. However, the inventors surprisingly found that with the compounds of the invention the yield is not impacted, or significantly impacted. Without wishing to be bound by theory the inventors believe that this is due to an increased rate of reaction between the compounds of the invention and the amino acids on the side chain of the antibody or antibody fragment e.g. lysine or cysteine. This favorable kinetic is likely linked to the dramatic increase of local concentration of the reactive centers in the vicinity of the targeted amino acid upon binding of the vector to the Fc fragment.

In one embodiment, the antibody fragment to be modified is incorporated into an Fc-fusion protein, which is preferably selected from belatacept, aflibercept, ziv-aflibercept, dulaglutide, rilonacept, romiplostim, abatacept and alefacept.

### 6. MODIFIED ANTIBODIES OR MODIFIED ANTIBODY FRAGMENTS

The modified antibodies and modified antibody fragments obtained by (or obtainable by) reacting the compound of the present invention with antibodies or antibody fragments (the antibody fragments being optionally incorporated into Fc-fusion proteins) comprise one or more payloads attached to an antibody or fragment thereof via a divalent group, which is a group derived from reactive moiety Y in formula (1) (i.e. it corresponds to reactive moiety Y in formula (1) which has been reacted with the side chain of an amino acid exposed at the surface of an antibody or fragment thereof).

According to one embodiment, the modified antibody or modified antibody fragment is represented by the following formula (10):

(P—W)ₚ-A (10)
wherein,
P is a payload as described above, preferably a moiety as specified under items (i) to (iii) above;
W is F1-RC′, wherein F1 is attached to P and RC′ is a moiety derived from a reactive center (RC) attached to A, F1 and RC being as defined in formulae (3a) and (3b);
A is moiety derived from an antibody or an antibody fragment optionally incorporated into an Fc-fusion protein, said antibody or antibody fragment being as defined above; and
p is an integer of 1 to 4. Preferably p is 1 to 2.

In those instances where the reactive moiety reacts with the side chain of Lys, the attachment of the payload to the antibody or antibody fragment occurs via a nitrogen atom-containing group such as an amide group, an urethane group, a thiourethane group, a dithiourethane group, etc. For example, if the compound of the invention includes a reactive moiety of formula (4a) or (4d) (or formula (4a′) or (4d′)), the divalent group W in formula (10) is an urethane group in which the nitrogen atom forms part of the Lys side chain. If the compound includes e.g. a reactive moiety of formula (4e), (4f) or (4j) (or formula (4e′), (4f′) or (4j′)), the divalent group W is a thiourethane group.

p represents the degree of conjugation (DoC; sometimes referred to as “drug-antibody-ratio” (DAR)) of the modified antibody or modified antibody fragment.

According to one embodiment, the modified antibody or modified antibody fragment is represented by the following formula (11):

(P1-L-W)ₚ-A (11)
wherein,
P¹, L, W, A and p are as defined above.

### 7. USE OF MODIFIED ANTIBODIES OR MODIFIED ANTIBODY FRAGMENTS FOR DIAGNOSTIC AND/OR THERAPEUTIC PURPOSES

The modified antibodies and modified antibody fragments obtained by (or obtainable by) reacting the compound of the present invention with antibodies or antibody fragments (the antibody fragments being optionally incorporated into Fc-fusion proteins) can be used to diagnose and/or treat disease, in particular, cancer. The treatment can be a therapeutic and/or prophylactic treatment, with the aim being to prevent, reduce or stop an undesired physiological change or disorder. In some instances, the treatment can prolong survival of a subject as compared to expected survival if not receiving the treatment.

The disease that is treated by the modified antibody or modified antibody fragment (e.g. the ADC) can be any disease that benefits from the treatment, including chronic and acute disorders or diseases and also those pathological conditions which predispose to the disorder. In some instances, the disease is a neoplastic disease such as cancer that can be treated via the targeted destruction of tumor cells. Non-limiting examples of cancers that may be treated include benign and malignant tumors, either solid or liquid; leukemia and lymphoid malignancies, as well as breast, ovarian, stomach, endometrial, salivary gland, lung, kidney, colon, thyroid, pancreatic, prostate or bladder cancer. The disease may be a neuronal, glial, astrocytal, hypothalamic or other glandular, macrophagal, epithelial, stromal and blastocoelic disease; or inflammatory, angiogenic or an immunologic disease. An exemplary disease is a solid, malignant tumor.

According to one embodiment, the disease or treatment thereof is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina (including unstable angina), Aortic aneurysm, Atherosclerosis, Cardiac transplant, Cardiotoxicity diagnosis, Coronary artery bypass graft, Heart failure (including atrial fibrillation terminated systolic heart failure), hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.

According to one embodiment, the disease to be treated involves cells selected from lymphoma cells, myeloma cells, renal cancer cells, breast cancer cells, prostate cancer cells, ovarian cancer cells, colorectal cancer cells, gastric cancer calls, squamous cancer cells, small-cell lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, small-cell lung cancer cells, lymphoma cells, colorectal cancer cells, and head-and-neck cancer cells.

According to one embodiment, the modified antibody or modified antibody fragment is used in a method of diagnosing, monitoring e.g. monitoring the effectiveness of a treatment e.g. over time, imaging and/or treating a disease (e.g. cancer) by administering the modified antibody or modified antibody fragment to a subject (e.g. a patient).

The molecule can be administered to a subject at one time or over a series of treatments. Depending on the type and severity of the disease, and/or on the payload, and/or on the antibody or antibody fragment, between about 0.1 μg/kg to 1 mg/kg of drug may be used as an initial candidate dosage for first administration in a first-in-human trial, e.g. by one or more separate administrations, or by continuous infusion. A typical daily dosage can range from about 0.1 mg/kg to 50 mg/kg or more, or from about 0.5 to about 30 mg/kg e.g. 0.5 to about 25 mg/kg of patient weight. However, typical dosages will depend on a variety of factors including the specific payload (active agent), the age, body weight, general health, sex and diet of the subject; whether administration is for imaging, monitoring or treatment purposes, and other factors well known in the medical art.

When treating cancer, the therapeutic effect that is observed can be a reduction in the number of cancer cells; a reduction in tumor size; inhibition or retardation of cancer cell infiltration into peripheral organs; inhibition of tumor growth; and/or relief of one or more of the symptoms associated with cancer.

According to a preferred embodiment, the modified antibody or modified antibody fragment is administered by injection, such as parenterally, intravenously, subcutaneously, intramuscularly.

According to one further embodiment, the modified antibody or modified antibody fragment is used in a method of diagnosing, monitoring e.g. monitoring the effectiveness of a treatment e.g. over time, imaging and/or treating a cancer, and is administered concurrently with one or more other therapeutic agents such as chemotherapeutic agents, radiation therapy, immunotherapy agents, autoimmune disorder agents, anti-infectious agents, or one or more other modified antibodies or modified antibody fragments. It is also possible to administer the other therapeutic agent before or after the modified antibody or modified antibody fragment.

### 8. PREPARATION OF THE COMPOUNDS OF THE INVENTION

In the following, methods are provided for the preparation of ligands, spacers, payload-linkers, and compounds (reactive conjugates) as well as for their use in the regioselective modification of therapeutic antibodies or therapeutic proteins (e.g. Fc-fusion proteins). The compounds of the present invention can be synthesized relying on standard chemical methods and Fmoc-based solid-phase peptide synthesis (SPPS), including on-resin peptide coupling and convergent strategies. The introduction of various payloads, as well as compound immobilization on a solid phase matrix, are also exemplified below. The general strategies and methodology which can be used for preparing the compounds of the present invention are known to the skilled person and illustrated in FIGS. 2, 5 and 9.

### 9. EXAMPLES

9.1 List of Abbreviations Used in the Examples:

ACN: acetonitrile

DCM: dichloromethane

DIC: diisopropylcarbodiimide

DIEA: diisopropylethylamine

DMF: dimethyl formamide

DMSO: dimethyl sulfoxide

FL or FITC: fluorescein

HATU: 1-[Bis(dimethylamino)methylene]-1H-1,2,3-triazolo[4,5-b]pyridinium 3-oxide hexafluorophosphate

HPLC: high-performance liquid chromatography

HRMS: high resolution mass spectrometry

PBS: phosphate-buffered saline

SDS-PAGE: sodium dodecyl sulfate-polyacrylamide gel electrophoresis

SPPS: solid-phase peptide synthesis

TFA: trifluoroacetic acid

TIS: triisopropylsilane

UPLC: ultra-performance liquid chromatography

WLC: worm-like-chain

9.2 Starting Materials and Chemicals:

The main starting materials and chemicals used in the following examples are listed below:
>Resins (Fmoc-Rink Amide AM resin, 4-Fmoc-hydrazinobenzoyl AM Novagel™) and protected amino acids for solid-phase peptide synthesis, N,N-diisopropylcarbodiimide (DIC), piperazine from Novabiochem (Switzerland) unless indicated otherwise;
>Solvents for synthesis, deprotection reagents, cleavage reagents from Merck or Fischer Scientific AG (Switzerland);
>Maleimidopropionic acid, 4-nitrophenyl chloroformate, TFA, TIS and DIEA from Sigma-Aldrich (Switzerland);
>Amino acids from Bachem AG (Switzerland), Novabiochem and Aapptec (USA);
>Solvents and chemicals for high-performance liquid chromatography (HPLC), ultra-performance liquid chromatography mass spectrometry (UPLC-MS) from Macherey-Nagel (Switzerland);
>Fluorescently labeled peptide Fc-III-FAM from Genscript (USA);
>GingisKhan®, Fabalactica® and Fabricator® proteases from Genovis (Sweden);
>IdeS® protease from Promega (Switzerland);
>EndoS® protease from BioConcept (Switzerland);
>Biotin-PEG4-amine and PEG linkers from BroadPharm (USA);
>Herceptin® (commercial trastuzumab) from Roche (Switzerland);
>p-SCN-Bn-CHX-A″-DTPA.3HCl, p-SCN-Bn-PCTA.3HCl from Macrocyclics (USA);
>p-NCS-Bz-DFO from Chematech (France).

Biosimilar monoclonal IgG1 antibodies (trastuzumab, alemtuzumab, bevacizumab, rituximab) were produced by cultivation of recombinant CHO cell lines in Dr. G. Hagens laboratory at the University of Applied Sciences (HES-SO Valais/Wallis, Switzerland).

GingisKhan and Fabalactica are cysteine proteases which site-specifically cleave IgG1 above the hinge, thereby generating two Fab fragments and one Fc fragment. Fabricator is a cysteine protease that site-specifically digest antibodies below the hinge, generating F(ab′)2 and Fc/2 fragments.

9.3 Methods:

The following methods were used to evaluate the compounds and conjugates of the present invention:

9.3.1 Determination of Spacer Lengths

The lengths of the spacer (moiety S of formula (1)) introduced at the N-terminus of the Fc-binding vectors were calculated by using the worm-like-chain (WLC) model, which considers the spacer as a continuously flexible rod and was shown to be a suitable model for biopolymers (Rubinstein and Colby (2003), Polymer Physics, Oxford University Press):

<R²>=2×Lₚ×L

wherein Lₚ is the persistence length (correlation length of the chain direction) and L is the contour length (length of the fully stretched chain). The persistence length value of 3.8 Å for polyethylene glycol spacers was used (Kienberger et al. Single Molecules 2000, 1(2), 123-128). The length of 20.8 Å for the SGGPPPPPP spacer was estimated based on the procedure described in the literature (Mahoney et al. Nature Chemical Biology 1997, 4(12), 953-960; Garbuio et al. Chemistry: A European Journal 2015, 21(30), 10747-10753).

9.3.2 Saturation FP Binding Assays

The saturation fluorescence polarization (FP) measurements were performed on SpectraMax Paradigm Multi-Mode Detection Platform (available from Molecular Devices) in flat-bottom 384-well Corning microplates (Merck KGaA), using excitation and emission wavelengths of 485 nm and 535 nm, respectively. The acquisition time was 700 milliseconds and the read height was 1 mm. All reagents used in the assay were diluted in PBS containing 0.05% Tween 20.

Fluorescently labeled peptide Fc-III-FAM (structure shown below) was mixed with a series of IgG1 dilutions in PBS with 0.05% tween to a final peptide concentration of 5 nM. Samples were incubated at 27° C. for 15 min and the fluorescence anisotropy was measured in triplicate.

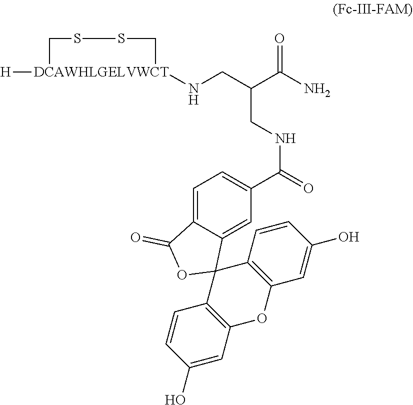

Fc-III is a 13-mer cyclic peptide known to bind with high affinity to the Fc region of IgG antibodies (DeLano et al. Science 2000, 287, 1279-1283; Nilsson et al. Protein Eng. 1987, 1, 107-113). The fluorescently labeled peptide Fc-III-FAM was prepared by GenScript using standard SPPS techniques and convergent strategies.

9.3.3 Competitive FP Binding Assays

The competitive FP measurements were performed on SpectraMax Paradigm Multi-Mode Detection Platform (Molecular Devices) in flat-bottom 384-well Corning microplates (Merck KGaA), using excitation and emission wavelengths of 485 nm and 535 nm, respectively. The acquisition time was 700 milliseconds and the read height was 1 mm. All reagents used in the assay were diluted in PBS containing 0.05% Tween 20.

Increasing concentrations of the peptide to be measured were mixed with the Fc-III-FAM peptide and added to the IgG1 in a total volume of 80 μL. The final concentration of Fc-III-FAM was kept constant at 5 nM and the final concentration of IgG1 was of 10-30 nM. The mixture was incubated at 27° C. for 15 min and the fluorescence signal was red on a Spectramax Paradigm. All sample preparations were done in PBS pH 7.4 or 7.0 containing 0.05% Tween. Each experiment was performed in triplicate.

9.3.4 Peptide and Conjugate Concentration Determination

Peptide samples were prepared by dissolving the purified peptide or reactive conjugate in DMSO. The concentrations were determined in 1× PBS pH 7.4 using the absorbance of the Trp (ε=5500 M⁻¹ cm⁻¹) residues, p-SCN-Bn-CHX-A″-DTPA (ε=13000 M⁻¹ cm⁻¹), p-SCN-Bn-PCTA (ε=13000 M⁻¹ cm⁻¹), p-NCS-Bz-DFO (ε=21000 M⁻¹ cm⁻¹) at 280 nm or the absorbance of FITC (ε=73000 M⁻¹ cm⁻¹) at 496 nm.

9.3.5 High-Resolution Mass Spectrometry

Prior to HRMS analysis, antibody-payload conjugates were desalted against 50 mM ammonium acetate solution buffered at pH 7.0 using four cycles of concentration/dilution on micro-concentrators (Vivaspin, 30 kD cutoff, Sartorius, Germany). Deglycosylation of the conjugates was achieved by incubating 1 unit of Endo S per μg of conjugate in the formulation buffer (37° C.—1 hour or overnight).

Direct injection HRMS for peptide/conjugate analysis was performed on a QExactive HF Orbitrap-FT-MS, (Thermo Fisher Scientific, Germany) coupled to an automated chip-based nanoelectrospray device (Triversa Nanomate, Advion, USA). Electrospray ionization was conducted at a capillary voltage of 1.4 kV and nitrogen nanoflow of 0.15 psi. MS experiments were performed with a nominal resolution of 45000 and in the positive ion mode. Data deconvolution was performed with Protein Deconvolution (Thermo Fischer Scientific, USA) using the Xtract algorithm with a 90% fit factor.

For both intact mass measurement (LC-MS) and middle-down analysis (LC-HCDMS/MS), samples were separated onto an Acquity UPLC Protein column BEH C4 (300 Å, 1.7 μm, 1×150 mm, Waters, USA) using a Dionex Ultimate 3000 analytical RSLC system (Dionex, Germany) coupled to a HESI source (Thermo Fisher Scientific, Germany). The separation was performed with a flow rate of 90 μL/min by applying a gradient of solvent B from 15 to 45% in 2 min, then from 45 to 60% within 10 min, followed by column washing and re-equilibration steps. Solvent A was composed of water with 0.1% formic acid, while solvent B consisted of acetonitrile with 0.1% TFA.

Eluting proteoforms were analyzed on a high-resolution QExactive HF-HT-Orbitrap-FTMS benchtop instrument (Thermo Fisher Scientific, Germany). For intact mass measurements MS1, the scan was performed in protein mode with 15000 resolution and averaging 10 μscans. Middle-down analysis for binding site localization was performed in PRM mode isolating species at 1356 m/z for Fc/2-mod, with 300 Th isolation window, 240000 resolution and averaging 10 μscans. HCD (high energy collision-induced dissociation) was used as a fragmentation method with a normalized collision energy of 12, 15 and 18%.

Intact mass measurement data were analyzed with Protein Deconvolution (Thermo Fischer Scientific, USA) using a Respect algorithm with 99% noise rejection confidence and 20 ppm accuracy of average mass identification. Middle-down data were deconvoluted using a MASH Suite software (Ge research group, University of Wisconsin). Data obtained with 3 different NCE values were combined together to create a fragmentation map with assigned b- and y-fragments using ProSight Lite software (Kelleher research group, Northwestern University) with 15 ppm mass tolerance.

9.3.6 Determination of the Degree of Conjugation from HRMS Analysis

The average Degree of Conjugation (DoC) values were calculated using the HRMS data and the Equation 1 (Eq. 1) below. These results were derived from the relative peak intensities in deconvoluted mass spectra.

$$DoC = \frac{{\sum }_{k = 0}^{k = n} k ⋆ I ( {DoC}_{k} )}{{\sum }_{k = 0}^{k = n} I ( {DoC}_{k} )} \qquad Eq . 1$$

where I(DoCₖ) is the relative peak intensity of conjugates with k add-on molecules per antibody.

9.3.7 SDS-PAGE

Reducing or non-reducing Bis-Tris SDS-PAGE was performed on Bolt 4-12% Bis-Tris Plus Gels (ThermoFisher, Germany). The loading buffer was added to the antibody conjugates (non-reducing Bolt Sample Buffer, ThermoFisher) and the samples were heated at 70° C. for 10 min. For reducing SDS-PAGE, reducing buffer was added to the samples prior loading buffer. The gel was run at constant voltage (200 V) for 25-30 min using Bolt MES Running Buffer. The fluorescence was visualized on FluoroM bio-imaging system (Syngene, United Kingdom) prior to staining with Coomassie Blue.

### Example 1

### Preparation and Characterization of Fc-Binding Vectors

The Fc-binding vectors and vector spacer constructs as described herein (moiety V or S-V of formula (1)) were prepared using standard Fmoc/tBu-based SPPS, including on-resin coupling and convergent strategies. The ligands prepared in Example 1 are shown in Table 1 below (bold-underlined indicates that a disulfide bond is present between the side chains of the respective Cys residues). The spacer lengths were calculated by using the WLC model as described above.

TABLE 1
Fc-binding vectors (moiety V/S-V according to formula (1))

| | | | Spacer |
|---|---|---|---|
| | | | length |
| Comp. | Structure | Spacer (S) | (Å) |
| 1 | H—DCAWHLGELVWCT—NH₂ | No spacer | — |
| (Fc-III) | | | |
| 2 | H—SGGPPPPPPDCAWHLGELVWCT—NH₂ | SGGPPPPPP | 20.8 |
| 3 | NH₂—(PEG)₂—CH₂CH₂—CO—DCAWHLGELVWCT—NH₂ | (PEG)₂ | 8.9 |
| 4 | NH₂—(PEG)₄—CH₂CH₂—CO—DCAWHLGELVWCT—NH₂ | (PEG)₄ | 11.7 |
| 5 | NH₂—(PEG)₆—CH₂CH₂—CO—DCAWHLGELVWCT—NH₂ | (PEG)₆ | 13.7 |
| 6 | NH₂—(PEG)₈—CH₂CH₂—CO—DCAWHLGELVWCT—NH₂ | (PEG)₈ | 16.0 |
| 7 | NH₂—(PEG)₁₀—CH₂CH₂—CO—DCAWHLGELVWCT—NH₂ | (PEG)₁₀ | 17.4 |
| 8 | NH₂—(PEG)₁₂—CH₂CH₂—CO—DCAWHLGELVWCT—NH₂ | (PEG)₁₂ | 18.9 |
| 9 | NH₂—(PEG)₁₅—CH₂CH₂—CO—DCAWHLGELVWCT—NH₂ | (PEG)₁₅ | 21 |
| 10 | NH₂—(PEG)₂₀—CH₂CH₂—CO—DCAWHLGELVWCT—NH₂ | (PEG)₂₀ | 23.8 |
| 11 | NH₂—(PEG)₂₄—CH₂CH₂—CO—DCAWHLGELVWCT—NH₂ | (PEG)₂₄ | 26.2 |
| 12 | NH₂—(PEG)₃₆—CH₂CH₂—CO—DCAWHLGELVWCT—NH₂ | (PEG)₃₆ | 32.3 |
| 13 | H—DCAWHLGELWACT—NH₂ | No spacer | |
| 14 | H—DAAWHLGELVWAT—NH₂ | No spacer | |
| 15 | NH₂—(PEG)₂₀—CH₂CH₂—CO—DCAWHLGELWACT—NH₂ | (PEG)₂₀ | 23.8 |
| 16 | NH₂—(PEG)₂₀—CH₂CH₂—CO—DAAWHLGELVWAT—NH₂ | (PEG)₂₀ | 23.8 |

The peptides were prepared by standard Fmoc/tBu-based SPPS using a Rink Amide AM resin (loading: 0.57 mmol/g) and a Liberty Blue™ automated microwave peptide synthesizer (available from CEM Corp., Germany).

Coupling reactions for amide bond formation were performed over 4 min at room temperature using 0.2 M of Fmoc-amino-acids pre-activated with 0.5 M DIC and 1 M OxymaPure® in DMF. Fmoc deprotection was performed with 10% piperazine in DMF (v/v).

After completion of the synthesis, the peptides were cleaved from the resin manually under gentle agitation over 1.5 hour at room temperature by treatment with TFA/TIS/water (90/5/5, v/v/v). After filtration and evaporation of the cleavage mixture with a nitrogen stream, the crude peptides were precipitated with cold diethyl ether, centrifuged, and washed with cold diethyl ether. The peptides were dried, dissolved in ultrapure water/ACN, frozen and lyophilized.

For disulfide bond formation, the crude lyophilized peptides were resuspended in a mixture of DMSO/ACN/water (2/3/3, v/v/v), then water was added until the peptide became soluble (about 35-50 mL) and the resulting solution was brought to pH 8.5 with NH₄HCO₃ or NaHCO₃ (concentration: 0.1-0.5 mM). The progress of the oxidation was monitored via analytical UPLC-MS. After completion of the reaction, salts were removed with a Sep-Pak C18 Plus Long Cartridge (820 mg sorbent per cartridge, particle size: 55-105 μm, available from Waters, Switzerland) and the peptide was lyophilized.

The peptides were purified by Preparative Reversed Phase-HPLC on a Kinetex® XB-C18 column (100 Å, 5 μm, 100×21.2 mm; Phenomenex Helvetia) using solvent system A (0.1% TFA in water) and B (0.1% TFA in ACN) at a flow rate of 35 mL/min and a gradient in a range of 15-55% of B over 25 min. Peptide elution was monitored at a wavelength of 214 nm. The appropriate fractions were analyzed by UPLC-MS prior to concentration and lyophilization.

For the synthesis of compounds 3-12 and 15-16, a solution of Fmoc-NH—(CH₂—CH₂—O)ₙ—CH₂—CH₂—COOH (with n=2, 4, 6, 8, 10, 12, 15, 20, 24 or 36; 1.3 eq, 4.7 μmol) and HATU (1.2 eq, 4.33 μmol) in DMF was stirred for 1 min, and DIEA (2 eq, 7.16 μmol) was added. After 3 min of pre-activation, the Fc-binding peptide (compound 1, 13 or 14) in DMF (1 eq, 3.58 μmol) was added to the reaction mixture and stirred for 1-2 hours at room temperature. Reaction completion was monitored by ULPC-MS. The peptide was then precipitated with cold diethyl ether. Fmoc deprotection was performed with 20% piperidine in DMF (v/v) for 30 min at room temperature, followed by precipitation of the peptide with cold diethyl ether (FIG. 2a). The peptides were isolated after HPLC purification (as described in the former paragraph).

The purity of the peptides was determined on a Waters Acquity UPLC system coupled to a Micromass Quattro micro API mass spectrometer with a Kinetex® XB-C18 column (100 Å, 1.7 μm, 50×2.1 mm; Phenomenex Helvetia) using solvent system using solvent system A (0.1% TFA in water) and B (0.1% TFA in ACN) at a flow rate of 0.6 mL/min and a 2-98% gradient of B over 4 min. Peptide elution was monitored at a wavelength of 214 nm. The results are shown in the table below.

TABLE 2
Characterization of compounds 1-16

| | Calculated | Calculated | Found | Retention | Purity |
|---|---|---|---|---|---|
| Compound | Exact Mass | M + H⁺ | M + H⁺ | time (min) | (%) |
| 1 | 1529.73 | 765.87 | 765.81 | 1.67 | >95 |
| 2 | 2313.63 | 1157.82 | 1158.02 | 2.08 | >95 |
| 3 | 1689.13 | 845.57 | 845.74 | 1.69 | >95 |
| 4 | 1777.27 | 889.64 | 889.87 | 1.70 | >95 |
| 5 | 1865.38 | 933.69 | 933.87 | 1.71 | >95 |
| 6 | 1953.53 | 977.77 | 977.84 | 1.74 | 91 |
| 7 | 2041.63 | 1021.82 | 1021.95 | 1.75 | >95 |
| 8 | 2129.73 | 1065.87 | 1065.95 | 1.77 | 95 |
| 9 | 2261.58 | 1131.79 | 1131.86 | 1.85 | >95 |
| 10 | 2481.91 | 828.30 | 828.50 | 1.80 | >95 |
| 11 | 2658.10 | 887.03 | 887.36 | 1.90 | >95 |
| 12 | 3187.03 | 1063.34 | 1063.46 | 1.9 | >95 |
| 13 | 1501.68 | 751.84 | 751.84 | 1.70 | >95 |
| 14 | 1467.63 | 734.82 | 734.71 | 1.87 | >95 |
| 15 | 2453.86 | 818.95 | 819.42 | 1.85 | >95 |
| 16 | 2419.83 | 807.61 | 807.53 | 1.87 | >95 |

### Example 2

### Saturation FP Binding Assay

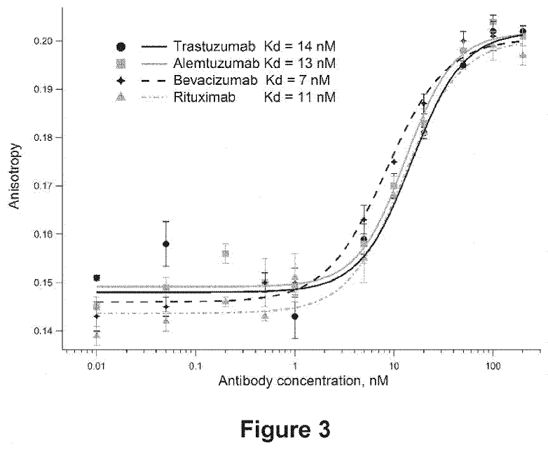

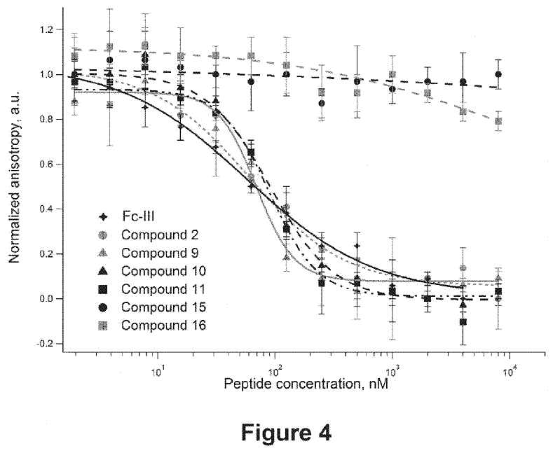

The propensity of the Fc-binding ligand Fc-III-FAM (structure shown above) to bind the Fc region of IgG1 antibodies, i.e. trastuzumab, alemtuzumab, bevacizumab and rituximab, was evaluated in the saturation FP binding assay described above. The results are shown in FIG. 3. It was confirmed that the Fc-binding ligand Fc-III-FAM binds the respective antibodies with high affinity (trastuzumab: 14 nM, alemtuzumab: 13 nm, bevacizumab: 7 nM, rituximab: 11 nM).

### Example 3

### Competitive FP Binding Assay

The propensity of the Fc-binding ligands prepared in Example 1 (compounds 1, 2, 9-11, 13, 15 and 16) to bind the Fc region of trastuzumab against Fc-III-FAM was evaluated in the competitive FP binding assay described above. The results are given in Table 3 below and shown in FIG. 4.

| Compound | IC₅₀ (nM) |
|---|---|
| 1 | 58 ± 15 |
| 2 | 61 ± 33 |
| 9 | 69 ± 7 |
| 10 | 91 ± 12 |
| 11 | 90 ± 7 |
| 13 | 36540 ± 15600 |
| 15 | N.A. |
| 16 | 10710 ± 11900 |

Table 3: IC₅₀ Values of Fc-Binding Ligands in Competitive FP Binding Assay for Trastuzumab Against Fc-III-FAM

These results confirm that the Fc-binding ligands of Example 1 (compounds 1, 2, 9, 10 and 11) and Fc-III-FAM compete for the same binding site on the Fc region of trastuzumab. Moreover, the results demonstrate that N-terminal modification of the Fc-III peptide (compound 1) with spacer moieties (e.g. a peptidic spacer such as Ser-(Gly)₂-(Pro)₆ or polyethylene glycol spacers) does not affect the binding of the modified peptide to the Fc region of the antibody. In particular, compounds 2, 9, 10 and 11 exhibited high affinity for trastuzumab in the competitive FP binding assay against Fc-III-FAM (FIG. 4).

On the other hand, modification of the Fc-III C-terminal sequence in compounds 15 and 16 (i.e. replacement of Val-Trp-Cys-Thr (VWCT) by Trp-Ala-Cys-Thr (WACT) or Val-Trp-Ala-Thr (VWAT)) impaired peptide binding to the antibody. In the following, compounds or conjugates bearing said modified C-terminal sequence (WACT or VWAT) are used as negative controls.

### Example 4

### Preparation of DOTA-, FL- and DBCO-Carbonate Derivatives and FL-Thioester Derivatives—Compounds 17, 18, 19, 20, 21, 22 and 23

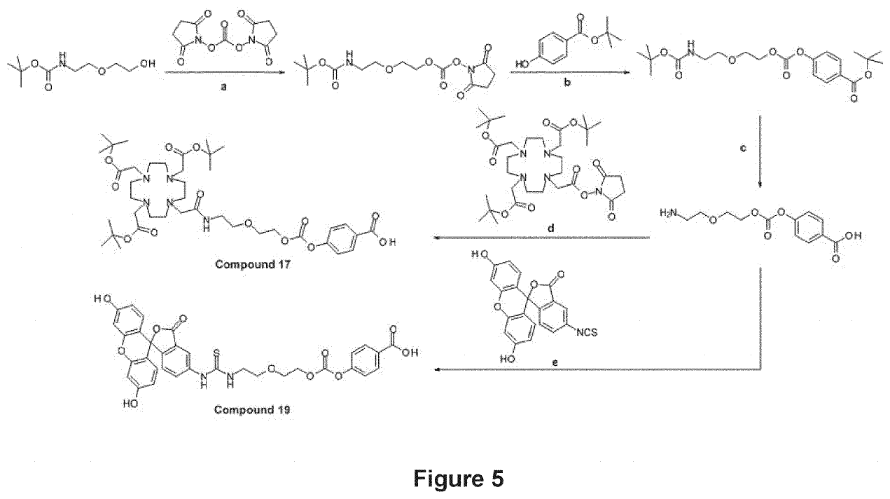

Compounds 17, 18, 19, 20, 21, 22 and 23 (moieties P-Y of formula (1)) were prepared according to the procedure described below and shown in FIG. 5. The respective structures of compounds 17-23 are shown in the table below.

| Compound | Structure |
|---|---|
| 17 | 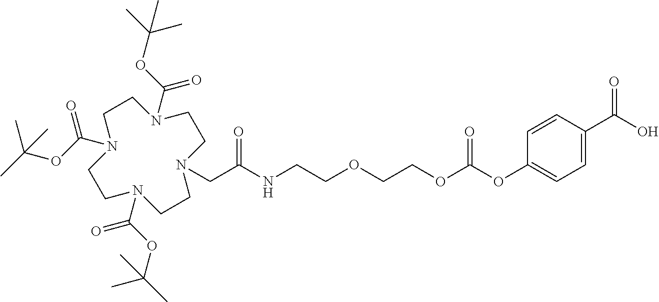 [SMILES: CC(C)(C)OC(=O)N1CCN(CC(=O)NCCOCCOC(=O)Oc2ccc(C(=O)O)cc2)CCN(C(=O)OC(C)(C)C)CCN(C(=O)OC(C)(C)C)CC1] |
| 18 | 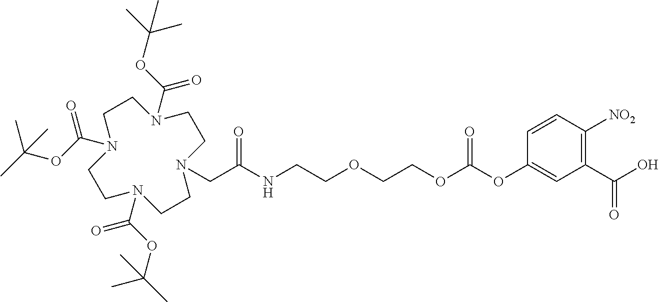 [SMILES: CC(C)(C)OC(=O)N1CCN(CC(=O)NCCOCCOC(=O)Oc2ccc([N+](=O)[O-])c(C(=O)O)c2)CCN(C(=O)OC(C)(C)C)CCN(C(=O)OC(C)(C)C)CC1] |
| 19 | 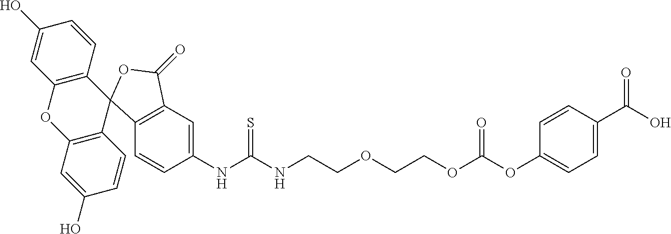 [SMILES: O=C(OCCOCCNC(=S)Nc1ccc2c(c1)C(=O)OC21c2ccc(O)cc2Oc2cc(O)ccc21)Oc1ccc(C(=O)O)cc1] |
| 20 | 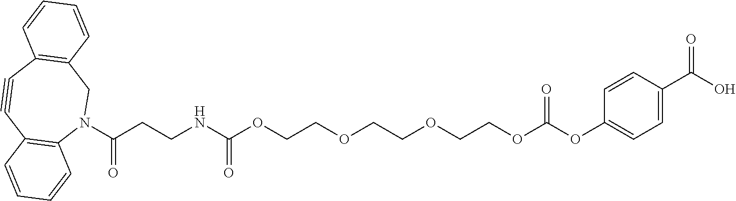 [SMILES: O=C(NCCC(=O)N1Cc2ccccc2C#Cc2ccccc21)OCCOCCOCCOC(=O)Oc1ccc(C(=O)O)cc1] |
| 21 | 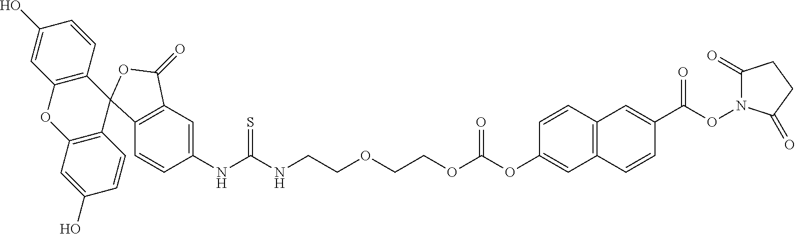 [SMILES: O=C(OCCOCCNC(=S)Nc1ccc2c(c1)C(=O)OC21c2ccc(O)cc2Oc2cc(O)ccc21)Oc1ccc2cc(C(=O)ON3C(=O)CCC3=O)ccc2c1] |
| 22 | 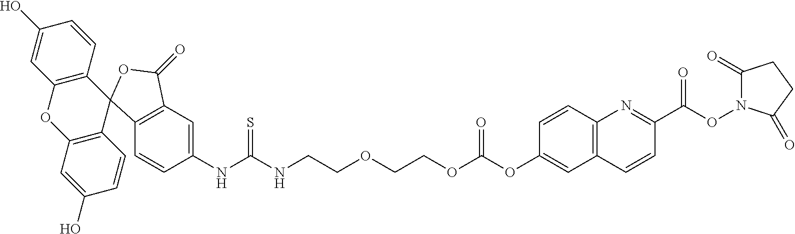 [SMILES: O=C(OCCOCCNC(=S)Nc1ccc2c(c1)C(=O)OC21c2ccc(O)cc2Oc2cc(O)ccc21)Oc1ccc2nc(C(=O)ON3C(=O)CCC3=O)ccc2c1] |
| 23 | 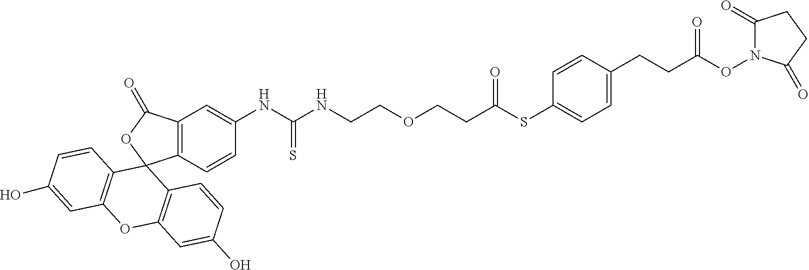 [SMILES: O=C(CCc1ccc(SC(=O)CCOCCNC(=S)Nc2ccc3c(c2)C(=O)OC32c3ccc(O)cc3Oc3cc(O)ccc32)cc1)ON1C(=O)CCC1=O] |

Preparation of Compound 17:

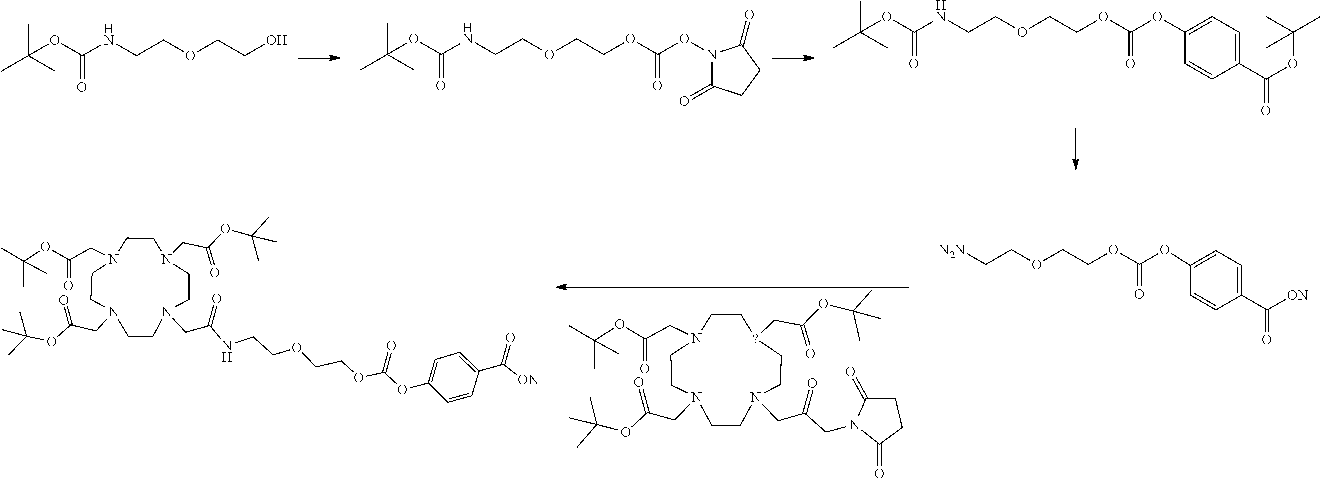

To a solution of 2.0 g of 2-(2-Boc-aminoethoxy)ethanol 1 (9.6 mmol) in 70 mL of acetonitrile were added 5.2 g of N,N′-disuccinimidyl carbonate (19 mmol, 2.0 eq) followed by 2.7 mL of triethylamine (19 mmol, 2.0 eq) and the suspension was stirred at 40° C. for 1 h 30. Solvent was removed in vacuo. The residue was dissolved in DCM and filtered through silica cartridge eluting with dichloromethane/ethyl acetate 80/20 to afford crude 2-[2-(tert-butoxycarbonylamino)ethoxy]ethyl (2,5-dioxopyrrolidin-1-yl) carbonate (Purity >80%, Yield: 99%). LCMS: m/z=247 [M−BOC+H]+, 369 [M+Na]+. 1H NMR (CDCl3): δ 4.52-4.40 (m, 2H), 3.77-3.68 (m, 2H), 3.55 (t, 2H), 3.32 (dd, 2H), 2.84 (s, 4H), 1.44 (s, 9H).

A solution of 1.5 g of 2-[2-(tert-butoxycarbonylamino)ethoxy]ethyl (2,5-dioxopyrrolidin-1-yl) carbonate (3.4 mmol, 2.0 eq) in 12 mL of DCM was treated with 0.35 g of tert-Butyl 4-hydroxybenzoate (1.7 mmol) then 0.43 g of 4-(dimethylamino)pyridine (3.4 mmol, 2 eq). The reaction mixture was stirred at room temperature for 30 min. 50 mL of water was added and extracted with 3×10 mL of dichloromethane. The organic layer was concentrated in vacuo. The residue was purified by Flash Chromatography (cyclohexane/ethyl acetate, 90/10 to 60/40) to afford 0.64 g of tert-butyl-4-[2-[2-(tertbutoxycarbonylamino)ethoxy] ethoxycarbonyloxy] benzoate as a colorless oil (Purity >98%, Yield: 88%). LCMS: m/z=326 [M−BOC+H]+, 448 [M+Na]+. 1H NMR (DMSO) δ 7.96 (d, 2H), 7.38 (d, 2H), 6.83 (s, 1H), 4.41-4.28 (m, 2H), 3.75-3.60 (m, 2H), 3.43 (t, J=6.0 Hz, 2H), 3.09 (q, 2H), 1.54 (s, 9H), 1.37 (s, 9H)

To a solution of 0.67 g of the compound obtained above (1.5 mmol) in 6.3 mL of DCM was added 2.1 ml of TFA (27 mmol, 17 eq) at 0° C. and the reaction mixture was stirred at room temperature for 3 h. The mixture was concentrated in vacuo to afford 0.73 g of compound 4-[2-(2-aminoethoxy)ethoxycarbonyloxy]benzoic acid; 2,2,2-trifluoroacetic acid as a white solid (Purity >80%, Yield: 97%). LCMS: m/z=270 [M+H]+. 1H NMR (DMSO) δ 8.01 (d, 1H), 7.87 (s, 2H), 7.37 (d, 2H), 4.38 (dd, 2H), 3.75 (dd, 2H), 3.65 (t, 2H), 3.06-2.97 (m, 2H).

To a solution of 0.70 g of DOTA-tris(tBu)ester NHS ester (0.83 mmol) in 3.5 mL of ACN were added 0.88 mL of DIEA (5.0 mmol, 6.0 eq) then 0.44 g of 4-[2-(2-aminoethoxy)ethoxycarbonyloxy]benzoic acid (0.92 mmol, 1.1 eq) and the reaction mixture was stirred at room temperature for 10 min (Note: a solid appeared immediately which was solubilized after sonification). The solution was diluted in 3.5 mL of water and purified by C18 cartridge Flash Chromatography (water/ACN, 90/10 to 0/100). Fractions were gathered, concentrated in vacuo and lyophilized to afford 0.66 g of compound 17 (4-[2-[2-[[2-[4,7,10-tris(2-tert-butoxy-2-oxo-ethyl)-1,4,7,10-tetrazacyclododec-1-yl]acetyl]amino]ethoxy]ethoxycarbonyloxy]benzoic acid) as a white solid (Purity >95%, Yield: 93%). LCMS: m/z=824 [M+H]+, 413 [M/2+H]+. 1H NMR (DMSO) δ 8.56 (s, 1H), 7.95 (d, 2H), 7.27 (d, 2H), 4.31 (s, 2H), 3.66 (s, 2H), 3.48-3.42 (m, 2H), 3.35-3.25 (m, 8H), 3.00 (s, 2H), 2.75 (s, 8H), 2.63 (s, 4H), 1.37 (s, 27H).

Preparation of Compound 18:

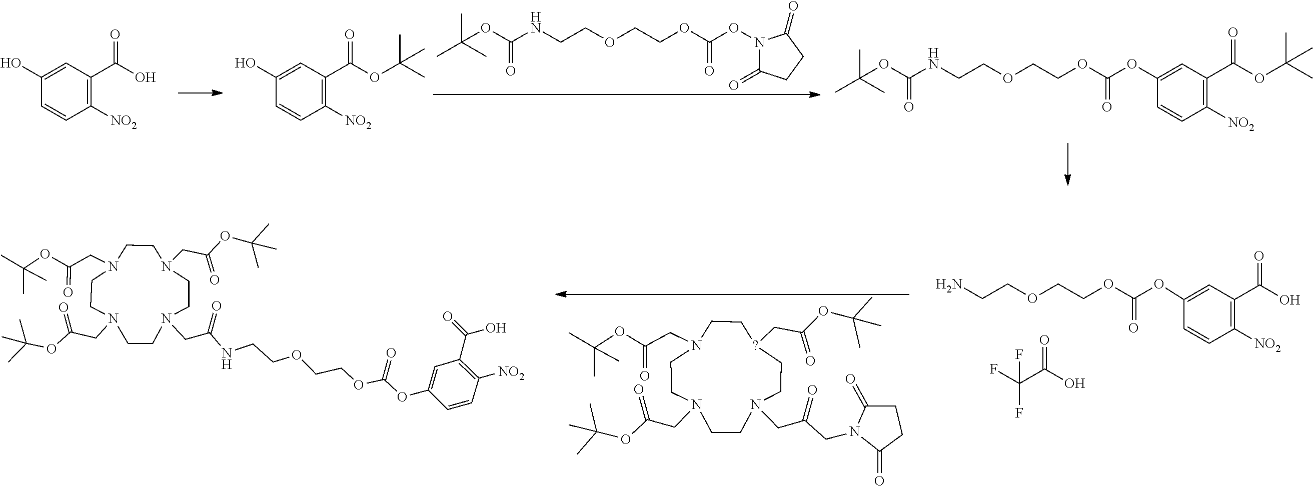

To a solution of 1.0 g of 5-hydroxy-2-nitrobenzoic acid (5.4 mmol) in 14 mL of toluene was added 7.6 mL of 2-methylpropan-2-ol (80 mmol, 15 eq) and the reaction mixture was heated at 85° C. 4.5 mL of N,N-dimethylformamide dineopentyl acetal (16 mmol, 3.0 eq) was slowly added and the reaction mixture was stirred at 85° C. for 3 h. Reaction was cooled down, then 10 mL of saturated aqueous solution of NaHCO₃ was added and the aqueous layer was extracted with 3×5 mL of ethyl acetate. Combined organic layers were washed with 10 mL of water and concentrated under vacuum to afford 1.1 g of crude tert-butyl 5-hydroxy-2-nitro-benzoate as a yellow oil (Purity: 89%, Yield: 73%). LCMS: m/z=238 [M−H]−. 1H NMR (DMSO): δ 8.00 (d, 1H), 7.00 (dd, 1H), 6.92 (d, 1H), 1.50 (s, 9H).

A solution of 0.35 g of crude tert-butyl 5-hydroxy-2-nitro-benzoate (1.3 mmol) in 5.0 mL of DCM was added to 0.90 g of 2-[2-(tert-butoxycarbonylamino)ethoxy]ethyl (2,5-dioxopyrrolidin-1-yl) carbonate (2.6 mmol, 2.0 eq; prepared as indicated above) then 0.46 mL of DIEA (2.6 mmol, 2.0 eq) was added. The reaction mixture was stirred at room temperature for 30 min. The mixture was purified by Flash Chromatography (cyclohexane/ethyl acetate 90/10 to 40/60) to yield 0.18 g of 5-((11,11-dimethyl-9-oxo-2,5,10-trioxa-8-azadodecanoyl)oxy)-2-nitrobenzoic acid as a yellow oil (Purity: 99%, Yield: 29%). LCMS: m/z=315 [M−Boc−(t−Bu)+H]+, 371 [M−Boc+H]+, 493 [M+Na]+. 1H NMR (DMSO): δ 8.15 (d, 1H), 7.77 (d, 1H), 7.69 (dd, 1H), 6.84 (s, 1H), 4.39-4.32 (m, 2H), 3.71-3.65 (m, 2H), 3.47-3.39 (m, 2H), 3.14-3.05 (m, 2H), 1.50 (s, 9H), 1.37 (s, 9H).

To a solution of 0.60 mL of TFA (7.8 mmol, 21 eq.) in 1.8 mL of DCM was added 0.17 g of 5-((11,11-dimethyl-9-oxo-2,5,10-trioxa-8-azadodecanoyl)oxy)-2-nitrobenzoic acid compound (0.37 mmol) and the reaction mixture was stirred at room temperature for 2 h. 0.30 mL of TFA was added and the mixture was stirred for 30 min at room temperature. Solvents were evaporated under vacuum to give 0.23 g of crude 5-[2-(2-aminoethoxy)ethoxycarbonyloxy]-2-nitro-benzoic acid; 2,2,2-trifluoroacetic acid as a yellow oil (Purity: 67%, Yield: quantitative). LCMS: m/z=315 [M+H]+. 1H NMR (DMSO): δ 8.13 (d, 1H), 7.78 (d, 1H), 7.67 (dd, 1H), 4.46-4.35 (m, 2H), 3.01 (q, 2H).

To a solution of 0.22 g of 5-((11,11-dimethyl-9-oxo-2,5,10-trioxa-8-azadodecanoyl)oxy)-2-nitrobenzoic acid (0.34 mmol, 1.15 eq.) in 1.3 mL of acetonitrile were added 0.25 g of tri-tert-butyl 2,2′,2″-(10-(2-((2,5-dioxopyrrolidin-1-yl)oxy)-2-oxoethyl)-1,4,7,10-tetraazacyclododecane-1,4,7-triyl)tracetate (0.30 mmol) followed by 0.31 mL of N,N-diisopropylethylamine (1.8 mmol, 6,0 eq.) and the reaction mixture was stirred at room temperature for 10 min. 1.5 mL of water was added and the solution was purified by C18 Flash Chromatography (water/acetonitrile 95/5 to 0/1) to yield 85 mg of compound 18 (2-nitro-5-[2-[2-[[2-[4,7,10-tris(2-tert-butoxy-2-oxo-ethyl)-1,4,7,10-tetrazacyclododec-1-yl]acetyl]amino]ethoxy]ethoxycarbonyloxy]benzoic acid) as a clear yellow solid (Purity >80%, Yield: 26%). LCMS: m/z=701 [M−3(t−Bu)+H]+, 757 [M−2(t−Bu)+H]+, 813 [M−(t−Bu)+H]+, 869 [M+H]+. 1H NMR (DMSO): δ 8.62 (s, 1H), 7.69 (d, 1H), 7.39 (d, 1H), 7.23 (dd, 1H), 4.38-4.31 (m, 2H), 3.70-3.67 (m, 2H), 1.42 (s, 6H), 1.41 (s, 27H).

Preparation of Compound 19:

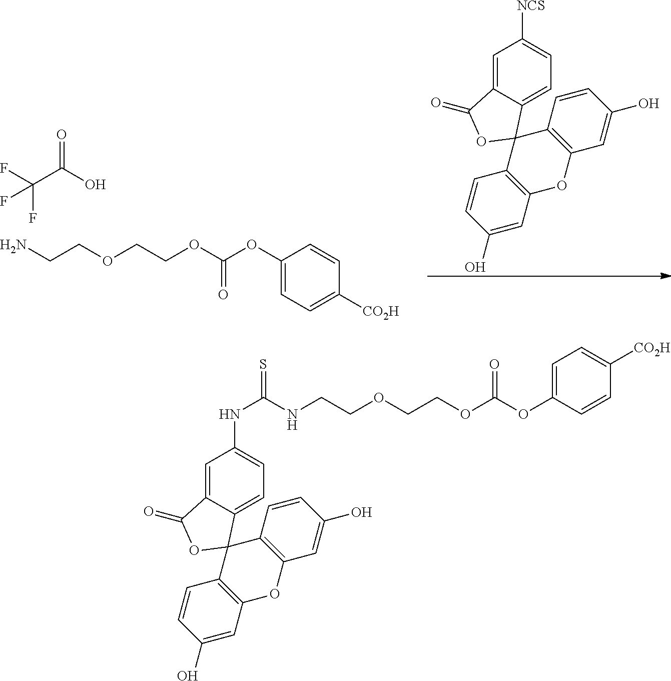

To a solution of 0.71 g of 4-[2-(2-aminoethoxy)ethoxycarbonyloxy]benzoic acid; 2,2,2-trifluoroacetic acid (1.2 mmol, 1.2 eq.) in 4.0 mL of ACN were added 0.40 g of fluorescein isothiocyanate isomer (1.0 mmol) and 4.0 mL of dimethylformamide followed by 1.1 mL of N,N-diisopropylethylamine (6.0 mmol, 6.0 eq.). This mixture was stirred for 10 min at room temperature. Solvents were evaporated under vacuum. The residue was purified by C₁₈ Flash Chromatography (water/acetonitrile 95/5 to 0/1) to yield 0.38 g of compound 19 (4-[2-[2-[(3′,6′-dihydroxy-3-oxo-spiro [isobenzofuran-1,9′-xanthene]-5-yl)carbamothioylamino]ethoxy] ethoxycarbonyloxy] benzoic acid as an orange solid) (Purity: 98%, Yield: 56%). LCMS: m/z=657 [M−H]−, 659 [M+H]+. 1H NMR (DMSO): δ 13.07 (s, 1H), 10.24-9.95 (m, 3H), 8.26 (s, 1H), 8.16 (s, 1H), 7.98 (d, 2H), 7.74 (d, 1H), 7.35 (d, 2H), 7.18 (d, 1H), 6.67 (d, 2H), 6.61-6.53 (m, 4H), 4.42-4.37 (m, 2H), 3.80-3.66 (m, 6H).

Preparation of Compound 20:

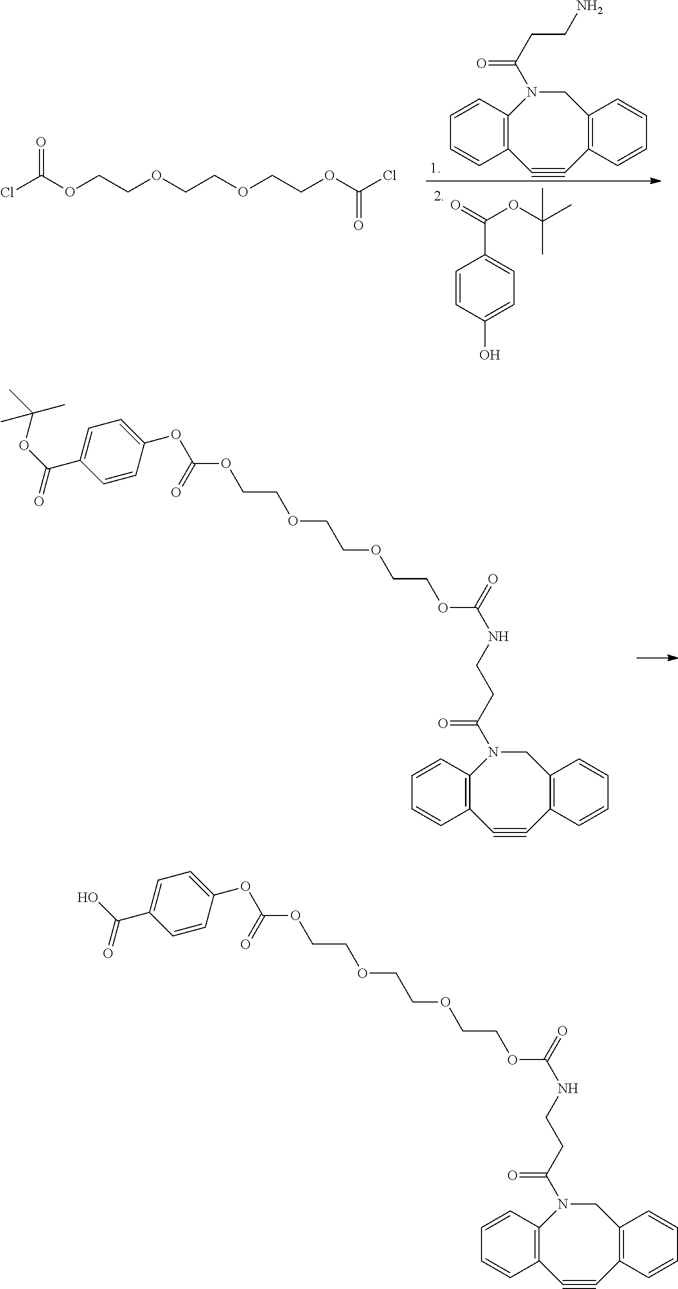

To a solution of 194 mg of tri(ethylene glycol) bis(chloroformate) (0.690 mmol, 2.0 eq) and 0.070 mL of DIEA (0.420 mmol, 1.2 eq) in dry DCM (1.20 mL), was added dropwise 3-amino-1-(11,12-didehydrodibenzo[b,f]azocin-5(6H)-yl)-1-propanone (100 mg, 0.350 mmol, 1.0 eq) over 10 min at 0° C. The reaction was stirred at room temperature. After 10 min, a solution of 0.3 mL of DIEA (1.73 mmol, 5.0 eq) in dry DCM (1.20 mL) and 336 mg of tert-butyl 4-hydroxybenzoate (1.73 mmol, 5.0 eq) were subsequently added to the reaction mixture. The reaction was stirred at room temperature for 30 min. A saturated aqueous solution of ammonium chloride was then added to the reaction mixture and the mixture was extracted with DCM (2×5 mL). The combined organic extracts were dried on MgSO₄. After filtration, the solvent was removed in vacuo and the residue was purified by Flash Chromatography (cyclohexane/ethyl acetate, 40/60 to 10/90), which gave 67.6 mg of tert-butyl 4-[2-[2-[2-[[3-(2-azatricyclo[10.4.0.04,9]hexadeca-1(12),4(9),5,7,13,15-hexaen-10-yn-2-yl)-3-oxo-propyl]carbamoyloxy]ethoxy]ethoxy]ethoxycarbonyloxy]benzoate. (Purity: 80%, Yield: 23%). LCMS: m/z=673.3 [M+H]+.

A solution of 67.6 mg of the tert-butyl ester compound in 1:1 DCM/TFA was stirred at room temperature for 5 h. The solution was concentrated under reduced pressure and the residue was purified by C₁₈ Flash Chromatography (water/ACN modified with 0.1% TFA 80/20 to 20/80), which gave 40.3 mg of compound 20 (Purity: 80%, Yield: 59%). LCMS: m/z=615 [M−H]−, 617 [M+H]+. 1H NMR (CDCl₃): δ 8.18-8.07 (m, 2H), 8.04-7.95 (m, 2H), 7.70-7.25 (m, 8H), 5.16 (s, 2H), 4.47-4.37 (m, 2H), 4.31-4.21 (m, 2H), 3.94-3.62 (m, 8H), 3.44-3.32 (m, 2H).

Preparation of Compound 21:

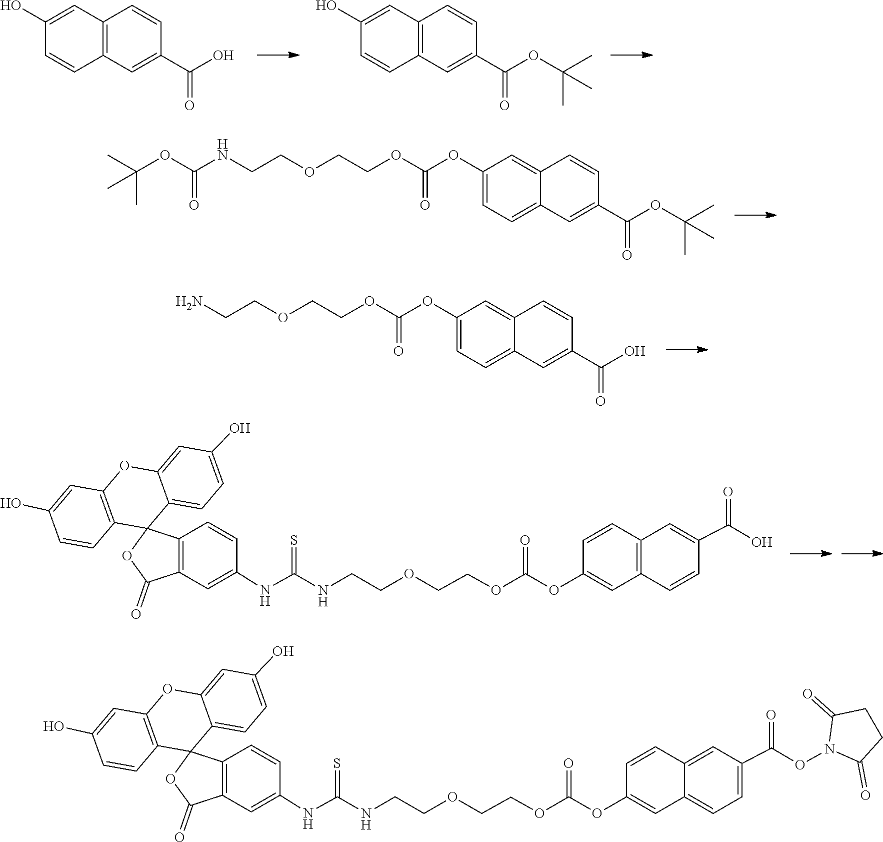 [SMILES: O=C(O)c1ccc2cc(O)ccc2c1 ; CC(C)(C)OC(=O)c1ccc2cc(O)ccc2c1 ; CC(C)(C)OC(=O)NCCOCCOC(=O)Oc1ccc2cc(C(=O)OC(C)(C)C)ccc2c1 ; NCCOCCOC(=O)Oc1ccc2cc(C(=O)O)ccc2c1 ; O=C(OCCOCCNC(=S)Nc1ccc2c(c1)C(=O)OC21c2ccc(O)cc2Oc2cc(O)ccc21)Oc1ccc2cc(C(=O)O)ccc2c1 ; O=C(OCCOCCNC(=S)Nc1ccc2c(c1)C(=O)OC21c2ccc(O)cc2Oc2cc(O)ccc21)Oc1ccc2cc(C(=O)ON3C(=O)CCC3=O)ccc2c1]

To a solution of 6-hydroxy-2-naphthoic acid (941 mg, 5.00 mmol) in 2-methyltetrahydrofuran (20.0 mL) was added a solution of 2-tert-butyl-1,3-diisopropylisourea (4.00 mL, 15.0 mmol) in 2-methyltetrahydrofuran (5.00 mL). The reaction mixture was stirred at room temperature for 18 hours. The reaction mixture was filtered through a plug of silica flushing with ethyl acetate. The filtrate was washed with saturated aqueous NaHCO₃ solution and brine, dried over Na₂SO₄ and concentrated under reduced pressure. The residue was purified by normal phase chromatography (Biotage Isolera, 40 g, Silicycle siliasep cartridge) using 0-40% ethyl acetate in heptane to give the desired compound (715 mg, yield 59%, purity 99%) as orange oil. ESI: m/z=243 (M−H)⁻. ¹H NMR (400 MHz, DMSO-d₆) δ [ppm]=1.59 (s, 9H), 7.14-7.21 (m, 2H), 7.75 (d, 1H), 7.82 (dd, 1H), 7.95 (d, 1H), 8.40 (s, 1H), 10.15 (br s, 1H).

To a solution of tert-butyl 6-hydroxy-2-naphthoate (200 mg, 0.82 mmol) and tert-butyl (2-(2-((((2,5-dioxopyrrolidin-1-yl)oxy)carbonyl)oxy)ethoxy)ethyl)carbamate (567 mg, 1.64 mmol) in dichloromethane (15.0 mL) was added 4-dimethylaminopyridine (200 mg, 1.64 mmol). The reaction mixture was stirred at ambient temperature for 2 hours. The reaction mixture was washed with water, the aqueous layer was washed with dichloromethane. The combined organic layers were concentrated under reduced pressure. The residue was purified by normal phase chromatography (Biotage Isolera, 60 g, Silicycle siliasep cartridge) using 10-90% ethyl acetate in heptane to give the title compound (190 mg, yield 49%, purity 98%) as a colourless solid. ESI: m/z=498 (M+Na)⁺. ¹H NMR (400 MHz, DMSO-d₆) δ [ppm]=1.38 (s, 9H), 1.61 (s, 9H), 3.11 (q, 2H), 3.46 (t, 2H), 3.68-3.72 (m, 2H), 4.34-4.39 (m, 2H), 6.81-6.86 (m, 1H), 7.53 (dd, 1H), 7.90 (d, 1H), 7.96-8.05 (m, 2H), 8.22 (d, 1H), 8.60 (br s, 1H).

To a solution of tert-butyl 6-((11,11-dimethyl-9-oxo-2,5,10-trioxa-8-azadodecanoyl)oxy)-2-naphthoate (190 mg, 0.400 mmol) in dichloromethane (10.0 mL) was added trifluoroacetic acid (1.00 mL) and the reaction mixture was stirred at ambient temperature for 22 hours. The mixture was concentrated under reduced pressure. The residue was dissolved in N,N-dimethylformamide (2.00 mL) and acetonitrile (2.00 mL), then fluorescein isothiocyanate isomer 1 (204 mg, 0.520 mmol) was added followed by DIPEA (343 μL, 1.97 mmol). The reaction mixture was stirred at room temperature for 90 minutes. The material was purified by reverse phase chromatography (Biotage Isolera, 60 g, C18 SNAP Ultra Biotage cartridge) using water containing 0.1% formic acid and acetonitrile containing 0.1% formic acid (90:10 to 0:100). The fractions containing product were freeze dried to give the desired compound (180 mg, yield 64%, purity 71%). ESI: m/z=707 (M−H)⁻. ¹H NMR (400 MHz, DMSO-d₆) δ [ppm]=3.68-3.83 (m, 6H), 4.41-4.46 (m, 2H), 6.52-6.70 (m, 6H), 7.19 (d, 1H), 7.50 (dd, 1H), 7.75 (d, 1H), 7.88 (d, 1H), 8.01 (s, 2H), 8.14-8.24 (m, 2H), 8.28 (d, 1H), 8.65 (s, 1H), 10.06 (br s, 1H), 10.11 (br s, 2H), 13.13 (br s, 1H).

To a solution of 6-(((2-(2-(3-(3′,6′-dihydroxy-3-oxo-3H-spiro[isobenzofuran-1,9′-xanthen]-5-yl)thioureido)ethoxy)ethoxy)carbonyl)oxy)-2-naphthoic acid (70.0 mg, 0.099 mmol) in N,N-dimethylformamide (1.00 mL) was added N-hydroxysuccinimide (34.0 mg, 0.300 mmol) followed by EDCI.HCl (57.0 mg, 0.300 mmol). The mixture was stirred at ambient temperature for 4 hours, then purified on a 60 g C18 column with a 5-95% acetonitrile (0.1% formic acid) in water (0.1% formic acid) eluent. The desired fractions were combined and freeze dried to afford the title compound (55.0 mg, yield 69%, purity 95%). ESI: m/z=806 (M+H)⁺. ¹H NMR (400 MHz, DMSO-d₆) δ [ppm]=2.94 (s, 4H), 3.68-3.84 (m, 6H), 4.42-4.48 (m, 2H), 6.51-6.63 (m, 4H), 6.66 (d, 2H), 7.19 (d, 1H), 7.61 (dd, 1H), 7.75 (d, 1H), 7.99 (d, 1H), 8.08 (dd, 1H), 8.16 (d, 2H), 8.28 (d, 1H), 8.34 (d, 1H), 8.91 (s, 1H), 10.00-10.15 (m, 3H).

Preparation of Compound 22:

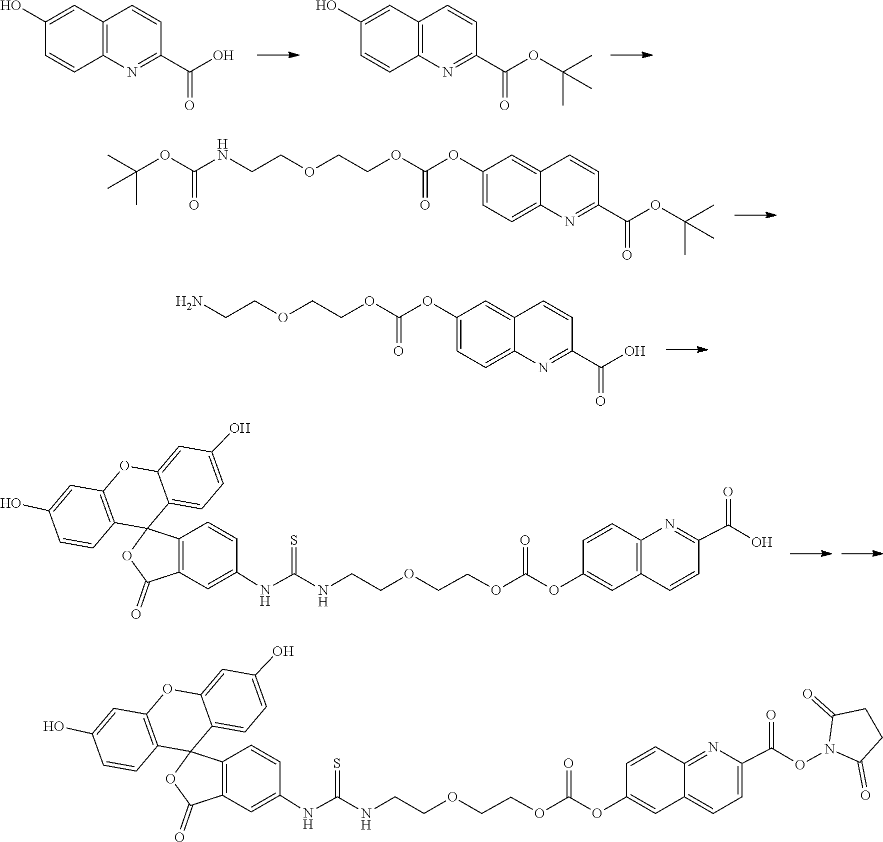 [SMILES: O=C(O)c1ccc2cc(O)ccc2n1 ; CC(C)(C)OC(=O)c1ccc2cc(O)ccc2n1 ; CC(C)(C)OC(=O)NCCOCCOC(=O)Oc1ccc2nc(C(=O)OC(C)(C)C)ccc2c1 ; NCCOCCOC(=O)Oc1ccc2nc(C(=O)O)ccc2c1 ; O=C(OCCOCCNC(=S)Nc1ccc2c(c1)C(=O)OC21c2ccc(O)cc2Oc2cc(O)ccc21)Oc1ccc2nc(C(=O)O)ccc2c1 ; O=C(OCCOCCNC(=S)Nc1ccc2c(c1)C(=O)OC21c2ccc(O)cc2Oc2cc(O)ccc21)Oc1ccc2nc(C(=O)ON3C(=O)CCC3=O)ccc2c1]

To a solution of 6-hydroxyquinoline-2-carboxylic acid (750 mg, 3.96 mmol) in tert-butanol (40.0 mL) was added a solution of 2-tert-butyl-1,3-diisopropylisourea (3.20 mL, 11.9 mmol) in tert-butanol (5.00 mL). The reaction mixture was stirred at room temperature for 3 days. The reaction mixture was concentrated under reduced pressure, the residue was suspended in ethyl acetate and filtered through a plug of silica flushing with ethyl acetate. The filtrate was concentrated under reduced pressure and then purified by normal phase chromatography (Biotage Isolera, 40 g, Silicycle siliasep cartridge) using 5-50% ethyl acetate in heptane to give the desired compound (378 mg, yield 39%, purity 95%) as an orange oil. ESI: m/z=244 (M−H)⁻. ¹H NMR (400 MHz, DMSO-d₆) δ [ppm]=1.60 (s, 9H), 7.21 (d, 1H), 7.40 (dd, 1H), 7.94 (d, 1H), 7.99 (d, 1H), 8.27 (d, 1H), 10.42 (br s, 1H).

To a solution of tert-butyl 6-hydroxyquinoline-2-carboxylate (200 mg, 0.820 mmol) and tert-butyl (2-(2-((((2,5-dioxopyrrolidin-1-yl)oxy)carbonyl)oxy)ethoxy)ethyl)carbamate (706 mg, 2.04 mmol) in dichloromethane (15.0 mL) was added 4-dimethylaminopyridine (199 mg, 1.64 mmol). The reaction mixture was stirred at room temperature for 2 hours. The reaction mixture was washed with water, the aqueous layer was washed with dichloromethane. The combined organic layers were concentrated under reduced pressure. The residue was purified by normal phase chromatography (Biotage Isolera, 40 g, Silicycle siliasep cartridge) using 10-90% ethyl acetate in heptane to give the desired compound (278 mg, yield 71%, purity 94%) as a colourless oil. ESI: m/z=499 (M+Na)⁺. ¹H NMR (400 MHz, DMSO-d₆) δ [ppm]=1.38 (s, 9H), 1.62 (s, 9H), 3.07-3.16 (m, 2H), 3.46 (t, 2H), 3.68-3.74 (m, 2H), 4.35-4.41 (m, 2H), 6.80-6.87 (m, 1H), 7.79 (dd, 1H), 8.00 (d, 1H), 8.10 (d, 1H), 8.22 (d, 1H), 8.56 (d, 1H).

To a solution of tert-butyl 6-((11,11-dimethyl-9-oxo-2,5,10-trioxa-8-azadodecanoyl)oxy)quinoline-2-carboxylate (290 mg, 0.61 mmol) in dichloromethane (5.00 mL) was added trifluoroacetic acid (1.50 mL) and the reaction mixture was stirred at room temperature for 26 hours. The mixture was concentrated under reduced pressure. The residue was dissolved in N,N-dimethylformamide (2.00 mL) and acetonitrile (2.00 mL), then fluorescein isothiocyanate isomer 1 (237 mg, 0.610 mmol) was added followed by DIPEA (530 μL, 3.04 mmol). The reaction mixture was stirred at room temperature for 2 hours. The material was purified by reverse phase chromatography (Biotage Isolera, 60 g, C18 SNAP Ultra Biotage cartridge) using water containing 0.1% formic acid and acetonitrile containing 0.1% formic acid (90:10 to 100:0). The fractions containing product were freeze dried to give the title compound (150 mg, yield 35%, purity 91%). ESI: m/z=710 (M+H)⁺. ¹H NMR (400 MHz, DMSO-d₆) δ [ppm]=3.68-3.83 (m, 6H), 4.41-4.46 (m, 2H), 6.52-6.69 (m, 6H), 7.18 (d, 1H), 7.72-7.80 (m, 2H), 7.97 (d, 1H), 8.12-8.22 (m, 3H), 8.27 (d, 1H), 8.53 (d, 1H), 10.00-10.20 (m, 3H), 13.49 (br s, 1H).

To a solution of 6-(((2-(2-(3-(3′,6′-dihydroxy-3-oxo-3H-spiro[isobenzofuran-1,9′-xanthen]-5-yl)thioureido)ethoxy)ethoxy)carbonyl)oxy)quinoline-2-carboxylic acid (70.0 mg, 0.099 mmol) in N,N-dimethylformamide (1.00 mL) was added N-hydroxysuccinimide (34.0 mg, 0.300 mmol) followed by EDCI.HCl (57.0 mg, 0.300 mmol). The mixture was stirred at ambient temperature for 2 hours, then purified on a 60 g C18 column with a 5-95% acetonitrile (0.1% formic acid) in water (0.1% formic acid) eluent. The desired fractions were combined and freeze dried to afford the title compound (55.0 mg, yield 69%, purity 92%). ESI: m/z=807 (M+H)⁺. ¹H NMR (400 MHz, DMSO-d₆) δ [ppm]=2.94 (s, 4H), 3.68-3.85 (m, 6H), 4.43-4.48 (m, 2H), 6.52-6.63 (m, 4H), 6.66 (d, 2H), 7.19 (d, 1H), 7.75 (d, 1H), 7.87 (dd, 1H), 8.09 (d, 1H), 8.13-8.21 (m, 1H), 8.25-8.33 (m, 3H), 8.72 (d, 1H), 10.00-10.13 (m, 3H).

Preparation of Compound 23:

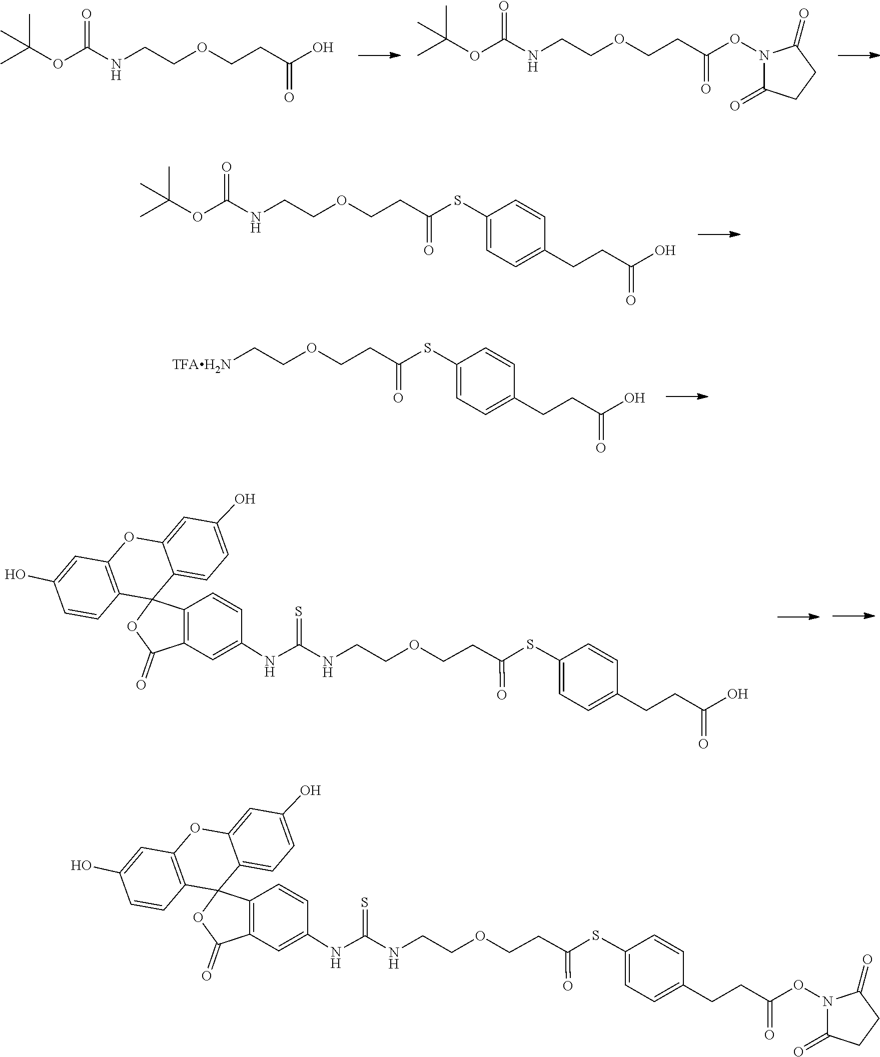

To a solution of 3-(2-((tert-butoxycarbonyl)amino)ethoxy)propanoic acid (300 mg, 1.29 mmol) in dichloromethane (3.00 mL) was added EDCI.HCl (296 mg, 1.54 mmol) followed by 1-hydroxypyrrolidine-2,5-dione (177 mg, 1.54 mmol). The reaction mixture was stirred at room temperature for 4 hours. The reaction mixture was diluted with dichloromethane and washed with water. The organic layer was passed through a 15.0 mL Telos phase separator cartridge and concentrated to give the desired product (313 mg, purity 79%) as colourless oil. Used without purification in the next step. ESI: m/z=353 (M+Na)⁺, 231 (M−Boc+H)⁺. ¹H NMR (400 MHz, DMSO-d₆) δ [ppm]=1.38 (s, 9H), 2.82 (s, 4H), 2.92 (t, 2H), 3.04-3.09 (m, 2H), 3.40 (t, 2H), 3.69 (t, 2H), 6.71-6.75 (m, 1H). NMR spectra contains unknown impurities: 2.50 (t), 3.50 (t).

To a suspension of 2,5-dioxopyrrolidin-1-yl 3-(2-((tert-butoxycarbonyl)amino)ethoxy)propanoate (200 mg, 0.606 mmol) in dichloromethane (4.00 mL) was added 4-mercaptohydrocinnamic acid (88.4 mg, 0.485 mmol) followed by 4-dimethylaminopyridine (148 mg, 1.21 mmol). The reaction mixture was stirred at room temperature for 2 hours. The reaction mixture was washed with 10% citric acid aqueous solution and then water. The organic layer was passed through a 15.0 mL Telos phase separator cartridge and the filtrate was concentrated under reduced pressure. The residue was purified by reverse phase chromatography (Biotage Isolera, 30 g, C18 SNAP Ultra Biotage cartridge) using water containing 0.1% formic acid and acetonitrile containing 0.1% formic acid (80:20 to 20:80). The appropriate fractions were freeze dried to give the desired product (94.0 mg, yield 29% over 2 steps, purity 97%) as a white solid. ESI: m/z=298 (M−Boc+H)⁺. ¹H NMR (400 MHz, CDCl₃) δ [ppm]=1.44 (s, 9H), 2.70 (t, 2H), 2.85-2.91 (m, 2H), 2.99 (t, 2H), 3.27-3.32 (m, 2H), 3.49 (t, 2H), 3.76 (t, 2H), 4.94 (br s, 1H), 7.27 (d, 2H) (overlaps with CHCl₃ peak), 7.36 (d, 2H),

To a solution of 3-(4-((3-(2-((tert-butoxycarbonyl)amino)ethoxy)propanoyl)thio)phenyl)propanoic acid (180 mg, 0.453 mmol) in dichloromethane (2.25 mL) was added trifluoroacetic acid (0.59 mL, 7.70 mmol) and the reaction mixture was stirred at room temperature for 1 hour. The mixture was concentrated under reduced pressure to give the desired product (205 mg, purity 80%) as a pale-yellow oil. Used in the next step without purification. ESI: m/z=298 (M+H)⁺. ¹H NMR (400 MHz, DMSO-d₆) δ [ppm]=2.57 (t, 2H), 2.87 (t, 2H), 2.96-3.01 (m, 4H), 3.58 (t, 2H), 3.74 (t, 2H), 7.31-7.36 (m, 4H), 7.76 (br s, 3H).

To a solution of 3-(4-((3-(2-aminoethoxy)propanoyl)thio)phenyl)propanoic acid trifluoroacetic acid salt (maximum 0.453 mmol) in N,N-dimethylformamide (3.70 mL) and acetonitrile (3.70 mL), fluorescein isothiocyanate isomer 1 (176 mg, 0.453 mmol) was added followed by DIPEA (0.12 mL, 0.680 mmol). The reaction mixture was stirred at room temperature for 1 hour then concentrated under reduced pressure. The material was purified by reverse phase chromatography (Biotage Isolera, 60 g, C18 SNAP Ultra Biotage cartridge) using water containing 0.1% formic acid and acetonitrile containing 0.1% formic acid (95:5 to 20:80). The fractions containing product were freeze dried to give the desired compound (145 mg, yield 51% over 2 steps, purity 68%) as an orange solid. ESI: m/z=687 (M+H)⁺. ¹H NMR (400 MHz, DMSO-d₆) δ [ppm]=2.55 (t, 2H) (overlaps with DMSO peak), 2.85 (t, 2H), 2.99 (t, 2H), 3.61 (t, 2H), 3.67-3.72 (m, 2H), 3.76 (t, 2H), 6.55-6.69 (m,6H), 7.18 (d, 1H), 7.29-7.34 (m, 4H), 7.74 (d, 1H), 8.10 (br s, 1H), 8.26 (s, 1H), 10.05 (br s, 1H), 10.13 (br s, 2H), 12.16 (br s, 1H).

To a solution of 3-(4-((3-(2-(3-(3′,6′-dihydroxy-3-oxo-3H-spiro[isobenzofuran-1,9′-xanthen]-5-yl)thioureido)ethoxy)propanoyl)thio)phenyl)propanoic acid (140 mg, 0.204 mmol) in N,N-dimethylformamide (4.70 mL), was added N-hydroxysuccinimide (117 mg, 1.02 mmol), followed by EDCI.HCl (196 mg (1.02 mmol). Stirring continued at room temperature for 1 hour. The reaction mixture was purified directly by reverse phase chromatography (Biotage Isolera, 60 g, C18 SNAP Ultra Biotage cartridge) using water containing 0.1% formic acid and acetonitrile containing 0.1% formic acid (80:20 to 30:70). The fractions containing product were freeze-dried to give the desired compound (25.3 mg, yield 16%, purity 81%) as an orange solid. ESI: m/z=784 (M+H)⁺. ¹H NMR (400 MHz, DMSO-d₆) δ [ppm]=2.81 (s, 4H), 2.96-3.06 (m, 6H), 3.62 (t, 2H), 3.68-3.71 (m, 2H), 3.76 (t, 2H), 6.56 (dd, 2H), 6.61 (d, 2H), 6.68 (d, 2H), 7.18 (d, 1H), 7.33 (d, 2H), 7.39 (d, 2H), 7.74 (d, 1H), 8.08 (br s, 1H), 8.26 (d, 1H), 10.03 (br s, 1H), 10.13 (br s, 2H).

### Example 5

### Preparation of DOTA-Containing Reactive Conjugates

The Fc-binding vectors prepared in Example 1 were converted into reactive conjugates of formula (1) by coupling of compound 17 (or compound 19) to the N-terminus of the respective Fc-binding vectors (FIG. 2b). The structures of the DOTA-containing reactive conjugates prepared in Example 5 are shown in the table below.

TABLE 4
Structures of DOTA-containing reactive conjugates of formula (1)

| Compound | Structure |
|---|---|
| 24 | 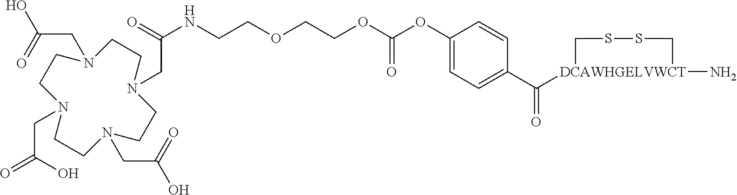 |
| 25 | 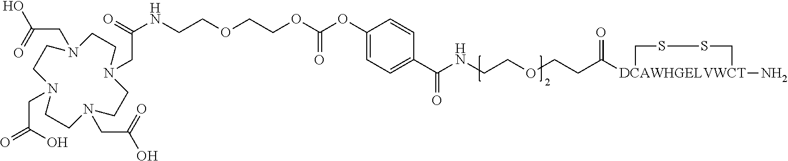 |
| 26 | 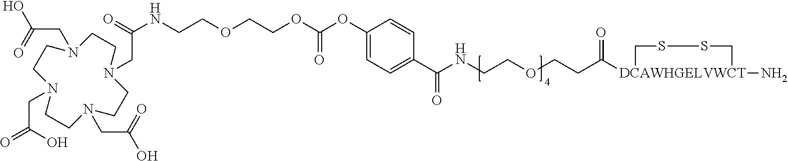 |
| 27 | 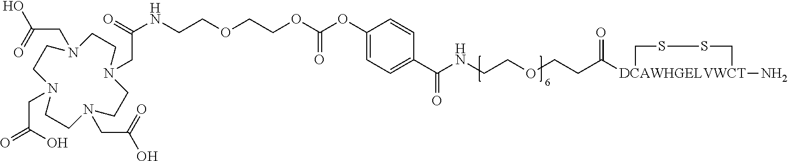 |
| 28 | 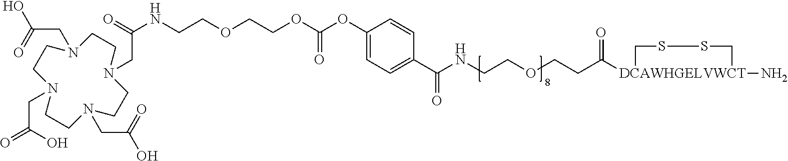 |
| 29 | 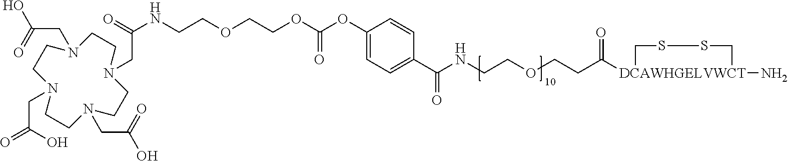 |
| 30 | 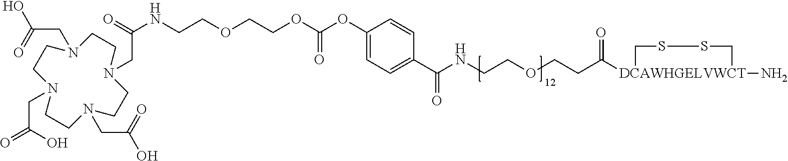 |
| 31 | 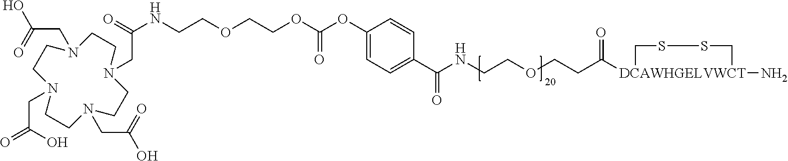 |
| 32 | 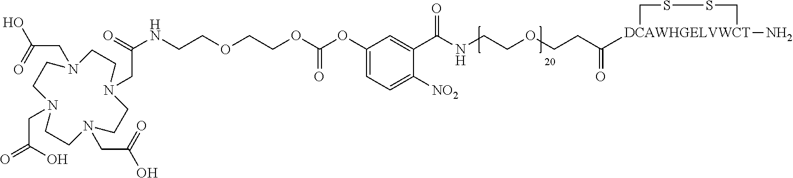 |
| 33 | 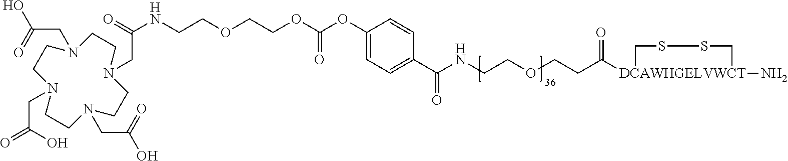 |
| 34 | 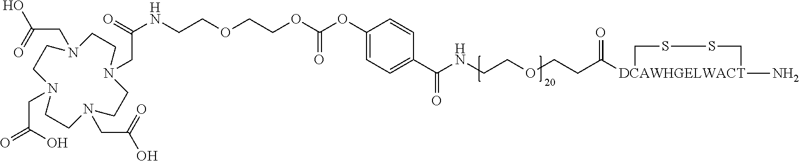 |

To prepare the reactive conjugates, a solution of carbonate derivative (1.2 eq; compound 17) in DMF was added to HATU (1.1 eq) and stirred for 1 min, followed by the addition of DIEA (2 eq). After 3 min, the pre-activated carbonate derivative to the Fc-binding vector and the reaction mixture was stirred for 2 to 4 hours at room temperature. Completion of the reaction was monitored by UPLC-MS. If the reaction did not go to completion, an additional amount of pre-activated carbonate derivative (about 1 to 3 eq) was added, and the mixture was further stirred for 1 to 2 hours. The reactive conjugate was precipitated with cold diethyl ether and purified by HPLC (as described above).

Subsequently, the tert-butyl protecting groups of the DOTA moiety were removed by treatment with TFA/TIS/water (95/2.5/2.5, v/v/v) over 2.5 hours at room temperature, followed by precipitation with cold diethyl ether and purification by HPLC (as described above).

The purity of the reactive conjugates was determined on a Waters Acquity UPLC system coupled to a Micromass Quattro micro API mass spectrometer with a Kinetex® XB-C18 column (100 Å, 1.7 μm, 50×2.1 mm; Phenomenex Helvetia) using solvent system using solvent system A (0.1% TFA in water) and B (0.1% TFA in ACN) at a flow rate of 0.6 mL/min and a 2-98% gradient of B over 4 min. Elution of the conjugates was monitored at a wavelength of 214 nm. The results of the shown in the table below.

TABLE 5
Characterization of reactive conjugates 24-34

| | Calculated | Calculated | Found | Retention | Purity |
|---|---|---|---|---|---|
| Compound | Exact Mass | M + H⁺ | M + H⁺ | time (min) | (%) |
| 24 | 2166.73 | 1084.37 | 1084.95 | 1.69 | >95 |
| 25 | 2326.13 | 1164.07 | 1164.28 | 1.70 | 95 |
| 26 | 2414.27 | 1208.14 | 1207.99 | 1.72 | 95 |
| 27 | 2502.13 | 1252.07 | 1252.46 | 1.73 | 95 |
| 28 | 2590.53 | 864.51 | 865.12 | 1.74 | >95 |
| 29 | 2678.63 | 893.88 | 894.64 | 1.75 | >90 |
| 30 | 2766.73 | 923.24 | 924.06 | 1.77 | 95 |
| 31 | 3119.91 | 1040.97 | 1040.69 | 1.81 | 94 |
| 32 | 3164.91 | 1055.97 | 1056.60 | 1.83 | 87 |
| 33 | 3824.03 | 1275.68 | 1275.55 | 1.88 | >95 |
| 34 | 3090.86 | 1031.29 | 1025.81 | 1.69 | 84 |

Indium chelation in the DOTA moiety was performed by dissolving InCl₃ in ultra-pure water (1.5 eq, 14.2 nmol, 2 μL), mixing with the reactive conjugates described above (9.45 nmol, 5 μL) in 50 mM sodium acetate buffer, pH 5 (3 μL), and incubating for 5-30 min at 37° C. The In chelation was monitored and analyzed by UPLC-MS.

### Example 6

### Preparation of Trastuzumab-DOTA Conjugates

The propensity of the reactive conjugates of Example 5 to react with an antibody was evaluated using trastuzumab as a model system. To prepare the trastuzumab-DOTA conjugates, 2 eq of reactive conjugate prepared in Example 5 (compounds 24-33; 1.62 nmol, 0.86 μL) in DMF was added to a solution of trastuzumab (1 eq, 0.81 nmol; commercial trastuzumab Herceptin® available from Roche which was buffer-exchanged into phosphate-buffered saline (PBS) prior to the conjugation) diluted in 50 mM NaHCO₃ pH 9.0 and the reaction mixture (24 μL) was stirred at room temperature for 2 hours.

After DOTA conjugation, the reaction buffer was diluted with 0.1 M glycine pH 2.5 or exchanged to 0.1M glycine pH 2.5 using a 30 kDa MWCO Vivaspin® 500 centrifugal concentrators. The antibody conjugate was then purified by gel filtration chromatography using a pre-equilibrated Bio-spin P-30 Column (bed height: 3.7 cm, overall length: 5 cm; available from Bio-Rad, USA) and then eluted with 0.1M glycine pH 2.5. The purified antibody conjugate fractions were neutralized with 1M PBS pH 8.5.

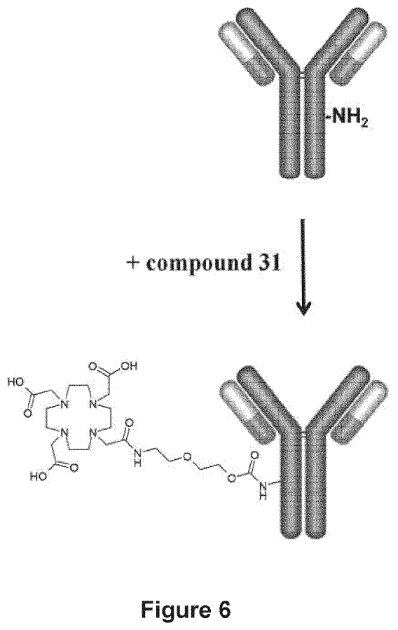

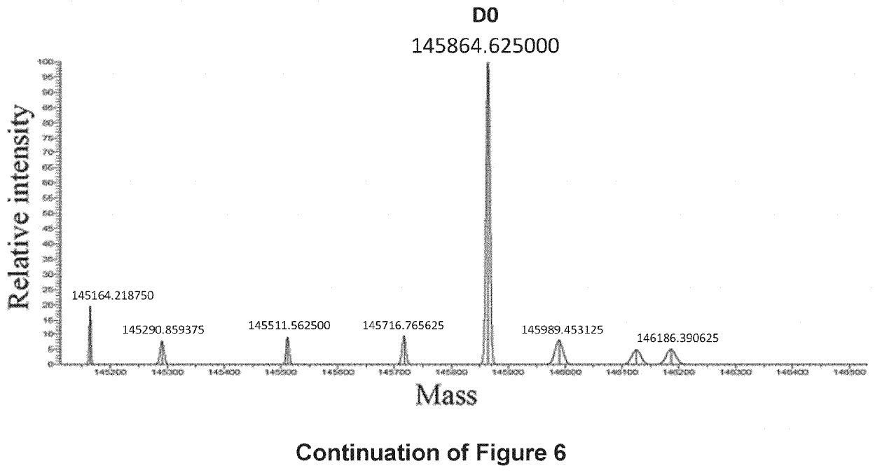

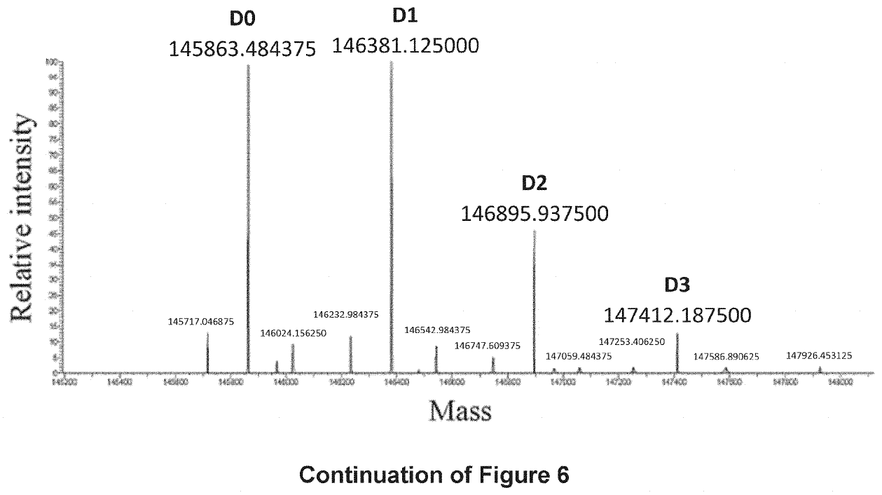

The conjugation of the DOTA moiety to trastuzumab was evaluated by HRMS analysis (as described above). An exemplary HRMS spectrum of a trastuzumab-DOTA conjugate prepared by reacting compound 31 with trastuzumab is shown in FIG. 6. The sample displayed +517 Da adducts (D1-D3), which are characteristic of DOTA incorporation.

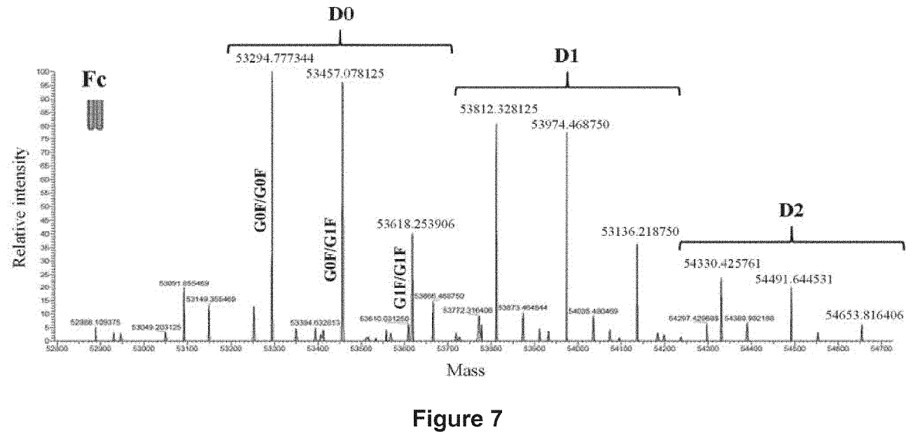

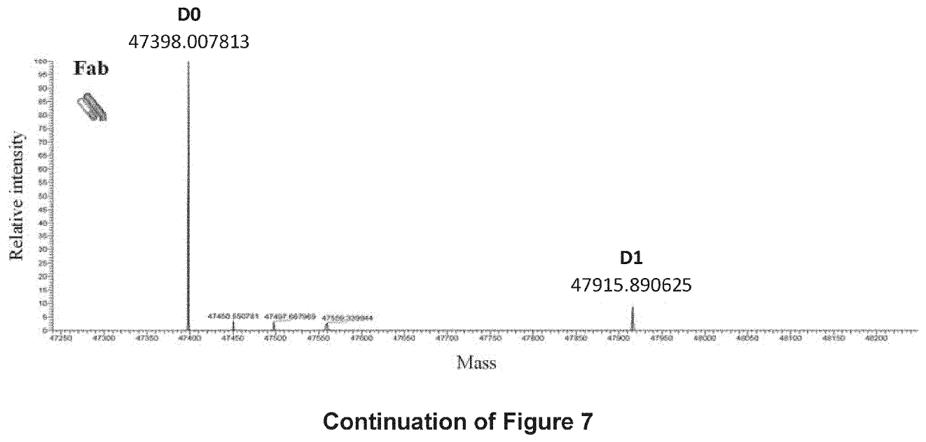

The payload loading ratios (selectivity) between Fc and F(ab)₂ were evaluated by digesting the conjugates with GingisKhan protease (1 unit per μg of antibody conjugate in the presence of 2 mM cysteine, 0.1M Tris, pH 8.0 for 1 hour at 37° C.), and subsequent HRMS analysis (as described above). An exemplary HRMS spectrum of a digested conjugate is shown in FIG. 7. The peaks D0-D2 correspond to the number of conjugated DOTA moieties, whereas G0F/G0F, G0F/G1F and G1F/G1F correspond to different glycans of the Fc domain.

The Degree of Conjugation (DoC) of the trastuzumab-DOTA conjugates was evaluated based on the results of the HRMS analysis (as described above). The results of the HRMS analysis are shown in Table 6 below.

TABLE 6
Characterization of trastuzumab-DOTA
conjugates prepared in Example 6

| | | | Labeled | Labeled | Labeled | Selec- |
|---|---|---|---|---|---|---|
| Conju- | | | mAb | Fc | F(ab)₂ | tivity |
| gate | Compound | DoC | (%) | (%) | (%) | Fc/F(ab)₂ |
| 1 | 24 | 0.53 | 43.0 | 3 | 36 | 0.1 |
| 2 | 25 | 0.39 | 33.5 | 9 | 18 | 0.47 |
| 3 | 26 | 0.47 | 39.4 | 13 | 19 | 0.68 |
| 4 | 27 | 0.68 | 51.0 | 31 | 20 | 1.7 |
| 5 | 28 | 0.98 | 66.0 | 52 | 20 | 2.9 |
| 6 | 29 | 1.20 | 72.0 | 63 | 20 | 4.1 |
| 7 | 30 | 1.31 | 77.0 | 64 | 23 | 3.5 |
| 8 | 31 | 1.09 | 71.0 | 65 | 26 | 3.2 |
| 9 | 33 | 1.31 | 77.0 | 67 | 32 | 2.8 |
| 10 | 34 | 0.27 | 25.0 | 3 | 13 | 0.22 |

These results indicate that compounds (reactive conjugates) 27 to 33 could produce trastuzumab-DOTA conjugates with excellent selectivities for the Fc region of the antibody. In particular, compounds 27, 30 and 31 produced the trastuzumab-DOTA conjugate with excellent selectivities and yields.

The trastuzumab-DOTA conjugates were analyzed by peptide mapping using HRMS in order determine the conjugation sites of the DOTA moieties on the antibody (data not shown). It was found that, in most conjugates, Lys317 of the Fc region was almost quantitatively labeled, while labeling of Lys326 was additionally observed in conjugates with higher DoCs bearing 3 DOTA moieties per Fc region.

### Example 7

### Affinity of Trastuzumab-DOTA Conjugate and Trastuzumab for SK-BR-3 (HER2+) and MD-MB-231 (HER2−) Cells

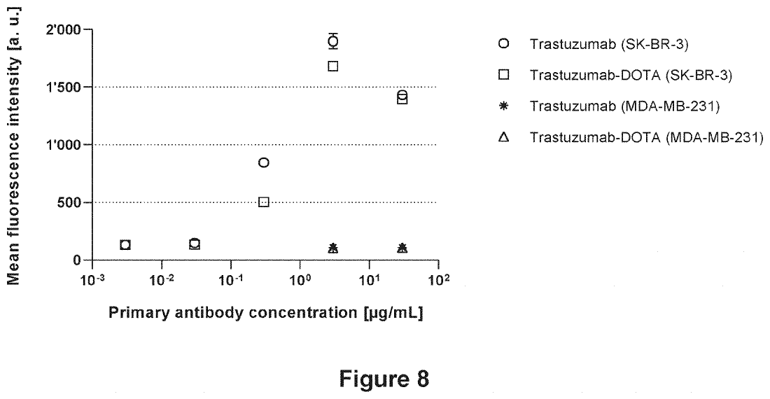

The propensity of the trastuzumab-DOTA conjugate to bind to adenocarcinoma cells was evaluated by measuring the affinity of the conjugate for SKBR-3 (HER2+) and MDA-MB-231 (HER2−) breast adenocarcinoma cell lines. In particular, the affinity of a trastuzumab-DOTA conjugate prepared in the same manner as in Example 6 (similar to conjugate 8; DoC=0.89) was measured using flow cytometry by incubating the trastuzumab-DOTA conjugate and (unlabeled) trastuzumab with the SKBR-3 or MDA-MB-231 cells. Subsequently, a fluorescent secondary antibody specific for trastuzumab was added to measure the binding by fluorescence. The results are shown in FIG. 8.

As can be seen in FIG. 8, the median fluorescence intensity (MFI) increased in a dose-response manner when unlabeled trastuzumab and the trastuzumab-DOTA conjugate were used, confirming that DOTA conjugation does not affect antibody binding to the SKBR-3 cells. The reduced mean fluorescence intensity for trastuzumab and the conjugate at a concentration of 30 μg/mL (SKBR-3 cells) may be explained by the high concentration of primary antibody. No binding to MDA-MB-231 was observed for both samples (negative controls).

### Example 8

### Preparation of FL-Containing Reactive Conjugates

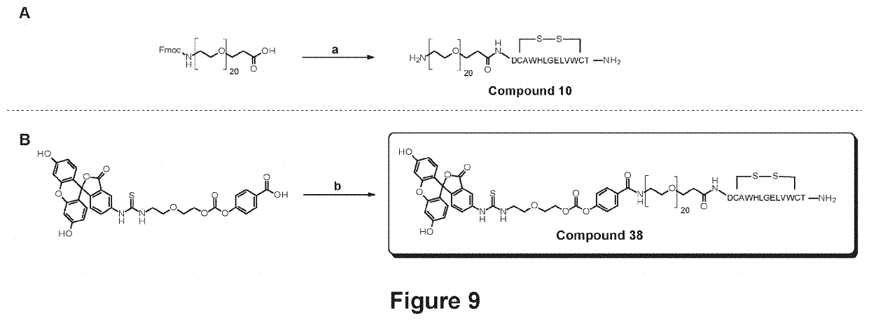

The Fc-binding vectors prepared in Example 1 were converted into reactive conjugates of formula (1) (compounds 35-42) by coupling of compound 19 to the N-terminus of the respective Fc-binding ligands (FIG. 9b) according to the same procedure as described in Example 5 above. The FL-containing reactive conjugates prepared in Example 8 are shown in the table below.

TABLE 7
Structures of FL-containinq reactive conjugates of formula ()

| Compound | Structure |
|---|---|
| 35 | 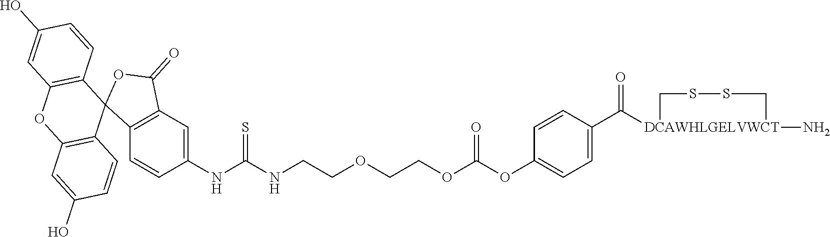 |
| 36 | 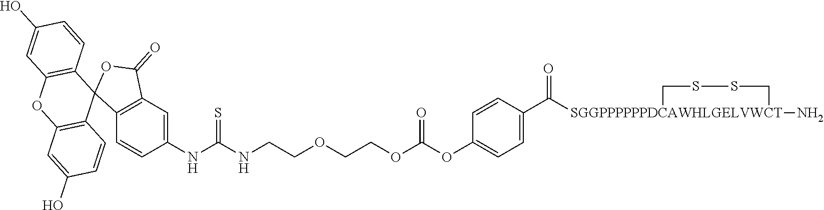 |
| 37 | 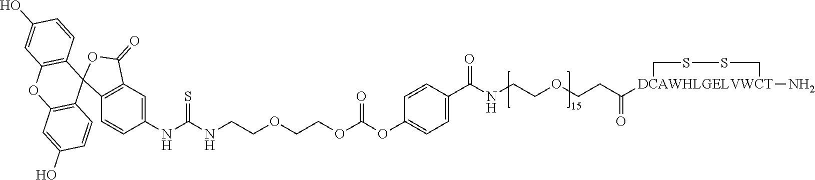 |
| 38 | 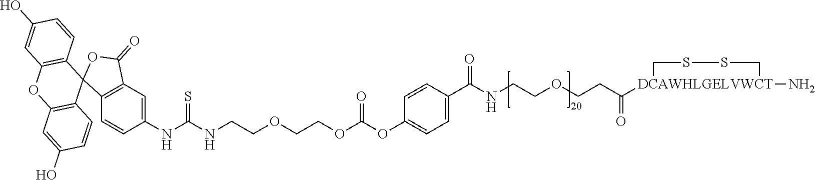 |
| 39 | 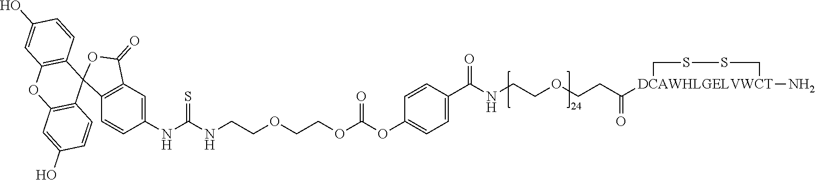 |
| 40 | 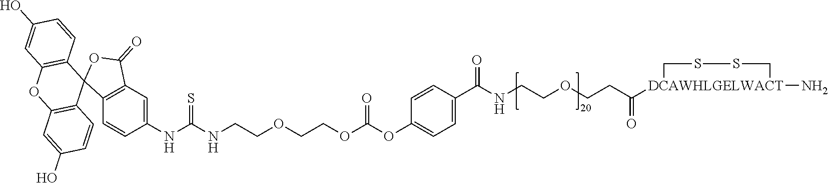 |
| 41 | 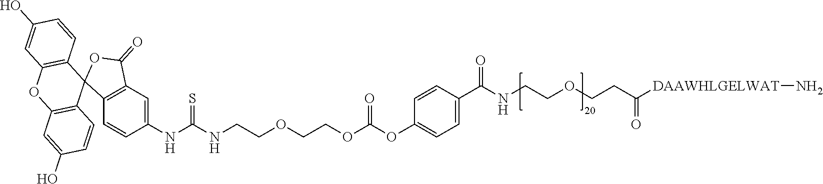 |
| 42 | 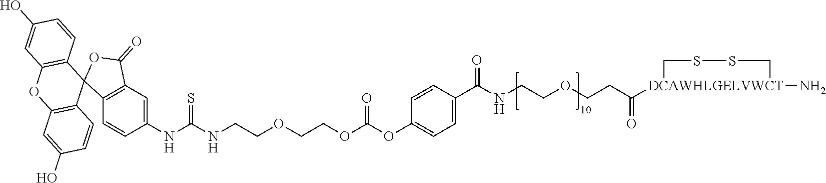 |

The purity of the reactive conjugates was determined by UPLC-MS (as described above). The results are shown in the table below.

TABLE 8
Characterization of reactive conjugates 35-42

| | Calculated | Calculated | Found | Retention | Purity |
|---|---|---|---|---|---|
| Compound | Exact Mass | M + H⁺ | M + H⁺ | time (min) | (%) |
| 35 | 2170.73 | 1086.37 | 1087.04 | 2.12 | 95 |
| 36 | 2954.63 | 985.88 | 985.91 | 2.53 | >95 |
| 37 | 2902.58 | 968.53 | 968.24 | 2.15 | >95 |
| 38 | 3122.91 | 1041.97 | 1041.91 | 2.17 | 93 |
| 39 | 3299.10 | 1100.70 | 1100.53 | 2.17 | >95 |
| 40 | 3094.86 | 1032.62 | 1033.23 | 2.58 | >95 |
| 41 | 3060.83 | 1021.28 | 1021.22 | 2.21 | >95 |
| 42 | 2682.63 | 1342.3 | 1341.7 | 2.90 | 88 |

### Example 9

### Preparation of Trastuzumab-FL Conjugates

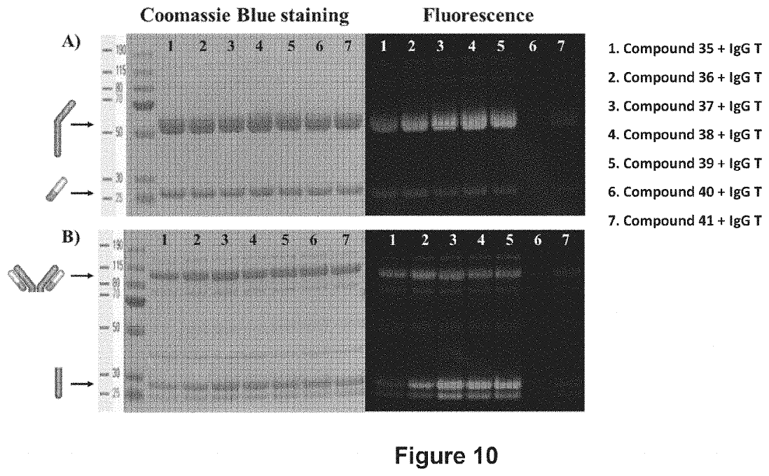

The propensity of the reactive conjugates of Example 8 to react with an antibody was evaluated using trastuzumab as a model system. Trastuzumab-FL conjugates were prepared according to the same procedure as described in Example 6 above using compounds 35-41. The obtained trastuzumab-FL conjugates were analyzed by SDS-PAGE (FIG. 10).

It was found that compounds 36-39 led to efficient trastuzumab labeling and good selectivity for the Fc region (lanes 2-5 in FIG. 10). No trastuzumab labeling was observed when compounds 40 and 41 were used (negative controls; lanes 6 and 7).

To prepare a trastuzumab-FITC (random) conjugate, 10 eq of FITC (0.47 μmol, 25.5 μL) in DMSO was added to a solution of trastuzumab (1 eq, 47 nmol; commercial trastuzumab Herceptin® available from Roche which was buffer-exchanged into phosphate-buffered saline (PBS) prior to the conjugation) diluted in 50 mM NaHCO₃ pH 9.0 and the reaction mixture (1.4 mL) was stirred at room temperature for 16 hours.

After FITC conjugation, the reaction buffer was diluted with 0.1 M glycine pH 2.5. The antibody conjugate was then purified by gel filtration chromatography using a pre-equilibrated column manually packed with Bio-spin P-30 fine beads (bed height: 5.0 cm) and then eluted with 0.1M glycine pH 2.5. The purified antibody conjugate fractions were neutralized with 1M phosphate buffer pH 8.5.

The conjugation of the moiety to trastuzumab was evaluated by HRMS analysis (as described above). The results of the HRMS analysis of trastuzumab-FITC and trastuzumab-FI are shown in Table 9 below.

TABLE 9
Characterization of trastuzumab-FI
and -FITC conjugates prepared in Example 9

| | | | Labeled | Labeled | Labeled | Selec- |
|---|---|---|---|---|---|---|
| Conju- | | DoC | mAb | Fc | F(ab)₂ | tivity |
| gate | Compound | mAb | (%) | (%) | (%) | Fc/F(ab)₂ |
| 11 | 42 | 1.17 | 67% | 53% | 16% | 3.94 |
| 12 | FITC | 3.68 | 100% | 85% | 65% | 0.82 |

### Example 10

### Affinity of Trastuzumab-FL, -FITC Conjugates (11, 12) for BT-474 (HER2+) and MDA-MB33 (HER2−) Cells

The propensity of the trastuzumab-FL, -FITC conjugates to bind to adenocarcinoma cells was evaluated by measuring the affinity of the conjugate for BT-474 (HER2+) and MDA-MB33 (HER2−) breast adenocarcinoma cell lines. In particular, the affinity of a trastuzumab-FITC conjugate prepared as described in Example 9 (MS data is shown in a Table 9) was measured using flow cytometry by incubating the trastuzumab-FL, -FITC conjugates and (unlabeled) trastuzumab with the SKBR-3 or MDA-MB-231 cells.

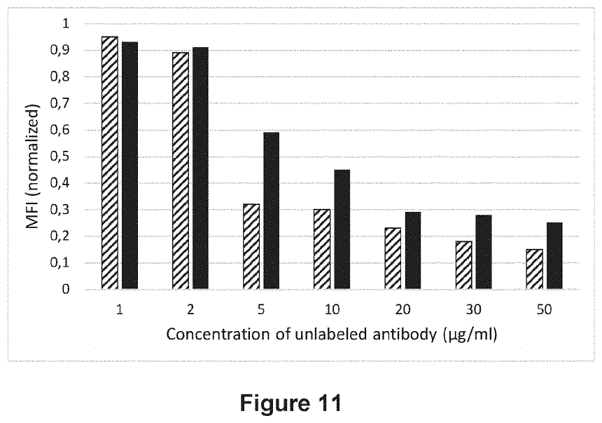

As can be seen in FIG. 11, the mean fluorescence index (MFI) of FITC-, Fl-conjugated antibodies decreased in a dose-response manner upon addition of unlabeled trastuzumab (competitor antibody). An MFI reduction of close to 50% was observed for conjugate 11 while the MFI decrease is close to 70% for conjugate 12 at equimolar concentration of labeled and unlabeled antibodies (10 μg/ml). These results suggest that Fluorescein conjugation does not affect antibody binding to HER2 cells while the random labelling of conjugate 12 impacts the affinity of the antibody. No binding to MDA-MB33 was observed for both samples (negative controls, data not shown).

### Example 11

### Preparation of Antibody-FL Conjugates Using Trastuzumab, Commercial Trastuzumab, Alemtuzumab, Bevacizumab and Rituximab

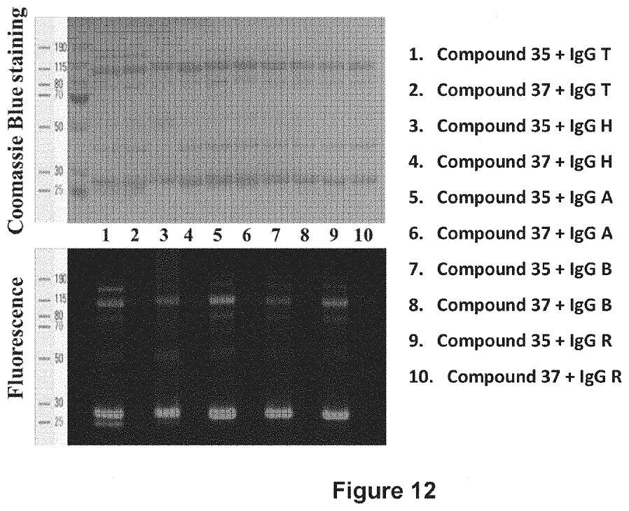

The propensity of the reactive conjugates of the present invention to react with different antibodies was evaluated using trastuzumab, commercially available trastuzumab (Herceptin®), alemtuzumab, bevacizumab and rituximab. Antibody-FL conjugates were prepared according to the same procedure as described in Example 6 above using compound 38 and the aforementioned antibodies. The conjugates were analyzed by SDS-PAGE (FIG. 12).

It was found that compound 38 led to efficient antibody labeling (lanes 1, 3, 5, 7 and 9 in FIG. 12). No trastuzumab labeling was observed when compound 40 was used (lanes 2, 4, 6, 8 and 10).

### Example 12

### Preparation of DBCO-Containing Reactive Conjugate and Trastuzumab-DBCO Conjugate

The Fc-binding ligand prepared in Example 1 (compound 10) was converted into the corresponding reactive conjugate of formula (1) by coupling of compound 20 to the N-terminus of the respective Fc-binding ligand. The structures of the DBCO-containing reactive conjugate prepared in Example 12 is shown in the table below.

TABLE 10
Structure of DBCO-containing reactive conjugate of formula (1)

| Compound | Structure |
|---|---|
| 43 | 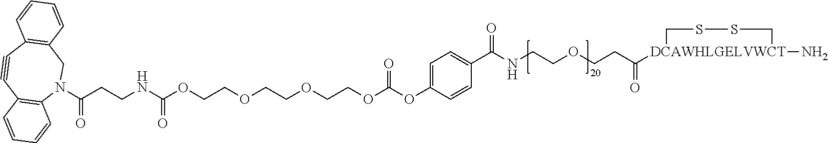 |

The purity of the reactive conjugate was determined by UPLC-MS (as described above). The results are shown in the table below.

TABLE 11
Characterization of reactive conjugate 43

| | Calculated | Calculated | Found | Retention | Purity |
|---|---|---|---|---|---|
| Compound | Exact Mass | M + H⁺ | M + H⁺ | time (min) | (%) |
| 43 | 3080.54 | 1027.85 | 1027.97 | 2.42 | >95 |

Trastuzumab-DBCO conjugates were prepared according to the same procedure as described in Example 6 above using compound 43 and commercial trastuzumab (Herceptin®). The conjugate was digested with GingisKhan and analyzed by HRMS as described above. The results are shown in the table below.

TABLE 12
Characterization of trastuzumab-DBCO
conjugate prepared in Example 12

| | | Labeled | Selectivity |
|---|---|---|---|
| Conjugate | DoC | mAb (%) | Fc/F(ab)₂ |
| 13 | 0.88 | 51 | 9.2 |

### Example 13

### Preparation of Immobilized FL-Containing Reactive Conjugate and Solid-Phase Modification of Trastuzumab

A reactive conjugate immobilized on a solid support was prepared, and its propensity to react with trastuzumab was evaluated.

A biotinylated Fc-binding vector was prepared by standard Fmoc/tBu-based SPPS using a 4-Fmoc-hydrazinobenzoyl AM NovaGel™ (loading 0.61 mmol/g) and a Liberty Blue™ automated microwave peptide synthesizer (available from CEM Corp., Germany). Coupling reactions for amide bond formation were performed over 4 min at room temperature using 0.2 M of Fmoc-amino-acids pre-activated with 0.5 M DIC and 1 M OxymaPure in DMF. Fmoc deprotection was performed with 10% piperazine in DMF (v/v).

After completion of the synthesis, the peptide was cleaved from the resin manually by resuspending the resin in DMF and mixing with 1.4 eq CuII(AcO)_{2*}H₂O, 3.5 eq biotin-PEG4-NH₂ and 3 eq pyridine. The reaction was stirred 4 hours at room temperature.

The cleavage mixture was filtered, the peptide was precipitated with water and filtered. The pellet was dissolved in the cleavage cocktail (TFA/TIS/water 90:5:5) and the side chains of the peptide were deprotected by stirring for 2 hours at room temperature. The mixture was concentrated and the crude peptide (compound 41) was precipitated with cold diethyl ether, centrifuged, washed with cold diethyl ether, dried, dissolved in ultrapure water/acetonitrile, lyophilized, and purified by HPLC.

A solution of Fmoc-NH-(PEG)₂₀-COOH (1.3 eq, 4.7 μmol) and HATU (1.2 eq, 4.33 μmol) in DMF was stirred for 1 min, and DIEA (10 eq, 35.8 μmol) was added. After 3 min of pre-activation, the biotinylated Fc-binding peptide (compound 44) in DMF (1 eq, 3.58 μmol) was added to the reaction mixture and stirred for 1-2 hours at room temperature to prepare compound 45. Reaction completion was monitored by ULPC-MS. The peptide was then precipitated with cold diethyl ether. Fmoc deprotection was performed with 20% piperidine in DMF (v/v) for 30 min at room temperature, followed by precipitation of the peptide with cold diethyl ether, and purification by HPLC.

The biotinylated Fc-binding ligand was converted into the reactive conjugate (compound 46) by coupling of compound 19 to the N-terminus of compound 45 according to the same procedure as described in Example 5 above. The structure of the compounds prepared in Example 13 are shown in the table below.

TABLE 13
Structures of the compounds prepared in Example 13

| Compound | Structure |
|---|---|
| 45 | 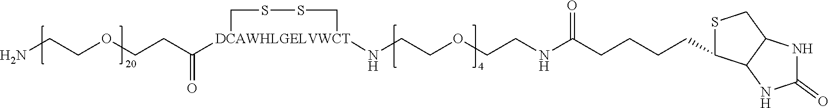 |
| 46 | 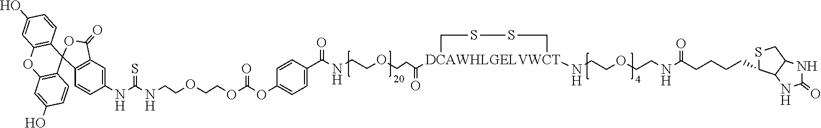 |

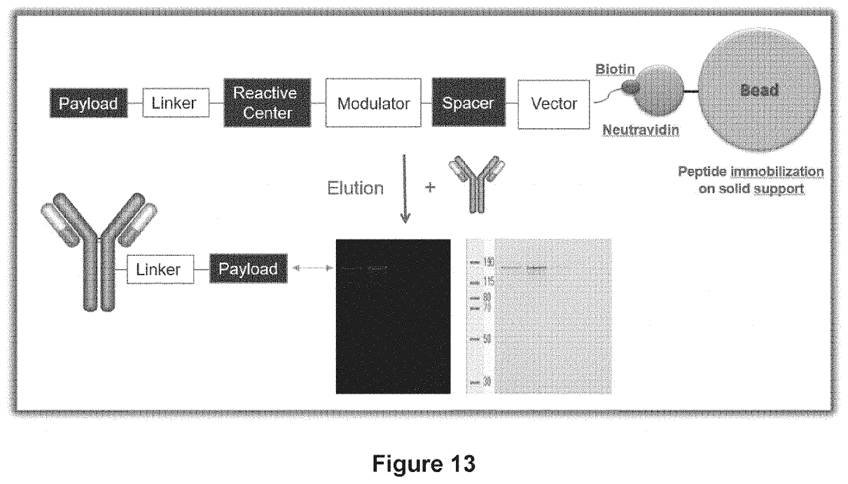

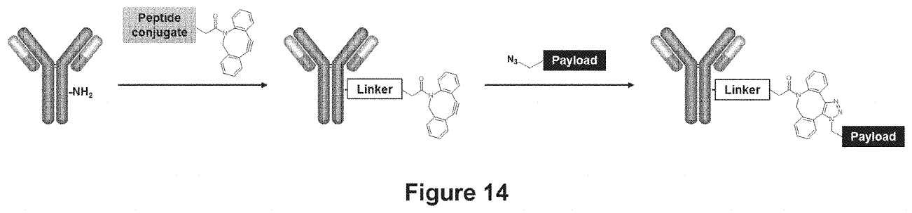

To immobilize the biotinylated reactive conjugate on a solid support, NeutrAvidin Agarose Resin (Thermo Fisher) was packed into a column (Fisher Scientific) and washed with binding buffer (0.1 M phosphate buffer, 0.15 M sodium chloride, pH 7.2). Compound 46 (2.1 nmol) was incubated with the washed NeutrAvidin agarose beads (40 μl beads: 7.5 μg peptide) for 30 min at room temperature (FIG. 13).

The beads were washed 4 times with binding buffer and then, 50 mM Bicine pH 9.0 was added to increase the pH. Trastuzumab in PBS pH 7.0 (2.1 nmol) was added to the beads, the mixture was stirred for 2 h at room temperature, and then washed 3-4 times with binding buffer. Labeled Trastuzumab was eluted (100 μl, 0.1 M glycine, pH 2.5) into a collection tube containing neutralization buffer (1M phosphate buffer pH 8.5) at a 1:10 volumetric ratio. The elution step was repeated, and fractions were combined. The eluted labeled Trastuzumab was then buffer exchanged with PBS pH 7.0 using a 30 kDa MWCO Vivaspin® 500 centrifugal concentrators.

The antibody was then analyzed by SDS-PAGE. The gel showed one fluorescent band indicating successful conjugation of the FL moiety to trastuzumab.

### Example 14

### Preparation of Other Payload-Carbonate-Containing Reactive Conjugates

The Fc-binding vectors prepared in Example 1 were converted into reactive conjugates of formula (1) (compounds 47-49) by coupling different payloads (DTPA, PCTA, DFO) to the NH₂-carbonate-PEG₁₀-Fc-III. The structures of these payload-containing reactive conjugates are shown in the table below.

TABLE 14
Structures of other payload-carbonate-containing reactive conjugates of
formula (1)

| Compound | Structure |
|---|---|
| 47 | 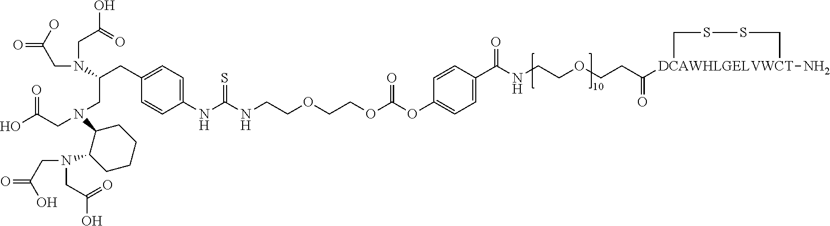 |
| 48 | 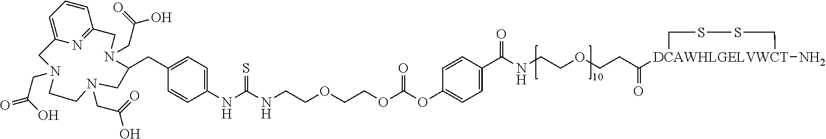 |
| 49 | 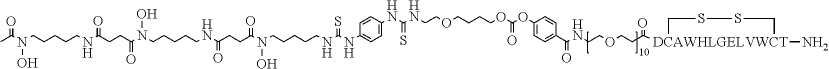 |

The purity of the reactive conjugates was determined by UPLC-MS (as described above). The results are shown in the table below.

TABLE 15
Characterization of peptide reactive conjugates

| | Calculated | Calculated | Found | Retention | Purity |
|---|---|---|---|---|---|
| Compound | Exact Mass | M + H⁺ | M + H⁺ | time (min) | (%) |
| 47 | 2886.63 | 1444.3 | 1444.1 | 2.58 | >95 |
| 48 | 2820.17 | 1411.1 | 1410.8 | 2.51 | 99 |
| 49 | 3045.52 | 1523.8 | 1524 | 2.54 | 86 |

Preparation of NH₂-Carbonate-PEG₁₀-Fc-III:

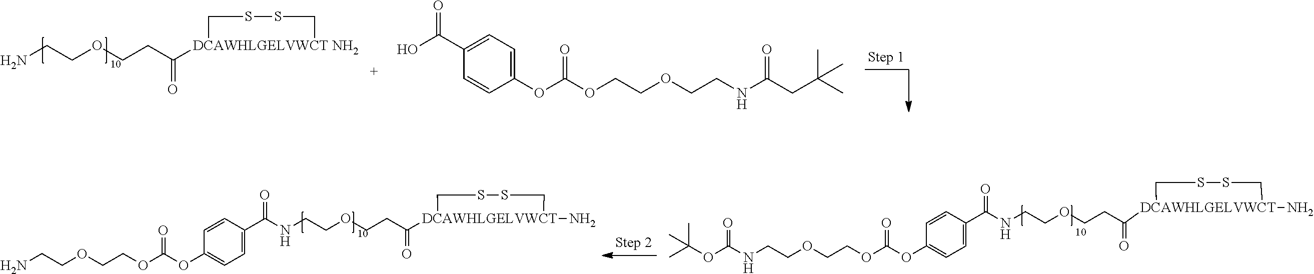

Step 1. DIEA was added to a solution of 4-[2-[2-(tert-butoxycarbonylamino)ethoxy]ethoxycarbonyloxy]benzoic acid (2.35 mg, 6.4 μmol, 1.3 eq.) in DMF (0.65 mL) at rt. After stirring at rt for 1 min, HATU.HPF₆ (2.81 mg, 5.4 μmol, 1.1 eq.) was added to the reaction mixture. After stirring at rt for 3 min, a solution of compound 7 (10.0 mg, 4.9 μmol, 1.0 eq.) in DMF (0.65 mL) was added to the reaction mixture. After stirring at rt for 18 h, 2 drops of a 0.1% TFA in water solution was added. Purification on C18 (12 g, 30 to 70% of ACN+0.1%TFA in water+0.1% TFA over 12 CV) afforded BocHN-carbonate-PEG₁₀-Fc-III (2.4 mg, 1.0 μmol, UV purity 95%, 20% yield) as a white powder after freeze-drying. UPLC-MS: Rt=2.78 min, m/z=1147 [M−Boc+2H]²⁺, 1195 [M−2H]²⁻.

Step 2. TFA was added to a solution of BocHN-carb-PEG10-FcIII (23.9 mg, 8.3 μmol, 1.0 eq.) in solution in DCM (0.5 mL). The reaction mixture was stirred at rt for 1.5 h then concentrated in vacuo. A mixture of ACN/Water (1:1, 5 mL) was added and the mixture was freeze dried to H₂N-carbonate-PEG10-Fc-III (23.7 mg, 8.3 μmol, UV purity 99%, quant. yield) as a white powder. UPLC-MS: Rt=2.20 min, m/z=1147 [M+2H]²⁺, 1145 [M−2H]²⁻.

Preparation of DTPA-Carbonate-PEG₁₀-Fc-III:

p-SCN-Bn-CHX-A″-DTPA.3HCl (4.47 mg, 6.0 μmol, 1.0 eq.) was added to a solution of NH₂-carbonate-PEG10-Fc-III (14.35 mg, 6.0 μmol, 1.0 eq.) in DMF (0.3 mL) at rt. The reaction mixture was stirred at rt for 5 min then triethylamine (4.0 Å, 30.0 μmol, 5.0 eq.) was added. After stirring at rt for 36 h, p-SCN-Bn-CHX-A″-DTPA.3HCl (0.90 mg, 1.2 μmol, 0.2 eq.) and triethylamine (0.5 Å, 3.6 μmol, 0.6 eq.) were added and the reaction mixture was stirred at rt for 18 h. Purification by preparative HPLC (30 to 60% of ACN+0.1% of FA in water+0.1% FA) afforded DTPA-carbonate-PEG₁₀-Fc-III (1.4 mg, 0.52 μmol, 8.7% yield) as white powder after freeze-drying.

Preparation of PCTA-Carbonate-PEG₁₀-Fc-III:

p-SCN-Bn-PCTA.3HCl (4.15 mg, 6.5 μmol, 1.05 eq.) was added to a solution of NH₂-carbonate-PEG₁₀-Fc-III (14.9 mg, 6.2 μmol, 1.0 eq.) in DMF (0.1 mL) at rt. The reaction mixture was stirred at rt for 5 min then triethylamine (4.2 Å, 30.0 μmol, 5.0 eq.) was added. After stirring at rt for 3 h, 1 p-SCN-Bn-PCTA.3HCl (4.15 mg, 6.5 μmol, 1.05 eq.) to the reaction mixture at rt. After stirring at rt for 16 h, purification by preparative HPLC (28 to 37% of ACN+0.1% of TFA in water+0.1% TFA) afforded PCTA-carbonate-PEG₁₀-Fc-III (2.53 mg, 8.97 μmol, 14% yield) as a white powder after freeze-drying.

Preparation of DFO-Carbonate-PEG₁₀-Fc-III:

DIEA (10 Å, 80.0 μmol, 16.0 eq.) was added to a solution of NH₂-carbonate-PEG₁₀-Fc-III (12.42 mg, 4.9 μmol, 1.0 eq.) and DFO-NHS (8.2 mg, 5.9 μmol, 1.2 eq.) in DMF (0.4 mL) at rt. After stirring at rt for 3.5 h, ACN/water/TFA (1:1:0.5%, 0.2 mL) was added and the reaction mixture was stirred at rt for 5 min. Purification by preparative HPLC (25 to 60% of ACN+0.1%FA in water+0.1%FA) afforded DFO-carbonate-PEG₁₀-Fc-III (1.6 mg, 0.45 μmol, UV purity 86%, 9% yield) as a white powder after freeze-drying.

### Example 15

### Preparation of Trastuzumab-DTPA/PCTA/DFO Conjugates

The propensity of the reactive conjugates of Example 14 to react with an antibody was evaluated using trastuzumab. Trastuzumab-DTPA/PCTA/DFO conjugates were prepared according to the same procedure as described in Example 6 above using compounds 47-49. The obtained trastuzumab-DTPA/PCTA/DFO conjugates were analyzed by HRMS (Table 16).

TABLE 16
Characterization of trastuzumab-DTPA/PCTA/DFO conjugates.

| | | | Labeled | Labeled | Labeled | Selec- |
|---|---|---|---|---|---|---|
| Conju- | | DoC | mAb | Fc | F(ab)₂ | tivity |
| gate | Compound | mAb | (%) | (%) | (%) | Fc/F(ab)₂ |
| 14 | 47 (DTPA) | 0.87 | 63% | 37% | 10% | 3.73* |
| 15 | 48 (PCTA) | 0.68 | 52% | 24% | 15% | 1.60* |
| 16 | 49 (DFO 2 eq) | 0.47 | 39% | 5% | 14% | 0.39* |
| 17 | 49 (DFO 6 eq) | 1.52 | 79% | 20% | 15% | 1.34* |

*Values were extrapolated: Selectivity Fc/F(ab)₂ = (DoC mAb − DoC F(ab)₂/Doc(Fab)₂

_*Values were extrapolated: Selectivity Fc/F(ab)₂=(DoC F (ab)₂)/Doc (Fab)₂

### Example 16

### Preparation of Fl-Containing Reactive Conjugates with Different Chemistry or Reactive Modulator

The Fc-binding vectors prepared in Example 1 were converted into reactive conjugates by coupling of FL-carbonate-naphtalene/-carbonate-isoquinoline/-CH₂CH₂-thioester (compounds 21-23) to the N-terminus of the Fc-binding ligand 7 according to the procedure described below. The structures of these payload-containing reactive conjugates prepared in Example 16 are shown in the table below.

TABLE 17
Structures of Fl-containing reactive conjugates reactive modulator

| Compound | Structure |
|---|---|
| 50 | 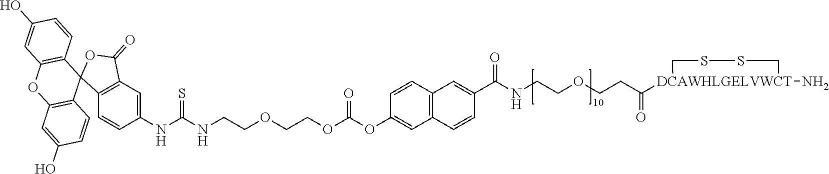 |
| 51 | 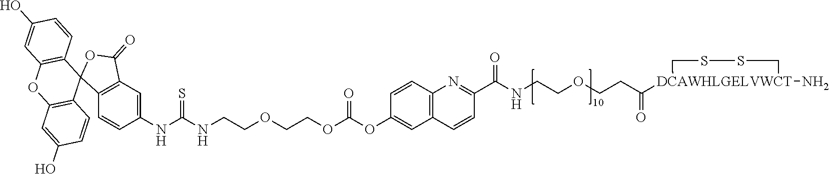 |
| 52 | 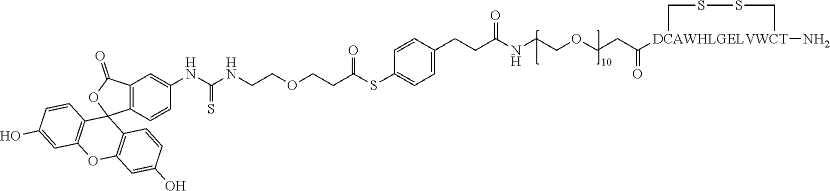 |

Preparation of Compound 50 (Naphtalene):

To a solution of 2,5-dioxopyrrolidin-1-yl 6-(((2-(2-(3-(3′,6′-dihydroxy-3-oxo-3H-spiro[isobenzofuran-1,9′-xanthen]-5-yl)thioureido)ethoxy)ethoxy)carbonyl)oxy)-2-naphthoate (19.0 mg, 0.023 mmol) and compound 7 (40.0 mg, 0.019 mmol) in N,N-dimethylformamide (1.00 mL) was added DIPEA (10.0 μL, 0.057 mmol) at ambient temperature. The mixture was stirred for 3 hours, then purified on a 60 g C18 column with a 5-95% acetonitrile (0.1% formic acid) in water (0.1% formic acid) eluent. Desired fractions were combined and freeze dried. The resulting material was combined with a similar batch obtained from the reaction of 2,5-dioxopyrrolidin-1-yl 6-(((2-(2-(3-(3′,6′-dihydroxy-3-oxo-3H-spiro[isobenzofuran-1,9′-xanthen]-5-yl)thioureido)ethoxy)ethoxy)carbonyl)oxy)-2-naphthoate (11.0 mg, 0.014 mmol) with TFA.PEG₁₀-FcIII (30.0 mg, 0.014 mmol) and DIPEA (7.00 μL, 0.042 mmol) in N,N-dimethylformamide (1.00 mL), and further purified on a 60 g C18 column with a 20-60% acetonitrile (0.1% formic acid) in water (0.1% formic acid) eluent. Desired fractions were combined and freeze dried to afford the tile compound (11.9 mg, 13% combined yield, purity 95%). UPLC4-MS: Rₜ=1.95 min., 94.5%. ESI: m/z=911.8 [M+3H]/3⁺.

Preparation of Compound 51 (Isoquinoline):

To a solution of 2,5-dioxopyrrolidin-1-yl 6-(((2-(2-(3-(3′,6′-dihydroxy-3-oxo-3H-spiro[isobenzofuran-1,9′-xanthen]-5-yl)thioureido)ethoxy)ethoxy)carbonyl)oxy)quinoline-2-carboxylate (11.0 mg, 0.014 mmol) and TFA.PEG₁₀-FcIII (30.0 mg, 0.014 mmol) in N,N-dimethylformamide (1.00 mL) was added DIPEA (7.00 μL, 0.042 mmol) at ambient temperature. The mixture was stirred for 2 hours, then purified on a 60 g C18 column with a 20-60% acetonitrile (0.1% formic acid) in water (0.1% formic acid) eluent. Desired fractions were combined and freeze dried to afford the title compound (8.70 mg, yield 23%, purity 93%) as a yellow powder. UPLC-MS: Rₜ=1.93 min. ESI: m/z=912.0 [M+3H]/3⁺.

Preparation of Compound 52 (Thioester):

To a solution of 2,5-dioxopyrrolidin-1-yl 3-(4-((3-(2-(3-(3′,6′-dihydroxy-3-oxo-3H-spiro[isobenzofuran-1,9′-xanthen]-5-yl)thioureido)ethoxy)propanoyl)thio)phenyl)propanoate (12.8 mg, 0.0163 mmol) and compound 7 (21.1 mg, 0.00980 mmol) in N,N-dimethylformamide (1.00 mL), was added DIPEA (8.52 μL, 0.0489 mmol). Stirring continued at room temperature for 2 hours. The reaction was purified directly by reverse phase chromatography (Biotage Isolera, 60 g, C18 SNAP Ultra Biotage cartridge) using water containing 0.1% formic acid and acetonitrile containing 0.1% formic acid (80:20 to 30:70). The fractions containing product were freeze dried to give the desired compound (4.88 mg, yield 4%, purity 87%) as a yellow solid. UPLC4-MS: Rₜ=1.67 min. ESI: m/z=904 [M+3H]/3⁺.

### Example 17

### Preparation of Trastuzumab-FL Conjugates

The propensity of the reactive conjugates of Example 16 to react with an antibody was evaluated using trastuzumab. Trastuzumab-FL conjugates were prepared according to the same procedure as described in Example 6 above using compounds 50-52. The obtained trastuzumab-FL conjugates were analyzed by HRMS (Table 18).

TABLE 18
Characterization of trastuzumab-FI conjugates.

| | | | Labeled | Labeled | Labeled | Selec- |
|---|---|---|---|---|---|---|
| Conju- | | DoC | mAb | Fc | F(ab)₂ | tivity |
| gate | Compound | mAb | (%) | (%) | (%) | Fc/F(ab)₂ |
| 18 | 50 | 0.47 | 40% | 14% | 18% | 0.80 |
| 19 | 51 | 1.10 | 70% | 22% | 20% | 1.17 |
| 20 | 52 | 1.04 | 67% | 36% | 32% | 1.09 |

1. Compound represented by the following formula (1):
P—Y—S—V (1)
wherein,
P is a payload;
Y is a reactive moiety capable of reacting with the side chain of an amino acid, preferably a moiety capable of reacting with the side chain of lysine;
V is a vector capable of interacting with the fragment crystallizable (Fc) region of an antibody or fragment thereof, said antibody fragment being optionally incorporated into an Fc-fusion protein;
S is a spacer having a length Z, wherein Z is a length such that when the vector V interacts with the Fc region of an antibody or fragment thereof, the reactive moiety Y is able to react with the side chain of an amino acid residue on said antibody or antibody fragment;

2. The compound of claim 1, wherein the payload comprises a moiety selected from:
(i) a moiety selected from
a labelling moiety which may include a radionuclide, preferably a chelating agent such as 1,4,7,10-tetraatacyclododecane-1, 4,7,10-tetraacetic acid (DOTA), diethylenetriamine pentaacetic acid (DTPA), cyclohexyl diethylenetriamine pentaacetic acid (CH-X-DTPA), 3,6,9,15-Tetraazabicyclo[9.3.1]pentadeca-1(15), 11,13-triene-3,6,9-triacetic acid (PCTA) or desferrioxamine (DFO), wherein said chelating agent optionally chelates a radionuclide;
a chromophore;
a fluorophore such as fluorescein or rhodamine; and
a labelling moiety containing a radionuclide such as ¹²⁵I, ¹²³I, ¹³¹I, ¹⁸F, ¹¹C, ¹⁵O, ¹⁸F, e.g. a moiety derived from 4-hydroxyphenylpropionate containing a radionuclide such as ¹²⁵I, ¹²³I or ¹³¹I;
(ii) a moiety selected from a moiety comprising a conjugation group including an optionally substituted conjugated diene; an optionally substituted tetrazine (TZ); an optionally substituted alkyne or azide; an optionally substituted dibenzocyclooctyne (DBCO); an optionally substituted trans-cyclooctene (TCO), an optionally substituted bicyclo[6.1.0]nonyne (BCN); an optionally substituted aldehyde; an optionally substituted ketone; and an optionally substituted hydrazine;
(iii) a moiety derived from a drug selected from
an antineoplastic agent such as a DNA-alkylating agent e.g. duocarmycin;
a topoisomerase inhibitor e.g. doxorubicin;
an RNA-polymerase II inhibitor e.g. alpha-amanitin;
a DNA cleaving agent e.g. calicheamicin;
an antimitotic agent or microtubule disruptor e.g. a taxane an auristatin or a maytansinoid;
an anti-metabolite;
a kinase inhibitor such as ipatasertib;
an immunomodulatory agent;
an anti-infectious disease agent;
and radioisotopes and/or pharmaceutically acceptable salts thereof;

3. The compound of claim 1, wherein the payload is a chelating agent that optionally chelates a radionuclide, which chelating agent is preferably a moiety derived from DTPA, CH-X-DTPA, DFO, 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), DOTA, 1,4,7-triazacyclononane-1,4,7-triacetic acid (NOTA), ethylenediaminetetraacetic acid (EDTA), ethylenediaminediacetic acid, triethylenetetraminehexaacetic acid (TTNA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)tracetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4, 7-bis((hydroxy(hydroxymethyl)phosphoryl)methyl)-1,4,7-triazonan-1-yl)methyl)(hydroxy)phosphoryl)propanoic acid (NOPO), PCTA, 2,2′,2″,2″′-(1,4,7,10-tetraazacyclotridecane-1,4,7,10-tetrayl)tetraacetic acid (TRITA), 2,2′,2″,2″′-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)tracetamide (TRITRAM), trans-N-dimethyl-cyclam, 2,2′,2″-(1,4,7-triazacyclononane-1,4,7-triyl)tracetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), or derivatives thereof; more preferably a moiety derived from DTPA, DOTA, DFO, NOTA, PCTA, CH-X-DTPA, NODAGA or DOTAGA.

4. The compound of claim 2, wherein the radionuclide is selected from ¹²⁴I, ¹³¹I, ⁸⁶Y, ⁹⁰Y, ¹⁷⁷Lu, ¹¹¹In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²_{Bi,} ²¹³Bi, ⁷²As, ²¹¹At, ²²⁵Ac, ²²³Ra, ⁹⁷Ru, ¹⁴⁹Tb, ¹⁵²Tb, ¹⁶¹Tb, ^{99m}Tc, ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F, preferably from ⁸⁹Zr, ¹¹¹In, ⁶⁴Cu, ¹⁷⁷Lu, ⁶⁸Ga, ^{99m}Tc, ²⁰³Pb, ⁷²As, ⁵⁵Co, ⁹⁷Ru, ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, in particular ¹¹¹In.

5. The compound of claim 1, wherein the payload is a moiety derived from exatecan, PNU-159682, amanitin, duocarmycin, auristatin, maytansine, tubulysin, calicheamicin, SN-38, taxol, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof.

6. The compound of claim 1, wherein P is represented by the following formula (2):
P¹-*′ (2)
wherein,
P¹ is a payload as defined in any of claims 2 to 5;
L is a linker, preferably a linker comprising one or more atoms selected from carbon, nitrogen, oxygen, and sulfur, which is optionally cleavable;
*′ indicates covalent attachment to the reactive moiety (Y).

7. The compound of claim 6, wherein the linker is selected from
(a1) an alkylene group having from 1 to 12 carbon atoms, preferably an alkylene group having from 2 to 6 carbon atoms such as a propylene group;
(b1) a polyalkylene oxide group with 2 or 3 carbon atoms having from 1 to 36 repeating units; preferably a group represented by the formula —NH—(CH₂CH₂O)ₙ₁—CH₂CH₂— wherein n1 is an integer of 0 to 35, e.g. 1 to 20;
(c1) a peptidic group having 2 to 12 amino acids.

8. The compound of claim 1, wherein the reactive moiety is represented by the following formula (3a):
**—(F1-RC—F2)-* (3a)
wherein,
RC is a reactive center, preferably an electrophilic reactive center, and more preferably a group selected from C═O and C═S;
F1 is a single covalent bond, an atom, or a group of atoms; preferably an atom selected from O and S, or a group of atoms comprising one or more atoms selected from C, N, O, and S; more preferably an atom selected from O and S;
F2 represents an atom, or a group of atoms; preferably an atom selected from O, and S, or a group of atoms comprising one or more atoms selected from C, N, O, and S; more preferably an atom selected from O and S;
* indicates covalent attachment to the spacer (S); and
** indicates covalent attachment to the payload (P).

9. The compound of claim 8, wherein the reactive moiety is represented by one of the following formulae (4a) to (4n): 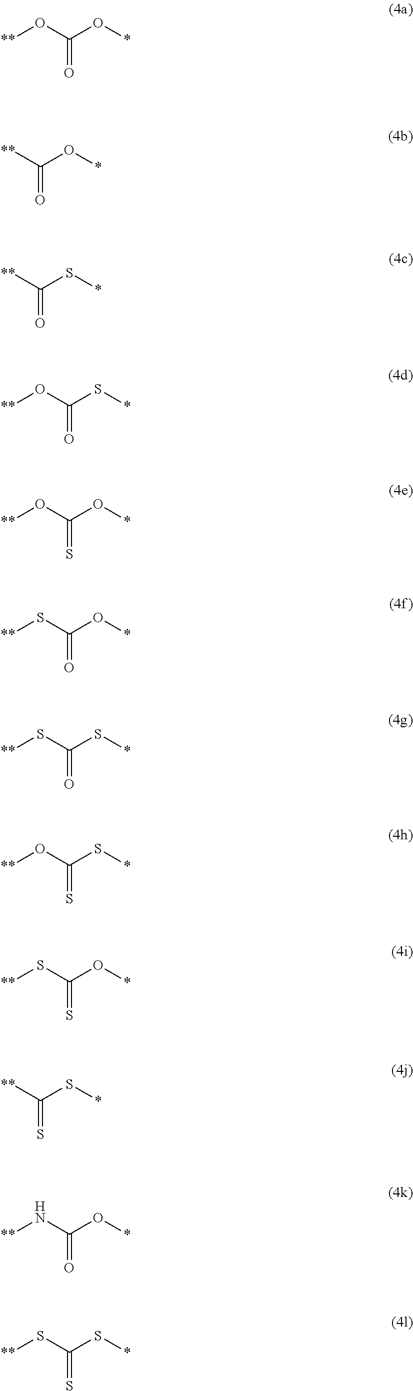 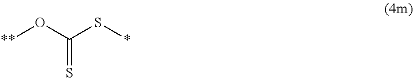
wherein * indicates covalent attachment to the spacer (S), and ** indicates covalent attachment to the payload (P).

10. The compound of claim 1, wherein the reactive moiety is represented by the following formula (3b):
**—(F1-RC—F2)-(M)-* (3b)
wherein,
RC is a reactive center, preferably an electrophilic reactive center, and more preferably a group selected from C═and C═S;
F1 is a single covalent bond, an atom, or a group of atoms; preferably an atom selected from O and S, or a group of atoms comprising one or more atoms selected from C, N, O, and S; more preferably an atom selected from O and S;
F2 represents an atom, or a group of atoms; preferably an atom selected from O, and S, or a group of atoms comprising one or more atoms selected from C, N, O, and S; more preferably an atom selected from O and S;
M is a group capable of modulating the electron density and stability of F2, preferably a group capable of withdrawing electrons;
* indicates covalent attachment to the spacer (S); and
** indicates covalent attachment to the payload (P).

11. The compound of claim 10, wherein the group capable of modulating the electron density and stability of F2 is represented by the following formula (3c):
***′-M′-B—C—* (3c)
wherein,
M′ is an aryl group having 6, 10 or 14 ring members and 1, 2 or 3 condensed rings, respectively, or a heteroaryl group having 5 to 20 ring members, 1, 2 or 3 condensed rings and 1 to 4 heteroatoms independently selected from N, O and S, which may be substituted with one or more substituents; preferably a phenyl group, a naphthyl group, a pyridyl group, a quinolinyl group, an isoquinolinyl group or a benzotriazolyl group, which may be substituted with one or more substituents, each substituent being preferably selected from —F, —Br, —Cl, —I, —NO₂, —CN, —C₁₋₆-alkoxy, —C₁₋₆-amido such as —C(O)NH₂, and combinations thereof such as —CCl₃, —CF₃ or —CH₂NO₂;
B is a single covalent bond, O, S, NR′ wherein R′ represents a hydrogen atom, OH, an alkyl group or a cycloalkyl group, a C₂₋₆-alkenylene, a C₂₋₆-alkynylene, a group having the general formula:
—(CH₂)ₙ₁—(H¹)ₓ₁—(CH₂)ₙ₂—(H²)ₓ₂—(CH₂)ₙ₃—(H³)ₓ₃—(CH₂)ₙ₄— (3c′)
wherein,
each of n1, n2, n3 and n4 represents an integer independently selected from 0 to 10 such that n1+n2+n3+n4 is 10 or less,
each of x1, x2 and x3 is independently selected from 0 and 1, and
each H¹, H² and H³ is an atom independently selected from N, O and S, provided that if x1+x2=2, n2≥1, if x2+x3=2, n3≥0, if x1+x3=2, n2≥1 or n3≥1, and if x1+x2+x3 is 3, n2≥1 and n3≥1;
or any combination thereof; preferably a single covalent bond, NH or a C₁₋₁₀-alkylene group; more preferably a single covalent bond;
C is C═O, C═S, C(═NR″) wherein R″ represents a hydrogen atom, OH, an alkyl group or a cycloalkyl group, S═O, or S(═O)₂; preferably C═O;
* indicates covalent attachment to the spacer (S); and
***′ indicates covalent attachment to F2.

12. The compound of claim 10, wherein the moiety (F1-RC—F2) is represented by one of the following formulae (4a′) to (4m′) and/or M is independently represented by one of the following formulae (5a) to (5j′): 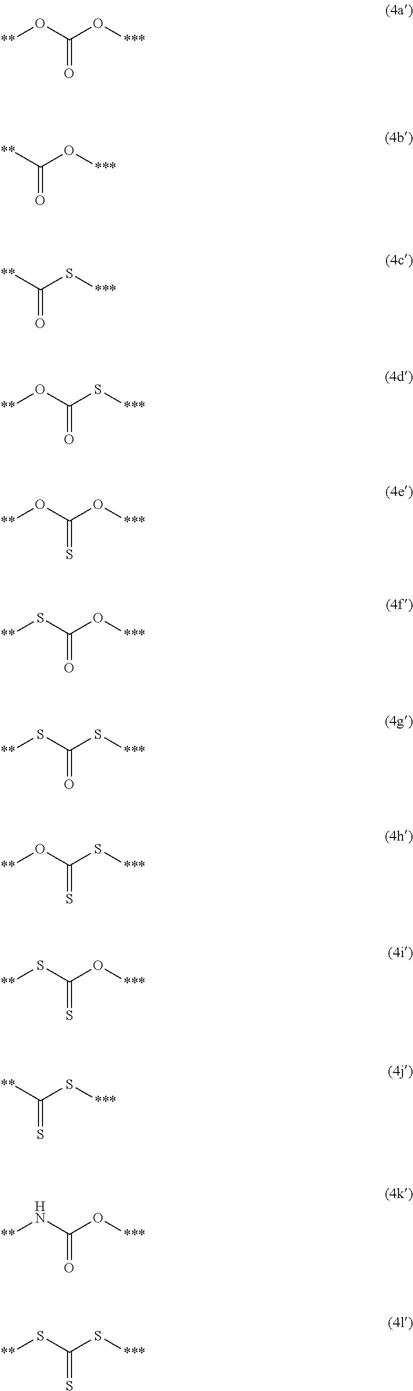 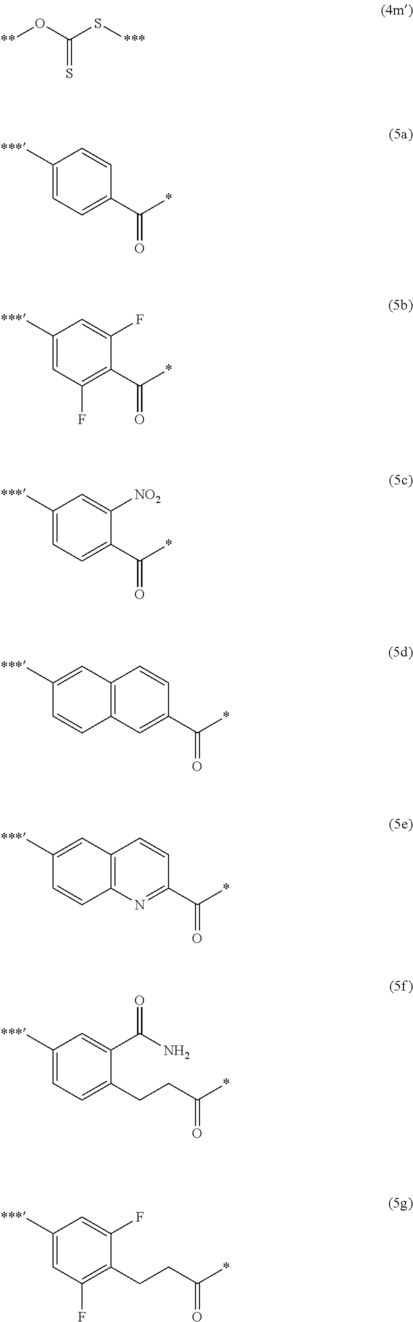 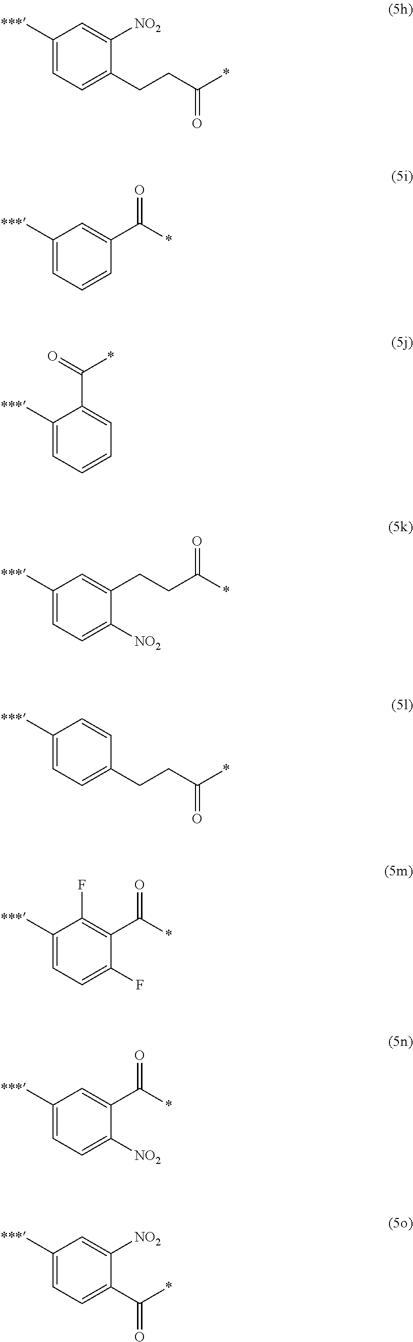 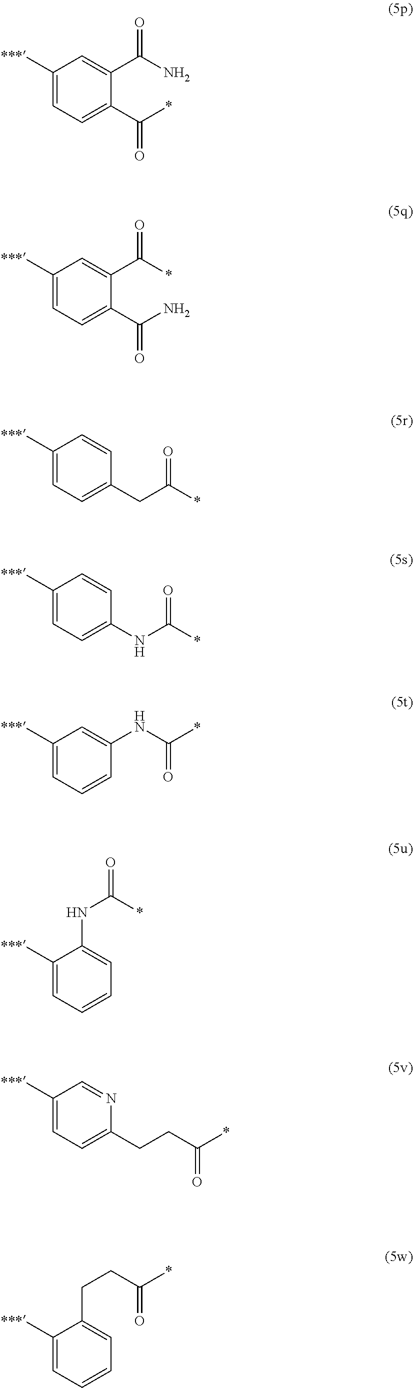 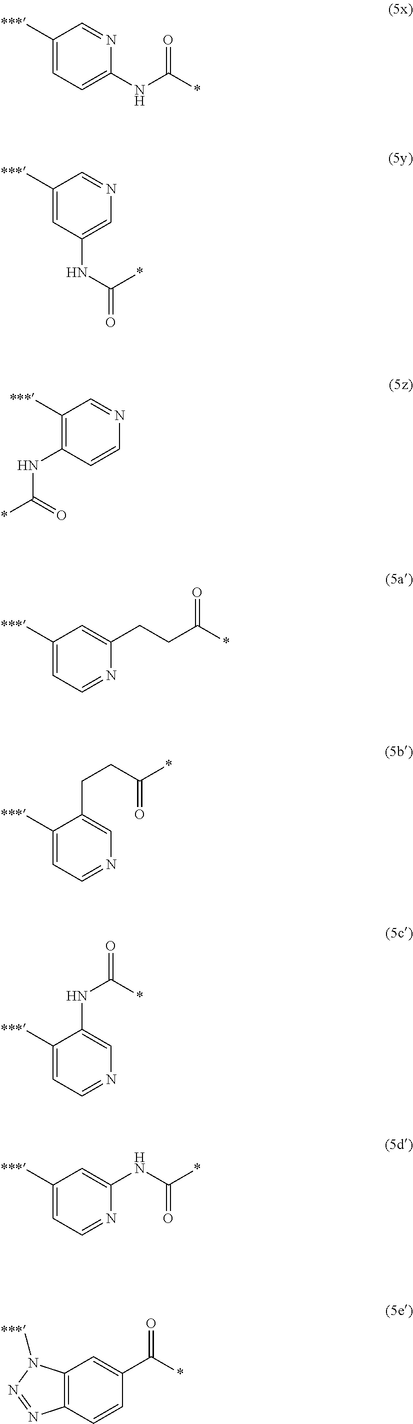 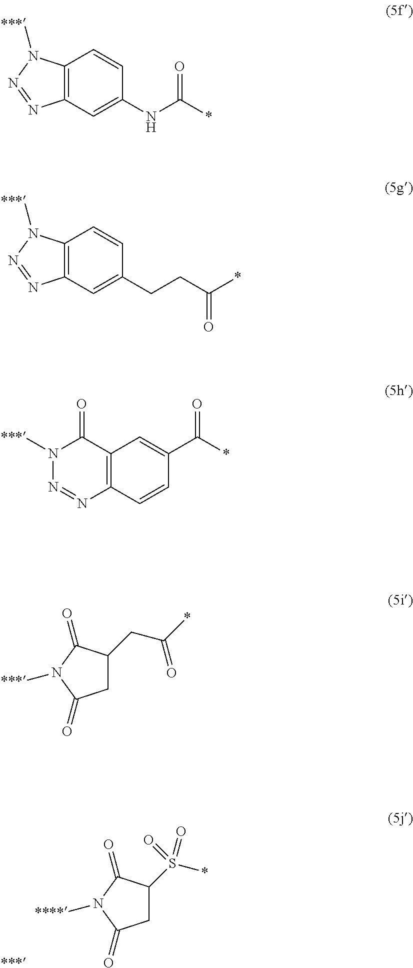
wherein * indicates covalent attachment to the spacer (S), ** indicates covalent attachment to the payload (P), *** indicates covalent attachment to M, and ***′ indicates covalent attachment to F2.

13. The compound of claim 10, wherein the reactive moiety is represented by one of the following formulae (6a) to (6l′): 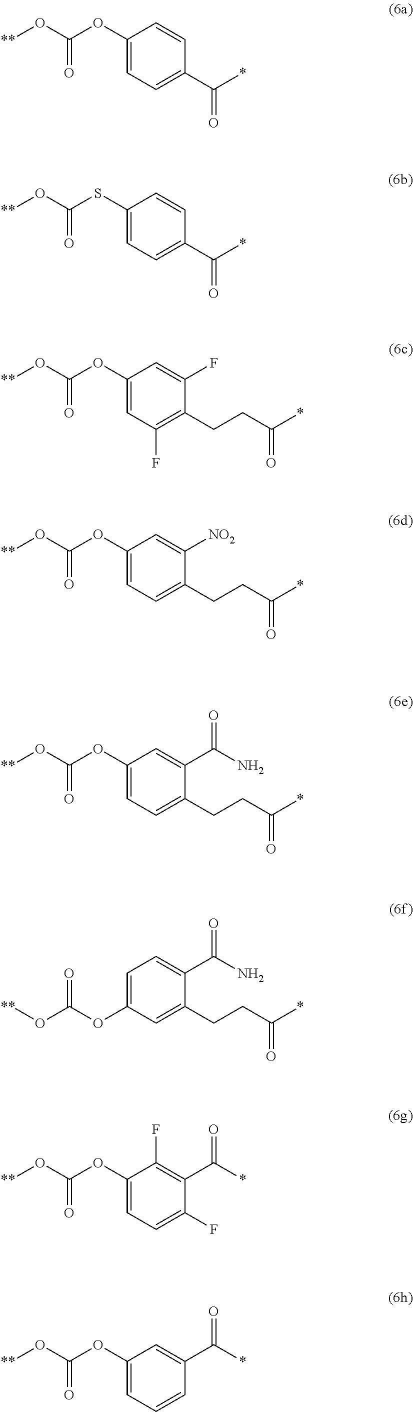 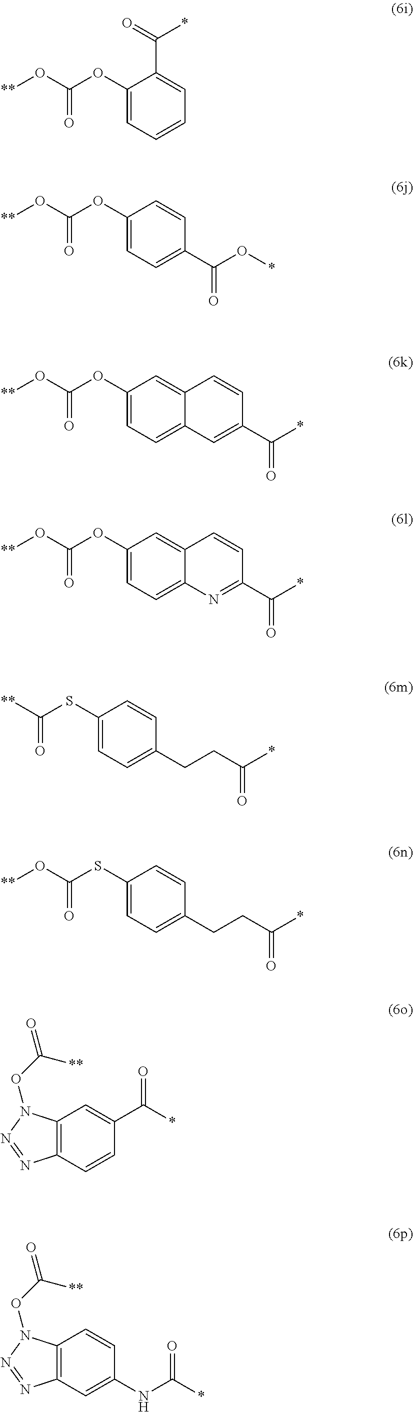 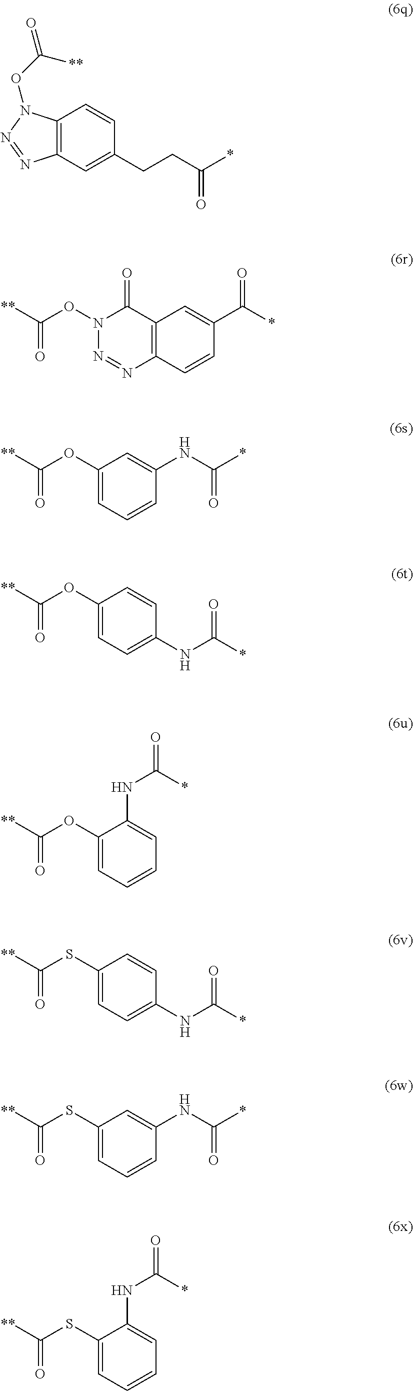 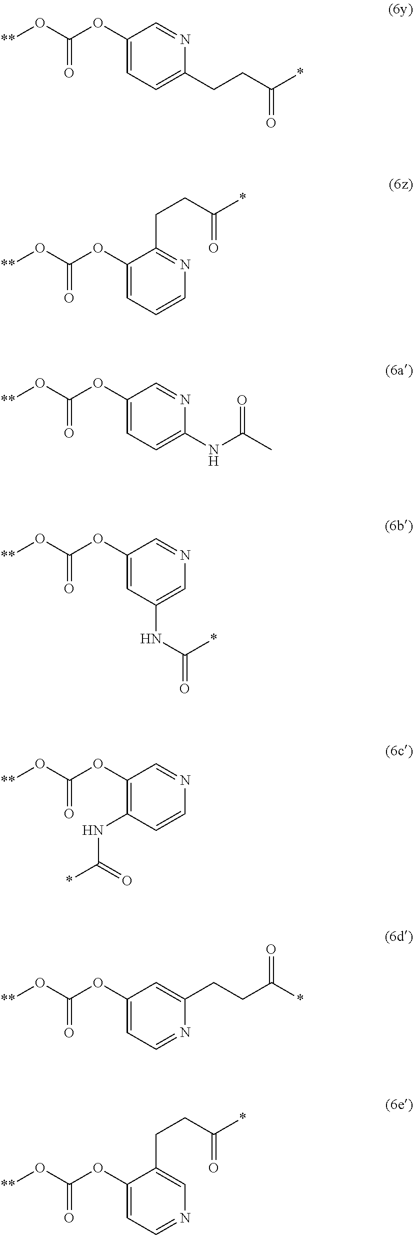 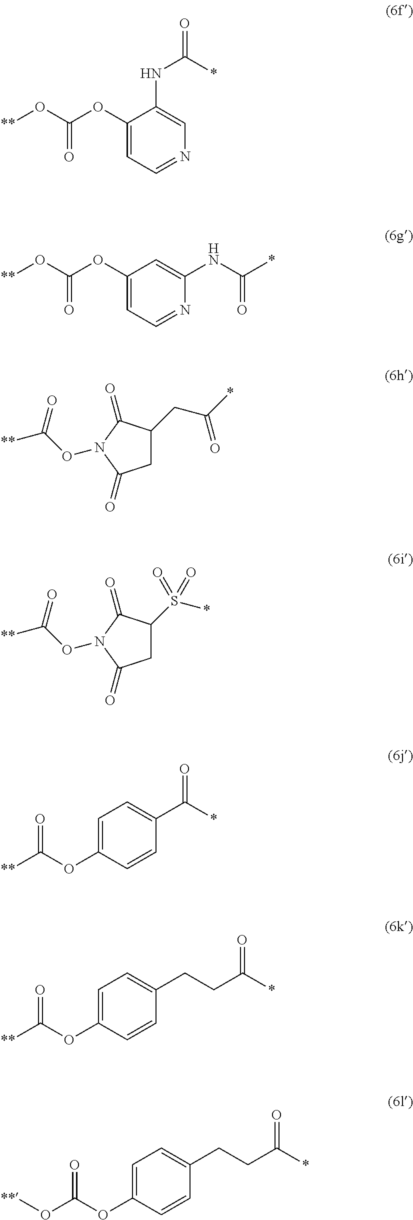
wherein * indicates covalent attachment to the spacer (S), and ** indicates covalent attachment to the payload (P).

14. The compound of claim 1, wherein the spacer has a length of 10 to 35 Å; and preferably is a group having in a main chain from 12 to 120 atoms, e.g. 16 to 80 atoms, said atoms being selected from carbon, nitrogen, oxygen, and sulfur; more preferably a group selected from:
(a2) a polyalkylene oxide group having from 6 to 36 repeating units, for instance 8 to 24 repeating units; preferably a group represented by the following formula (7):
—X¹—(CH₂CH₂O)ₙ₂—CH₂CH₂—X²— (7)
wherein
X¹ is NH, O or S; preferably NH;
X² is NH or C═O, preferably C═O if X2 is covalently bonded to the vector; and
n2 is an integer of 4 to 28, preferably 6 to 20, e.g. 10;
(b2) a peptidic group having 6 to 25 amino acids in the main chain, e.g. 9 amino acids in the main chain, each amino acid being preferably selected from Pro, Gly, Ala, Asn, Asp, Thr, Glu, Gln, and Ser; more preferably Pro, Gly or Ser.

15. The compound of claim 1, wherein the spacer comprises a polyethylene oxide group having 4 to 36 repeating units, preferably 6 to 28 repeating units, and more preferably 7 to 24 repeating units.

16. The compound claim 1, wherein the vector is a peptide comprising a sequence of 11 to 17 amino acids, e.g. 13 to 17 amino acids, preferably a peptide represented by one of the following formulae (8a) and (8b): 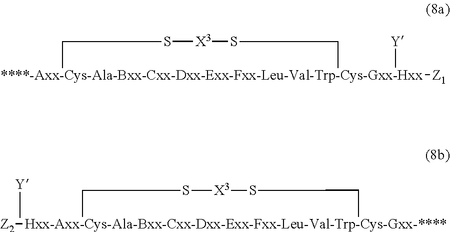
wherein,
Bxx, Cxx, Dxx, Exx, Fxx each independently represent an amino acid;
Axx represents an amino acid, a dicarboxylic acid, or a peptide moiety represented by the following formula (9a):
-Axx1-Axx2-Axx3- (9a)
wherein, in formula (9a),
Axx1 represents a single covalent bond, or an amino acid such as Arg;
Axx2 represents an amino acid such as Gly or Cys; and
Axx3 represents an amino acid such Asp or Asn;
Gxx represents an amino acid, or a peptide moiety represented by the following formula (9b):
-Gxx1-Gxx2-Gxx3- (9b)
wherein, in formula (9b),
Gxx1 represents an amino acid such as Thr;
Gxx2 represents an amino acid such as Tyr or Cys; and
Gxx3 represents a single covalent bond, or an amino acid such His; and
the side chain of Axx2 may be covalently bonded to the side of Gxx2 to form a ring;
if Axx2 is Cys, and Gxx2 is Cys preferably the side chains of Axx2 and Gxx2 are linked together to form a group of formula —(S—X⁴—S)—, wherein X⁴ represents a
single covalent bond or a divalent group comprising one or more atoms selected from carbon, nitrogen and oxygen such as a divalent maleimide group, a divalent acetone group or a divalent arylene group, preferably a single covalent bond;
Hxx represents a single covalent bond, or a trifunctional amino acid such as a diamino-carboxylic acid;
Z₁ represents
a group covalently bonded to the C-terminus of Gxx if Hxx is a single covalent bond, which is selected from —N(H)(R), wherein R represents a hydrogen atom, an alkyl group or a cycloalkyl group, and a moiety derived from a compound containing a conjugation group selected from biotin, DBCO, TCO, BCN, an alkyne, an azide, a bromoacetamide, a maleimide and a thiol;
a group covalently bonded to the C-terminus of Hxx if Hxx is a trifunctional amino acid and Y′ is bonded to the side chain of Hxx, preferably N(H)(R), wherein R represents a hydrogen atom, an alkyl group or a cycloalkyl group, if Z₁ is covalently bonded to the C-terminus of Hxx; or
a hydrogen atom bonded to the side chain of Hxx if Hxx is trifunctional amino acid and Y′ is bonded to the C-terminus of Hxx.
Z₂ represents
a group covalently bonded to the N-terminus of Axx if Hxx is a single covalent bond, which is selected from a hydrogen atom, a carbonyl-containing group such as an acetyl group, and a group containing a conjugation moiety such as biotin;
a group covalently bonded to the N-terminus of Hxx if Hxx is a trifunctional amino acid and Y′ is bonded to the side chain of Hxx, which is selected from a hydrogen atom and a carbonyl-containing group such as an acetyl group; or
a hydrogen atom bonded to the side chain of Hxx if Hxx is trifunctional amino acid and Y′ is bonded to the N-terminus of Hxx.
Y′ is present only if Hxx is a trifunctional amino acid and it represents a moiety covalently bonded to
the side chain of Hxx if Z₁ is bonded to the C-terminus of Hxx, or if Z₂ is bonded to the N-terminus of Hxx,
the C-terminus of Hxx if Z₁ is bonded to the side chain of Hxx, or
the N-terminus of Hxx if Z₂ is bonded to the side chain of Hxx;
Y′ is derived from a compound containing a conjugation group, which is preferably selected from biotin, DBCO, TCO, BCN, an alkyne, an azide, a bromoacetamide, a maleimide, and a thiol;
X³ represents a single covalent bond or a divalent group comprising one or more atoms selected from carbon, nitrogen and oxygen such as a divalent maleimide group, a divalent acetone group or a divalent arylene group, preferably a single covalent bond;
**** indicates covalent attachment to the spacer (S).

17. The compound of claim 16, wherein at least one of Axx, Bxx, Cxx, Dxx, Exx, Fxx, Gxx and Hxx is defined as follows:
Axx represents an amino acid selected from Ala, 2,3-diamino-propionic acid (Dap), Asp, Glu, 2 amino suberic acid, α-amino butyric acid, Asn and Gln, a dicarboxylic acid selected from succinic acid, glutaric acid and adipic acid; preferably Ala, Asp or Asn; more preferably Asp; or a peptide moiety of formula (9a), wherein Axx1 is a single covalent bond, Axx2 is Cys, and Axx3 is Asp;
Bxx represents an amino acid selected from Trp, Phe, Tyr, phenyl glycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;
Cxx represents an amino acid selected from His, Ala, 3-pyridin-2-yl-L-alanine, meta-tyrosine (mTyr) and Phe; preferably His, Ala or mTyr; more preferably His;
Dxx represents an amino acid selected from Ala, Abu, Gly, Leu, Ile, Val, Met, cyclohexyl alanine (Cha), Phe, Thr, Cys, Tyr, and norleucine (Nle); preferably Ala, Nle or Leu; more preferably Leu;
Exx represents an amino acid selected from Ala, Gly, Asn, Ser, Abu, and Asp; preferably Ala or Gly; more preferably Gly;
Fxx represents an amino acid selected from Ala, Glu, Asp, Gln, His, Arg, Ser, and Asn;
preferably Asp or Glu; more preferably Glu;
Gxx represents an amino acid selected from Thr, Ser, Ala, Asn, Val, 2-amino-butyric acid (Abu), Ile, Met, Leu, Pro, Gln, and Cys; preferably Thr or Ser; more preferably Thr; or a peptide moiety of formula (9b), wherein Gxx1 is Thr, Gxx2 is Cys, and Gxx3 is a single covalent bond; and
Hxx represents an amino acid selected from Dap, Dab, Lys, Orn and homo-lysine (homo-Lys), preferably an amino acid selected from Dap, Dab, Lys, Orn and homo-Lys.

18. The compound of claim 1, wherein the vector is a peptide represented by one of the following formulae (8a′) to (8d′): 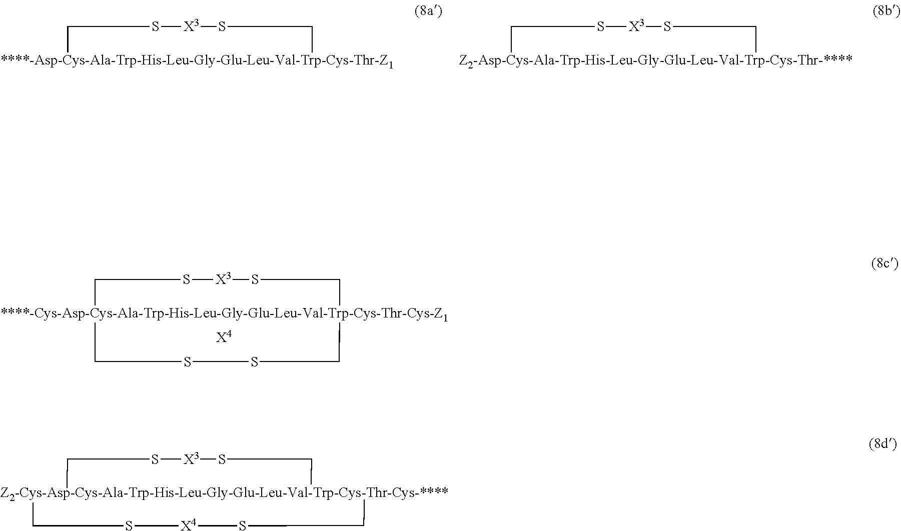
wherein,
Z₁, Z₂, X³, X⁴ and **** are as defined in claim 16;
preferably a peptide represented by formula (8a′) or (8b′).

19. The compound of claim 1, which is selected from 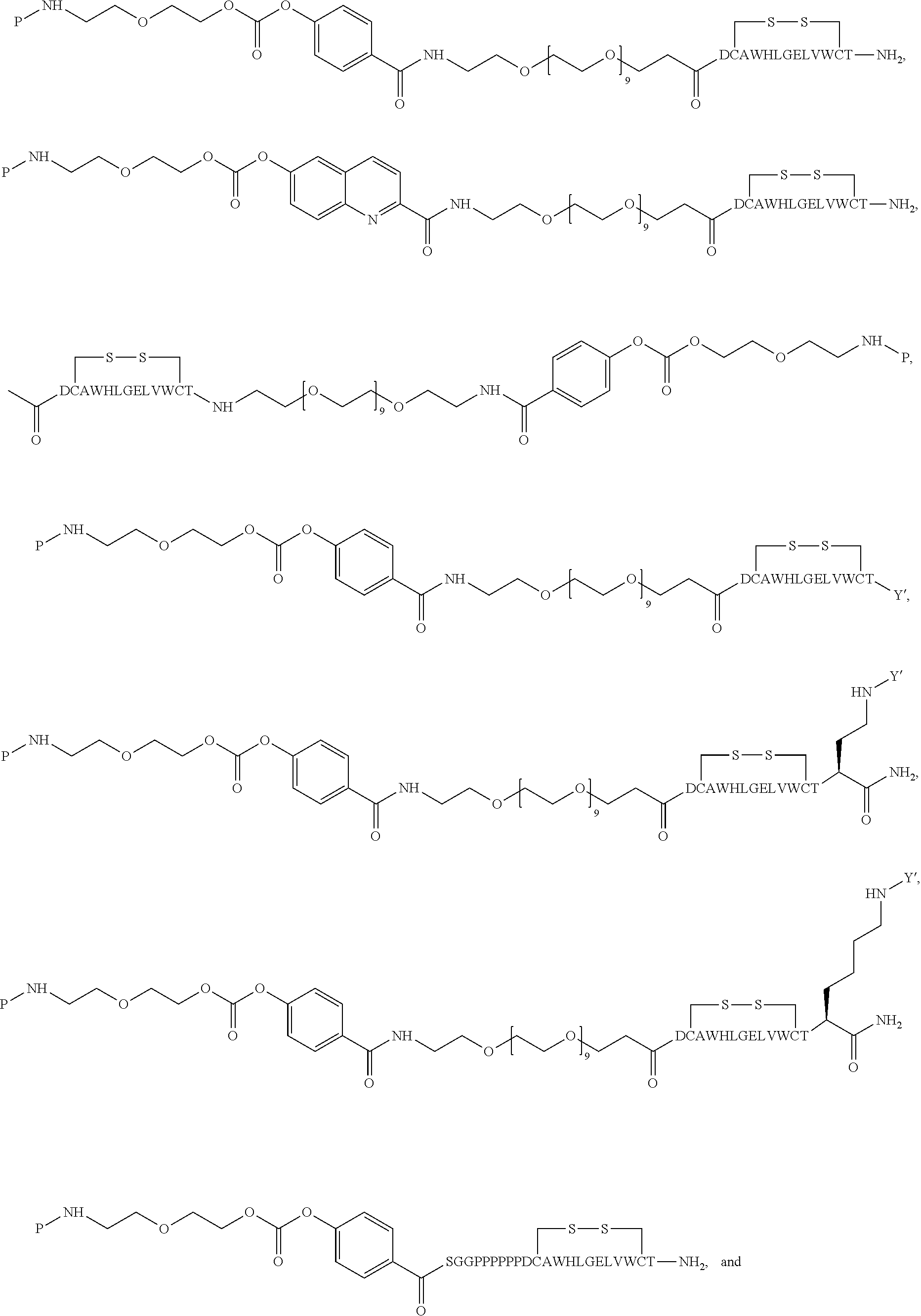 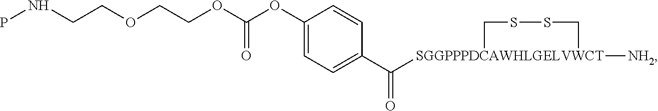
wherein,
P is as defined in any of claims 1 to 5, and
Y′ is as defined in claim 16.

20. The compound of claim 1, which is selected from

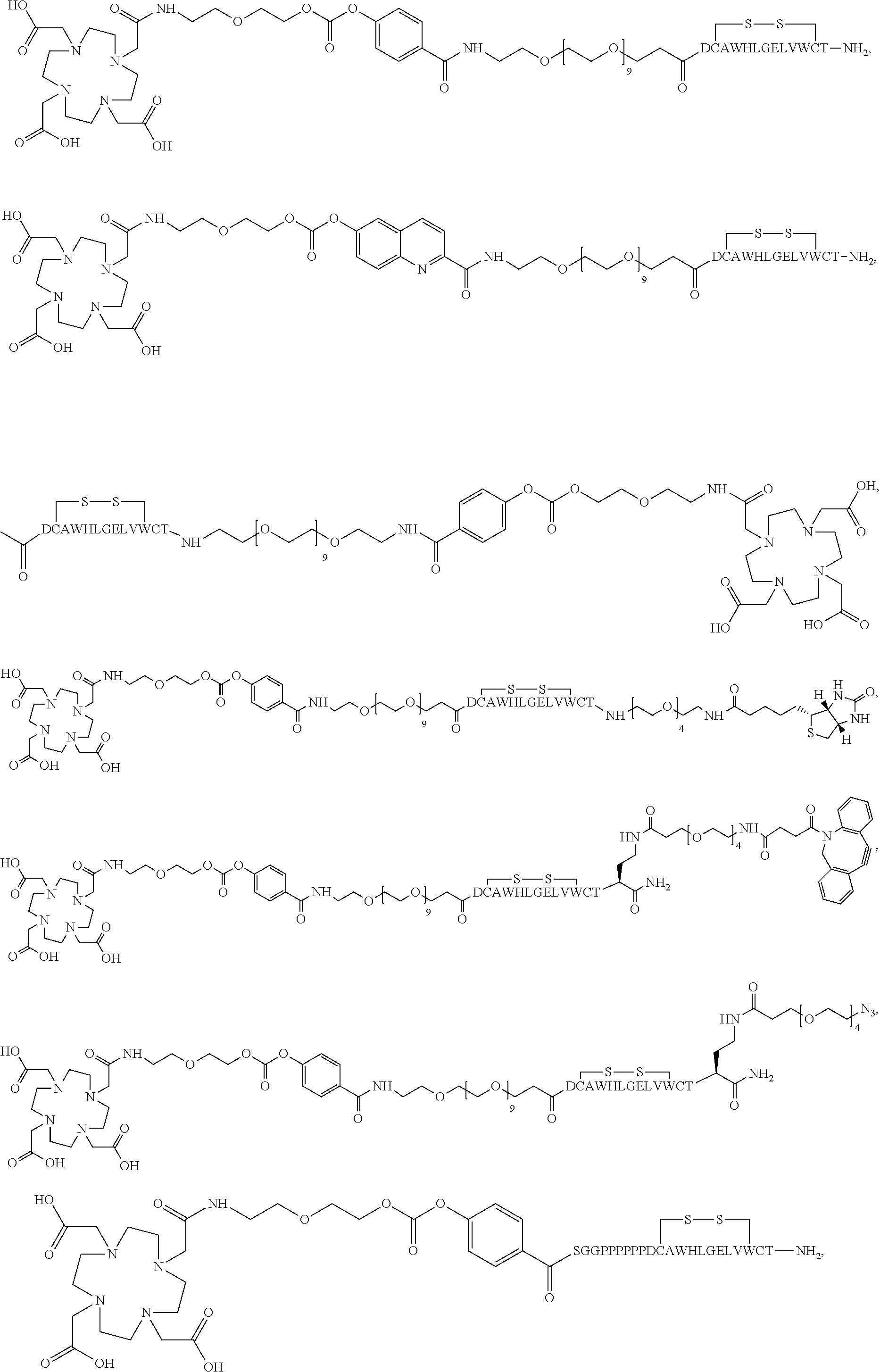

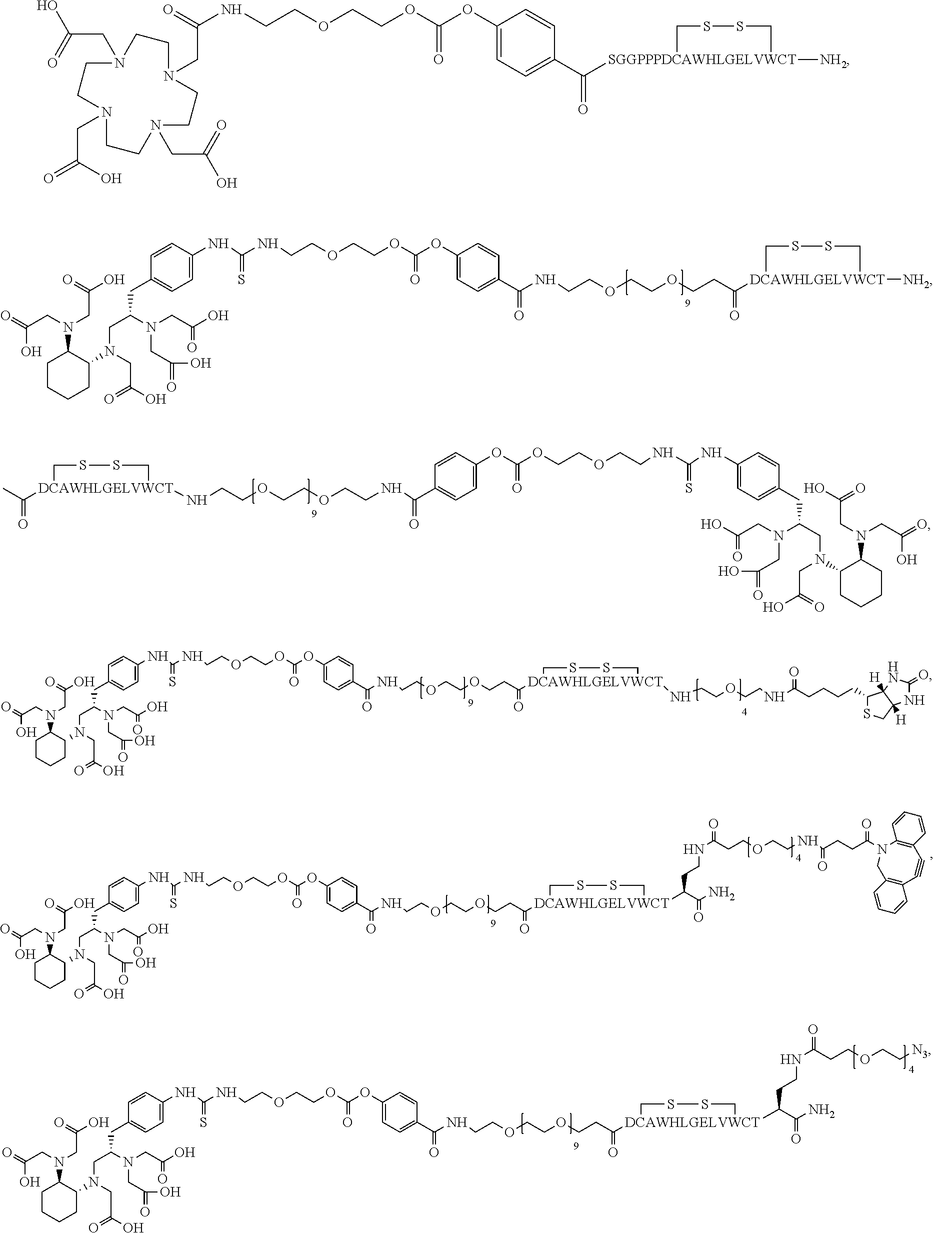

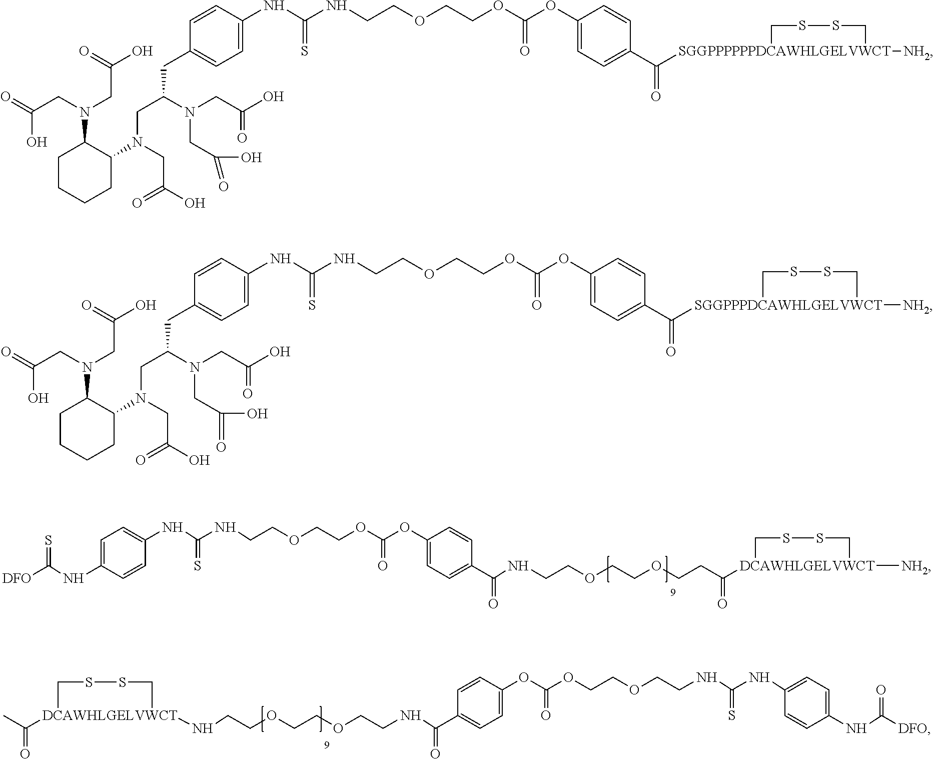

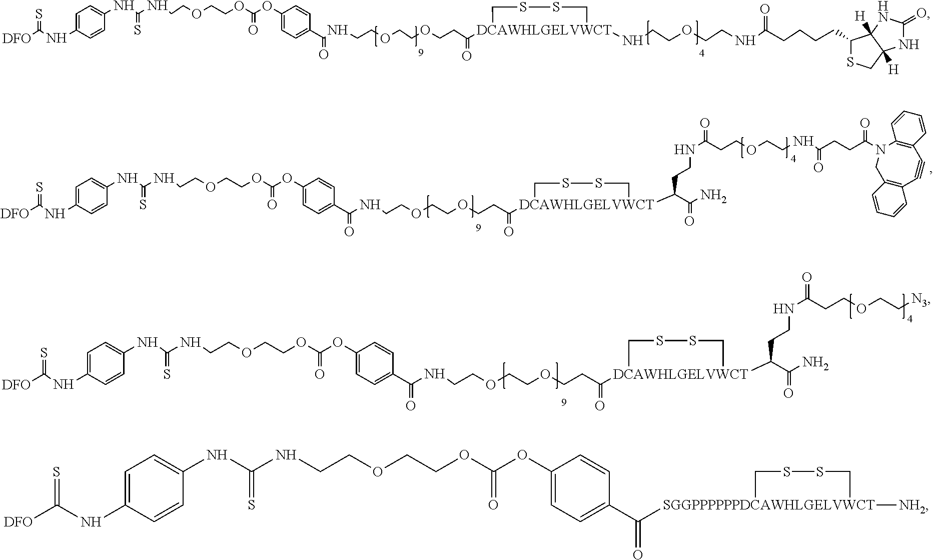

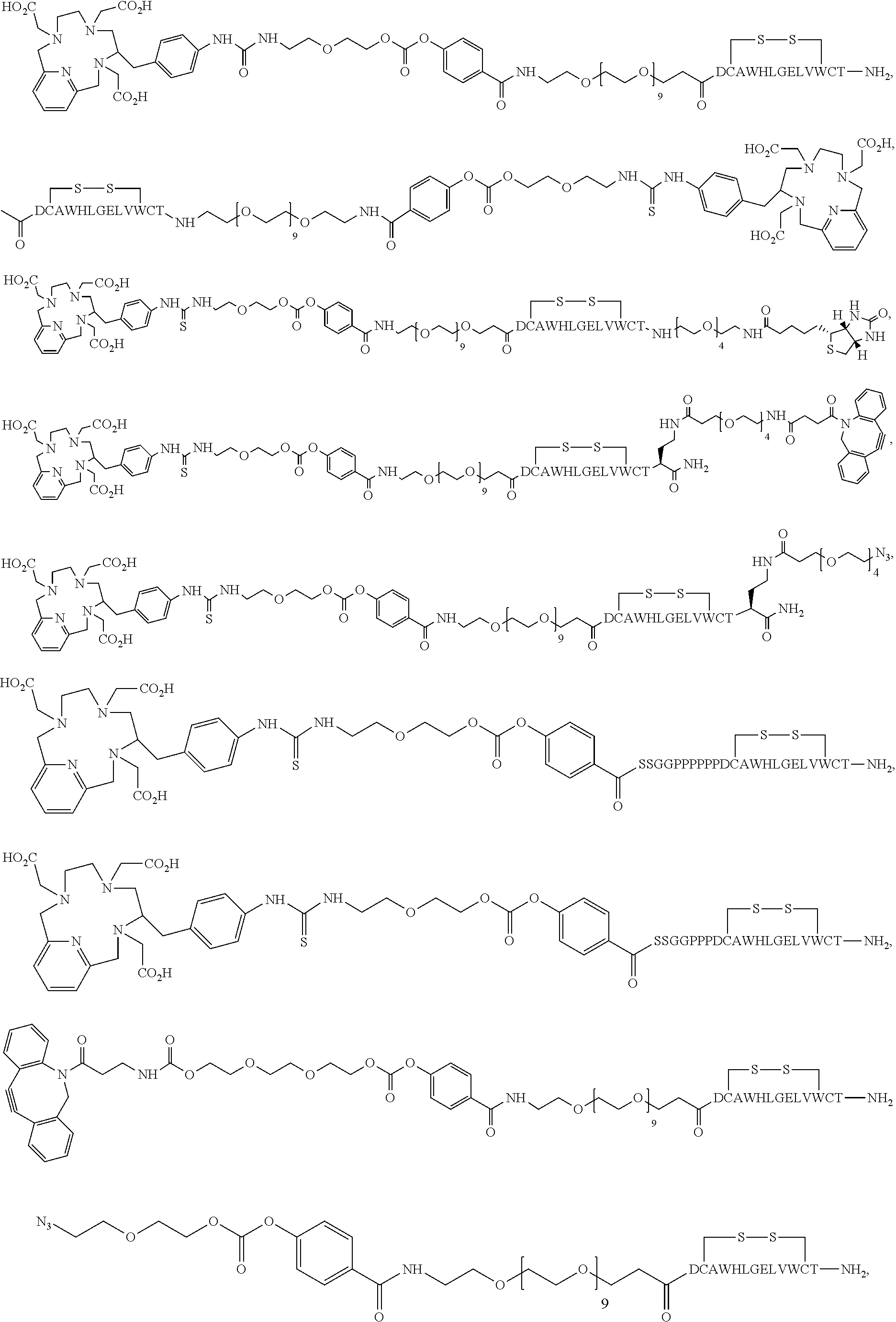

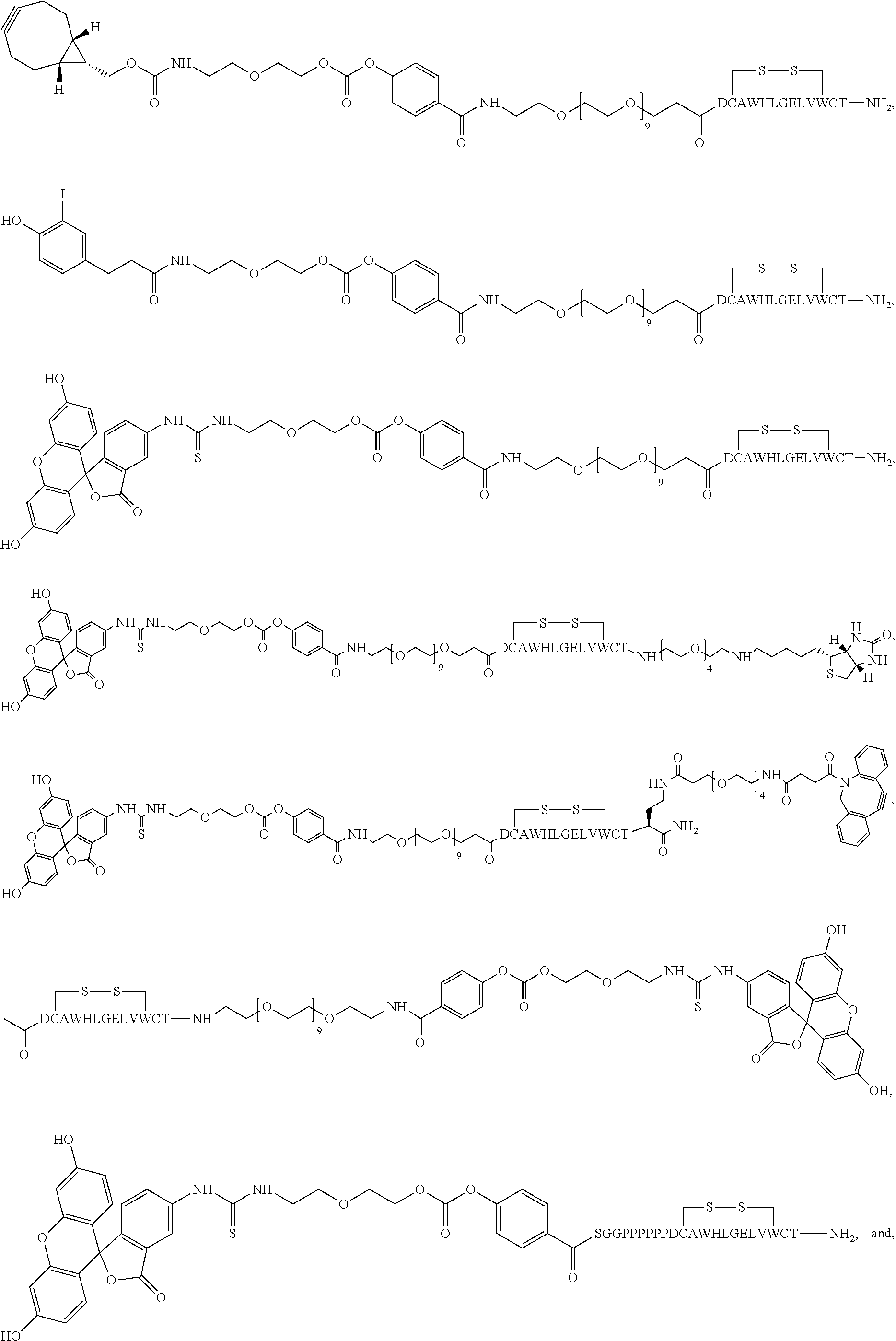

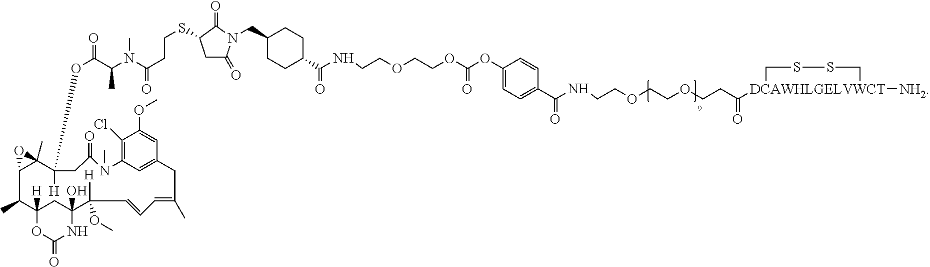

21. Kit for the site-specific modification of an antibody or fragment thereof, the antibody fragment being optionally incorporated into an Fc-fusion protein, comprising the compound of claim 1 and a buffer; wherein the buffer has preferably a pH of 5.5 to 11, more preferably of 7.5 to 9.5.

22. The kit for the regioselective modification of an antibody or fragment thereof of claim 21, wherein the compound is immobilized on a solid phase matrix, e.g. beads.

23. A method for the regioselective modification of an antibody or fragment thereof, the antibody fragment being optionally incorporated into an Fc-fusion protein, comprising reacting an antibody or fragment thereof with a compound according to claim 1.

24. The method of claim 23, wherein
the antibody is a monoclonal antibody, preferably an antibody selected from the group consisting of adalimumab, aducanumab, alemtuzumab, altumomab pentetate, atezolizumab, anetumab, avelumab, bapineuzumab, basiliximab, bectumomab, bermekimab, besilesomab, bevacizumab, bezlotoxumab, brentuximab, brentuximab vedotin, brodalumab, blinatumomab, catumaxomab, cemiplimab, cetuximab, cinpanemab, clivatuzumab, clivatuzumab tetraxetan, crenezumab tetraxetan, daclizumab, daratumumab, denosumab, dinutuximab, durvalumab, edrecolomab, elotuzumab, emapalumab, enfortunab, enfortunab vedotin, epratuzumab, epratuzumab-SN38, etaracizumab, gemtuzumab, gemtuzumab ozogamycin, girentuximab, gosuranemab, ibritumomab, inebilizumab, infliximab, inotuzumab, inotuzumab ozogamicin, ipilimumab, isatuximab, ixekizumab, J591 PSMA-antibody, labetuzumab, lecanemab, mogamulizumab, necitumumab, nimotuzumab, natalizumab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, siltuximab, sacituzumab, sacituzumab govitecan, semorinemab, siltuximab, solanezumab, tacatuzumab, tetrotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably atezolizumab, durvalumab, prembolizumab, rituximab and trastuzumab; or
the antibody fragment is incorporated into an Fc-fusion protein, which is preferably selected from belatacept, aflibercept, ziv-aflibercept, dulaglutide, rilonacept, romiplostim, abatacept, and alefacept.

25. A modified antibody or modified antibody fragment obtainable by reacting an antibody or antibody fragment, the antibody fragment being optionally incorporated into an Fc-fusion protein, with a compound according to claim 1.

26. (canceled)

27. A method for diagnosing, monitoring, imaging or treating a disease comprising administering the modified antibody or modified antibody fragment according to claim 25 to a subject in need thereof.

28. The method according to claim 27, wherein the disease is a neurological disease, a cardiovascular disease, an auto-immune disease or a cancer.

29. The method according to claim 28, wherein the disease or treatment thereof is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina including unstable angina, Aortic aneurysm, Atherosclerosis, Cardiac transplant, Cardiotoxicity diagnosis, Coronary artery bypass graft, Heart failure including atrial fibrillation terminated systolic heart failure, hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.

30. The method according to claim 27, wherein the disease involves cells selected from lymphoma cells, myeloma cells, renal cancer cells, breast cancer cells, prostate cancer cells, ovarian cancer cells, colorectal cancer cells, gastric cancer calls, squamous cancer cells, lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, lung cancer cells, lymphoma cells, colorectal cancer cells, and head-and-neck cancer cells.


SEQUENCE LISTING (3 sequences)

SEQ ID NO: 1 - protein, 14 aa, Artificial Sequence
  peptide portion of compound, as shown in formula (8a) of the application
  MOD_RES (1)..(1): P-Y-S as defined in item 1
  VARIANT (1)..(1): represents an amino acid, a dicarboxylic acid, or a peptide moiety represented by formula (9a) on p. 16 of the application
  DISULFID (2)..(12)
  VARIANT (4)..(8): Each independently represent an amino acid
  VARIANT (13)..(13): Represents an amino acid, or a peptide moiety represented by formula (9b) on p. 17 of the application
  VARIANT (14)..(14): Represents a single covalent bond, or a trifunctional amino acid such as diamino-carboxylic acid
  MOD_RES (14)..(14): other groups or atoms, Z1 and Y′, as described on p. 17-18 of the application
XCAXXXXXLVWCXX

SEQ ID NO: 2 - protein, 14 aa, Artificial Sequence
  peptide portion of compound
  VARIANT (1)..(1): Represents a single covalent bond, or a trifunctional amino acid such as diamino-carboxylic acid
  MOD_RES (1)..(1): other groups or atoms, Z2 and Y′, as described on p. 17-18 of the application
  VARIANT (2)..(2): represents an amino acid, a dicarboxylic acid, or a peptide moiety represented by formula (9a) on p. 17 of the application
  DISULFID (3)..(13)
  VARIANT (5)..(9): Each independently represent an amino acid
  VARIANT (14)..(14): Represents an amino acid, or a peptide moiety represented by formula (9b) on p. 17 of the application
  MOD_RES (14)..(14): P-Y-S as defined in item 1
XXCAXXXXXLVWCX

SEQ ID NO: 3 - protein, 13 aa, Artificial Sequence
  Fc-III-FAM, as shown on p.128 of the application
  DISULFID (2)..(12)
  MOD_RES (13)..(13): FAM label, as shown on p. 128 of the application
DCAWHLGELVWCT